(19) United States
(12) Patent Application Publication
(10) Pub. No.: US 2026/0002849 A1
(43) Pub. Date: Jan. 1, 2026

(54) SEQUENTIAL CROSSLINKING WITH HYDROPHOBIC AND HYDROPHILIC CHEMICAL CROSSLINKERS FOR SAMPLE PRESERVATION

(71) Applicant: 10X GENOMICS, INC., Pleasanton, CA (US)

(72) Inventors: Yang YANG, Fremont, CA (US); Kelly MARTIN, Livermore, CA (US); Connor KUNIHIRO, Pleasanton, CA (US); Dagmar WALTER, San Francisco, CA (US); Josephine Y. LEE, Castro Valley, CA (US); Yi LUO, Dublin, CA (US); Yinzhou ZHU, Dublin, CA (US); Ruijiao XIN, Pleasanton, CA (US)

(73) Assignee: 10X GENOMICS, INC., Pleasanton, CA (US)

(21) Appl. No.: 19/252,211

(22) Filed: Jun. 27, 2025

Related U.S. Application Data
(60) Provisional application No. 63/666,064, filed on Jun. 28, 2024.
(60) Provisional application No. 63/666,055, filed on Jun. 28, 2024.
(60) Provisional application No. 63/681,687, filed on Aug. 9, 2024.

(51) Int. Cl. G01N 1/30; C12N 5/00

(52) U.S. Cl. CPC G01N 1/30; C12N 5/528; G01N 2001/305

(57) ABSTRACT

Methods and kits for sample preparation are provided, wherein a sample comprising at least one cell or tissue is contacted sequentially with a hydrophobic crosslinker, and then a hydrophilic crosslinker. The method prevents cell and tissue analytes from diffusing out of the at least one cell or tissue, such that the cell and tissue analytes can be analyzed and/or imaged.

### CROSS-REFERENCE

This application claims priority to U.S. Provisional Patent Application No. 63/666,064, filed Jun. 28, 2024, U.S. Provisional Patent Application No. 63/666,055, filed Jun. 28, 2024, and U.S. Provisional Patent Application No. 63/681,687, filed Aug. 9, 2024, each of which is entirely incorporated herein by reference for all purposes.

### SUMMARY

In one aspect, the present disclosure provides a method for sample preparation comprising the following steps in order: (i) contacting a sample comprising at least one cell or tissue with a hydrophobic crosslinker; (ii) removing the hydrophobic crosslinker from the sample; (iii) contacting the sample with a hydrophilic crosslinker; and (iv) removing the hydrophilic crosslinker from the sample.

In some embodiments, the hydrophobic crosslinker is ethylene glycol bis(succinimidyl succinate) (EGS), disuccinimidyl suberate (DSS), or dithiobis(succinimidyl propionate) (DSP). In some embodiments, the hydrophobic crosslinker is dithiobis(succinimidyl propionate) (DSP). In some embodiments, the hydrophilic crosslinker is bis(sulfosuccinimidyl)suberate (BS3), ethylene glycol bis(sulfosuccinimidyl succinate) (sulfo-EGS), 3,3′-dithiobis(sulfosuccinimidyl propionate) (DTSSP), bis-succinimide ester-activated PEG (BS(PEG)9) or dimethyl 3,3′-dithiobispropionimidate dihydrochloride (DTBP). In some embodiments, the hydrophilic crosslinker is 3,3′-dithiobis(sulfosuccinimidyl propionate) (DTSSP).

In some embodiments, the sample preparation reduces leakage of biological components from cells. In some embodiments, the leakage is reduced about 2-10-fold relative to a control cell or tissue that has not been contacted with the hydrophobic and hydrophilic crosslinkers.

In some embodiments, the hydrophobic crosslinker is in a first buffer and the hydrophilic crosslinker is in a second buffer. In some embodiments, the first and second buffers are the same. In some embodiments, first and second buffers are not the same.

In some embodiments, the method further comprises a step v.) suspending the cells from the sample in a third buffer. In some embodiments, the third buffer is a storage buffer.

In some embodiments, the hydrophobic crosslinker comprises about 0.05 mM to 10 mM DSP. In some embodiments, the hydrophobic crosslinker comprises about 0.25 mM to about 7.5 mM DSP. In some embodiments, the hydrophobic crosslinker comprises about 0.5 mM to about 5 mM DSP. In some embodiments, the hydrophobic crosslinker comprises about 2.5 mM DSP. In some embodiments, the hydrophilic crosslinker comprises about 0.25 mM to about 10 mM DTSSP. In some embodiments, the hydrophilic crosslinker comprises about 0.5 mM to about 7 mM DTSSP. In some embodiments, the hydrophilic crosslinker comprises about 1.0 mM to about 7.0 mM DTSSP. In some embodiments, the hydrophilic crosslinker comprises about 2.5 mM DTSSP.

In some embodiments, the method further comprises storing the sample at room temperature. In some embodiments, the method further comprises storing the sample at about 2 to about 8 degrees Celsius. In some embodiments, the method further comprises cryopreserving the sample.

In some embodiments, the first and/or the second buffer is a phosphate buffered saline (PBS)-based buffer. In some embodiments, the first and/or the second buffer is a saline sodium citrate (SSC) based buffer.

In some embodiments, contacting the sample with the hydrophobic crosslinker comprises incubating at about 2 to about 25 degrees Celsius for a time period ranging from about 10 minutes to about 24 hours. In some embodiments, contacting the sample with the hydrophobic crosslinker comprises incubating at about 4 to about 23 degrees Celsius for a time period ranging from about 15 minutes to about 4 hours. In some embodiments, contacting the sample with the hydrophobic crosslinker comprises incubating at about 4 to about 20 degrees Celsius for a time period ranging from about 20 to about 60 minutes. In some embodiments, contacting the sample with the hydrophobic crosslinker comprises incubating at about 20 degrees Celsius for a time period of about 30 minutes. In some embodiments, contacting the sample with the hydrophilic crosslinker comprises incubating at about 2 to about 25 degrees Celsius for a time period ranging from about 10 minutes to about 24 hours. In some embodiments, contacting the sample with the hydrophilic crosslinker comprises incubating at about 4 to about 23 degrees Celsius for a time period ranging from about 15 minutes to about 4 hours. In some embodiments, contacting the sample with the hydrophilic crosslinker comprises incubating at about 4 to about 20 degrees Celsius for a time period ranging from about 15 to about 60 minutes. In some embodiments, contacting the sample with the hydrophilic crosslinker comprises incubating at about 20 degrees Celsius for a time period of about 30 minutes. In some embodiments, the sample is maintained in the storage buffer for a time period of about 1 hour to about 5 days.

In some embodiments, the method further comprises determining the sequence of one or more nucleic acid sequences from one or more cells from the sample. In some embodiments, the method further comprises partitioning the cells.

In some embodiments, the first buffer, the second buffer, and/or the storage buffer further comprises a cryoprotectant reagent. In some embodiments, the first buffer, the second buffer, and/or the storage buffer further comprises a stabilizing reagent. In some embodiments, the first buffer, the second buffer, and/or the storage buffer further comprises an RNA stabilizer. In some embodiments, the first buffer, the second buffer, and/or the storage buffer further comprises a ribonucleoside vanadyl complex (RVC). In some embodiments, the first buffer, the second buffer, and/or the storage buffer further comprises an RNase inhibitor.

In another aspect, the disclosure provides a kit for sample preparation comprising: i) a hydrophobic crosslinker; ii) a hydrophilic crosslinker; iii) a storage buffer; and iv) instructions for use thereof.

In some embodiments, the hydrophobic crosslinker is ethylene glycobis (succinimidylsuccinate) (EGS), disuccinimidyl suberate (DSS) or dithiobis(succinimidylpriopionate) (DSP). In some embodiments, the hydrophobic crosslinker is dithiobis(succinimidylpriopionate) (DSP). In some embodiments, the hydrophilic crosslinker is bis(sulfosuccinimidyl)suberate (BS3), ethylene glycobis(sulfosuccinimidylsuccinate) (sulfo-EGS), 3,3′-dithiobis(sulfosuccinimidyl priopionate) (DTSSP), bis(sulfosuccinimidyl)suberate (DSS), bis-succinimide ester-activated PEG (BS(PEG)9) or dimethyl 3,3′-dithiobispropionimidate·2HCl (DTBP). In some embodiments, the hydrophilic crosslinker is 3,3′-dithiobis(sulfosuccinimidyl priopionate) (DTSSP).

In some embodiments, the hydrophobic crosslinker comprises about 0.05 mM to about 10 mM DSP. In some embodiments, the hydrophobic crosslinker comprises about 0.25 mM to about 7.5 mM DSP. In some embodiments, the hydrophobic crosslinker comprises about 0.5 mM to about 5 mM DSP. In some embodiments, the hydrophobic crosslinker comprises about 1.25 to about 2.5 mM DSP. In some embodiments, the hydrophilic crosslinker comprises about 0.25 mM to about 20 mM DTSSP. In some embodiments, the hydrophilic crosslinker comprises about 0.5 mM to about 10 mM DTSSP. In some embodiments, the second hydrophilic crosslinker comprises about 1.0 mM to about 10 mM DTSSP. In some embodiments, the second hydrophilic crosslinker comprises about 2.5 to about 5 mM DTSSP.

In some embodiments, the kit can be stored at room temperature. In some embodiments, the storage buffer is a phosphate buffered saline (PBS)-based storage buffer. In some embodiments, the storage buffer is a saline sodium citrate (SSC) based storage buffer. In some embodiments, the hydrophobic crosslinker, the hydrophilic crosslinker, and/or the storage buffer further comprises a cryoprotectant reagent. In some embodiments, the hydrophobic crosslinker, the hydrophilic crosslinker, and/or the storage buffer further comprises a stabilizing reagent. In some embodiments, the hydrophobic crosslinker, the hydrophilic crosslinker, and/or the storage buffer further comprises an RNA stabilizer. In some embodiments, the hydrophobic crosslinker, the hydrophilic crosslinker, and/or the storage buffer further comprises a ribonucleoside vanadyl complex (RVC). In some embodiments, the hydrophobic crosslinker, the hydrophilic crosslinker, and/or the storage buffer further comprises an RNase inhibitor.

In another aspect, the disclosure provides a kit for sample preparation comprising: i) a hydrophobic crosslinker comprising about 2.5 mM DSP; ii) a hydrophilic crosslinker comprising about 2.5 mM DTSSP; iii) a storage buffer; and iv) instructions for use thereof.

In some embodiments, the kit instructions comprise instructions for preserving and storing a plurality of cells or tissue in a manner that reduces leakage of biological components.

In another aspect, the disclosure provides a method for sample preparation comprising the following steps in order: i) contacting a plurality of cells with a hydrophobic crosslinker comprising about 2.5 mM DSP for about 30 minutes at about 20 degrees C.; ii) removing the hydrophobic crosslinker from the cells; iii) contacting the cells with a hydrophilic crosslinker comprising about 2.5 mM DTSSP for about 30 minutes at about 20 degrees C.; iv) removing the hydrophilic crosslinker from the cells; and v) suspending the cells in a storage buffer.

In another aspect, the disclosure provides a method for sample preparation comprising contacting a plurality of cells with a solution comprising DSP and methanol. In some embodiments, the DSP comprises a final concentration between about 0.25 mM and about 5 mM. In some embodiments, the DSP comprises a final concentration of about 3.125 mM. In some embodiments, the method further comprises storing the cells in a storage buffer at −80 degrees C. In some embodiments, the method further comprises the steps of: ii) thawing the cells; iii) adding methanol to the cells; iv) adding a wash buffer and centrifuging the cells; v) resuspending the cells in the wash buffer; vi) centrifuging the cells; and v.) resuspending the cells in a resuspension buffer.

In another aspect, the disclosure provides a method for sample preparation comprising the following steps: i) incubating a plurality of cells in a solution comprising about 3.125 mM DSP and 100% methanol; ii) adding a buffer (e.g. TrisHCl) to the cells; iii) adding methanol to the cells; iv) adding a wash buffer and centrifuging the cells; v) resuspending the cells in the wash buffer; vi) centrifuging the cells; and v.) resuspending the cells in a resuspension buffer. In some embodiments, the method further comprises partitioning the cells in a plurality of partitions, wherein a partition of the plurality of partitions comprises a single cell. In some embodiments, the partition is a droplet.

In another aspect, the disclosure provides a method for reducing leakage of biological components from cells during preservation and storage. The method comprises: i) contacting a plurality of cells with a fixative buffer comprising a poloxamine/Tetronic® polymer; ii) removing the fixative buffer from the cells; and iii) suspending the cells in a storage buffer.

In another aspect, the disclosure provides a method for reducing leakage of biological components from cells during preservation and storage, comprising: i) contacting a plurality of cells with a fixative buffer; ii) removing the fixative buffer from the cells; iii) contacting the cells with a treatment buffer comprising a poloxamine/Tetronic® polymer; iv) removing the treatment buffer from the cells; and v) suspending the cells in a storage buffer.

In another aspect, the disclosure provides a method for reducing leakage of biological components from cells during preservation and storage, the method comprising: i) contacting a plurality of cells with a fixative buffer; ii) removing the fixative buffer from the cells; and iii) suspending the cells in a storage buffer comprising a poloxamine/Tetronic® polymer.

In another aspect, the disclosure provides a method for reducing leakage of biological components from cells during preservation and storage, the method comprising: i) contacting a plurality of cells with a fixative buffer comprising a poloxamine/Tetronic® polymer; ii) removing the fixative buffer from the cells; and iii) suspending the cells in a storage buffer comprising a poloxamine/Tetronic® polymer.

In another aspect, the disclosure provides a method for reducing leakage of biological components from cells during preservation and storage, the method comprising: i) contacting a plurality of cells with a fixative buffer comprising a poloxamine/Tetronic® polymer; ii) removing the fixative buffer from the cells; iii) contacting the cells with a treatment buffer comprising a poloxamine/Tetronic® polymer; iv) removing the treatment buffer from the cells; and v) suspending the cells in a storage buffer.

In another aspect, the disclosure provides a method for reducing leakage of biological components from cells during preservation and storage, the method comprising: i) contacting a plurality of cells with a fixative buffer; ii) removing the fixative buffer from the cells; iii) contacting the cells with a treatment buffer comprising a poloxamine/Tetronic® polymer; iv) removing the treatment buffer from the cells; and v) suspending the cells in a storage buffer comprising a poloxamine/Tetronic® polymer.

In another aspect, the disclosure provides a method for reducing leakage of biological components from cells during preservation and storage, the method comprising: i) contacting a plurality of cells with a fixative buffer comprising a poloxamine/Tetronic® polymer; ii) removing the fixative buffer from the cells; iii) contacting the cells with a treatment buffer comprising a poloxamine/Tetronic® polymer; iv) removing the treatment buffer from the cells; and v) suspending the cells in a storage buffer comprising a poloxamine/Tetronic® polymer.

In some embodiments, the poloxamine/Tetronic® polymer comprises a poloxamine 1107/T1107 Tetronic® polymer.

In some embodiments, the fixative buffer comprises from about 0.25 mM to about 1.25 mM of a poloxamine 1107/T1107 Tetronic® polymer. In some embodiments, the fixative buffer comprises a crosslinking fixative. In some embodiments, the fixative buffer comprises glyoxal. In some embodiments, the fixative buffer comprises from about 0.25% glyoxal to about 3.25% glyoxal. In some embodiments, the fixative buffer comprises DSP or DTSSP.

In some embodiments, the storage buffer comprises from about 0.25 mM to about 1.25 mM of a poloxamine 1107/T1107 Tetronic® polymer. In some embodiments, the storage buffer is a phosphate buffered saline (PBS)-based storage buffer. In some embodiments, the storage buffer is a saline sodium citrate (SSC) based storage buffer.

In some embodiments, the method further comprises storing the cells at room temperature. In some embodiments, the method further comprises storing the cells at about 2 to about 8 degrees Celsius. In some embodiments, the method further comprises cryopreserving the cells.

In some embodiments, the contacting step comprises incubating for 1 hour at room temperature or incubating overnight at 2-8 degrees Celsius.

In some embodiments, the cells are maintained in the storage buffer for a time period of about 1 hour to about 5 days. In some embodiments, the treatment buffer is a PBS based buffer comprising from about 0.25 mM to about 1.25 mM of a poloxamine 1107/T1107 Tetronic® polymer.

In some embodiments, the method further comprises analyzing one or more of the cells. In some embodiments, analyzing one or more of the cells comprises conducting a molecular analysis on one or more of the cells. In some embodiments, analyzing one or more of the cells comprises conducting a nucleic acid or protein analysis of one or more of the cells. In some embodiments, analyzing one or more of the cells comprises conducting a ribonucleic acid (RNA) analysis of one or more of the cells. In some embodiments, analyzing one or more of the cells comprises conducting a single cell RNA analysis of one or more of the cells. In some embodiments, analyzing one or more of the cells comprises conducting a deoxyribonucleic acid (DNA) analysis of one or more of the cells. In some embodiments, analyzing one or more of the cells comprises conducting a single cell deoxyribonucleic acid (DNA) analysis of one or more of the cells. In some embodiments, analyzing one or more of the cells comprises conducting a protein analysis of one or more of the cells. In some embodiments, analyzing one or more of the cells comprises conducting a single cell protein analysis of one or more of the cells.

In some embodiments, the method further comprises conducting a molecular analysis of biological components of one or more of the cells. In some embodiments, the molecular analysis is single cell molecular analysis.

In some embodiments, the method further comprises partitioning of the biological components for single cell analysis. In some embodiments, the partition is an aqueous droplet in oil emulsion. In some embodiments, the partition further comprises a gel bead.

In some embodiments, the method further comprises generating single cell barcoded biological components (analytes) for single cell data processing.

In some embodiments, the fixative buffer, treatment buffer, or storage buffer further comprises a cryoprotectant reagent. In some embodiments, the fixative buffer, treatment buffer, or storage buffer further comprises a stabilizing reagent. In some embodiments, the fixative buffer, treatment buffer, or storage buffer further comprises an RNA stabilizer. In some embodiments, the fixative buffer, treatment buffer, or storage buffer further comprises a ribonucleoside vanadyl complex (RVC). In some embodiments, the fixative buffer, treatment buffer, or storage buffer further comprises an RNase inhibitor.

In some embodiments, the method comprises: i) contacting the plurality of cells with a fixative buffer for 1 hour at room temperature or overnight at about 2 to about 8 degrees Celsius, wherein the fixative buffer comprises about 0.25 mM to about 1.25 mM of a poloxamine 1107/T1107 Tetronic® polymer; ii) removing the fixative buffer from the cells; and iii) suspending the cells in a storage buffer.

In some embodiments, the method comprises: i) contacting the plurality of cells with a fixative buffer for 1 hour at room temperature or overnight at about 2 to about 8 degrees Celsius; ii) removing the fixative buffer from the cells; and iv) suspending the cells in a storage buffer comprising about 0.25 mM to about 1.25 mM of a poloxamine 1107/T1107 Tetronic® polymer.

In some embodiments, the method comprises: i) contacting the plurality of cells with a fixative buffer for 1 hour at room temperature or overnight at about 2 to about 8 degrees Celsius, wherein the fixative buffer comprises about 0.25 mM to about 1.25 mM of a poloxamine 1107/T1107 Tetronic® polymer; ii) removing the fixative buffer from the cells; and iv) suspending the cells in a storage buffer comprising about 0.25 mM to about 1.25 mM of a poloxamine 1107/T1107 Tetronic® polymer.

In some embodiments, the biological components comprise RNA. In some embodiments, the biological components comprise DNA. In some embodiments, the biological components comprise protein.

In another aspect, the disclosure provides a kit for reducing leakage of biological components from cells during preservation and storage, the kit comprising: i) a fixative buffer comprising a poloxamine/Tetronic® polymer; ii) a storage buffer; and iii) instructions for preserving and storing a plurality of cells in a manner that reduces leakage of biological components.

In another aspect, the disclosure provides a kit for reducing leakage of biological components from cells during preservation and storage, the kit comprising: i) a fixative buffer; ii) a storage buffer comprising a poloxamine/Tetronic® polymer; and iii) instructions for preserving and storing a plurality of cells in a manner that reduces leakage of biological components.

In another aspect, the disclosure provides a kit for reducing leakage of biological components from cells during preservation and storage, the kit comprising: i) a fixative buffer comprising a poloxamine/Tetronic® polymer; ii) a storage buffer comprising a poloxamine/Tetronic® polymer; and iii) instructions for preserving and storing a plurality of cells in a manner that reduces leakage of biological components.

In another aspect, the disclosure provides a kit for reducing leakage of biological components from cells during preservation and storage, the kit comprising: i) a fixative buffer; ii) a treatment buffer comprising a poloxamine/Tetronic® polymer; iii) a storage buffer; and iv) instructions for preserving and storing a plurality of cells in a manner that reduces leakage of biological components.

In another aspect, the disclosure provides a kit for reducing leakage of biological components from cells during preservation and storage, the kit comprising: i) a fixative buffer comprising a poloxamine/Tetronic® polymer; ii) a treatment buffer comprising a poloxamine/Tetronic® polymer; iii) a storage buffer; and iv) instructions for preserving and storing a plurality of cells in a manner that reduces leakage of biological components.

In another aspect, the disclosure provides a kit for reducing leakage of biological components from cells during preservation and storage, the kit comprising: i) a fixative buffer; ii) a treatment buffer comprising a poloxamine/Tetronic® polymer; iii) a storage buffer comprising a poloxamine/Tetronic® polymer; and iv) instructions for preserving and storing a plurality of cells in a manner that reduces leakage of biological components.

In another aspect, the disclosure provides a kit for reducing leakage of biological components from cells during preservation and storage, the kit comprising: i) a fixative buffer comprising a poloxamine/Tetronic® polymer; ii) a treatment buffer comprising a poloxamine/Tetronic® polymer; iii) a storage buffer comprising a poloxamine/Tetronic® polymer; and iv) instructions for preserving and storing a plurality of cells in a manner that reduces leakage of biological components.

In some embodiments, the poloxamine/Tetronic® polymer comprises a poloxamine 1107/T1107 Tetronic® polymer.

In some embodiments, the fixative buffer comprises from about 0.25 mM to about 1.25 mM of a poloxamine 1107/T1107 Tetronic® polymer. In some embodiments, the fixative buffer comprises a crosslinking fixative. In some embodiments, the fixative buffer comprises glyoxal. In some embodiments, the fixative buffer comprises from about 0.25% glyoxal to about 3.25% glyoxal. In some embodiments, the fixative buffer comprises DSP/DTSSP.

In some embodiments, the storage buffer comprises from about 0.25 mM to about 1.25 mM of a poloxamine 1107/T1107 Tetronic® polymer. In some embodiments, the storage buffer is a phosphate buffered saline (PBS)-based storage buffer. In some embodiments, the storage buffer is a saline sodium citrate (SSC) based storage buffer.

In some embodiments, the instructions provide for storing the cells at room temperature. In some embodiments, the instructions provide for storing the cells at about 2 to about 8 degrees Celsius. In some embodiments, the instructions provide for cryopreserving the cells. In some embodiments, the instructions provide for the contacting step to comprise incubating for 1 hour at room temperature or incubating overnight at 2-8 degrees Celsius. In some embodiments, the instructions provide for the cells to be maintained in the storage buffer for a time period of about 1 hour to about 5 days.

In some embodiments, the treatment buffer is a PBS based buffer comprising from about 0.25 mM to about 1.25 mM of a poloxamine 1107/T1107 Tetronic® polymer.

In some embodiments, the instructions further provide for analyzing one or more of the cells. In some embodiments, the instructions provide that the analyzing one or more of the cells comprises conducting a molecular analysis on one or more of the cells. In some embodiments, the instructions provide that analyzing one or more of the cells comprises conducting a nucleic acid or protein analysis of one or more of the cells. In some embodiments, the instructions provide that analyzing one or more of the cells comprises conducting a ribonucleic acid (RNA) analysis of one or more of the cells. In some embodiments, the instructions provide that analyzing one or more of the cells comprises conducting a single cell RNA analysis of one or more of the cells. In some embodiments, the instructions provide that analyzing one or more of the cells comprises conducting a deoxyribonucleic acid (DNA) analysis of one or more of the cells. In some embodiments, the instructions provide that analyzing one or more of the cells comprises conducting a single cell deoxyribonucleic acid (DNA) analysis of one or more of the cells. In some embodiments, the instructions provide that analyzing one or more of the cells comprises conducting a protein analysis of one or more of the cells. In some embodiments, the instructions provide that analyzing one or more of the cells comprises conducting a single cell protein analysis of one or more of the cells. In some embodiments, the instructions further provide for conducting a molecular analysis of biological components of one or more of the cells. In some embodiments, the instructions provide that the molecular analysis is single cell molecular analysis.

In some embodiments, the instructions further provide for partitioning of the biological components for single cell analysis. In some embodiments, the kit further comprises an oil for partitioning in an aqueous droplet in oil emulsion. In some embodiments, the kit further comprises gel beads for the partitioning. In some embodiments, the instructions further provide for generating single cell barcoded biological components (analytes) for single cell data processing.

In some embodiments, the fixative buffer, treatment buffer, or storage buffer further comprises a cryoprotectant reagent. In some embodiments, the fixative buffer, treatment buffer, or storage buffer further comprises a stabilizing reagent. In some embodiments, the fixative buffer, treatment buffer, or storage buffer further comprises an RNA stabilizer. In some embodiments, the fixative buffer, treatment buffer, or storage buffer further comprises a ribonucleoside vanadyl complex (RVC). In some embodiments, the fixative buffer, treatment buffer, or storage buffer further comprises an RNase inhibitor.

In some embodiments, the instructions comprise: i) contacting the plurality of cells with a fixative buffer for 1 hour at room temperature or overnight at about 2 to about 8 degrees Celsius, wherein the fixative buffer comprises about 0.25 mM to about 1.25 mM of a poloxamine 1107/T1107 Tetronic® polymer; ii) removing the fixative buffer from the cells; and iii) suspending the cells in a storage buffer.

In some embodiments, the instructions comprise: i) contacting the plurality of cells with a fixative buffer for 1 hour at room temperature or overnight at about 2 to about 8 degrees Celsius; ii) removing the fixative buffer from the cells; and iv) suspending the cells in a storage buffer comprising about 0.25 mM to about 1.25 mM of a poloxamine 1107/T1107 Tetronic® polymer.

In some embodiments, the instructions comprise: i) contacting the plurality of cells with a fixative buffer for 1 hour at room temperature or overnight at about 2 to about 8 degrees Celsius, wherein the fixative buffer comprises about 0.25 mM to about 1.25 mM of a poloxamine 1107/T1107 Tetronic® polymer; ii) removing the fixative buffer from the cells; and iv) suspending the cells in a storage buffer comprising about 0.25 mM to about 1.25 mM of a poloxamine 1107/T1107 Tetronic® polymer.

In some embodiments, the biological components comprise RNA. In some embodiments, the biological components comprise DNA. In some embodiments, the biological components comprise protein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein entirely incorporated by reference for all purposes to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a microfluidic channel structure for partitioning individual analyte carriers.

FIG. 2 shows an example of a microfluidic channel structure for the controlled partitioning of beads into discrete droplets.

FIG. 3 shows an exemplary microfluidic channel structure for delivering barcode carrying beads to droplets.

FIG. 4 illustrates an example of a barcode carrying bead.

FIG. 5 illustrates another example of a barcode carrying bead.

FIG. 6 schematically illustrates an example microwell array.

FIG. 7 schematically illustrates an example workflow for processing nucleic acid molecules.

FIG. 8 schematically illustrates example labelling agents with nucleic acid molecules attached thereto.

FIG. 9A schematically shows an example of labelling agents. FIG. 9B schematically shows another example workflow for processing nucleic acid molecules. FIG. 9C schematically shows another example workflow for processing nucleic acid molecules.

FIG. 10 schematically shows another example of a barcode-carrying bead.

FIG. 11 is a schematic showing a sequential two-step cross-linking strategy.

FIG. 12 shows properties of various cross-linkers used in a sequential crosslinking test.

FIG. 13 is an experimental set-up for a sequential crosslinking test.

FIG. 14 shows results from the sequential crosslinking test described in FIG. 14.

FIG. 15 shows results of an mRNA pulldown experiment performed under conditions described in FIG. 13.

FIG. 16 is an experimental set-up for a sequential crosslinking experiment in PBMCs.

FIG. 17 shows results from an mRNA pulldown experiment from PBMCs treated as described in FIG. 16.

FIG. 18 shows flow cytometry data from cells treated as described in FIG. 16.

FIG. 19 shows flow cytometry data from cells treated as described in FIG. 16.

FIG. 20 shows flow cytometry flow data from cells treated as described in FIG. 16.

FIG. 21 shows flow cytometry data from cells treated as described in FIG. 16.

FIG. 22 is an experimental set-up for a sequential crosslinking experiment.

FIG. 23 shows results from the experiment described in FIG. 22.

FIGS. 24A-24C show data from an mRNA pulldown experiment from PBMCs treated as described in FIG. 22.

FIG. 25 shows flow cytometry data from cells treated as described in FIG. 22.

FIG. 26 shows flow cytometry data cells treated as described in FIG. 22.

FIG. 27 shows flow cytometry data from cells treated as described in FIG. 22.

FIG. 28 shows flow cytometry data from cells treated as described in FIG. 22.

FIG. 29 shows flow cytometry data from cells treated as described in FIG. 22.

FIGS. 30A-30C provide single-cell metrics from a sequential crosslinking experiment.

FIGS. 31A-31B show single-cell metrics from a sequential crosslinking experiment.

FIG. 32 shows an exemplary fixation protocol.

FIG. 33 shows single-cell data following fixation as performed in FIG. 32.

FIG. 34 shows single-cell data following fixation as performed in FIG. 32.

FIG. 35 is a schematic illustration showing that T1107 Tetronic® polymer can act like a cell “Band-Aid” to reduce cell leakage and improve sequencing metrics.

FIG. 36A shows results of using T1107 Tetronic® polymer to prevent RNA leakage during cellular storage at 4° C. FIG. 36B shows results of using T1107 Tetronic® polymer to prevent RNA leakage during storage at −20° C.

FIG. 37 is a summary of experimental conditions where Tetronic® polymer (T1107) may be used to decrease cell leakage, including a description of the experimental steps (such as crosslinking or storage) as well as the cell types tested.

FIG. 38A is a schematic illustration showing how Tetronic® polymers interact with cellular membranes to prevent RNA leakage. FIG. 38B is a graph showing drug-induced hemolysis and its inhibition using various Tetronic® polymers. Polymers T908, T1107, and T1307 have been previously published as significantly mitigating cyclodextrin-induced haemolysis from red blood cells (striped bars).

FIGS. 39A-39B show that adding T1107 Tetronic® polymer to crosslinked Jurkat (FIG. 39A) or PBMC (FIG. 39B) cells effectively reduces RNA leakage over 4 days storage at −20° C.

FIGS. 40A-40B show that the addition of T1107 improves cell quantity recovery at each step (post-crosslinking [FIG. 40A] and post-storage [FIG. 40B]).

FIGS. 41A-41B show that T1107 polymer in storage buffers mitigates RNA leakage (FIG. 41A) and does not impact reverse transcriptase (RT) activity (FIG. 41B).

FIGS. 42A-42B show that T1107 polymer in the cellular fixation and storage workflow (in crosslinking and storage buffers) reduces leakage (FIG. 42A) and increases the median cell assignment confidence score (FIG. 42B) even in fragile cells (mouse splenocytes).

FIGS. 43A-43B show that T1107 polymer in both crosslinking and storage of splenocytes preserves cell-type-specific VDJ metrics (VDJ-B number of B cells [FIGS. 43A] and B cell with productive VJ spanning pairs [FIG. 43B]) on both day 0 and day 5.

FIG. 44 shows T1107 polymer in both crosslinking and storage of splenocytes preserves cell-type-specific VDJ metrics (productive VJ spanning pairs: % of B cells) on both day 0 and day 5.

FIG. 45 shows inclusion of T1107 polymer in crosslinking and storage reagents improves single cell sequencing analysis metrics (increases the median gene per cell reads per cell and the median unique molecular identifiers detected per cell) of mouse splenocytes fixed/crosslinked in DSP/DTSSP and stored for 5 days.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The terms “a,” “an,” and “the,” as used herein, generally refers to singular and plural references unless the context clearly dictates otherwise. “A and/or B” is used herein to include all of the following alternatives: “A”, “B”, “A or B”, and “A and B”.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value or rounded to the nearest significant figure, in all cases inclusive of the provided value.

Headings, e.g., (a), (b), (i) etc., are presented merely for ease of reading the specification and claims. The use of headings in the specification or claims does not require the steps or elements be performed in alphabetical or numerical order or the order in which they are presented.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, the use of these terms in the specification does not by itself connote any required priority, precedence, or order.

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include polynucleotide barcodes, random nucleic acid and/or amino acid sequences, and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

The term “real time,” as used herein, can refer to a response time of less than about 1 second, a tenth of a second, a hundredth of a second, a millisecond, or less. The response time may be greater than 1 second. In some instances, real time can refer to simultaneous or substantially simultaneous processing, detection or identification.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses). The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.; as well as invertebrates, such as annelids, echinoderms, cnidarians, gastropods, crustaceans, cephalopods, mollusks, Porifera sponges, arachnids, and insects.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively, or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

As used herein, the term “barcoded nucleic acid molecule” generally refers to a nucleic acid molecule that results from, for example, the processing of a nucleic acid barcode molecule with a nucleic acid sequence (e.g., nucleic acid sequence complementary to a nucleic acid primer sequence encompassed by the nucleic acid barcode molecule). The nucleic acid sequence may be a targeted sequence or a non-targeted sequence. The nucleic acid barcode molecule may be coupled to or attached to the nucleic acid molecule comprising the nucleic acid sequence. For example, a nucleic acid barcode molecule described herein may be hybridized to an analyte (e.g., a messenger RNA (mRNA) molecule) of a cell. Reverse transcription can generate a barcoded nucleic acid molecule that has a sequence corresponding to the nucleic acid sequence of the mRNA and the barcode sequence (or a reverse complement thereof). The processing of the nucleic acid molecule comprising the nucleic acid sequence, the nucleic acid barcode molecule, or both, can include a nucleic acid reaction, such as, in non-limiting examples, reverse transcription, nucleic acid extension, ligation, etc. The nucleic acid reaction may be performed prior to, during, or following barcoding of the nucleic acid sequence to generate the barcoded nucleic acid molecule. For example, the nucleic acid molecule comprising the nucleic acid sequence may be subjected to reverse transcription and then be attached to the nucleic acid barcode molecule to generate the barcoded nucleic acid molecule, or the nucleic acid molecule comprising the nucleic acid sequence may be attached to the nucleic acid barcode molecule and subjected to a nucleic acid reaction (e.g., extension, ligation) to generate the barcoded nucleic acid molecule. A barcoded nucleic acid molecule may serve as a template, such as a template polynucleotide, that can be further processed (e.g., amplified) and sequenced to obtain the target nucleic acid sequence. For example, in the methods and systems described herein, a barcoded nucleic acid molecule may be further processed (e.g., amplified) and sequenced to obtain the nucleic acid sequence of the nucleic acid molecule (e.g., mRNA).

The term “sample,” as used herein, generally refers to a biological sample of a subject. The biological sample may comprise any number of macromolecules, for example, cellular macromolecules. The sample may be a cell sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “biological particle” may be used herein to generally refer to a discrete biological system derived from a biological sample. The biological particle may be a macromolecule. The biological particle may be a small molecule. The biological particle may be a virus. The biological particle may be a cell or derivative of a cell. The biological particle may be an organelle. The biological particle may be a nucleus of a cell. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be a constituent of a cell. The biological particle may be or may include DNA, RNA, organelles, proteins, or any combination thereof. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell but may not include other constituents of the cell. An example of such constituents is a nucleus or an organelle. A cell may be a live cell. The live cell may be capable of being cultured, for example, being cultured when enclosed in a gel or polymer matrix or cultured when comprising a gel or polymer matrix.

The term “macromolecular constituent,” as used herein, generally refers to a macromolecule contained within or from a biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “partition,” as used herein, generally, refers to a space or volume that may be suitable to contain one or more species or conduct one or more reactions. A partition can be a physical container, compartment, or vessel, such as a droplet, a flowcell, a reaction chamber, a reaction compartment, a tube, a well, or a microwell. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

### Overview

This disclosure relates to methods for sample preparation, the method comprising the following steps in order: (i) contacting a sample comprising at least one cell or tissue with a hydrophobic crosslinker; (ii) removing the hydrophobic crosslinker from the sample; (iii) contacting the sample with a hydrophilic crosslinker; and (iv) removing the hydrophilic crosslinker from the sample. This disclosure also relates to kits for performing such methods. This disclosure also relates to systems and methods for sample processing and handling, including, for example, sample compartmentalization and related analysis methods, as described further herein.

This disclosure further relates to methods and kits for reducing leakage of biological components from cells during preservation and storage of such cells. In one or more embodiments, the methods may include contacting cells with a fixative buffer, removing the fixative buffer from the cells, and suspending the cells in a storage buffer. In one or more embodiments, the kits may include a fixative buffer, a storage buffer, and instructions for preserving and storing a plurality of cells in a manner that reduces leakage of biological components. This disclosure also relates to systems and methods for sample processing and handling, including, for example, sample compartmentalization and related analysis methods, as described further herein. In embodiments, reagents of the present technology include a poloxamine/Tetronic® polymer, such as poloxamine 1107/T1107 Tetronic® polymer or the like.

### Systems and Methods for Sample Compartmentalization

In an aspect, the systems and methods described herein provide for the compartmentalization, depositing, or partitioning of one or more particles (e.g., biological particles, macromolecular constituents of biological particles, beads, reagents, etc.) into discrete compartments or partitions (referred to interchangeably herein as partitions), where each partition maintains separation of its own contents from the contents of other partitions. The partition can be a droplet in an emulsion or a well. A partition may comprise one or more other partitions.

A partition may include one or more particles. A partition may include one or more types of particles. For example, a partition of the present disclosure may comprise one or more biological particles and/or macromolecular constituents thereof. A partition may comprise one or more beads. A partition may comprise one or more gel beads. A partition may comprise one or more cell beads. A partition may include a single gel bead, a single cell bead, or both a single cell bead and single gel bead. A partition may include one or more reagents. Alternatively, a partition may be unoccupied. For example, a partition may not comprise a bead.

Unique identifiers, such as barcodes, may be injected into the droplets previous to, subsequent to, or concurrently with droplet generation, such as via a bead, as described elsewhere herein.

The methods and systems of the present disclosure may comprise methods and systems for generating one or more partitions such as droplets. The droplets may comprise a plurality of droplets in an emulsion. In some examples, the droplets may comprise droplets in a colloid. In some cases, the emulsion may comprise a microemulsion or a nanoemulsion. In some examples, the droplets may be generated with aid of a microfluidic device and/or by subjecting a mixture of immiscible phases to agitation (e.g., in a container). In some cases, a combination of the mentioned methods may be used for droplet and/or emulsion formation.

The partitions described herein may comprise small volumes, for example, less than about 10 microliters (μL), 5 μL, 1 μL, 10 nanoliters (nL), 5 nL, 1 nL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less.

For example, in the case of droplet-based partitions, the droplets may have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. Where co-partitioned with beads, it will be appreciated that the sample fluid volume, e.g., including co-partitioned biological particles and/or beads, within the partitions may be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions.

Droplets can be formed by creating an emulsion by mixing and/or agitating immiscible phases. Mixing or agitation may comprise various agitation techniques, such as vortexing, pipetting, tube flicking, or other agitation techniques. In some cases, mixing or agitation may be performed without using a microfluidic device. In some examples, the droplets may be formed by exposing a mixture to ultrasound or sonication. Systems and methods for droplet and/or emulsion generation by agitation are described in International Patent Application No. PCT/US2020/17785 and U.S. Patent Application Publication No. US20220025438, which are entirely incorporated herein by reference for all purposes.

### Microfluidic Systems

Microfluidic devices or platforms comprising microfluidic channel networks (e.g., on a chip) can be utilized to generate partitions such as droplets and/or emulsions as described herein. Methods and systems for generating partitions such as droplets, methods of encapsulating biological particles in partitions, methods of increasing the throughput of droplet generation, and various geometries, architectures, and configurations of microfluidic devices and channels are described in U.S. Patent Application Publication Nos. 2019/0367997 and 2019/0064173, each of which is entirely incorporated herein by reference for all purposes.

In some examples, individual particles can be partitioned to discrete partitions by introducing a flowing stream of particles in an aqueous fluid into a flowing stream or reservoir of a non-aqueous fluid, such that droplets may be generated at the junction of the two streams/reservoir, such as at the junction of a microfluidic device provided elsewhere herein.

The methods of the present disclosure may comprise generating partitions and/or encapsulating particles, such as biological particles, in some cases, individual biological particles such as single cells. In some examples, reagents may be encapsulated and/or partitioned (e.g., co-partitioned with biological particles) in the partitions. Various mechanisms may be employed in the partitioning of individual particles. An example may comprise porous membranes through which aqueous mixtures of cells may be extruded into fluids (e.g., non-aqueous fluids).

The partitions can be flowable within fluid streams. The partitions may comprise, for example, micro-vesicles that have an outer barrier surrounding an inner fluid center or core. In some cases, the partitions may comprise a porous matrix that is capable of entraining and/or retaining materials within its matrix. The partitions can be droplets of a first phase within a second phase, wherein the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions may be provided in a water-in-oil emulsion or oil-in-water emulsion. A variety of different vessels are described in, for example, U.S. Patent Application Publication No. 2014/0155295, which is entirely incorporated herein by reference for all purposes. Emulsion systems for creating stable droplets in non-aqueous or oil continuous phases are described in, for example, U.S. Patent Application Publication No. 2010/0105112, which is entirely incorporated herein by reference for all purposes.

Fluid properties (e.g., fluid flow rates, fluid viscosities, etc.), particle properties (e.g., volume fraction, particle size, particle concentration, etc.), microfluidic architectures (e.g., channel geometry, etc.), and other parameters may be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions may contain less than one biological particle per partition to ensure that those partitions that are occupied are primarily singly occupied. In some cases, partitions among a plurality of partitions may contain at most one biological particle (e.g., bead, DNA, cell or cellular material). In some embodiments, the various parameters (e.g., fluid properties, particle properties, microfluidic architectures, etc.) may be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

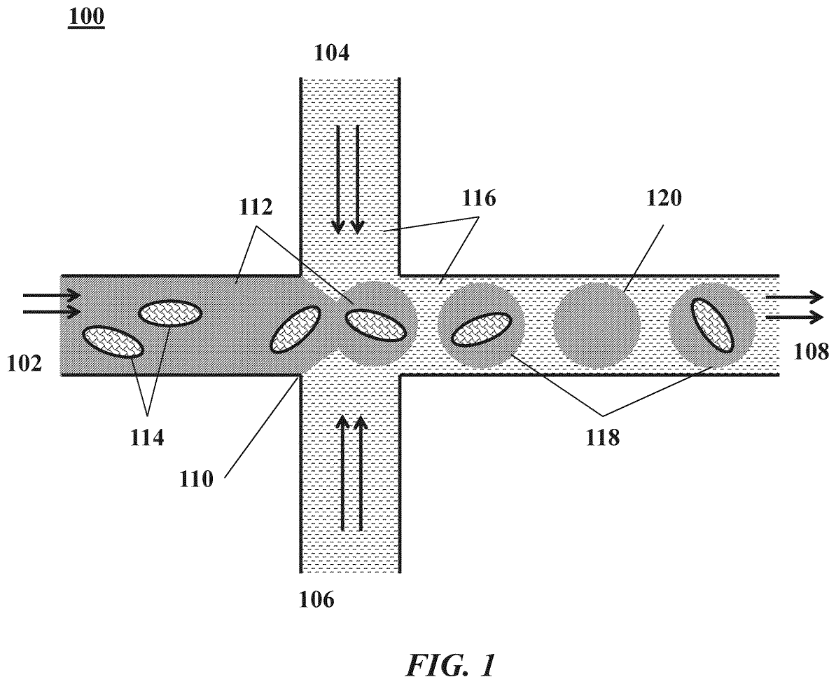

FIG. 1 shows an example of a microfluidic channel structure 100 for partitioning individual biological particles. The channel structure 100 can include channel segments 102, 104, 106 and 108 communicating at a channel junction 110. In operation, a first aqueous fluid 112 that includes suspended biological particles (or cells) 114 may be transported along channel segment 102 into junction 110, while a second fluid 116 that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 may be fluidically coupled to an outlet reservoir where the discrete droplets can be stored and/or harvested. A discrete droplet generated may include an individual biological particle 114 (such as droplets 118). A discrete droplet generated may include more than one individual biological particle 114 (not shown in FIG. 1). A discrete droplet may contain no biological particle 114 (such as droplet 120). Each discrete partition may maintain separation of its own contents (e.g., individual biological particle 114) from the contents of other partitions.

The second fluid 116 can comprise an oil, such as a fluorinated oil, that includes a fluorosurfactant for stabilizing the resulting droplets, for example, inhibiting subsequent coalescence of the resulting droplets 118, 120. Examples of particularly useful partitioning fluids and fluorosurfactants are described, for example, in U.S. Patent Application Publication No. 2010/0105112, which is entirely incorporated herein by reference for all purposes.

As will be appreciated, the channel segments described herein may be coupled to any of a variety of different fluid sources or receiving components, including reservoirs, tubing, manifolds, or fluidic components of other systems. As will be appreciated, the microfluidic channel structure 100 may have other geometries. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid may be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

The generated droplets may comprise two subsets of droplets: (1) occupied droplets 118, containing one or more biological particles 114, and (2) unoccupied droplets 120, not containing any biological particles 114. Occupied droplets 118 may comprise singly occupied droplets (having one biological particle) and multiply occupied droplets (having more than one biological particle). As described elsewhere herein, in some cases, the majority of occupied partitions can include no more than one biological particle per occupied partition and some of the generated partitions can be unoccupied (of any biological particle). In some cases, though, some of the occupied partitions may include more than one biological particle. In some cases, the partitioning process may be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it may be desirable to minimize the creation of excessive numbers of empty partitions, such as to reduce costs and/or increase efficiency. While this minimization may be achieved by providing a sufficient number of biological particles (e.g., biological particles 114) at the partitioning junction 110, such as to ensure that at least one biological particle is encapsulated in a partition, the Poissonian distribution may expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, flows can be controlled so as to present a non-Poissonian distribution of single-occupied partitions while providing lower levels of unoccupied partitions (e.g., no more than about 50%, about 25%, or about 10% unoccupied). The above noted ranges of unoccupied partitions can be achieved while still providing any of the single occupancy rates described above.

As will be appreciated, the above-described occupancy rates are also applicable to partitions that include both biological particles and additional reagents, such as beads (e.g., gel beads) carrying nucleic acid barcode molecules (e.g., oligonucleotides).

In some examples, a partition of the plurality of partitions may comprise a single biological particle (e.g., a single cell or a single nucleus of a cell). In some examples, a partition of the plurality of partitions may comprise multiple biological particles. Such partitions may be referred to as multiply occupied partitions, and may comprise, for example, two, three, four or more cells and/or beads (e.g., beads) comprising nucleic acid barcode molecules within a single partition. Accordingly, as noted above, the flow characteristics of the biological particle and/or bead containing fluids and partitioning fluids may be controlled to provide for such multiply occupied partitions. In particular, the flow parameters may be controlled to provide a given occupancy rate at greater than about 50% of the partitions, greater than about 75%, and in some cases greater than about 80%, 90%, 95%, or higher.

Microfluidic systems for partitioning are further described in U.S. Patent Application Pub. No. US 2015/0376609, which is hereby incorporated by reference in its entirety.

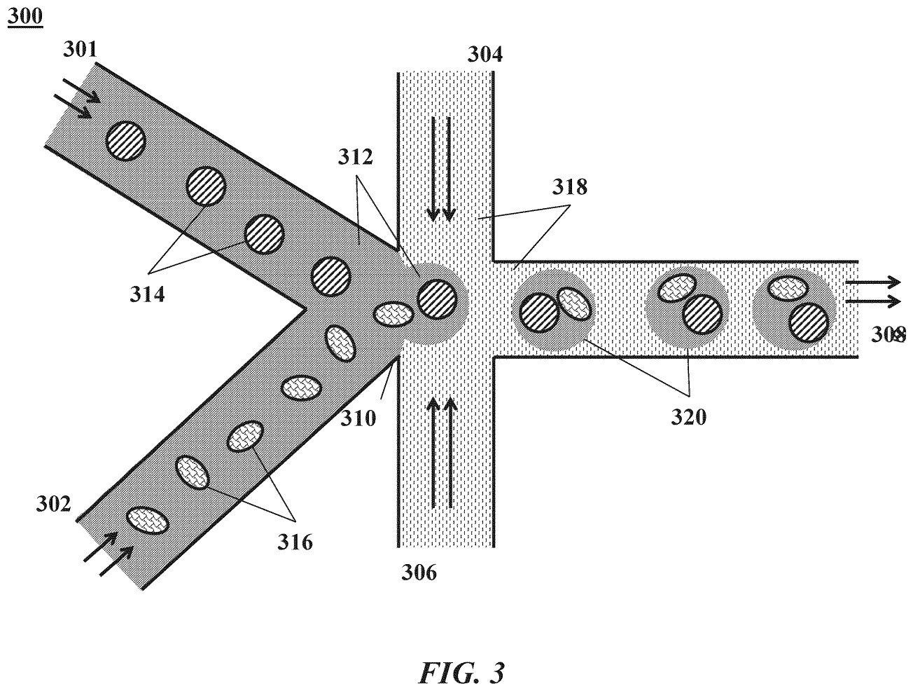

FIG. 3 shows an example of a microfluidic channel structure 300 for delivering barcode carrying beads to droplets. The channel structure 300 can include channel segments 301, 302, 304, 306 and 308 communicating at a channel junction 310. In operation, the channel segment 301 may transport an aqueous fluid 312 that includes a plurality of beads 314 (e.g., with nucleic acid molecules, e.g., nucleic acid barcode molecules or barcoded oligonucleotides, molecular tags) along the channel segment 301 into junction 310. The plurality of beads 314 may be sourced from a suspension of beads. For example, the channel segment 301 may be connected to a reservoir comprising an aqueous suspension of beads 314. The channel segment 302 may transport the aqueous fluid 312 that includes a plurality of biological particles 316 along the channel segment 302 into junction 310. The plurality of biological particles 316 may be sourced from a suspension of biological particles. For example, the channel segment 302 may be connected to a reservoir comprising an aqueous suspension of biological particles 316. In some instances, the aqueous fluid 312 in either the first channel segment 301 or the second channel segment 302, or in both segments, can include one or more reagents, as further described below. A second fluid 318 that is immiscible with the aqueous fluid 312 (e.g., oil) can be delivered to the junction 310 from each of channel segments 304 and 306. Upon meeting of the aqueous fluid 312 from each of channel segments 301 and 302 and the second fluid 318 from each of channel segments 304 and 306 at the channel junction 310, the aqueous fluid 312 can be partitioned as discrete droplets 320 in the second fluid 318 and flow away from the junction 310 along channel segment 308. The channel segment 308 may deliver the discrete droplets to an outlet reservoir fluidly coupled to the channel segment 308, where they may be harvested. As an alternative, the channel segments 301 and 302 may meet at another junction upstream of the junction 310. At such junction, beads and biological particles may form a mixture that is directed along another channel to the junction 310 to yield droplets 320. The mixture may provide the beads and biological particles in an alternating fashion, such that, for example, a droplet comprises a single bead and a single biological particle.

### Controlled Partitioning

In some aspects, provided are systems and methods for controlled partitioning. Droplet size may be controlled by adjusting certain geometric features in channel architecture (e.g., microfluidics channel architecture). For example, an expansion angle, width, and/or length of a channel may be adjusted to control droplet size.

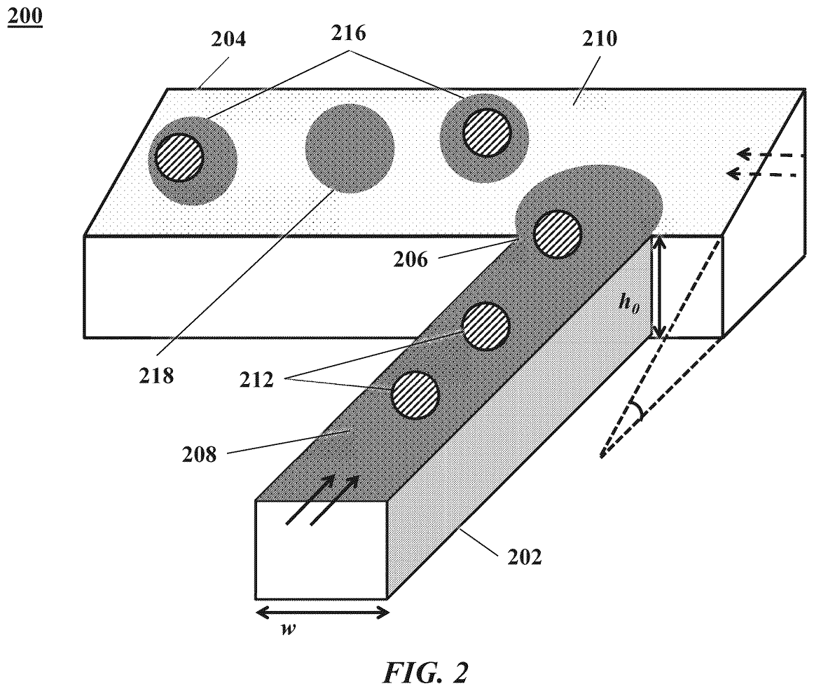

FIG. 2 shows an example of a microfluidic channel structure for the controlled partitioning of beads into discrete droplets. A channel structure 200 can include a channel segment 202 communicating at a channel junction 206 (or intersection) with a reservoir 204. The reservoir 204 can be a chamber. Any reference to “reservoir,” as used herein, can also refer to a “chamber.” In operation, an aqueous fluid 208 that includes suspended beads 212 may be transported along the channel segment 202 into the junction 206 to meet a second fluid 210 that is immiscible with the aqueous fluid 208 in the reservoir 204 to create droplets 216, 218 of the aqueous fluid 208 flowing into the reservoir 204. At the junction 206 where the aqueous fluid 208 and the second fluid 210 meet, droplets can form based on factors such as the hydrodynamic forces at the junction 206, flow rates of the two fluids 208, 210, fluid properties, and certain geometric parameters (e.g., w, h₀, α, etc.) of the channel structure 200. A plurality of droplets can be collected in the reservoir 204 by continuously injecting the aqueous fluid 208 from the channel segment 202 through the junction 206.

In some instances, the aqueous fluid 208 can have a substantially uniform concentration or frequency of beads 212. The beads 212 can be introduced into the channel segment 202 from a separate channel (not shown in FIG. 2). The frequency of beads 212 in the channel segment 202 may be controlled by controlling the frequency in which the beads 212 are introduced into the channel segment 202 and/or the relative flow rates of the fluids in the channel segment 202 and the separate channel. In some instances, the beads can be introduced into the channel segment 202 from a plurality of different channels, and the frequency controlled accordingly.

In some instances, the aqueous fluid 208 in the channel segment 202 can comprise biological particles. In some instances, the aqueous fluid 208 can have a substantially uniform concentration or frequency of biological particles. As with the beads, the biological particles can be introduced into the channel segment 202 from a separate channel. The frequency or concentration of the biological particles in the aqueous fluid 208 in the channel segment 202 may be controlled by controlling the frequency in which the biological particles are introduced into the channel segment 202 and/or the relative flow rates of the fluids in the channel segment 202 and the separate channel. In some instances, the biological particles can be introduced into the channel segment 202 from a plurality of different channels, and the frequency controlled accordingly. In some instances, a first separate channel can introduce beads and a second separate channel can introduce biological particles into the channel segment 202. The first separate channel introducing the beads may be upstream or downstream of the second separate channel introducing the biological particles.

The second fluid 210 can comprise an oil, such as a fluorinated oil, that includes a fluorosurfactant for stabilizing the resulting droplets, for example, inhibiting subsequent coalescence of the resulting droplets.

In some instances, the second fluid 210 may not be subjected to and/or directed to any flow in or out of the reservoir 204. For example, the second fluid 210 may be substantially stationary in the reservoir 204. In some instances, the second fluid 210 may be subjected to flow within the reservoir 204, but not in or out of the reservoir 204, such as via application of pressure to the reservoir 204 and/or as affected by the incoming flow of the aqueous fluid 208 at the junction 206. Alternatively, the second fluid 210 may be subjected and/or directed to flow in or out of the reservoir 204. For example, the reservoir 204 can be a channel directing the second fluid 210 from upstream to downstream, transporting the generated droplets.

Systems and methods for controlled partitioning are described further in International Patent Application No. PCT/US2018/047551 and U.S. Patent Application Publication No. US2020/0290048, which are hereby incorporated by reference in their entirety.

### Cell Beads

In another aspect, in addition to or as an alternative to droplet-based partitioning, biological particles (e.g., cells) may be comprised within (e.g., encapsulated within) a particulate material to form a “cell bead”. Methods and compositions drawn to cell beads and the like are described further in International Patent Application No. PCT/US2018/016019 and U.S. Patent Application No. US2018/0216162, which are hereby incorporated by reference in their entirety.

A cell bead can contain a biological particle (e.g., a cell) or macromolecular constituents (e.g., RNA, DNA, proteins, etc.) of a biological particle. A cell bead may include a single cell or multiple cells, or a derivative of the single cell or multiple cells. For example, after lysing and washing the cells, inhibitory components from cell lysates can be washed away and the macromolecular constituents can be bound as cell beads. Systems and methods disclosed herein can be applicable to both cell beads (and/or droplets or other partitions) containing biological particles and cell beads (and/or droplets or other partitions) containing macromolecular constituents of biological particles. Cell beads may be or include a cell, cell derivative, cellular material and/or material derived from the cell in, within, or encased in a matrix, such as a polymeric matrix. In some cases, a cell bead may comprise a live cell. In some instances, the live cell may be capable of being cultured when enclosed in a gel or polymer matrix, or of being cultured when comprising a gel or polymer matrix. In some instances, the polymer or gel may be diffusively permeable to certain components and diffusively impermeable to other components (e.g., macromolecular constituents).

Cell beads can provide certain potential advantages of being more storable and more portable than droplet-based partitioned biological particles. Furthermore, in some cases, it may be desirable to allow biological particles to incubate for a select period of time before analysis, such as in order to characterize changes in such biological particles over time, either in the presence or absence of different stimuli (or reagents).

Suitable polymers or gels may include one or more of disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, hyaluronic acid, collagen, fibrin, gelatin, or elastin. The polymer or gel may comprise any other polymer or gel.

Encapsulation of biological particles may be performed by a variety of processes. Such processes may combine an aqueous fluid containing the biological particles with a polymeric precursor material that may be capable of being formed into a gel or other solid or semi-solid matrix upon application of a particular stimulus to the polymer precursor. The conditions sufficient to polymerize or gel the precursors may comprise any conditions sufficient to polymerize or gel the precursors. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), electromagnetic radiation, mechanical stimuli, or any combination thereof.

In some cases, air knife droplet or aerosol generators may be used to dispense droplets of precursor fluids into gelling solutions in order to form cell beads that include individual biological particles or small groups of biological particles. Likewise, membrane-based encapsulation systems may be used to generate cell beads comprising encapsulated biological particles as described herein. Microfluidic systems of the present disclosure, such as that shown in FIG. 1, may be readily used in encapsulating biological particles (e.g., cells) as described herein. Exemplary methods for encapsulating biological particles (e.g., cells) are also further described in U.S. Patent Application Pub. No. US 2015/0376609 and International Patent Application No. PCT/US2018/016019, which are hereby incorporated by reference in their entirety. In particular, and with reference to FIG. 1, the aqueous fluid 112 comprising (i) the biological particles 114 and (ii) the polymer precursor material (not shown) is flowed into channel junction 110, where it is partitioned into droplets 118, 120 through the flow of non-aqueous fluid 116. In the case of encapsulation methods, non-aqueous fluid 116 may also include an initiator (not shown) to cause polymerization and/or crosslinking of the polymer precursor to form the bead that includes the entrained biological particles. Examples of polymer precursor/initiator pairs include those described in U.S. Patent Application Publication No. 2014/0378345, which is entirely incorporated herein by reference for all purposes.

In some cases, encapsulated biological particles can be selectively releasable from the cell bead, such as through passage of time or upon application of a particular stimulus, that degrades the bead sufficiently to allow the biological particles (e.g., cell), or its other contents to be released from the bead, such as into a partition (e.g., droplet). Exemplary stimuli suitable for degradation of the bead are described in U.S. Patent Application Publication No. 2014/0378345, which is entirely incorporated herein by reference for all purposes.

The polymer or gel may be diffusively permeable to chemical or biochemical reagents. The polymer or gel may be diffusively impermeable to macromolecular constituents of the biological particle. In this manner, the polymer or gel may act to allow the biological particle to be subjected to chemical or biochemical operations while spatially confining the macromolecular constituents to a region of the droplet defined by the polymer or gel.

The polymer or gel may be functionalized to bind to targeted analytes, such as nucleic acids, proteins, carbohydrates, lipids or other analytes. The polymer or gel may be polymerized or gelled via a passive mechanism. The polymer or gel may be stable in alkaline conditions or at elevated temperature. The polymer or gel may have mechanical properties similar to the mechanical properties of the bead. For instance, the polymer or gel may be of a similar size to the bead. The polymer or gel may have a mechanical strength (e.g. tensile strength) similar to that of the bead. The polymer or gel may be of a lower density than an oil. The polymer or gel may be of a density that is roughly similar to that of a buffer. The polymer or gel may have a tunable pore size. The pore size may be chosen to, for instance, retain denatured nucleic acids. The pore size may be chosen to maintain diffusive permeability to exogenous chemicals such as sodium hydroxide (NaOH) and/or endogenous chemicals such as inhibitors. The polymer or gel may be biocompatible. The polymer or gel may maintain or enhance cell viability. The polymer or gel may be biochemically compatible. The polymer or gel may be polymerized and/or depolymerized thermally, chemically, enzymatically, and/or optically.

The encapsulation of biological particles may constitute the partitioning of the biological particles into which other reagents are co-partitioned. Alternatively, or in addition, encapsulated biological particles may be readily deposited into other partitions (e.g., droplets) as described above.

### Beads

Nucleic acid barcode molecules may be delivered to a partition (e.g., a droplet or well) via a solid support or carrier (e.g., a bead). In some cases, nucleic acid barcode molecules are initially associated with the solid support and then released from the solid support upon application of a stimulus, which allows the nucleic acid barcode molecules to dissociate or to be released from the solid support. In specific examples, nucleic acid barcode molecules are initially associated with the solid support (e.g., bead) and then released from the solid support upon application of a biological stimulus, a chemical stimulus, a thermal stimulus, an electrical stimulus, a magnetic stimulus, and/or a photo stimulus.

The solid support may be a bead. A solid support, e.g., a bead, may be porous, non-porous, hollow, solid, semi-solid, and/or a combination thereof. Beads may be solid, semi-solid, semi-fluidic, fluidic, and/or a combination thereof. In some instances, a solid support, e.g., a bead, may be at least partially dissolvable, disruptable, and/or degradable. In some cases, a solid support, e.g., a bead, may not be degradable. In some cases, the solid support, e.g., a bead, may be a gel bead. A gel bead may be a hydrogel bead. A gel bead may be formed from molecular precursors, such as a polymeric or monomeric species. A semi-solid support, e.g., a bead, may be a liposomal bead. Solid supports, e.g., beads, may comprise metals including iron oxide, gold, and silver. In some cases, the solid support, e.g., the bead, may be a silica bead. In some cases, the solid support, e.g., a bead, can be rigid. In other cases, the solid support, e.g., a bead, may be flexible and/or compressible.

A partition may comprise one or more unique identifiers, such as barcodes. Barcodes may be previously, subsequently or concurrently delivered to the partitions that hold the compartmentalized or partitioned biological particle. For example, barcodes may be injected into droplets or deposited in microwells previous to, subsequent to, or concurrently with droplet generation or providing of reagents in the microwells, respectively. The delivery of the barcodes to a particular partition allows for the later attribution of the characteristics of the individual biological particle to the particular partition. Barcodes may be delivered, for example on a nucleic acid molecule (e.g., via a nucleic acid barcode molecule), to a partition via any suitable mechanism. Nucleic acid barcode molecules can be delivered to a partition via a bead. Beads are described in further detail below.

In some cases, nucleic acid barcode molecules can be initially associated with the bead and then released from the bead. Release of the nucleic acid barcode molecules can be passive (e.g., by diffusion out of the bead). In addition, or alternatively, release from the bead can be upon application of a stimulus which allows the nucleic acid barcode molecules to dissociate or to be released from the bead. Such stimulus may disrupt the bead, an interaction that couples the nucleic acid barcode molecules to or within the bead, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof.

Methods and systems for partitioning barcode carrying beads into droplets are provided herein, and in in US. Patent Publication Nos. 2019/0367997 and 2019/0064173, and International Patent Application No. PCT/US20/17785, each of which is herein entirely incorporated by reference for all purposes.

A bead may be porous, non-porous, solid, semi-solid, semi-fluidic, fluidic, and/or a combination thereof. In some instances, a bead may be dissolvable, disruptable, and/or degradable. Degradable beads, as well as methods for degrading beads, are described in International Patent Application Publication No. PCT/US2014/044398, which is hereby incorporated by reference in its entirety. In some cases, any combination of stimuli, e.g., stimuli described in PCT/US2014/044398 and US Patent Application Pub. No. 2015/0376609, hereby incorporated by reference in its entirety, may trigger degradation of a bead. For example, a change in pH may enable a chemical agent (e.g., DTT) to become an effective reducing agent. In other examples, a reducing agent (e.g., DTT) may be used to degrade the bead.

In some cases, a bead may not be degradable. In some cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible.

A bead may be of any suitable shape. Examples of bead shapes include, but are not limited to, spherical, non-spherical, oval, oblong, amorphous, circular, cylindrical, and variations thereof.

Beads may be of uniform size or heterogeneous size. In some cases, the diameter of a bead may be at least about 10 nanometers (nm), 100 nm, 500 nm, 1 micrometer (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a bead may have a diameter of less than about 10 nm, 100 nm, 500 nm, 1 μm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In particular, the beads described herein may have size distributions that have a coefficient of variation in their cross-sectional dimensions of less than 50%, less than 40%, less than 30%, less than 20%, and in some cases less than 15%, less than 10%, less than 5%, or less.

A bead may comprise natural and/or synthetic materials. For example, a bead can comprise a natural polymer, a synthetic polymer or both natural and synthetic polymers. See, e.g., PCT/US2014/044398, which is hereby incorporated by reference in its entirety. Beads may also be formed from materials other than polymers, including lipids, micelles, ceramics, glass-ceramics, material composites, metals, other inorganic materials, and others.

In some cases, the bead may comprise covalent or ionic bonds between polymeric precursors (e.g., monomers, oligomers, linear polymers), nucleic acid barcode molecules (e.g., oligonucleotides), primers, and other entities. In some cases, the covalent bonds can be carbon-carbon bonds, thioether bonds, or carbon-heteroatom bonds.

In some cases, a plurality of nucleic acid barcode molecules may be attached to a bead. The nucleic acid barcode molecules may be attached directly or indirectly to the bead. In some cases, the nucleic acid barcode molecules may be covalently linked to the bead. In some cases, the nucleic acid barcode molecules are covalently linked to the bead via a linker. In some cases, the linker is a degradable linker. In some cases, the linker comprises a labile bond configured to release the nucleic acid barcode molecule of said plurality of nucleic acid barcode molecules. In some cases, the labile bond comprises a disulfide linkage.

Activation or disruption of disulfide linkages within a bead can be controlled such that only a small number of disulfide linkages are activated or disrupted. Methods of controlling activation of disulfide linkages within a bead are described in International Patent Application No. PCT/US2014/044398, which is hereby incorporated by reference in its entirety.

In some cases, a bead may comprise an acrydite moiety, which in certain aspects may be used to attach one or more nucleic acid barcode molecules (e.g., barcode sequence, nucleic acid barcode molecule, barcoded oligonucleotide, primer, or other oligonucleotide) to the bead. Acrydite moieties, as well as their uses in attaching nucleic acid molecules to beads, are described in PCT/US2014/044398, which is hereby incorporated by reference in its entirety.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule, e.g., a nucleic acid barcode molecule described herein.

In some cases, precursors comprising a functional group that is reactive or capable of being activated such that it becomes reactive can be polymerized with other precursors to generate gel beads comprising the activated or activatable functional group. The functional group may then be used to attach additional species (e.g., disulfide linkers, primers, other oligonucleotides, etc.) to the gel beads. Exemplary precursors comprising functional groups are described in PCT/US2014/044398, which is hereby incorporated by reference in its entirety.

Other non-limiting examples of labile bonds that may be coupled to a precursor or bead are described in PCT/US2014/044398, which is hereby incorporated by reference in its entirety. A bond may be cleavable via other nucleic acid molecule targeting enzymes, such as restriction enzymes (e.g., restriction endonucleases), as described further below.

In some cases, a plurality of nucleic acid barcode molecules may be attached to a bead via non-covalent bonds. For example, the plurality of nucleic acid barcode molecules may be associated with a bead via an ionic interaction, electrostatic interactions, metallic bond, hydrogen bonding, van der Waals interactions, etc. In some cases, the non-covalent bond may be degraded upon application of a stimulus, e.g., a thermal, photo, magnetic, electrical, chemical stimulus (e.g., change in pH, ion concentration, etc.).

Species may be encapsulated in beads during bead generation (e.g., during polymerization of precursors). Such species may or may not participate in polymerization. See, e.g., PCT/US2014/044398, which is hereby incorporated by reference in its entirety. Such species may include, for example, nucleic acid molecules (e.g., oligonucleotides), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species may include one or more enzymes described herein, including without limitation, polymerase, reverse transcriptase, restriction enzymes (e.g., endonuclease), transposase, ligase, proteinase K, DNAse, etc. Such species may include one or more reagents described elsewhere herein (e.g., lysis agents, inhibitors, inactivating agents, chelating agents, stimulus). Alternatively, or in addition, species may be partitioned in a partition (e.g., droplet) during or subsequent to partition formation. Such species may include, without limitation, the abovementioned species that may also be encapsulated in a bead.

In some cases, beads can be non-covalently loaded with and/or coupled to one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads may be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads may be accomplished by various swelling methods. The de-swelling of the beads may be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads may be accomplished by various de-swelling methods. Transferring the beads may cause pores in the bead to shrink. The shrinking may then hinder reagents within the beads from diffusing out of the interiors of the beads. The hindrance may be due to steric interactions between the reagents and the interiors of the beads. The transfer may be accomplished microfluidically. For instance, the transfer may be achieved by moving the beads from one co-flowing solvent stream to a different co-flowing solvent stream. The swellability and/or pore size of the beads may be adjusted by changing the polymer composition of the bead.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing oligonucleotide bearing beads.

### Nucleic Acid Barcode Molecules

A nucleic acid barcode molecule may contain one or more barcode sequences. A plurality of nucleic acid barcode molecules may be coupled to a bead. The one or more barcode sequences may include sequences that are the same for all or a portion of the nucleic acid molecules coupled to a given bead and/or sequences that are different across all (or a portion of the) nucleic acid molecules coupled to the given bead. The nucleic acid molecule may be incorporated into the bead.

Nucleic acid barcode molecules can comprise one or more functional sequences for coupling to an analyte or analyte tag such as a reporter oligonucleotide. Such functional sequences can include, e.g., a template switch oligonucleotide (TSO) sequence, a primer sequence (e.g., a poly T sequence, or a nucleic acid primer sequence complementary to a target nucleic acid sequence and/or for amplifying a target nucleic acid sequence, a random primer, and a primer sequence for messenger RNA).

In some cases, the nucleic acid barcode molecule can further comprise a unique molecular identifier (UMI). In some cases, the nucleic acid barcode molecule can comprise one or more functional sequences, for example, for attachment to a sequencing flow cell, such as, for example, a P5 sequence (or a portion thereof) for Illumina® sequencing. In some cases, the nucleic acid barcode molecule or derivative thereof (e.g., oligonucleotide or polynucleotide generated from the nucleic acid molecule) can comprise another functional sequence, such as, for example, a P7 sequence (or a portion thereof) for attachment to a sequencing flow cell for Illumina sequencing.

In some cases, the nucleic acid molecule can comprise an R1 primer sequence for Illumina sequencing. In some cases, the nucleic acid molecule can comprise an R2 primer sequence for Illumina sequencing. In some cases, a functional sequence can comprise a partial sequence, such as a partial barcode sequence, partial anchoring sequence, partial sequencing primer sequence (e.g., partial R1 sequence, partial R2 sequence, etc.), a partial sequence configured to attach to the flow cell of a sequencer (e.g., partial P5 sequence, partial P7 sequence, etc.), or a partial sequence of any other type of sequence described elsewhere herein. A partial sequence may contain a contiguous or continuous portion or segment, but not all, of a full sequence, for example. In some cases, a downstream procedure may extend the partial sequence, or derivative thereof, to achieve a full sequence of the partial sequence, or derivative thereof.

Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference.

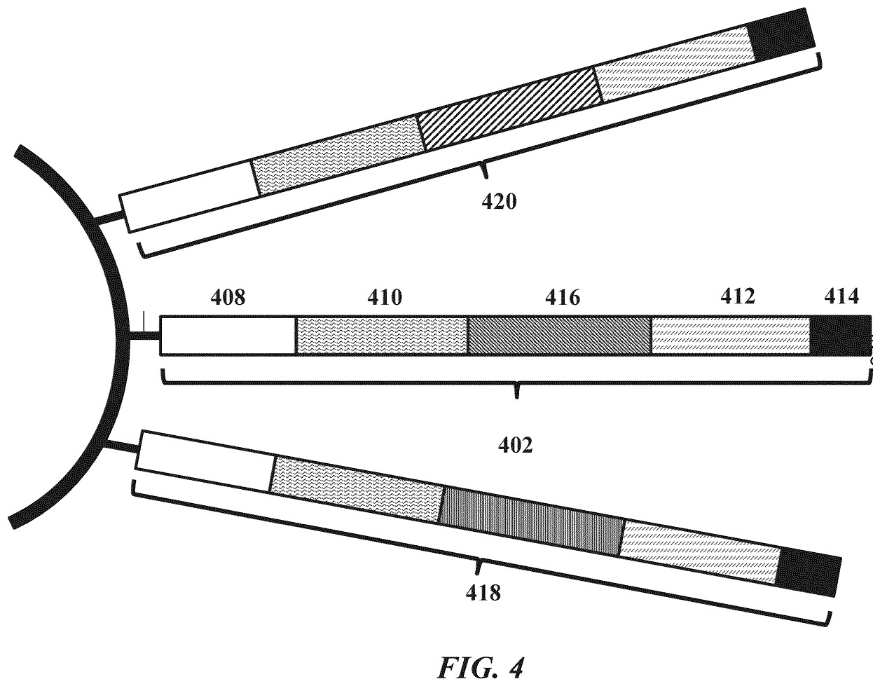

FIG. 4 illustrates an example of a barcode carrying bead. A nucleic acid barcode molecule 402 can be coupled to a bead 404 by a releasable linkage 406, such as, for example, a disulfide linker. The same bead 404 may be coupled (e.g., via releasable linkage) to one or more other nucleic acid barcode molecules 418, 420. The nucleic acid barcode molecule 402 may be or comprise a barcode. As noted elsewhere herein, the structure of the barcode may comprise a number of sequence elements. The nucleic acid barcode molecule 402 may comprise a functional sequence 408 that may be used in subsequent processing. For example, the functional sequence 408 may include one or more of a sequencer specific flow cell attachment sequence (e.g., a P5 sequence for Illumina® sequencing systems) and a sequencing primer sequence (e.g., a R1 primer for Illumina® sequencing systems), or partial sequence(s) thereof. The nucleic acid barcode molecule 402 may comprise a barcode sequence 410 for use in barcoding the sample (e.g., DNA, RNA, protein, etc.). In some cases, the barcode sequence 410 can be bead-specific such that the barcode sequence 410 is common to all nucleic acid barcode molecules (e.g., including nucleic acid barcode molecule 402) coupled to the same bead 404. Alternatively, or in addition, the barcode sequence 410 can be partition-specific such that the barcode sequence 410 is common to all nucleic acid barcode molecules coupled to one or more beads that are partitioned into the same partition. The nucleic acid barcode molecule 402 may comprise sequence 412 complementary to an analyte of interest, e.g., a priming sequence. Sequence 412 can be a poly-T sequence complementary to a poly-A tail of an mRNA analyte, a targeted priming sequence, and/or a random priming sequence. The nucleic acid barcode molecule 402 may comprise an anchoring sequence 414 to ensure that the specific priming sequence 412 hybridizes at the sequence end (e.g., of the mRNA). For example, the anchoring sequence 414 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid barcode molecule 402 may comprise a unique molecular identifying sequence 416 (e.g., unique molecular identifier (UMI)). In some cases, the unique molecular identifying sequence 416 may comprise from about 5 to about 8 nucleotides. Alternatively, the unique molecular identifying sequence 416 may compress less than about 5 or more than about 8 nucleotides. The unique molecular identifying sequence 416 may be a unique sequence that varies across individual nucleic acid barcode molecules (e.g., 402, 418, 420, etc.) coupled to a single bead (e.g., bead 404). In some cases, the unique molecular identifying sequence 416 may be a random sequence (e.g., such as a random N-mer sequence). For example, the UMI may provide a unique identifier of the starting analyte (e.g., mRNA) molecule that was captured, in order to allow quantitation of the number of original expressed RNA molecules. As will be appreciated, although FIG. 4 shows three nucleic acid barcode molecules 402, 418, 420 coupled to the surface of the bead 404, an individual bead may be coupled to any number of individual nucleic acid barcode molecules, for example, from one to tens to hundreds of thousands, millions, or even a billion of individual nucleic acid barcode molecules. The respective barcodes for the individual nucleic acid barcode molecules can comprise both common sequence segments or relatively common sequence segments (e.g., 408, 410, 412, etc.) and variable or unique sequence segments (e.g., 416) between different individual nucleic acid barcode molecules coupled to the same bead.

In operation, a biological particle (e.g., cell, DNA, RNA, etc.) can be co-partitioned along with a barcode bearing bead 404. The nucleic acid barcode molecules 402, 418, 420 can be released from the bead 404 in the partition. By way of example, in the context of analyzing sample RNA, the poly-T segment (e.g., 412) of one of the released nucleic acid barcode molecules (e.g., 402) can hybridize to the poly-A tail of a mRNA molecule. Reverse transcription may result in a cDNA transcript of the mRNA, but which transcript includes each of the sequence segments 408, 410, 416 of the nucleic acid barcode molecule 402. Because the nucleic acid barcode molecule 402 comprises an anchoring sequence 414, it will more likely hybridize to and prime reverse transcription at the sequence end of the poly-A tail of the mRNA. Within any given partition, all of the cDNA transcripts of the individual mRNA molecules may include a common barcode sequence segment 410. However, the transcripts made from the different mRNA molecules within a given partition may vary at the unique molecular identifying sequence 412 segment (e.g., UMI segment). Beneficially, even following any subsequent amplification of the contents of a given partition, the number of different UMIs can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in some cases, the nucleic acid barcode molecules bound to the bead (e.g., gel bead) may be used to hybridize and capture the mRNA on the solid phase of the bead, for example, in order to facilitate the separation of the RNA from other cell contents. In such cases, further processing may be performed, in the partitions or outside the partitions (e.g., in bulk). For instance, the RNA molecules on the beads may be subjected to reverse transcription or other nucleic acid processing, additional adapter sequences may be added to the barcoded nucleic acid molecules, or other nucleic acid reactions (e.g., amplification, nucleic acid extension) may be performed. The beads or products thereof (e.g., barcoded nucleic acid molecules) may be collected from the partitions, and/or pooled together and subsequently subjected to clean up and further characterization (e.g., sequencing).

The operations described herein may be performed at any useful or convenient step. For instance, the beads comprising nucleic acid barcode molecules may be introduced into a partition (e.g., well or droplet) prior to, during, or following introduction of a sample into the partition. The nucleic acid molecules of a sample may be subjected to barcoding, which may occur on the bead (in cases where the nucleic acid molecules remain coupled to the bead) or following release of the nucleic acid barcode molecules into the partition. In cases where the nucleic acid molecules from the sample remain attached to the bead, the beads from various partitions may be collected, pooled, and subjected to further processing (e.g., reverse transcription, adapter attachment, amplification, clean up, sequencing). In other instances, the processing may occur in the partition. For example, conditions sufficient for barcoding, adapter attachment, reverse transcription, or other nucleic acid processing operations may be provided in the partition and performed prior to clean up and sequencing.

In some instances, a bead may comprise a capture sequence or binding sequence configured to bind to a corresponding capture sequence or binding sequence. In some instances, a bead may comprise a plurality of different capture sequences or binding sequences configured to bind to different respective corresponding capture sequences or binding sequences. For example, a bead may comprise a first subset of one or more capture sequences each configured to bind to a first corresponding capture sequence, a second subset of one or more capture sequences each configured to bind to a second corresponding capture sequence, a third subset of one or more capture sequences each configured to bind to a third corresponding capture sequence, and etc. A bead may comprise any number of different capture sequences. In some instances, a bead may comprise at least 2, 3, 4, 5, 6, 7, 8, 9, 10 or more different capture sequences or binding sequences configured to bind to different respective capture sequences or binding sequences, respectively. Alternatively, or in addition, a bead may comprise at most about 10, 9, 8, 7, 6, 5, 4, 3, or 2 different capture sequences or binding sequences configured to bind to different respective capture sequences or binding sequences. In some instances, the different capture sequences or binding sequences may be configured to facilitate analysis of a same type of analyte. In some instances, the different capture sequences or binding sequences may be configured to facilitate analysis of different types of analytes (with the same bead). The capture sequence may be designed to attach to a corresponding capture sequence. Beneficially, such corresponding capture sequence may be introduced to, or otherwise induced in, an biological particle (e.g., cell, cell bead, etc.) for performing different assays in various formats (e.g., barcoded antibodies comprising the corresponding capture sequence, barcoded MHC dextramers comprising the corresponding capture sequence, barcoded guide RNA molecules comprising the corresponding capture sequence, etc.), such that the corresponding capture sequence may later interact with the capture sequence associated with the bead. In some instances, a capture sequence coupled to a bead (or other support) may be configured to attach to a linker molecule, such as a splint molecule, wherein the linker molecule is configured to couple the bead (or other support) to other molecules through the linker molecule, such as to one or more analytes or one or more other linker molecules.

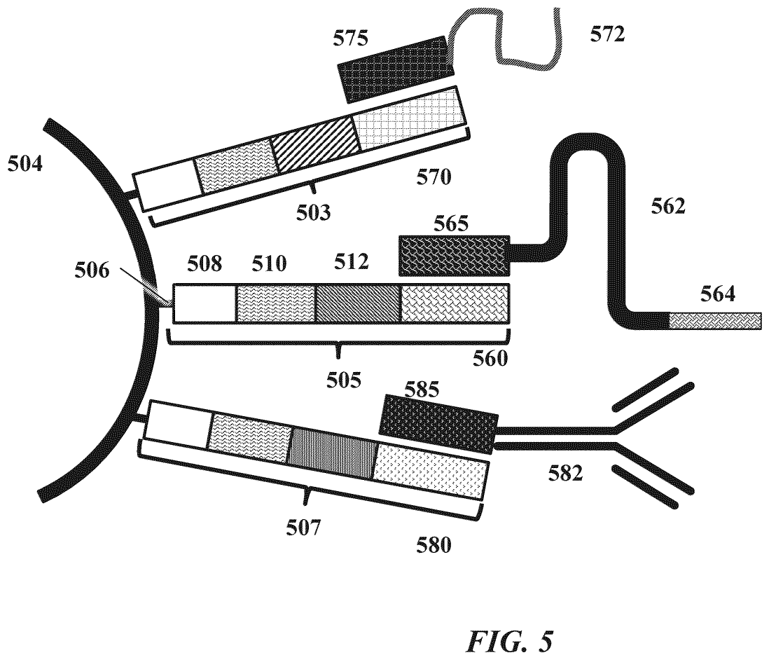

FIG. 5 illustrates another example of a barcode carrying bead. A nucleic acid barcode molecule 505, such as an oligonucleotide, can be coupled to a bead 504 by a releasable linkage 506, such as, for example, a disulfide linker. The nucleic acid barcode molecule 505 may comprise a first capture sequence 560. The same bead 504 may be coupled (e.g., via releasable linkage) to one or more other nucleic acid molecules 503, 507 comprising other capture sequences. The nucleic acid barcode molecule 505 may be or comprise a barcode. As noted elsewhere herein, the structure of the barcode may comprise a number of sequence elements, such as a functional sequence 508 (e.g., flow cell attachment sequence, sequencing primer sequence, etc.), a barcode sequence 510 (e.g., bead-specific sequence common to bead, partition-specific sequence common to partition, etc.), and a unique molecular identifier 512 (e.g., unique sequence within different molecules attached to the bead), or partial sequences thereof. The capture sequence 560 may be configured to attach to a corresponding capture sequence 565. In some instances, the corresponding capture sequence 565 may be coupled to another molecule that may be an analyte or an intermediary carrier. For example, as illustrated in FIG. 5, the corresponding capture sequence 565 is coupled to a guide RNA molecule 562 comprising a target sequence 564, wherein the target sequence 564 is configured to attach to the analyte. Another oligonucleotide molecule 507 attached to the bead 504 comprises a second capture sequence 580 which is configured to attach to a second corresponding capture sequence 585. As illustrated in FIG. 5, the second corresponding capture sequence 585 is coupled to an antibody 582. In some cases, the antibody 582 may have binding specificity to an analyte (e.g., surface protein). Alternatively, the antibody 582 may not have binding specificity. Another oligonucleotide molecule 503 attached to the bead 504 comprises a third capture sequence 570 which is configured to attach to a third corresponding capture sequence 575. As illustrated in FIG. 5, the third corresponding capture sequence 575 is coupled to a molecule 572. The molecule 572 may or may not be configured to target an analyte. The other oligonucleotide molecules 503, 507 may comprise the other sequences (e.g., functional sequence, barcode sequence, UMI, etc.) described with respect to oligonucleotide molecule 505. While a single oligonucleotide molecule comprising each capture sequence is illustrated in FIG. 5, it will be appreciated that, for each capture sequence, the bead may comprise a set of one or more oligonucleotide molecules each comprising the capture sequence. For example, the bead may comprise any number of sets of one or more different capture sequences. Alternatively, or in addition, the bead 504 may comprise other capture sequences. Alternatively, or in addition, the bead 504 may comprise fewer types of capture sequences (e.g., two capture sequences). Alternatively, or in addition, the bead 504 may comprise oligonucleotide molecule(s) comprising a priming sequence, such as a specific priming sequence such as an mRNA specific priming sequence (e.g., poly-T sequence), a targeted priming sequence, and/or a random priming sequence, for example, to facilitate an assay for gene expression.

In operation, the barcoded oligonucleotides may be released (e.g., in a partition), as described elsewhere herein. Alternatively, the nucleic acid molecules bound to the bead (e.g., gel bead) may be used to hybridize and capture analytes (e.g., one or more types of analytes) on the solid phase of the bead.

A bead injected or otherwise introduced into a partition may comprise releasably, cleavably, or reversibly attached barcodes. A bead injected or otherwise introduced into a partition may comprise activatable barcodes. A bead injected or otherwise introduced into a partition may be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

As will be appreciated from the above disclosure, the degradation of a bead may refer to the disassociation of a bound or entrained species from a bead, both with and without structurally degrading the physical bead itself. For example, the degradation of the bead may involve cleavage of a cleavable linkage via one or more species and/or methods described elsewhere herein. In another example, entrained species may be released from beads through osmotic pressure differences due to, for example, changing chemical environments. See, e.g., PCT/US2014/044398, which is hereby incorporated by reference in its entirety.

A degradable bead may be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) may interact with other reagents contained in the partition. See, e.g., PCT/US2014/044398, which is hereby incorporated by reference in its entirety.

As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

In some cases, a species (e.g., oligonucleotide molecules comprising barcodes) that are attached to a solid support (e.g., a bead) may comprise a U-excising element that allows the species to release from the bead. In some cases, the U-excising element may comprise a single-stranded DNA (ssDNA) sequence that contains at least one uracil. The species may be attached to a solid support via the ssDNA sequence containing the at least one uracil. The species may be released by a combination of uracil-DNA glycosylase (e.g., to remove the uracil) and an endonuclease (e.g., to induce an ssDNA break). If the endonuclease generates a 5′ phosphate group from the cleavage, then additional enzyme treatment may be included in downstream processing to eliminate the phosphate group, e.g., prior to ligation of additional sequencing handle elements, e.g., Illumina full P5 sequence, partial P5 sequence, full R1 sequence, and/or partial R1 sequence.

The barcodes that are releasable as described herein may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

The co-partitioned nucleic acid molecules can also comprise other functional sequences useful in the processing of the nucleic acids from the co-partitioned biological particles. These sequences include, e.g., targeted or random/universal amplification primer sequences for amplifying nucleic acids (e.g., mRNA, the genomic DNA) from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences (e.g., restriction sites, transposition sites). Other mechanisms of co-partitioning oligonucleotides may also be employed, including, e.g., coalescence of two or more droplets, where one droplet contains oligonucleotides, or microdispensing of oligonucleotides (e.g., attached to a bead) into partitions, e.g., droplets within microfluidic systems.

In an example, beads are provided that include large numbers of the above-described nucleic acid barcode molecules releasably attached to the beads, where all or at least a subset of the nucleic acid barcode molecules attached to a particular bead will include a common nucleic acid barcode sequence, but where a large number of diverse barcode sequences are represented across the population of beads used. In some embodiments, hydrogel beads, e.g., comprising polyacrylamide polymer matrices, are used as a solid support and delivery vehicle for the nucleic acid barcode molecules into the partitions, as they are capable of carrying large numbers of nucleic acid barcode molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. In some cases, the population of beads provides a diverse barcode sequence library that includes about 1,000 to about 10,000 different barcode sequences, about 5,000 to about 50,000 different barcode sequences, about 10,000 to about 100,000 different barcode sequences, about 50,000 to about 1,000,000 different barcode sequences, or about 100,000 to about 10,000,000 different barcode sequences.

Additionally, beads can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. In some embodiments, the number of nucleic acid molecules including the barcode sequence on an individual bead is between about 1,000 to about 10,000 nucleic acid molecules, about 5,000 to about 50,000 nucleic acid molecules, about 10,000 to about 100,000 nucleic acid molecules, about 50,000 to about 1,000,000 nucleic acid molecules, about 100,000 to about 10,000,000 nucleic acid molecules, about 1,000,000 to about 1 billion nucleic acid molecules.

Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid barcode molecules, at least about 5,000 nucleic acid barcode molecules, at least about 10,000 nucleic acid barcode molecules, at least about 50,000 nucleic acid barcode molecules, at least about 100,000 nucleic acid barcode molecules, at least about 500,000 nucleic acids, at least about 1,000,000 nucleic acid barcode molecules, at least about 5,000,000 nucleic acid barcode molecules, at least about 10,000,000 nucleic acid barcode molecules, at least about 50,000,000 nucleic acid barcode molecules, at least about 100,000,000 nucleic acid barcode molecules, at least about 250,000,000 nucleic acid barcode molecules and in some cases at least about 1 billion nucleic acid barcode molecules.

In some cases, the resulting population of partitions provides a diverse barcode sequence library that includes about 1,000 to about 10,000 different barcode sequences, about 5,000 to about 50,000 different barcode sequences, about 10,000 to about 100,000 different barcode sequences, about 50,000 to about 1,000,000 different barcode sequences, or about 100,000 to about 10,000,000 different barcode sequences. Additionally, each partition of the population can include between about 1,000 to about 10,000 nucleic acid barcode molecules, about 5,000 to about 50,000 nucleic acid barcode molecules, about 10,000 to about 100,000 nucleic acid barcode molecules, about 50,000 to about 1,000,000 nucleic acid barcode molecules, about 100,000 to about 10,000,000 nucleic acid barcode molecules, about 1,000,000 to about 1 billion nucleic acid barcode molecules.

In some cases, it may be desirable to incorporate multiple different barcodes within a given partition, either attached to a single or multiple beads within the partition. For example, in some cases, a mixed, but known set of barcode sequences may provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules from the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles and may be degraded for release of the attached nucleic acid molecules through exposure to a reducing agent, such as DTT.

### Reagents

In accordance with certain aspects, biological particles may be partitioned along with lysis reagents in order to release the contents of the biological particles within the partition. In such cases, the lysis agents can be contacted with the biological particle suspension concurrently with, or immediately prior to, the introduction of the biological particles into the partitioning junction/droplet generation zone (e.g., junction 210), such as through an additional channel or channels upstream of the channel junction. In accordance with other aspects, additionally or alternatively, biological particles may be partitioned along with other reagents, as will be described further below.

The methods and systems of the present disclosure may comprise microfluidic devices and methods of use thereof, which may be used for co-partitioning biological particles with reagents. Such systems and methods are described in U.S. Patent Publication No. US/20190367997, which is herein incorporated by reference in its entirety for all purposes.

Beneficially, when lysis reagents and biological particles are co-partitioned, the lysis reagents can facilitate the release of the contents of the biological particles within the partition. The contents released in a partition may remain discrete from the contents of other partitions.

As will be appreciated, the channel segments of the microfluidic devices described elsewhere herein may be coupled to any of a variety of different fluid sources or receiving components, including reservoirs, tubing, manifolds, or fluidic components of other systems. As will be appreciated, the microfluidic channel structures may have various geometries and/or configurations. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, MO), as well as other commercially available lysis enzymes. Other lysis agents may additionally or alternatively be co-partitioned with the biological particles to cause the release of the biological particle's contents into the partitions. For example, in some cases, surfactant-based lysis solutions may be used to lyse cells. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion-based partitioning such as encapsulation of biological particles that may be in addition to or in place of droplet partitioning, where any pore size of the encapsulate is sufficiently small to retain nucleic acid fragments of a given size, following cellular disruption.

Alternatively, or in addition to, the lysis agents co-partitioned with the biological particles described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles (e.g., a cell or a nucleus in a polymer matrix), the biological particles may be exposed to an appropriate stimulus to release the biological particles or their contents from a co-partitioned bead. For example, in some cases, a chemical stimulus may be co-partitioned along with an encapsulated biological particle to allow for the degradation of the bead and release of the cell or its contents into the larger partition. In some cases, this stimulus may be the same as the stimulus described elsewhere herein for release of nucleic acid molecules (e.g., oligonucleotides) from their respective bead. In alternative examples, this may be a different and non-overlapping stimulus, in order to allow an encapsulated biological particle to be released into a partition at a different time from the release of nucleic acid molecules into the same partition. For a description of methods, compositions, and systems for encapsulating cells (also referred to as a “cell bead”), see, e.g., U.S. Pat. No. 10,428,326 and U.S. Pat. Pub. 20190100632, which are each incorporated by reference in their entirety.

Additional reagents may also be co-partitioned with the biological particle, such as endonucleases to fragment a biological particle's DNA, DNA polymerase enzymes and dNTPs used to amplify the biological particle's nucleic acid fragments and to attach the barcode molecular tags to the amplified fragments. Other enzymes may be co-partitioned, including without limitation, polymerase, transposase, ligase, proteinase K, DNAse, restriction enzymes, etc. Additional reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching.

In some cases, template switching can be used to increase the length of a cDNA. In some cases, template switching can be used to append a predefined nucleic acid sequence to the cDNA. Template switching is further described in International Patent Application No. PCT/US2017/068320 and U.S. Pat. No. 10,011,872, which are hereby incorporated by reference in their entirety. Template switching oligonucleotides may comprise a hybridization region and a template region. Template switching oligonucleotides are further described in PCT/US2017/068320 and U.S. Pat. No. 10,011,872 which are hereby incorporated by reference in their entirety.

Any of the reagents described in this disclosure may be encapsulated in, or otherwise coupled to, a droplet, or bead, with any chemicals, particles, and elements suitable for sample processing reactions involving biomolecules, such as, but not limited to, nucleic acid molecules and proteins. For example, a bead or droplet used in a sample preparation reaction for DNA sequencing may comprise one or more of the following reagents: enzymes, restriction enzymes (e.g., multiple cutters), ligase, polymerase, fluorophores, oligonucleotide barcodes, adapters, buffers, nucleotides (e.g., dNTPs, ddNTPs) and the like.

Additional examples of reagents include, but are not limited to buffers, acidic solution, basic solution, temperature-sensitive enzymes, pH-sensitive enzymes, light-sensitive enzymes, metals, metal ions, magnesium chloride, sodium chloride, manganese, aqueous buffer, mild buffer, ionic buffer, inhibitor, enzyme, protein, polynucleotide, antibodies, saccharides, lipid, oil, salt, ion, detergents, ionic detergents, non-ionic detergents, and oligonucleotides.

Once the contents of the cells are released into their respective partitions, the macromolecular components (e.g., macromolecular constituents of biological particles, such as RNA, DNA, or proteins) contained therein may be further processed within the partitions. In accordance with the methods and systems described herein, the macromolecular component contents of individual biological particles can be provided with unique identifiers such that, upon characterization of those macromolecular components they may be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles. In some aspects, this is performed by co-partitioning the individual biological particle or groups of biological particles with the unique identifiers, such as described above (with reference to FIG. 1 or 2).

In some cases, additional beads can be used to deliver additional reagents to a partition. In such cases, it may be advantageous to introduce different beads into a common channel or droplet generation junction, from different bead sources (e.g., containing different associated reagents) through different channel inlets into such common channel or droplet generation junction. In such cases, the flow and frequency of the different beads into the channel or junction may be controlled to provide for a certain ratio of beads from each source, while ensuring a given pairing or combination of such beads into a partition with a given number of biological particles (e.g., one biological particle and one bead per partition).

In some embodiments, following the generation of barcoded nucleic acid molecules according to methods disclosed herein, subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations may occur in bulk (e.g., outside the partition). In the case where a partition is a droplet in an emulsion, the emulsion can be broken, and the contents of the droplet pooled for additional operations.

### Wells

As described herein, one or more processes may be performed in a partition, which may be a well. The well may be a well of a plurality of wells of a substrate, such as a microwell of a microwell array or plate, or the well may be a microwell or microchamber of a device (e.g., microfluidic device) comprising a substrate. The well may be a well of a well array or plate, or the well may be a well or chamber of a device (e.g., fluidic device). In some embodiments, a well of a fluidic device is fluidically connected to another well of the fluidic device. Accordingly, the wells or microwells may assume an “open” configuration, in which the wells or microwells are exposed to the environment (e.g., contain an open surface) and are accessible on one planar face of the substrate, or the wells or microwells may assume a “closed” or “sealed” configuration, in which the microwells are not accessible on a planar face of the substrate. In some instances, the wells or microwells may be configured to toggle between “open” and “closed” configurations. For instance, an “open” microwell or set of microwells may be “closed” or “sealed” using a membrane (e.g., semi-permeable membrane), an oil (e.g., fluorinated oil to cover an aqueous solution), or a lid, as described elsewhere herein.

The well may have a volume of less than 1 milliliter (mL). For instance, the well may be configured to hold a volume of at most 1000 microliters (μL), at most 100 μL, at most 10 μL, at most 1 μL, at most 100 nanoliters (nL), at most 10 nL, at most 1 nL, at most 100 picoliters (pL), at most 10 (pL), or less. The well may be configured to hold a volume of about 1000 μL, about 100 μL, about 10 μL, about 1 μL, about 100 nL, about 10 nL, about 1 nL, about 100 pL, about 10 pL, etc. The well may be configured to hold a volume of at least 10 pL, at least 100 pL, at least 1 nL, at least 10 nL, at least 100 nL, at least 1 μL, at least 10 μL, at least 100 μL, at least 1000 μL, or more. The well may be configured to hold a volume in a range of volumes listed herein, for example, from about 5 nL to about 20 nL, from about 1 nL to about 100 nL, from about 500 pL to about 100 μL, etc. The well may be of a plurality of wells that have varying volumes and may be configured to hold a volume appropriate to accommodate any of the partition volumes described herein.

In some instances, a microwell array or plate comprises a single variety of microwells. In some instances, a microwell array or plate comprises a variety of microwells. For instance, the microwell array or plate may comprise one or more types of microwells within a single microwell array or plate. The types of microwells may have different dimensions (e.g., length, width, diameter, depth, cross-sectional area, etc.), shapes (e.g., circular, triangular, square, rectangular, pentagonal, hexagonal, heptagonal, octagonal, nonagonal, decagonal, etc.), aspect ratios, or other physical characteristics. The microwell array or plate may comprise any number of different types of microwells. For example, the microwell array or plate may comprise 1, 2, 3, 4, 5, 6, 7, 8, 9, 10, 20, 30, 40, 50, 60, 70, 80, 90, 100, 200, 300, 400, 500, 600, 700, 800, 900, 1000 or more different types of microwells. A well may have any dimension (e.g., length, width, diameter, depth, cross-sectional area, volume, etc.), shape (e.g., circular, triangular, square, rectangular, pentagonal, hexagonal, heptagonal, octagonal, nonagonal, decagonal, other polygonal, etc.), aspect ratios, or other physical characteristics described herein with respect to any well.

In certain instances, the microwell array or plate comprises different types of microwells that are located adjacent to one another within the array or plate. For instance, a microwell with one set of dimensions may be located adjacent to and in contact with another microwell with a different set of dimensions. Similarly, microwells of different geometries may be placed adjacent to or in contact with one another. The adjacent microwells may be configured to hold different articles; for example, one microwell may be used to contain a cell, cell bead, or other sample (e.g., cellular components, nucleic acid molecules, etc.) while the adjacent microwell may be used to contain a droplet, bead, or other reagent. In some cases, the adjacent microwells may be configured to merge the contents held within, e.g., upon application of a stimulus, or spontaneously, upon contact of the articles in each microwell.

As is described elsewhere herein, a plurality of partitions may be used in the systems, compositions, and methods described herein. For example, any suitable number of partitions (e.g., wells or droplets) can be generated or otherwise provided. For example, in the case when wells are used, at least about 1,000 wells, at least about 5,000 wells, at least about 10,000 wells, at least about 50,000 wells, at least about 100,000 wells, at least about 500,000 wells, at least about 1,000,000 wells, at least about 5,000,000 wells at least about 10,000,000 wells, at least about 50,000,000 wells, at least about 100,000,000 wells, at least about 500,000,000 wells, at least about 1,000,000,000 wells, or more wells can be generated or otherwise provided. Moreover, the plurality of wells may comprise both unoccupied wells (e.g., empty wells) and occupied wells.

A well may comprise any of the reagents described herein, or combinations thereof. These reagents may include, for example, barcode molecules, enzymes, adapters, and combinations thereof. The reagents may be physically separated from a sample (e.g., a cell, cell bead, or cellular components, e.g., proteins, nucleic acid molecules, etc.) that is placed in the well. This physical separation may be accomplished by containing the reagents within, or coupling to, a bead that is placed within a well. The physical separation may also be accomplished by dispensing the reagents in the well and overlaying the reagents with a layer that is, for example, dissolvable, meltable, or permeable prior to introducing the polynucleotide sample into the well. This layer may be, for example, an oil, wax, membrane (e.g., semi-permeable membrane), or the like. The well may be sealed at any point, for example, after addition of the bead, after addition of the reagents, or after addition of either of these components. The sealing of the well may be useful for a variety of purposes, including preventing escape of beads or loaded reagents from the well, permitting select delivery of certain reagents (e.g., via the use of a semi-permeable membrane), for storage of the well prior to or following further processing, etc.

Once sealed, the well may be subjected to conditions for further processing of a cell (or cells) in the well. For instance, reagents in the well may allow further processing of the cell, e.g., cell lysis, as further described herein. Alternatively, the well (or wells such as those of a well-based array) comprising the cell (or cells) may be subjected to freeze-thaw cycling to process the cell (or cells), e.g., cell lysis. The well containing the cell may be subjected to freezing temperatures (e.g., 0° C., below 0° C., −5° C., −10° C., −15° C., −20° C., −25° C., −30° C., −35° C., −40° C., −45° C., −50° C., −55° C., −60° C., −65° C., −70° C., −80° C., or −85° C.). Freezing may be performed in a suitable manner, e.g., sub-zero freezer or a dry ice/ethanol bath. Following an initial freezing, the well (or wells) comprising the cell (or cells) may be subjected to freeze-thaw cycles to lyse the cell (or cells). In one embodiment, the initially frozen well (or wells) are thawed to a temperature above freezing (e.g., 4° C. or above, 8° C. or above, 12° C. or above, 16° C. or above, 20° C. or above, room temperature, or 25° C. or above). In another embodiment, the freezing is performed for less than 10 minutes (e.g., 5 minutes or 7 minutes) followed by thawing at room temperature for less than 10 minutes (e.g., 5 minutes or 7 minutes). This freeze-thaw cycle may be repeated a number of times, e.g., 2, 3, 4 or more times, to obtain lysis of the cell (or cells) in the well (or wells). In one embodiment, the freezing, thawing and/or freeze/thaw cycling is performed in the absence of a lysis buffer. Additional disclosure related to freeze-thaw cycling is provided in WO2019165181A1, which is incorporated herein by reference in its entirety.

A well may comprise free reagents and/or reagents encapsulated in, or otherwise coupled to or associated with, beads, beads, or droplets.

The wells may be provided as a part of a kit. For example, a kit may comprise instructions for use, a microwell array or device, and reagents (e.g., beads). The kit may comprise any useful reagents for performing the processes described herein, e.g., nucleic acid reactions, barcoding of nucleic acid molecules, sample processing (e.g., for cell lysis, fixation, and/or permeabilization).

In some cases, a well comprises a bead, or droplet that comprises a set of reagents that has a similar attribute (e.g., a set of enzymes, a set of minerals, a set of oligonucleotides, a mixture of different barcode molecules, a mixture of identical barcode molecules). In other cases, a bead or droplet comprises a heterogeneous mixture of reagents. In some cases, the heterogeneous mixture of reagents can comprise all components necessary to perform a reaction. In some cases, such mixture can comprise all components necessary to perform a reaction, except for 1, 2, 3, 4, 5, or more components necessary to perform a reaction. In some cases, such additional components are contained within, or otherwise coupled to, a different droplet or bead, or within a solution within a partition (e.g., microwell) of the system.

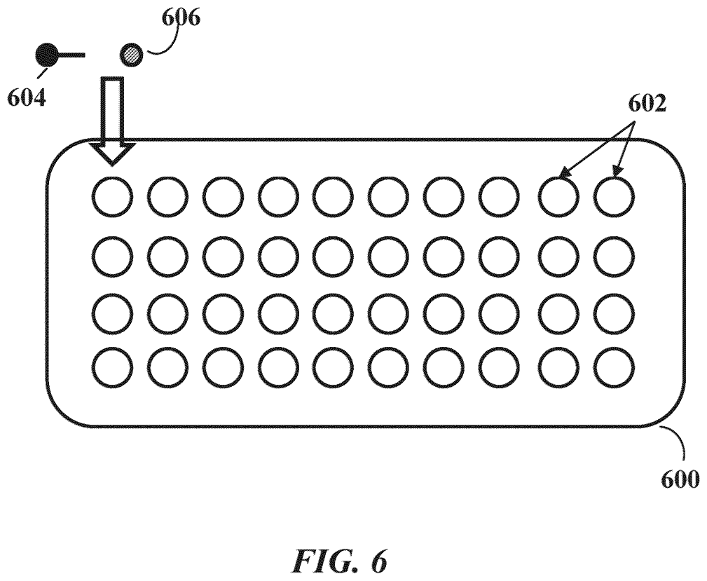

FIG. 6 schematically illustrates an example of a microwell array. The array can be contained within a substrate 600. The substrate 600 comprises a plurality of wells 602. The wells 602 may be of any size or shape, and the spacing between the wells, the number of wells per substrate, as well as the density of the wells on the substrate 600 can be modified, depending on the particular application. In one such example application, a sample molecule 606, which may comprise a cell or cellular components (e.g., nucleic acid molecules) is co-partitioned with a bead 604, which may comprise a nucleic acid barcode molecule coupled thereto. The wells 602 may be loaded using gravity or other loading technique (e.g., centrifugation, liquid handler, acoustic loading, optoelectronic, etc.). In some instances, at least one of the wells 602 contains a single sample molecule 606 (e.g., cell) and a single bead 604.

Reagents may be loaded into a well either sequentially or concurrently. In some cases, reagents are introduced to the device either before or after a particular operation. In some cases, reagents (which may be provided, in certain instances, in droplets, or beads) are introduced sequentially such that different reactions or operations occur at different steps. The reagents (or droplets, or beads) may also be loaded at operations interspersed with a reaction or operation step. For example, beads (or droplets) comprising reagents for fragmenting polynucleotides (e.g., restriction enzymes) and/or other enzymes (e.g., transposases, ligases, polymerases, etc.) may be loaded into the well or plurality of wells, followed by loading of droplets, or beads comprising reagents for attaching nucleic acid barcode molecules to a sample nucleic acid molecule. Reagents may be provided concurrently or sequentially with a sample, e.g., a cell or cellular components (e.g., organelles, proteins, nucleic acid molecules, carbohydrates, lipids, etc.). Accordingly, use of wells may be useful in performing multi-step operations or reactions.

As described elsewhere herein, the nucleic acid barcode molecules and other reagents may be contained within a bead, or droplet. These beads, or droplets may be loaded into a partition (e.g., a microwell) before, after, or concurrently with the loading of a cell, such that each cell is contacted with a different bead, or droplet. This technique may be used to attach a unique nucleic acid barcode molecule to nucleic acid molecules obtained from each cell. Alternatively, or in addition to, the sample nucleic acid molecules may be attached to a support. For instance, the partition (e.g., microwell) may comprise a bead which has coupled thereto a plurality of nucleic acid barcode molecules. The sample nucleic acid molecules, or derivatives thereof, may couple or attach to the nucleic acid barcode molecules on the support. The resulting barcoded nucleic acid molecules may then be removed from the partition, and in some instances, pooled and sequenced. In such cases, the nucleic acid barcode sequences may be used to trace the origin of the sample nucleic acid molecule. For example, polynucleotides with identical barcodes may be determined to originate from the same cell or partition, while polynucleotides with different barcodes may be determined to originate from different cells or partitions.

The samples or reagents may be loaded in the wells or microwells using a variety of approaches. The samples (e.g., a cell, cell bead, or cellular component) or reagents (as described herein) may be loaded into the well or microwell using an external force, e.g., gravitational force, electrical force, magnetic force, or using mechanisms to drive the sample or reagents into the well, e.g., via pressure-driven flow, centrifugation, optoelectronics, acoustic loading, electrokinetic pumping, vacuum, capillary flow, etc. In certain cases, a fluid handling system may be used to load the samples or reagents into the well. The loading of the samples or reagents may follow a Poissonian distribution or a non-Poissonian distribution, e.g., super Poisson or sub-Poisson. The geometry, spacing between wells, density, and size of the microwells may be modified to accommodate a useful sample or reagent distribution; for instance, the size and spacing of the microwells may be adjusted such that the sample or reagents may be distributed in a super-Poissonian fashion.

In one particular non-limiting example, the microwell array or plate comprises pairs of microwells, in which each pair of microwells is configured to hold a droplet (e.g., comprising a single cell) and a single bead (such as those described herein, which may, in some instances, also be encapsulated in a droplet). The droplet and the bead (or droplet containing the bead) may be loaded simultaneously or sequentially, and the droplet and the bead may be merged, e.g., upon contact of the droplet and the bead, or upon application of a stimulus (e.g., external force, agitation, heat, light, magnetic or electric force, etc.). In some cases, the loading of the droplet and the bead is super-Poissonian. In other examples of pairs of microwells, the wells are configured to hold two droplets comprising different reagents and/or samples, which are merged upon contact or upon application of a stimulus. In such instances, the droplet of one microwell of the pair can comprise reagents that may react with an agent in the droplet of the other microwell of the pair. For instance, one droplet can comprise reagents that are configured to release the nucleic acid barcode molecules of a bead contained in another droplet, located in the adjacent microwell. Upon merging of the droplets, the nucleic acid barcode molecules may be released from the bead into the partition (e.g., the microwell or microwell pair that are in contact), and further processing may be performed (e.g., barcoding, nucleic acid reactions, etc.). In cases where intact or live cells are loaded in the microwells, one of the droplets may comprise lysis reagents for lysing the cell upon droplet merging.

A droplet or bead may be partitioned into a well. The droplets may be selected or subjected to pre-processing prior to loading into a well. For instance, the droplets may comprise cells, and only certain droplets, such as those containing a single cell (or at least one cell), may be selected for use in loading of the wells. Such a pre-selection process may be useful in efficient loading of single cells, such as to obtain a non-Poissonian distribution, or to pre-filter cells for a selected characteristic prior to further partitioning in the wells. Additionally, the technique may be useful in obtaining or preventing cell doublet or multiplet formation prior to or during loading of the microwell.

In some instances, the wells can comprise nucleic acid barcode molecules attached thereto. The nucleic acid barcode molecules may be attached to a surface of the well (e.g., a wall of the well). The nucleic acid barcode molecules may be attached to a droplet or bead that has been partitioned into the well. The nucleic acid barcode molecule (e.g., a partition barcode sequence) of one well may differ from the nucleic acid barcode molecule of another well, which can permit identification of the contents contained with a single partition or well. In some cases, the nucleic acid barcode molecule can comprise a spatial barcode sequence that can identify a spatial coordinate of a well, such as within the well array or well plate. In some cases, the nucleic acid barcode molecule can comprise a unique molecular identifier for individual molecule identification. In some instances, the nucleic acid barcode molecules may be configured to attach to or capture a nucleic acid molecule within a sample or cell distributed in the well. For example, the nucleic acid barcode molecules may comprise a capture sequence that may be used to capture or hybridize to a nucleic acid molecule (e.g., RNA, DNA) within the sample. In some instances, the nucleic acid barcode molecules may be releasable from the microwell. In some instances, the nucleic acid barcode molecules may be releasable from the bead or droplet. For instance, the nucleic acid barcode molecules may comprise a chemical cross-linker which may be cleaved upon application of a stimulus (e.g., photo-, magnetic, chemical, biological, stimulus). The nucleic acid barcode molecules, which may be hybridized or configured to hybridize to a sample nucleic acid molecule, may be collected and pooled for further processing, which can include nucleic acid processing (e.g., amplification, extension, reverse transcription, etc.) and/or characterization (e.g., sequencing). In some instances, nucleic acid barcode molecules attached to a bead in a well may be hybridized to sample nucleic acid molecules, and the bead with the sample nucleic acid molecules hybridized thereto may be collected and pooled for further processing, which can include nucleic acid processing (e.g., amplification, extension, reverse transcription, etc.) and/or characterization (e.g., sequencing). In such cases, the unique partition barcode sequences may be used to identify the cell or partition from which a nucleic acid molecule originated.

Characterization of samples within a well may be performed. Such characterization can include, in non-limiting examples, imaging of the sample (e.g., cell, cell bead, or cellular components) or derivatives thereof. Characterization techniques such as microscopy or imaging may be useful in measuring sample profiles in fixed spatial locations. For instance, when cells are partitioned, optionally with beads, imaging of each microwell and the contents contained therein may provide useful information on cell doublet formation (e.g., frequency, spatial locations, etc.), cell-bead pair efficiency, cell viability, cell size, cell morphology, expression level of a biomarker (e.g., a surface marker, a fluorescently labeled molecule therein, etc.), cell or bead loading rate, number of cell-bead pairs, etc. In some instances, imaging may be used to characterize live cells in the wells, including, but not limited to dynamic live-cell tracking, cell-cell interactions (when two or more cells are co-partitioned), cell proliferation, etc. Alternatively, or in addition to, imaging may be used to characterize a quantity of amplification products in the well.

In operation, a well may be loaded with a sample and reagents, simultaneously or sequentially. When cells or cell beads are loaded, the well may be subjected to washing, e.g., to remove excess cells from the well, microwell array, or plate. Similarly, washing may be performed to remove excess beads or other reagents from the well, microwell array, or plate. In the instances where live cells are used, the cells may be lysed in the individual partitions to release the intracellular components or cellular analytes. Alternatively, the cells may be fixed or permeabilized in the individual partitions. The intracellular components or cellular analytes may couple to a support, e.g., on a surface of the microwell, on a solid support (e.g., bead), or they may be collected for further downstream processing. For instance, after cell lysis, the intracellular components or cellular analytes may be transferred to individual droplets or other partitions for barcoding. Alternatively, or in addition to, the intracellular components or cellular analytes (e.g., nucleic acid molecules) may couple to a bead comprising a nucleic acid barcode molecule; subsequently, the bead may be collected and further processed, e.g., subjected to nucleic acid reaction such as reverse transcription, amplification, or extension, and the nucleic acid molecules thereon may be further characterized, e.g., via sequencing. Alternatively, or in addition to, the intracellular components or cellular analytes may be barcoded in the well (e.g., using a bead comprising nucleic acid barcode molecules that are releasable or on a surface of the microwell comprising nucleic acid barcode molecules). The barcoded nucleic acid molecules or analytes may be further processed in the well, or the barcoded nucleic acid molecules or analytes may be collected from the individual partitions and subjected to further processing outside the partition. Further processing can include nucleic acid processing (e.g., performing an amplification, extension) or characterization (e.g., fluorescence monitoring of amplified molecules, sequencing). At any convenient or useful step, the well (or microwell array or plate) may be sealed (e.g., using an oil, membrane, wax, etc.), which enables storage of the assay or selective introduction of additional reagents.

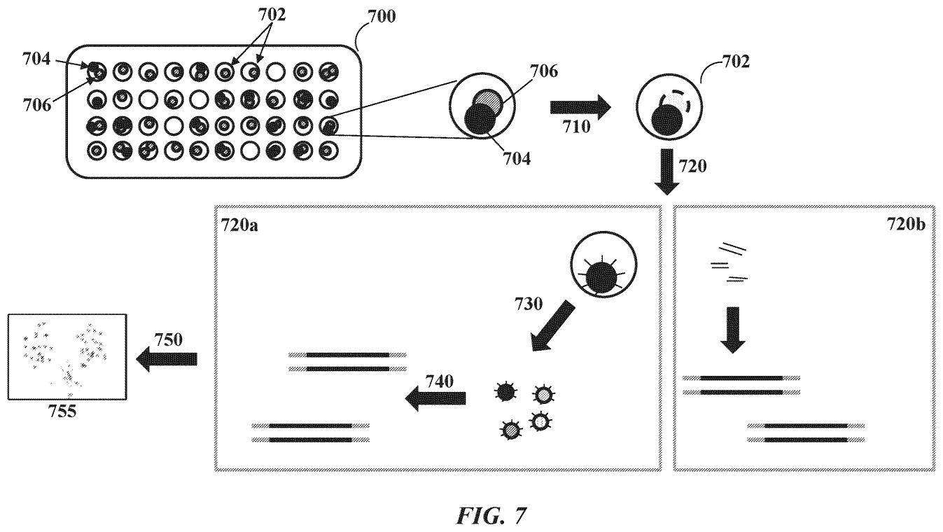

FIG. 7 schematically shows an example workflow for processing nucleic acid molecules within a sample. A substrate 700 comprising a plurality of microwells 702 may be provided. A sample 706 which may comprise a cell, cell bead, cellular components or analytes (e.g., proteins and/or nucleic acid molecules) can be co-partitioned, in a plurality of microwells 702, with a plurality of beads 704 comprising nucleic acid barcode molecules. During process 710, the sample 706 may be processed within the partition. For instance, in the case of live cells, the cell may be subjected to conditions sufficient to lyse the cells and release the analytes contained therein. In process 720, the bead 704 may be further processed. By way of example, processes 720a and 720b schematically illustrate different workflows, depending on the properties of the bead 704.

In 720a, the bead comprises nucleic acid barcode molecules that are attached thereto, and sample nucleic acid molecules (e.g., RNA, DNA) may attach, e.g., via hybridization of ligation, to the nucleic acid barcode molecules. Such attachment may occur on the bead. In process 730, the beads 704 from multiple wells 702 may be collected and pooled. Further processing may be performed in process 740. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 750, further characterization, such as sequencing may be performed to generate sequencing reads. The sequencing reads may yield information on individual cells or populations of cells, which may be represented visually or graphically, e.g., in a plot 755.

In 720b, the bead comprises nucleic acid barcode molecules that are releasably attached thereto, as described below. The bead may degrade or otherwise release the nucleic acid barcode molecules into the well 702; the nucleic acid barcode molecules may then be used to barcode nucleic acid molecules within the well 702. Further processing may be performed either inside the partition or outside the partition. For example, one or more nucleic acid reactions may be performed, such as reverse transcription, nucleic acid extension, amplification, ligation, transposition, etc. In some instances, adapter sequences are ligated to the nucleic acid molecules, or derivatives thereof, as described elsewhere herein. For instance, sequencing primer sequences may be appended to each end of the nucleic acid molecule. In process 750, further characterization, such as sequencing may be performed to generate sequencing reads. The sequencing reads may yield information on individual cells or populations of cells, which may be represented visually or graphically, e.g., in a plot 755.

### Sample and Cell Processing

A sample may derive from any useful source including any subject, such as a human subject. A sample may comprise material (e.g., one or more biological particles) from one or more different sources, such as one or more different subjects. Multiple samples, such as multiple samples from a single subject (e.g., multiple samples obtained in the same or different manners from the same or different bodily locations, and/or obtained at the same or different times (e.g., seconds, minutes, hours, days, weeks, months, or years apparat)), or multiple samples from different subjects, may be obtained for analysis as described herein. For example, a first sample may be obtained from a subject at a first time and a second sample may be obtained from the subject at a second time later than the first time. The first time may be before a subject undergoes a treatment regimen or procedure (e.g., to address a disease or condition), and the second time may be during or after the subject undergoes the treatment regimen or procedure. In another example, a first sample may be obtained from a first bodily location or system of a subject (e.g., using a first collection technique) and a second sample may be obtained from a second bodily location or system of the subject (e.g., using a second collection technique), which second bodily location or system may be different than the first bodily location or system. In another example, multiple samples may be obtained from a subject at a same time from the same or different bodily locations. Different samples, such as different subjects collected from different bodily locations of a same subject, at different times, from multiple different subjects, and/or using different collection techniques, may undergo the same or different processing (e.g., as described herein). For example, a first sample may undergo a first processing protocol and a second sample may undergo a second processing protocol. In another example, a portion of a sample may undergo a first processing protocol and a second portion of the sample may undergo a second processing protocol.

A sample may be a biological sample, such as a cell sample (e.g., as described herein). A sample may include one or more biological particles, such as one or more cells and/or cellular constituents, such as one or more cell nuclei. A sample may be a tissue sample. For example, a sample may comprise a plurality of biological particles, such as a plurality of cells and/or cellular constituents. Biological particles (e.g., cells or cellular constituents, such as cell nuclei) of a sample may be of a single type or a plurality of different types. For example, cells of a sample may include one or more different types or blood cells.

Cells and cellular constituents of a sample may be of any type. For example, a cell or cellular constituent may be a vertebral, mammalian, fungal, plant, bacterial, or other cell type. In some cases, the cell is a mammalian cell, such as a human cell. The cell may be, for example, a stem cell, liver cell, nerve cell, bone cell, blood cell, reproductive cell, skin cell, skeletal muscle cell, cardiac muscle cell, smooth muscle cell, hair cell, hormone-secreting cell, or glandular cell. The cell may be, for example, an erythrocyte (e.g., red blood cell), a megakaryocyte (e.g., platelet precursor), a monocyte (e.g., white blood cell), a leukocyte, a B cell, a T cell (such as a helper, suppressor, cytotoxic, or natural killer T cell), an osteoclast, a dendritic cell, a connective tissue macrophage, an epidermal Langerhans cell, a microglial cell, a granulocyte, a hybridoma cell, a mast cell, a natural killer cell, a reticulocyte, a hematopoietic stem cell, a myoepithelial cell, a myeloid-derived suppressor cell, a platelet, a thymocyte, a satellite cell, an epithelial cell, an endothelial cell, an epididymal cell, a kidney cell, a liver cell, an adipocyte, a lipocyte, or a neuron cell. In some cases, the cell may be associated with a cancer, tumor, or neoplasm. In some cases, the cell may be associated with a fetus. In some cases, the cell may be a Jurkat cell.

A biological sample may include a plurality of cells having different dimensions and features. In some cases, processing of the biological sample, such as cell separation and sorting (e.g., as described herein), may affect the distribution of dimensions and cellular features included in the sample by depleting cells having certain features and dimensions and/or isolating cells having certain features and dimensions.

A sample may undergo one or more processes in preparation for analysis (e.g., as described herein), including, but not limited to, filtration, selective precipitation, purification, centrifugation, permeabilization, isolation, agitation, heating, and/or other processes. For example, a sample may be filtered to remove a contaminant or other materials. In an example, a filtration process may comprise the use of microfluidics (e.g., to separate biological particles of different sizes, types, charges, or other features).

In an example, a sample comprising one or more cells may be processed to separate the one or more cells from other materials in the sample (e.g., using centrifugation and/or another process). In some cases, cells and/or cellular constituents of a sample may be processed to separate and/or sort groups of cells and/or cellular constituents, such as to separate and/or sort cells and/or cellular constituents of different types. Examples of cell separation include, but are not limited to, separation of white blood cells or immune cells from other blood cells and components, separation of circulating tumor cells from blood, and separation of bacteria from bodily cells and/or environmental materials. A separation process may comprise a positive selection process (e.g., targeting of a cell type of interest for retention for subsequent downstream analysis, such as by use of a monoclonal antibody that targets a surface marker of the cell type of interest), a negative selection process (e.g., removal of one or more cell types and retention of one or more other cell types of interest), and/or a depletion process (e.g., removal of a single cell type from a sample, such as removal of red blood cells from peripheral blood mononuclear cells).

Separation of one or more different types of cells may comprise, for example, centrifugation, filtration, microfluidic-based sorting, flow cytometry, fluorescence-activated cell sorting (FACS), magnetic-activated cell sorting (MACS), buoyancy-activated cell sorting (BACS), or any other useful method.

For example, a flow cytometry method may be used to detect cells and/or cellular constituents based on a parameter such as a size, morphology, or protein expression. Flow cytometry-based cell sorting may comprise injecting a sample into a sheath fluid that conveys the cells and/or cellular constituents of the sample into a measurement region one at a time. In the measurement region, a light source such as a laser may interrogate the cells and/or cellular constituents and scattered light and/or fluorescence may be detected and converted into digital signals. A nozzle system (e.g., a vibrating nozzle system) may be used to generate droplets (e.g., aqueous droplets) comprising individual cells and/or cellular constituents. Droplets including cells and/or cellular constituents of interest (e.g., as determined via optical detection) may be labeled with an electric charge (e.g., using an electrical charging ring), which charge may be used to separate such droplets from droplets including other cells and/or cellular constituents. For example, FACS may comprise labeling cells and/or cellular constituents with fluorescent markers (e.g., using internal and/or external biomarkers). Cells and/or cellular constituents may then be measured and identified one by one and sorted based on the emitted fluorescence of the marker or absence thereof. MACS may use micro- or nano-scale magnetic particles to bind to cells and/or cellular constituents (e.g., via an antibody interaction with cell surface markers) to facilitate magnetic isolation of cells and/or cellular constituents of interest from other components of a sample (e.g., using a column-based analysis). BACS may use microbubbles (e.g., glass microbubbles) labeled with antibodies to target cells of interest. Cells and/or cellular components coupled to microbubbles may float to a surface of a solution, thereby separating target cells and/or cellular components from other components of a sample. Cell separation techniques may be used to enrich for populations of cells of interest (e.g., prior to partitioning, as described herein). For example, a sample comprising a plurality of cells including a plurality of cells of a given type may be subjected to a positive separation process. The plurality of cells of the given type may be labeled with a fluorescent marker (e.g., based on an expressed cell surface marker or another marker) and subjected to a FACS process to separate these cells from other cells of the plurality of cells. The selected cells may then be subjected to subsequent partition-based analysis (e.g., as described herein) or other downstream analysis. The fluorescent marker may be removed prior to such analysis or may be retained. The fluorescent marker may comprise an identifying feature, such as a nucleic acid barcode sequence and/or unique molecular identifier.

In another example, a first sample comprising a first plurality of cells including a first plurality of cells of a given type (e.g., immune cells expressing a particular marker or combination of markers) and a second sample comprising a second plurality of cells including a second plurality of cells of the given type may be subjected to a positive separation process. The first and second samples may be collected from the same or different subjects, at the same or different types, from the same or different bodily locations or systems, using the same or different collection techniques. For example, the first sample may be from a first subject and the second sample may be from a second subject different than the first subject. The first plurality of cells of the first sample may be provided a first plurality of fluorescent markers configured to label the first plurality of cells of the given type. The second plurality of cells of the second sample may be provided a second plurality of fluorescent markers configured to label the second plurality of cells of the given type. The first plurality of fluorescent markers may include a first identifying feature, such as a first barcode, while the second plurality of fluorescent markers may include a second identifying feature, such as a second barcode, that is different than the first identifying feature. The first plurality of fluorescent markers and the second plurality of fluorescent markers may fluoresce at the same intensities and over the same range of wavelengths upon excitation with a same excitation source (e.g., light source, such as a laser). The first and second samples may then be combined and subjected to a FACS process to separate cells of the given type from other cells based on the first plurality of fluorescent markers labeling the first plurality of cells of the given type and the second plurality of fluorescent markers labeling the second plurality of cells of the given type. Alternatively, the first and second samples may undergo separate FACS processes and the positively selected cells of the given type from the first sample and the positively selected cells of the given type from the second sample may then be combined for subsequent analysis. The encoded identifying features of the different fluorescent markers may be used to identify cells originating from the first sample and cells originating from the second sample. For example, the first and second identifying features may be configured to interact (e.g., in partitions, as described herein) with nucleic acid barcode molecules (e.g., as described herein) to generate barcoded nucleic acid products detectable using, e.g., nucleic acid sequencing.

### Fixed Samples

A sample may be a fixed sample. For example, a sample may comprise a plurality of fixed samples, such as a plurality of fixed cells or fixed nuclei. Alternatively, or in addition, a sample may comprise a fixed tissue. Fixation of a cell or cellular constituent, or a tissue comprising a plurality of cells or nuclei, may comprise application of a chemical species or chemical stimulus. The term “fixed” as used herein with regard to biological samples generally refers to the state of being preserved from decay and/or degradation. “Fixation” generally refers to a process that results in a fixed sample, and in some instances can include contacting the biomolecules within a biological sample with a fixative (or fixation reagent) for some amount of time, whereby the fixative results in covalent bonding interactions such as crosslinks between biomolecules in the sample. A “fixed biological sample” may generally refer to a biological sample that has been contacted with a fixation reagent or fixative. For example, a formaldehyde-fixed biological sample has been contacted with the fixation reagent formaldehyde. “Fixed cells” or “fixed tissues” generally refer to cells or tissues that have been in contact with a fixative under conditions sufficient to allow or result in the formation of intra- and inter-molecular covalent crosslinks between biomolecules in the biological sample. Generally, contact of biological sample (e.g., a cell or nucleus) with a fixation reagent (e.g., paraformaldehyde or PFA) results in the formation of intra- and inter-molecular covalent crosslinks between biomolecules in the biological sample. In some cases, provision of the fixation reagent, such as formaldehyde, may result in covalent aminal crosslinks within RNA, DNA, and/or protein molecules. For example, the widely used fixative reagent, paraformaldehyde or PFA, fixes tissue samples by catalyzing crosslink formation between basic amino acids in proteins, such as lysine and glutamine. Both intra-molecular and inter-molecular crosslinks can form in the protein. These crosslinks can preserve protein secondary structure and also eliminate enzymatic activity in the preserved tissue sample. Examples of fixation reagents include but are not limited to aldehyde fixatives (e.g., formaldehyde, also commonly referred to as “paraformaldehyde,” “PFA,” and “formalin”; glutaraldehyde; etc.), imidoesters, NHS (N-Hydroxysuccinimide) esters, and the like.

Other examples of fixation reagents include, for example, organic solvents such as alcohols (e.g., methanol, ethanol, isopropanol, butanol), ketones (e.g., acetone), and aldehydes (e.g., paraformaldehyde, formaldehyde (e.g., formalin), or glutaraldehyde). Organic solvents may be used in combination with other fixatives or crosslinkers. For example, the combination of methanol and dithio-bis(-succinimidyl propionate) (DSP) may be used. Combinations of any alcohol with one or more fixatives or crosslinkers is envisioned. Examples of combinations include, but are not limited to, i) methanol and DSP, ii) methanol, DSP, and DTSSP, iii) ethanol and DSP, iv) ethanol, DSP, and DTSSP, v.) methanol and EGS, vi.) ethanol and EGS, v.) DSP and acetone, vi) paraformaldehyde and methanol, and vii) formaldehyde and methanol.

In some embodiments, a sample (e.g., tissue, whole blood, bone marrow aspirate, cells, e.g., peripheral blood mononuclear cells (PBMCs), leukocytes, bone marrow mononuclear cells (BMMCs), healthy cells, diseased cells, e.g., cells from a subject having a disease or disorder, e.g., cancer) is fixed using a fixation buffer comprising DSP and methanol. In some embodiments, a DSP solution is prepared by dissolving DSP in a solvent, e.g. DMSO. In some embodiments, a fixation buffer is prepared by mixing the dissolved DSP with methanol. In some embodiments, the fixation buffer comprises about 10%, 20%, 30%, 40%, 50%, 60%, 70%, 80%, 90%, 99%, 99.9%, 100% methanol. In some embodiments, the fixation buffer comprises 10%, 20%, 30%, 40%, 50%, 60%, 70%, 80%, 90%, 99%, 99.9%, 100% methanol. In some embodiments, the fixation buffer comprises a final concentration of DSP between about 0.25 mM and about 5 mM, and methanol. In some embodiments, the fixation buffer comprises a final concentration of DSP between about 0.5 mM and about 10 mM, and methanol. In some embodiments, the fixation buffer comprises a final concentration of DSP between about 5 mM and about 20 mM, and methanol. In some embodiments, the fixation buffer comprises a final concentration of about 3.125 mM DSP. In some embodiments, the fixation buffer comprises a final concentration of about 3.125 mM DSP and 100% methanol. In some embodiments, the fixation buffer comprises a final concentration of DSP between 0.25 mM-5 mM, and methanol. In some embodiments, the fixation buffer comprises a final concentration of DSP between 0.5 mM-10 mM, and methanol. In some embodiments, the fixation buffer comprises a final concentration of DSP between 5 mM-20 mM, and methanol. In some embodiments, the fixation buffer comprises a final concentration of 3.125 mM DSP. In some embodiments, the fixation buffer comprises a final concentration of 3.125 mM DSP and 100% methanol. In some embodiments, the fixation buffer comprises a final concentration of DSP and methanol as listed in Table 1.

TABLE 1
Exemplary combinations of concentrations ([ ])
of DSP and methanol (MeOH) in a fixation buffer.

| DSP | | DSP | | DSP | | DSP | |
|---|---|---|---|---|---|---|---|
| final | MeOH | final | MeOH | final | MeOH | final | MeOH |
| [ ] (mM) | [ ] | [ ] (mM) | [ ] | [ ] | [ ] | [ ] (mM) | [ ] |
| 0.5 | 100% | 0.5 | 90% | 0.5 | 80% | 0.5 | 70% |
| 1.0 | 100% | 1.0 | 90% | 1.0 | 80% | 1.0 | 70% |
| 1.5 | 100% | 1.5 | 90% | 1.5 | 80% | 1.5 | 70% |
| 2.0 | 100% | 2.0 | 90% | 2.0 | 80% | 2.0 | 70% |
| 2.5 | 100% | 2.5 | 90% | 2.5 | 80% | 2.5 | 70% |
| 3.0 | 100% | 3.0 | 90% | 3.0 | 80% | 3.0 | 70% |
| 3.125 | 100% | 3.125 | 90% | 3.125 | 80% | 3.125 | 70% |
| 3.2 | 100% | 3.2 | 90% | 3.2 | 80% | 3.2 | 70% |
| 3.3 | 100% | 3.3 | 90% | 3.3 | 80% | 3.3 | 70% |
| 3.5 | 100% | 3.5 | 90% | 3.5 | 80% | 3.5 | 70% |
| 4 | 100% | 4 | 90% | 4 | 80% | 4 | 70% |
| 5 | 100% | 5 | 90% | 5 | 80% | 5 | 70% |
| 6 | 100% | 6 | 90% | 6 | 80% | 6 | 70% |
| 7 | 100% | 7 | 90% | 7 | 80% | 7 | 70% |
| 8 | 100% | 8 | 90% | 8 | 80% | 8 | 70% |
| 9 | 100% | 9 | 90% | 9 | 80% | 9 | 70% |
| 10 | 100% | 10 | 90% | 10 | 80% | 10 | 70% |
| 15 | 100% | 15 | 90% | 15 | 80% | 15 | 70% |
| 20 | 100% | 20 | 90% | 20 | 80% | 20 | 70% |

In some embodiments, a sample (e.g., tissue, e.g., whole blood, e.g., bone marrow aspirate, e.g., cells, e.g., peripheral blood mononuclear cells (PBMCs), leukocytes, bone marrow mononuclear cells (BMMCs)) is fixed using a fixation buffer comprising DSP and ethanol. In some embodiments, the fixation buffer comprises about 10%, 20%, 30%, 40%, 50%, 60%, 70%, 80%, 90%, 99%, 99.9%, 100% ethanol. In some embodiments, the fixation buffer comprises 10%, 20%, 30%, 40%, 50%, 60%, 70%, 80%, 90%, 99%, 99.9%, 100% ethanol. In some embodiments, the fixation buffer comprises a final concentration of DSP between about 0.25 mM and about 5 mM, and ethanol. In some embodiments, the fixation buffer comprises a final concentration of DSP between about 0.5 mM and about 10 mM, and ethanol. In some embodiments, the fixation buffer comprises a final concentration of DSP between about 5 mM and about 20 mM, and ethanol. In some embodiments, the fixation buffer comprises a final concentration of about 3.125 mM DSP. In some embodiments, the fixation buffer comprises a final concentration of about 3.125 mM DSP and 100% ethanol. In some embodiments, the fixation buffer comprises a final concentration of DSP between 0.25 mM-5 mM, and ethanol. In some embodiments, the fixation buffer comprises a final concentration of DSP between 0.5 mM-10 mM, and ethanol. In some embodiments, the fixation buffer comprises a final concentration of DSP between 5 mM-20 mM, and ethanol. In some embodiments, the fixation buffer comprises a final concentration of 3.125 mM DSP. In some embodiments, the fixation buffer comprises a final concentration of 3.125 mM DSP and 100% ethanol.

As described herein, cross-linking agents may also be used for fixation including, without limitation, disuccinimidyl suberate (DSS), dimethylsuberimidate (DMS), formalin, and dimethyladipimidate (DMA), dithio-bis(-succinimidyl propionate) (DSP), disuccinimidyl tartrate (DST), and ethylene glycol bis(succinimidyl succinate) (EGS), ethylene glycol bis(sulfosuccinimidyl succinate) (sulfo-EGS), bis(sulfosuccinimidyl)suberate (BS3), 3,3′-dithiobis(sulfosuccinimidyl propionate) (DTSSP), bis-succinimide ester-activated PEG (BS(PEG)9) and dimethyl 3,3′-dithiobispropionimidate dihydrochloride (DTBP). In some cases, a cross-linking agent may be a cleavable cross-linking agent (e.g., thermally cleavable, photocleavable, etc.). In some cases, more than one fixation reagent can be used in combination when preparing a fixed biological sample. Changes to a characteristic or a set of characteristics of a cell or cellular constituents (e.g., incurred upon interaction with one or more fixation agents) may be at least partially reversible (e.g., via rehydration or de-crosslinking). Alternatively, changes to a characteristic or set of characteristics of a cell or cellular constituents (e.g., incurred upon interaction with one or more fixation agents) may be substantially irreversible. In some embodiments, fixation may be performed by a single incubation of a sample in a fixative, such as a cross linking fixative. In some embodiments, as described herein, fixation may be performed by the sequential administration of two or more crosslinkers, e.g., by first contacting a cell or tissue with a hydrophobic crosslinker, then with a hydrophilic crosslinker.

### RNA Templated Ligation Module

The methods described herein may comprise templated ligation. A templated ligation process may comprise contacting a nucleic acid molecule (e.g., an RNA molecule) with a probe molecule, such as a DNA probe, RNA probe, or a probe comprising both DNA and RNA. The probe molecule may interact with one or more other nucleic acid molecules, for example, those comprising a barcode sequence, to generate a probe-barcode complex. An extension reaction may be performed on at least a portion of the probe-barcode complex to generate a nucleic acid product that comprises the barcode sequence and is associated with a sequence of the nucleic acid molecule. Beneficially, the methods described herein may allow barcoding of the nucleic acid molecule without performing reverse transcription on the nucleic acid molecule. The methods herein may comprise ligation-mediated reactions.

A method may comprise contacting a nucleic acid molecule (e.g., an RNA molecule) with a first probe molecule, comprising a first sequence and a second sequence, under conditions sufficient for the first sequence to hybridize to a sequence of the nucleic acid molecule. A second probe molecule comprising a third sequence may hybridize to the second sequence of the first probe molecule. The first probe or the second probe molecule may comprise a barcode sequence (e.g., as described herein). For example, the second probe molecule may be a nucleic acid molecule (e.g., as described herein). In some cases, a splint molecule may be used to link the first and second probe molecules. For example, a fourth sequence of the splint molecule may hybridize to the second sequence of the first probe molecule and a fifth sequence of the splint molecule may hybridize to the third sequence of the second probe molecule.

In another example, a first probe molecule with a first reactive moiety and a second probe molecule with a second reactive moiety may be used. A first sequence of the first probe molecule may hybridize to a first sequence of a nucleic acid molecule and a second sequence of the second probe molecule may hybridize to a second sequence of the nucleic acid molecule. The first sequence of the nucleic acid molecule and the second sequence of the nucleic acid molecule may be on the same nucleic acid strand. The first and second sequences of the nucleic acid molecule may be adjacent or may be separated by a gap of one or more nucleotides, which gap may optionally be filled (e.g., using a polymerase or one or more other relevant enzymes). The first reactive moiety of the first probe molecule and the second reactive moiety of the second probe molecule may be subjected to conditions sufficient for the first and second reactive moieties to react to provide a linking moiety. For example, a click chemistry reaction involving an alkyne moiety and an azide moiety may be used to provide a triazole linking moiety. In other examples, an iodide moiety may be chemically ligated to a phosphorothioate moiety to form a phosphorothioate bond, an acid may be ligated to an amine to form an amide bond, or a phosphate may be ligated to an amine to form a phosphoramidate bond. In some cases, the probes may be subjected to an enzymatic ligation reaction, using a ligase, e.g., SplintR ligases, T4 ligases, KOD ligases, PBCV1 enzymes, etc. to form a probe-linked nucleic acid molecule. Where the two probes are non-adjacent, gap regions between the probes may be filled prior to ligation. In some instances, ribonucleotides or deoxyribonucleotides are ligated between the first and second probes.

Prior to, in parallel, or subsequent to linking of the first and second probe molecules (e.g., via reaction between their respective reactive moieties), a third probe molecule (e.g., a nucleic acid barcode molecule) may be subjected to conditions sufficient to hybridize to a third sequence of the first probe molecule. The third probe molecule may comprise a barcode sequence. In some cases, a splint molecule may be used to link the first and third probe molecules. In some cases, the first and second probe molecules may be linked to one another such that a loop or “padlock” is formed after hybridization of the first sequence of the first probe molecule to the first sequence of the nucleic acid molecule and the second sequence of the second probe molecule to the second sequence of the nucleic acid molecule. A linkage between the first and second probe molecules may be generated after hybridization of the first and second probe molecules to the nucleic acid molecule, such as via reaction between two reactive moieties to form a linking moiety. Alternatively, the first and second probe molecules may be linked to one another before the first and second probe molecules hybridize to the nucleic acid molecule.

All or a portion of the templated ligation processes described herein may be performed within a partition (e.g., as described herein). Alternatively, one or more such processes may be performed within a bulk solution. For example, one or more probe molecules may be subjected to conditions sufficient to hybridize to a nucleic acid molecule (e.g., a nucleic acid molecule included in a biological particle such as a cell) within a bulk solution. The nucleic acid molecule may be partitioned within various reagents (e.g., as described herein) including a nucleic acid barcode molecule, such as a nucleic acid barcode molecule releasably coupled to a bead (e.g., as described herein). Within the partition, the nucleic acid barcode molecule may hybridize to a sequence of a probe molecule hybridized to the nucleic acid molecule, thereby generated a barcode-linked nucleic acid molecule. The ligation or linking of the first probe molecule and the second probe molecule may occur prior to partitioning, during partitioning, or after partitioning. The ligation or linking of the first probe molecule and the second probe molecule may occur while in the partition. Templated ligation processes may permit indirect barcoding of a nucleic acid molecule without the use of reverse transcription. Details of such processes and additional schemes are included in, for example, International Patent Application Publication Nos. WO2019/165318 and WO2021/041974, U.S. Patent Application Publication Nos. US20200239874, and U.S. Pat. No. 11,639,928, which are herein entirely incorporated by reference for all purposes.

### Targeting GEX

The methods provided herein may comprise the use of a targeting process to, e.g., enrich selected nucleic acid molecules within a sample.

An exemplary target enrichment method may comprise providing a plurality of barcoded nucleic acid molecules and hybridizing barcoded nucleic acid molecules comprising targeted regions of interest to oligonucleotide probes (“baits”) which are complementary to the targeted regions of interest (or to regions near or adjacent to the targeted regions of interest). Baits may be attached to a capture molecule, including without limitation a biotin molecule. The capture molecule (e.g., biotin) can be used to selectively pull down the targeted regions of interest (for example, with magnetic streptavidin beads) to thereby enrich the resultant population of barcoded nucleic acid molecules for those containing the targeted regions of interest.

Another exemplary enrichment method may comprise providing a plurality of barcoded nucleic acid molecules comprising a plurality of different barcode sequences, identifying a barcode sequence of the plurality of different barcode sequences, and enriching barcoded nucleic acid molecules comprising the barcode sequence. Enriching may comprise performing a nucleic acid extension reaction using a barcoded nucleic acid molecule comprising the barcode sequence and a primer comprising a sequence specific for the barcode sequence to generate an enriched plurality of barcoded nucleic acid molecules comprising the barcode sequence of interest. Details of such processes and additional schemes are included in, for example, International Patent Application No. PCT/US2020/012413, U.S. Patent Application Publication No. US2022/0025435, and U.S. Pat. No. 11,000,049, and herein entirely incorporated by reference for all purposes.

The present disclosure provides methods and systems for multiplexing, and otherwise increasing throughput in, analysis. For example, a single or integrated process workflow may permit the processing, identification, and/or analysis of more or multiple analytes, more or multiple types of analytes, and/or more or multiple types of analyte characterizations. For example, in the methods and systems described herein, one or more labelling agents capable of binding to or otherwise coupling to one or more cell features may be used to characterize biological particles and/or cell features. In some instances, cell features include cell surface features. Cell surface features may include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof. A labelling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labelling agents can include (e.g., are attached to) a reporter oligonucleotide that is indicative of the cell surface feature to which the binding group binds. For example, the reporter oligonucleotide may comprise a barcode sequence that permits identification of the labelling agent. For example, a labelling agent that is specific to one type of cell feature (e.g., a first cell surface feature) may have a first reporter oligonucleotide coupled thereto, while a labelling agent that is specific to a different cell feature (e.g., a second cell surface feature) may have a different reporter oligonucleotide coupled thereto. For a description of exemplary labelling agents, reporter oligonucleotides, and methods of use, see, e.g., U.S. Pat. No. 10,550,429; U.S. Pat. Pub. 20190177800; and U.S. Pat. Pub. 20190367969, each of which is herein entirely incorporated by reference for all purposes.

In a particular example, a library of potential cell feature labelling agents may be provided, where the respective cell feature labelling agents are associated with nucleic acid reporter molecules, such that a different reporter oligonucleotide sequence is associated with each labelling agent capable of binding to a specific cell feature. In some aspects, different members of the library may be characterized by the presence of a different oligonucleotide sequence label. For example, an antibody capable of binding to a first protein may have associated with it a first reporter oligonucleotide sequence, while an antibody capable of binding to a second protein may have a different reporter oligonucleotide sequence associated with it. The presence of the particular oligonucleotide sequence may be indicative of the presence of a particular antibody or cell feature which may be recognized or bound by the particular antibody.

Labelling agents capable of binding to or otherwise coupling to one or more biological particles may be used to characterize a biological particle as belonging to a particular set of biological particles. For example, labeling agents may be used to label a sample of cells or a group of cells. In this way, a group of cells may be labeled as different from another group of cells. In an example, a first group of cells may originate from a first sample and a second group of cells may originate from a second sample. Labelling agents may allow the first group and second group to have a different labeling agent (or reporter oligonucleotide associated with the labeling agent). This may, for example, facilitate multiplexing, where cells of the first group and cells of the second group may be labeled separately and then pooled together for downstream analysis. The downstream detection of a label may indicate analytes as belonging to a particular group.

For example, a reporter oligonucleotide may be linked to an antibody or an epitope binding fragment thereof, and labeling a biological particle may comprise subjecting the antibody-linked barcode molecule or the epitope binding fragment-linked barcode molecule to conditions suitable for binding the antibody to a molecule present on a surface of the biological particle. The binding affinity between the antibody or the epitope binding fragment thereof and the molecule present on the surface may be within a desired range to ensure that the antibody or the epitope binding fragment thereof remains bound to the molecule. For example, the binding affinity may be within a desired range to ensure that the antibody or the epitope binding fragment thereof remains bound to the molecule during various sample processing steps, such as partitioning and/or nucleic acid amplification or extension. A dissociation constant (Kd) between the antibody or an epitope binding fragment thereof and the molecule to which it binds may be less than about 100 μM, 90 μM, 80 μM, 70 μM, 60 μM, 50 μM, 40 μM, 30 μM, 20 μM, 10 μM, 9 μM, 8 μM, 7 μM, 6 μM, 5 μM, 4 μM, 3 μM, 2 μM, 1 μM, 900 nM, 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, 900 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, 10 pM, 9 pM, 8 pM, 7 pM, 6 pM, 5 pM, 4 pM, 3 pM, 2 pM, or 1 pM. For example, the dissociation constant may be less than about 10 μM.

In another example, a reporter oligonucleotide may be coupled to a cell-penetrating peptide (CPP), and labeling cells may comprise delivering the CPP coupled reporter oligonucleotide into a biological particle. Labeling biological particles may comprise delivering the CPP conjugated oligonucleotide into a cell and/or cell bead by the cell-penetrating peptide. A cell-penetrating peptide that can be used in the methods provided herein can comprise at least one non-functional cysteine residue, which may be either free or derivatized to form a disulfide link with an oligonucleotide that has been modified for such linkage. Non-limiting examples of cell-penetrating peptides that can be used in embodiments herein include penetratin, transportan, plsl, TAT(48-60), pVEC, MTS, and MAP. Cell-penetrating peptides useful in the methods provided herein can have the capability of inducing cell penetration for at least about 30%, 40%, 50%, 60%, 70%, 80%, 90%, 95%, 96%, 97%, 98%, 99%, or 100% of cells of a cell population. The cell-penetrating peptide may be an arginine-rich peptide transporter. The cell-penetrating peptide may be Penetratin or the Tat peptide.

In another example, a reporter oligonucleotide may be coupled to a fluorophore or dye, and labeling cells may comprise subjecting the fluorophore-linked barcode molecule to conditions suitable for binding the fluorophore to the surface of the biological particle. In some instances, fluorophores can interact strongly with lipid bilayers and labeling biological particles may comprise subjecting the fluorophore-linked barcode molecule to conditions such that the fluorophore binds to or is inserted into a membrane of the biological particle. In some cases, the fluorophore is a water-soluble, organic fluorophore. In some instances, the fluorophore is Alexa 532 maleimide, tetramethylrhodamine-5-maleimide (TMR maleimide), BODIPY-TMR maleimide, Sulfo-Cy3 maleimide, Alexa 546 carboxylic acid/succinimidyl ester, Atto 550 maleimide, Cy3 carboxylic acid/succinimidyl ester, Cy3B carboxylic acid/succinimidyl ester, Atto 565 biotin, Sulforhodamine B, Alexa 594 maleimide, Texas Red maleimide, Alexa 633 maleimide, Abberior STAR 635P azide, Atto 647N maleimide, Atto 647 SE, or Sulfo-Cy5 maleimide. See, e.g., Hughes L D, et al. PLoS One. 2014 Feb. 4; 9(2):e87649, which is hereby incorporated by reference in its entirety for all purposes, for a description of organic fluorophores.

A reporter oligonucleotide may be coupled to a lipophilic molecule, and labeling biological particles may comprise delivering the nucleic acid barcode molecule to a membrane of the biological particle or a nuclear membrane by the lipophilic molecule. Lipophilic molecules can associate with and/or insert into lipid membranes such as cell membranes and nuclear membranes. In some cases, the insertion can be reversible. In some cases, the association between the lipophilic molecule and biological particle may be such that the biological particle retains the lipophilic molecule (e.g., and associated components, such as nucleic acid barcode molecules, thereof) during subsequent processing (e.g., partitioning, cell permeabilization, amplification, pooling, etc.). The reporter nucleotide may enter into the intracellular space and/or a cell nucleus.

A reporter oligonucleotide may be part of a nucleic acid molecule comprising any number of functional sequences, as described elsewhere herein, such as a target capture sequence, a random primer sequence, and the like, and coupled to another nucleic acid molecule that is, or is derived from, the analyte.

Prior to partitioning, the cells may be incubated with the library of labelling agents, that may be labelling agents to a broad panel of different cell features, e.g., receptors, proteins, etc., and which include their associated reporter oligonucleotides. Unbound labelling agents may be washed from the cells, and the cells may then be co-partitioned (e.g., into droplets or wells) along with partition-specific barcode oligonucleotides (e.g., attached to a support, such as a bead or gel bead) as described elsewhere herein. As a result, the partitions may include the cell or cells, as well as the bound labelling agents and their known, associated reporter oligonucleotides.

In other instances, e.g., to facilitate sample multiplexing, a labelling agent that is specific to a particular cell feature may have a first plurality of the labelling agent (e.g., an antibody or lipophilic moiety) coupled to a first reporter oligonucleotide and a second plurality of the labelling agent coupled to a second reporter oligonucleotide. For example, the first plurality of the labeling agent and second plurality of the labeling agent may interact with different cells, cell populations or samples, allowing a particular report oligonucleotide to indicate a particular cell population (or cell or sample) and cell feature. In this way, different samples or groups can be independently processed and subsequently combined together for pooled analysis (e.g., partition-based barcoding as described elsewhere herein). See, e.g., U.S. Pat. Pub. US2019/0323088, which is hereby entirely incorporated by reference for all purposes.

As described elsewhere herein, libraries of labelling agents may be associated with a particular cell feature as well as be used to identify analytes as originating from a particular biological particle, population, or sample. The biological particles may be incubated with a plurality of libraries and a given biological particle may comprise multiple labelling agents. For example, a cell may comprise coupled thereto a lipophilic labeling agent and an antibody. The lipophilic labeling agent may indicate that the cell is a member of a particular cell sample, whereas the antibody may indicate that the cell comprises a particular analyte. In this manner, the reporter oligonucleotides and labelling agents may allow multi-analyte, multiplexed analyses to be performed.

In some instances, these reporter oligonucleotides may comprise nucleic acid barcode sequences that permit identification of the labelling agent which the reporter oligonucleotide is coupled to. The use of oligonucleotides as the reporter may provide advantages of being able to generate significant diversity in terms of sequence, while also being readily attachable to most biomolecules, e.g., antibodies, etc., as well as being readily detected, e.g., using sequencing or array technologies.

Attachment (coupling) of the reporter oligonucleotides to the labelling agents may be achieved through any of a variety of direct or indirect, covalent or non-covalent associations or attachments. For example, oligonucleotides may be covalently attached to a portion of a labelling agent (such a protein, e.g., an antibody or antibody fragment), e.g., via a linker, using chemical conjugation techniques (e.g., LIGHTNING-LINK® antibody labelling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry such as a Methyltetrazine-PEG5-NHS Ester reaction, a TCO-PEG4-NHS Ester reaction, or the like, may be used to couple reporter oligonucleotides to labelling agents. Commercially available kits, such as those from Thunderlink and Abcam, and techniques common in the art may be used to couple reporter oligonucleotides to labelling agents as appropriate. In another example, a labelling agent is indirectly (e.g., via hybridization) coupled to a reporter oligonucleotide comprising a barcode sequence that identifies the label agent. For instance, the labelling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labelling agent to the reporter oligonucleotide. In some embodiments, the reporter oligonucleotides are releasable from the labelling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein. In some instances, the reporter oligonucleotides described herein may include one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, a sequencing primer or primer biding sequence (such as an R1, R2, or partial R1 or R2 sequence).

In some cases, the labelling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labelling agent (or reporter oligonucleotide) either directly or indirectly (e.g., the label can be conjugated to a molecule that can bind to the labelling agent or reporter oligonucleotide). In some cases, a label is conjugated to an oligonucleotide that is complementary to a sequence of the reporter oligonucleotide, and the oligonucleotide may be allowed to hybridize to the reporter oligonucleotide.

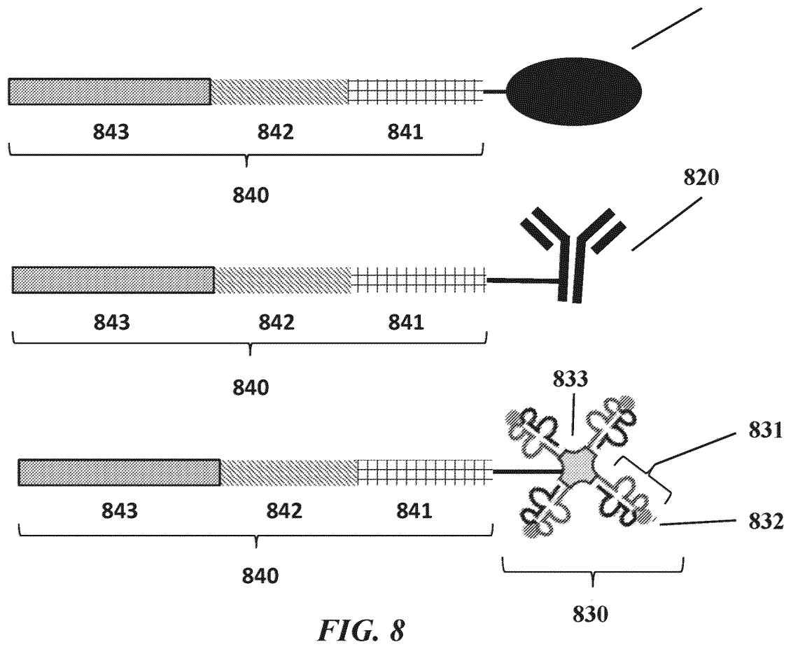

FIG. 8 describes exemplary labelling agents (810, 820, 830) comprising reporter oligonucleotides (840) attached thereto. Labelling agent 810 (e.g., any of the labelling agents described herein) is attached (either directly, e.g., covalently attached, or indirectly) to reporter oligonucleotide 840. Reporter oligonucleotide 840 may comprise barcode sequence 842 that identifies labelling agent 810. Reporter oligonucleotide 840 may also comprise one or more functional sequences that can be used in subsequent processing, such as an adapter sequence, a unique molecular identifier (UMI) sequence, a sequencer specific flow cell attachment sequence (such as an P5, P7, or partial P5 or P7 sequence), a primer or primer binding sequence, or a sequencing primer or primer biding sequence (such as an R1, R2, or partial R1 or R2 sequence).

Referring to FIG. 8, in some instances, reporter oligonucleotide 840 conjugated to a labelling agent (e.g., 810, 820, 830) comprises a functional sequence 841 (e.g., a primer sequence), a barcode sequence that identifies the labelling agent (e.g., 810, 820, 830), and functional sequence 843. Functional sequence 843 can be a reporter capture handle sequence configured to hybridize to a complementary sequence, such as a complementary sequence present on a nucleic acid barcode molecule 890 (not shown), such as those described elsewhere herein. In some instances, nucleic acid barcode molecule 890 is attached to a support (e.g., a bead, such as a gel bead), such as those described elsewhere herein. For example, nucleic acid barcode molecule 890 may be attached to the support via a releasable linkage (e.g., comprising a labile bond), such as those described elsewhere herein. In some instances, reporter oligonucleotide 840 comprises one or more additional functional sequences, such as those described above.

In some instances, the labelling agent 810 is a protein or polypeptide (e.g., an antigen or prospective antigen) comprising reporter oligonucleotide 840. Reporter oligonucleotide 840 comprises barcode sequence 842 that identifies polypeptide 810 and can be used to infer the presence of an analyte, e.g., a binding partner of polypeptide 810 (i.e., a molecule or compound to which polypeptide 810 can bind). In some instances, the labelling agent 810 is a lipophilic moiety (e.g., cholesterol) comprising reporter oligonucleotide 840, where the lipophilic moiety is selected such that labelling agent 810 integrates into a membrane of a cell or nucleus. Reporter oligonucleotide 840 comprises barcode sequence 842 that identifies lipophilic moiety 810 which in some instances is used to tag cells (e.g., groups of cells, cell samples, etc.) and may be used for multiplex analyses as described elsewhere herein. In some instances, the labelling agent is an antibody 820 (or an epitope binding fragment thereof) comprising reporter oligonucleotide 840. Reporter oligonucleotide 840 comprises barcode sequence 842 that identifies antibody 820 and can be used to infer the presence of, e.g., a target of antibody 820 (i.e., a molecule or compound to which antibody 820 binds). In other embodiments, labelling agent 830 comprises an MHC molecule 831 comprising peptide 832 and reporter oligonucleotide 840 that identifies peptide 832. In some instances, the MHC molecule is coupled to a support 833. In some instances, support 833 may be a polypeptide, such as streptavidin, or a polysaccharide, such as dextran. In some instances, reporter oligonucleotide 840 may be directly or indirectly coupled to MHC labelling agent 830 in any suitable manner. For example, reporter oligonucleotide 840 may be coupled to MHC molecule 831, support 833, or peptide 832. In some embodiments, labelling agent 830 comprises a plurality of MHC molecules, (e.g. is an MHC multimer, which may be coupled to a support (e.g., 833)). There are many possible configurations of Class I and/or Class II MHC multimers that can be utilized with the compositions, methods, and systems disclosed herein, e.g., MHC tetramers, MHC pentamers (MHC assembled via a coiled-coil domain, e.g., Pro5® MHC Class I Pentamers, (ProImmune, Ltd.), MHC octamers, MHC dodecamers, MHC decorated dextran molecules (e.g., MHC Dextramer® (Immudex)), etc. For a description of exemplary labelling agents, including antibody and MHC-based labelling agents, reporter oligonucleotides, and methods of use, see, e.g., U.S. Pat. No. 10,550,429 and U.S. Pat. Pub. 20190367969, each of which is herein entirely incorporated by reference for all purposes.

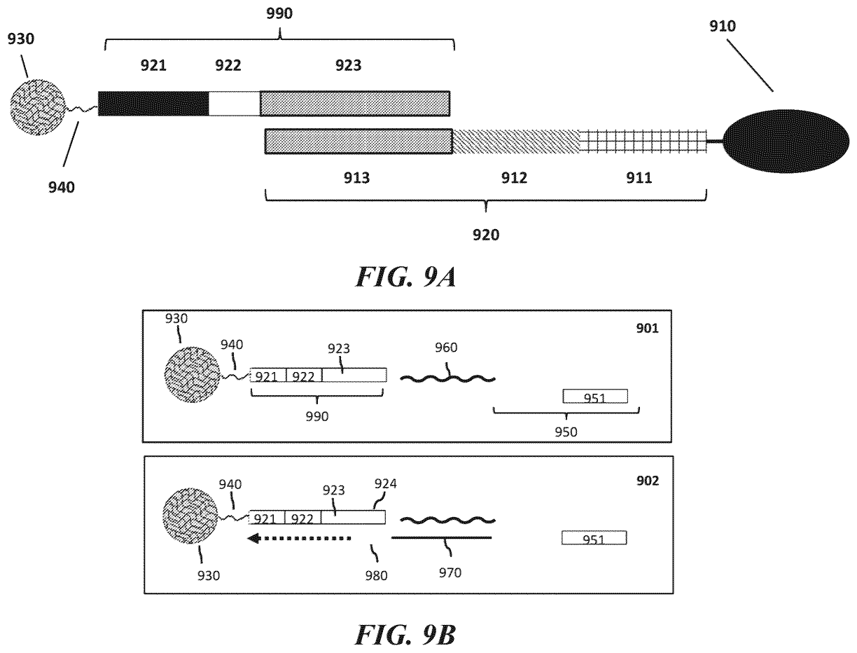

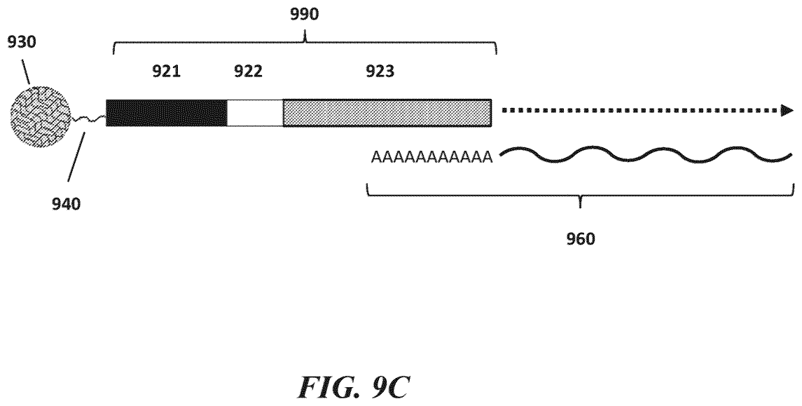

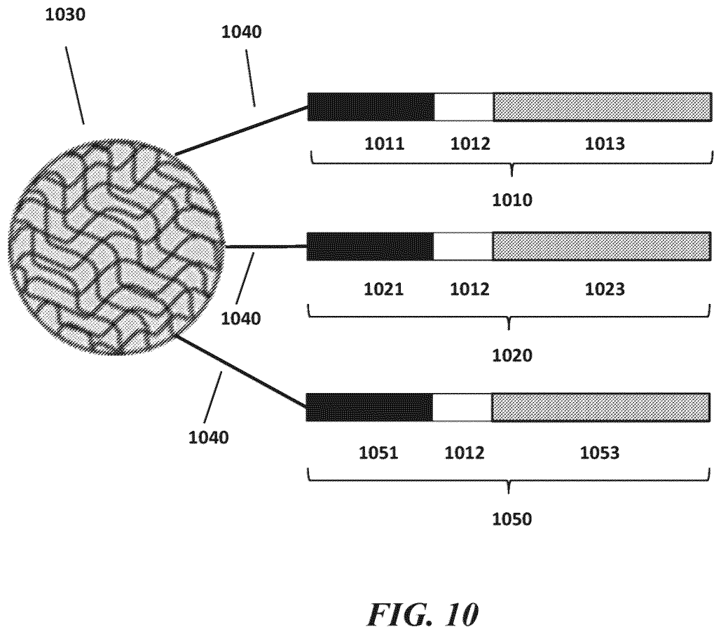

FIG. 10 illustrates another example of a barcode carrying bead. In some embodiments, analysis of multiple analytes (e.g., RNA and one or more analytes using labelling agents described herein) may comprise nucleic acid barcode molecules as generally depicted in FIG. 10. In some embodiments, nucleic acid barcode molecules 1010 and 1020 are attached to support 1030 via a releasable linkage 1040 (e.g., comprising a labile bond) as described elsewhere herein. Nucleic acid barcode molecule 1010 may comprise adapter sequence 1011, barcode sequence 1012 and capture sequence 1013. Nucleic acid barcode molecule 1020 may comprise adapter sequence 1021, barcode sequence 1012, and capture sequence 1023, wherein capture sequence 1023 comprises a different sequence than capture sequence 1013. In some instances, adapter 1011 and adapter 1021 comprise the same sequence. In some instances, adapter 1011 and adapter 1021 comprise different sequences. Although support 1030 is shown comprising nucleic acid barcode molecules 1010 and 1020, any suitable number of barcode molecules comprising common barcode sequence 1012 are contemplated herein. For example, in some embodiments, support 1030 further comprises nucleic acid barcode molecule 1050. Nucleic acid barcode molecule 1050 may comprise adapter sequence 1051, barcode sequence 1012 and capture sequence 1053, wherein capture sequence 1053 comprises a different sequence than capture sequence 1013 and 1023. In some instances, nucleic acid barcode molecules (e.g., 1010, 1020, 1050) comprise one or more additional functional sequences, such as a UMI or other sequences described herein. The nucleic acid barcode molecules 1010, 1020 or 1050 may interact with analytes as described elsewhere herein, for example, as depicted in FIGS. 9A-C.

Referring to FIG. 9A, in an instance where cells are labelled with labeling agents, capture sequence 923 may be complementary to an adapter sequence of a reporter oligonucleotide. Cells may be contacted with one or more reporter oligonucleotide 920 conjugated labelling agents 910 (e.g., polypeptide, antibody, or others described elsewhere herein). In some cases, the cells may be further processed prior to barcoding. For example, such processing steps may include one or more washing and/or cell sorting steps. In some instances, a cell that is bound to labelling agent 910 which is conjugated to oligonucleotide 920 and support 930 (e.g., a bead, such as a gel bead) comprising nucleic acid barcode molecule 990 is partitioned into a partition amongst a plurality of partitions (e.g., a droplet of a droplet emulsion or a well of a microwell array). In some instances, the partition comprises at most a single cell bound to labelling agent 910. In some instances, reporter oligonucleotide 920 conjugated to labelling agent 910 (e.g., polypeptide, an antibody, pMHC molecule such as an MHC multimer, etc.) comprises a first adapter sequence 911 (e.g., a primer sequence), a barcode sequence 912 that identifies the labelling agent 910 (e.g., the polypeptide, antibody, or peptide of a pMHC molecule or complex), and a capture handle sequence 913. Capture handle sequence 913 may be configured to hybridize to a complementary sequence, such as a capture sequence 923 present on a nucleic acid barcode molecule 990. In some instances, oligonucleotide 920 comprises one or more additional functional sequences, such as those described elsewhere herein.

Barcoded nucleic may be generated (e.g., via a nucleic acid reaction, such as nucleic acid extension or ligation) from the constructs described in FIGS. 9A-C. For example, capture handle sequence 913 may then be hybridized to complementary sequence, such as capture sequence 923 to generate (e.g., via a nucleic acid reaction, such as nucleic acid extension or ligation) a barcoded nucleic acid molecule comprising cell (e.g., partition specific) barcode sequence 922 (or a reverse complement thereof) and reporter barcode sequence 912 (or a reverse complement thereof). In some embodiments, the nucleic acid barcode molecule 990 (e.g., partition-specific barcode molecule) further includes a UMI (not shown). Barcoded nucleic acid molecules can then be optionally processed as described elsewhere herein, e.g., to amplify the molecules and/or append sequencing platform specific sequences to the fragments. See, e.g., U.S. Pat. Pub. 2018/0105808, which is hereby entirely incorporated by reference for all purposes. Barcoded nucleic acid molecules, or derivatives generated therefrom, can then be sequenced on a suitable sequencing platform.

In some instances, analysis of multiple analytes (e.g., nucleic acids and one or more analytes using labelling agents described herein) may be performed. For example, the workflow may comprise a workflow as generally depicted in any of FIGS. 9A-C, or a combination of workflows for an individual analyte, as described elsewhere herein. For example, by using a combination of the workflows as generally depicted in FIGS. 9A-C, multiple analytes can be analyzed.

In some instances, analysis of an analyte (e.g. a nucleic acid, a polypeptide, a carbohydrate, a lipid, etc.) comprises a workflow as generally depicted in FIG. 9A. A nucleic acid barcode molecule 990 may be co-partitioned with the one or more analytes. In some instances, nucleic acid barcode molecule 990 is attached to a support 930 (e.g., a bead, such as a gel bead), such as those described elsewhere herein. For example, nucleic acid barcode molecule 990 may be attached to support 930 via a releasable linkage 940 (e.g., comprising a labile bond), such as those described elsewhere herein. Nucleic acid barcode molecule 990 may comprise a functional sequence 921 and optionally comprise other additional sequences, for example, a barcode sequence 922 (e.g., common barcode, partition-specific barcode, or other functional sequences described elsewhere herein), and/or a UMI sequence (not shown). The nucleic acid barcode molecule 990 may comprise a capture sequence 923 that may be complementary to another nucleic acid sequence, such that it may hybridize to a particular sequence, e.g., capture handle sequence 913.

For example, capture sequence 923 may comprise a poly-T sequence and may be used to hybridize to mRNA. Referring to FIG. 9C, in some embodiments, nucleic acid barcode molecule 990 comprises capture sequence 923 complementary to a sequence of RNA molecule 960 from a cell. In some instances, capture sequence 923 comprises a sequence specific for an RNA molecule. Capture sequence 923 may comprise a known or targeted sequence or a random sequence. In some instances, a nucleic acid extension reaction may be performed, thereby generating a barcoded nucleic acid product comprising capture sequence 923, the functional sequence 921, barcode sequence 922, any other functional sequence, and a sequence corresponding to the RNA molecule 960.

In another example, capture sequence 923 may be complementary to an overhang sequence or an adapter sequence that has been appended to an analyte. For example, referring to FIG. 9B, panel 901, in some embodiments, primer 950 comprises a sequence complementary to a sequence of nucleic acid molecule 960 (such as an RNA encoding for a BCR sequence) from a biological particle. In some instances, primer 950 comprises one or more sequences 951 that are not complementary to RNA molecule 960. Sequence 951 may be a functional sequence as described elsewhere herein, for example, an adapter sequence, a sequencing primer sequence, or a sequence the facilitates coupling to a flow cell of a sequencer. In some instances, primer 950 comprises a poly-T sequence. In some instances, primer 950 comprises a sequence complementary to a target sequence in an RNA molecule. In some instances, primer 950 comprises a sequence complementary to a region of an immune molecule, such as the constant region of a TCR or BCR sequence. Primer 950 is hybridized to nucleic acid molecule 960 and complementary molecule 970 is generated (see Panel 902). For example, complementary molecule 970 may be cDNA generated in a reverse transcription reaction. In some instances, an additional sequence may be appended to complementary molecule 970. For example, the reverse transcriptase enzyme may be selected such that several non-templated bases 980 (e.g., a poly-C sequence) are appended to the cDNA. In another example, a terminal transferase may also be used to append the additional sequence. Nucleic acid barcode molecule 990 comprises a sequence 924 complementary to the non-templated bases, and the reverse transcriptase performs a template switching reaction onto nucleic acid barcode molecule 990 to generate a barcoded nucleic acid molecule comprising cell (e.g., partition specific) barcode sequence 922 (or a reverse complement thereof) and a sequence of complementary molecule 970 (or a portion thereof). In some instances, sequence 923 comprises a sequence complementary to a region of an immune molecule, such as the constant region of a TCR or BCR sequence. Sequence 923 is hybridized to nucleic acid molecule 960 and a complementary molecule 970 is generated. For example, complementary molecule 970 may be generated in a reverse transcription reaction generating a barcoded nucleic acid molecule comprising cell (e.g., partition specific) barcode sequence 922 (or a reverse complement thereof) and a sequence of complementary molecule 970 (or a portion thereof). Additional methods and compositions suitable for barcoding cDNA generated from mRNA transcripts including those encoding, V(D)J regions of an immune cell receptor and/or barcoding methods and composition including a template switch oligonucleotide are described in International Patent Application WO2018/075693, US. Patent Publication No. 2018/0105808, U.S. Patent Publication No, 2015/0376609, filed Jun. 26, 2015, and U.S. Patent Publication No. 2019/0367969, each of which applications is herein entirely incorporated by reference for all purposes.

In some embodiments, biological particles (e.g., cells, nuclei) from a plurality of samples (e.g., a plurality of subjects) can be pooled, sequenced, and demultiplexed by identifying mutational profiles associated with individual samples and mapping sequence data from single biological particles to their source based on their mutational profile. See, e.g., Xu J. et al., Genome Biology Vol. 20, 290 (2019); Huang Y. et al., Genome Biology Vol. 20, 273 (2019); and Heaton et al., Nature Methods volume 17, pages 615-620(2020).

Gene expression data can reflect the underlying genome and mutations and structural variants therein. As a result, the variation inherent in the captured and sequenced RNA molecules can be used to identify genotypes de novo or used to assign molecules to genotypes that were known a priori. In some embodiments, allelic variation that is present due to haplotypic states (including linkage disequilibrium of the human leucocyte antigen loci (HLA), immune receptor loci (BCR), and other highly polymorphic regions of the genome), can also be used for demultiplexing. Expressed B cell receptors can be used to infer germline alleles from unrelated individuals, which information may be used for demultiplexing.

### Combinatorial Barcoding Module

In some instances, barcoding of a nucleic acid molecule may be done using a combinatorial approach. In such instances, one or more nucleic acid molecules (which may be comprised in a cell or cell bead) may be partitioned (e.g., in a first set of partitions, e.g., wells or droplets) with one or more first nucleic acid barcode molecules (optionally coupled to a bead). The first nucleic acid barcode molecules or derivative thereof (e.g., complement, reverse complement) may then be attached to the one or more nucleic acid molecules, thereby generating first barcoded nucleic acid molecules, e.g., using the processes described herein. The first nucleic acid barcode molecules may be partitioned to the first set of partitions such that a nucleic acid barcode molecule, of the first nucleic acid barcode molecules, that is in a partition comprises a barcode sequence that is unique to the partition among the first set of partitions. Each partition may comprise a unique barcode sequence. For example, a set of first nucleic acid barcode molecules partitioned to a first partition in the first set of partitions may each comprise a common barcode sequence that is unique to the first partition among the first set of partitions, and a second set of first nucleic acid barcode molecules partitioned to a second partition in the first set of partitions may each comprise another common barcode sequence that is unique to the second partition among the first set of partitions. Such barcode sequence (unique to the partition) may be useful in determining the cell or partition from which the one or more nucleic acid molecules (or derivatives thereof) originated.

The first barcoded nucleic acid molecules from multiple partitions of the first set of partitions may be pooled and re-partitioned (e.g., in a second set of partitions, e.g., one or more wells or droplets) with one or more second nucleic acid barcode molecules. The second nucleic acid barcode molecules or derivative thereof may then be attached to the first barcoded nucleic acid molecules, thereby generating second barcoded nucleic acid molecules. As with the first nucleic acid barcode molecules during the first round of partitioning, the second nucleic acid barcode molecules may be partitioned to the second set of partitions such that a nucleic acid barcode molecule, of the second nucleic acid barcode molecules, that is in a partition comprises a barcode sequence that is unique to the partition among the second set of partitions. Such barcode sequence may also be useful in determining the cell or partition from which the one or more nucleic acid molecules or first barcoded nucleic acid molecules originated. The second barcoded nucleic acid molecules may thus comprise two barcode sequences (e.g., from the first nucleic acid barcode molecules and the second nucleic acid barcode molecules).

Additional barcode sequences may be attached to the second barcoded nucleic acid molecules by repeating the processes any number of times (e.g., in a split-and-pool approach), thereby combinatorically synthesizing unique barcode sequences to barcode the one or more nucleic acid molecules. For example, combinatorial barcoding may comprise at least 1, 2, 3, 4, 5, 6, 7, 8, 9, 10 or more operations of splitting (e.g., partitioning) and/or pooling (e.g., from the partitions). Additional examples of combinatorial barcoding may also be found in International Patent Application Publication No. WO2019/165318 and US Patent Application Publication No. US20200239874, each of which is herein entirely incorporated by reference for all purposes.

Beneficially, the combinatorial barcode approach may be useful for generating greater barcode diversity and synthesizing unique barcode sequences on nucleic acid molecules derived from a cell or partition. For example, combinatorial barcoding comprising three operations, each with 100 partitions, may yield up to 10⁶ unique barcode combinations. In some instances, the combinatorial barcode approach may be helpful in determining whether a partition contained only one cell or more than one cell. For instance, the sequences of the first nucleic acid barcode molecule and the second nucleic acid barcode molecule may be used to determine whether a partition comprised more than one cell. For instance, if two nucleic acid molecules comprise different first barcode sequences but the same second barcode sequences, it may be inferred that the second set of partitions comprised two or more cells.

In some instances, combinatorial barcoding may be achieved in the same compartment. For instance, a unique nucleic acid molecule comprising one or more nucleic acid bases may be attached to a nucleic acid molecule (e.g., a sample or target nucleic acid molecule) in successive operations within a partition (e.g., droplet or well) to generate a first barcoded nucleic acid molecule. A second unique nucleic acid molecule comprising one or more nucleic acid bases may be attached to the first barcoded nucleic acid molecule, thereby generating a second barcoded nucleic acid molecule. In some instances, all the reagents for barcoding and generating combinatorially barcoded molecules may be provided in a single reaction mixture, or the reagents may be provided sequentially.

In some instances, cell beads comprising nucleic acid molecules may be barcoded. Methods and systems for barcoding cell beads are further described in International Patent Application No. PCT/US2018/067356 and U.S. Patent Application Publication No. 2019/0330694, which are hereby incorporated by reference in its entirety.

Devices, systems, compositions and methods of the present disclosure may be used for various applications, such as, for example, processing a single analyte (e.g., RNA, DNA, or protein) or multiple analytes (e.g., DNA and RNA, DNA and protein, RNA and protein, or RNA, DNA and protein) from a single cell. For example, a biological particle (e.g., a cell or cell bead) is partitioned in a partition (e.g., droplet), and multiple analytes from the biological particle are processed for subsequent processing. The multiple analytes may be from the single cell. This may enable, for example, simultaneous proteomic, transcriptomic and genomic analysis of the cell.

### Cell Preservation and Storage, and Methods of Implementing the Same

The present disclosure includes one or more methods of fixing and/or storing cells, including but not limited to, methods of fixing and/or storing cells in a manner that results in reduced leakage of biological components from cells during preservation and storage of such cells.

In one or more embodiments, the methods of fixing and/or storing cells in a manner that reduces leakage includes reducing leakage from fragile cells. Fragile cells have the characteristics of being labile or unstable e.g., their plasma membrane degrades quickly or easily, e.g., pores are easily formed in the membrane allowing the intracellular contents (e.g., nucleic acids) to leak out. Fragile cells are unstable, less durable, and/or have a lower survival rate in environments outside of their native environment (e.g., cells dissociated from tissue; cells in culture). Crosslinking or fixing fragile cells prevents and/or decreases loss of original intracellular materials (e.g., nucleic acids), which is crucial for obtaining optimal data in downstream analyses, such as single cell analyses, Examples of fragile cells may include, but are not limited to, neutrophils, adipocytes, plasma cells, certain tumor cells or cancerous cells (e.g., lymphocytes in patients with chronic lymphocytic leukemia (CLL)), erythrocytes, splenocytes, and neurons.

### Sample Preparation and Methods of Implementing the Same

The present disclosure provides methods for sample preparation, including but not limited to, methods of sequentially crosslinking one or more cells and/or tissues, first with a hydrophobic crosslinker, and then with a hydrophilic crosslinker.

### Implementation #1

In accordance with one or more embodiments, a method of sample preparation is described. The method of sample preparation may include the following steps in order: i) contacting a sample comprising at least one cell or tissue with a hydrophobic crosslinker; ii) removing the hydrophobic crosslinker from the sample; iii) contacting the sample with a hydrophilic crosslinker; and iv) removing the hydrophilic crosslinker from the sample.

In one or more embodiments, the hydrophobic crosslinkers may be EGS, DSS, DSP and disulfanediylbis(ethane-2,1-diyl)bis(1H-imidzaole-1-carboxylate) (DSEB).

In one or more embodiments, the hydrophilic crosslinkers may be bis(sulfosuccinimidyl)suberate (BS3), sulfo-EGS, bis-succinimide ester-activated PEG (Bs(PEG)9), 3.3′-dithiobis(sulfosuccinimidyl propionate) (DTSSP) and dimethyl 3,3′-dithiobispropionimidate·2HCl (DTBP).

In one or more embodiments, the hydrophilic crosslinkers may be glyoxal or paraformaldehyde (PFA), which are also water soluble (hydrophilic), but are membrane permeable in contrast to the foregoing hydrophilic crosslinkers.

In one or more embodiments, fragile cells are used. Fragile cells have the characteristics of being fragile or unstable e.g., their plasma membrane degrades quickly or easily, e.g., pores are easily formed in the membrane causing the intracellular contents (e.g., nucleic acids) to leak out. Fragile cells are unstable, less durable, and/or have a lower survival rate in environments outside of their native environment (e.g., cells dissociated from tissue; cells in culture). Crosslinking or fixing fragile cells prevents loss of original material (e.g., nucleic acids), which is crucial for obtaining optimal data in downstream analyses, such as single cell analyses. Examples of fragile cells may include, but are not limited to, neutrophils, adipocytes, plasma cells, certain tumor cells or cancerous cells (e.g., lymphocytes in patients with chronic lymphocytic leukemia (CLL)), erythrocytes, splenocytes, and neurons.

### Conditions for Specific Crosslinkers

EGS has a molecular weight of 456.36, NHS ester reactive groups, an arm space length of 16.1A, a solubility in DMSO of 10 to 25 mM, can be stored at 4 degrees Celsius, can be quenched with, e.g., 20 mM Tris pH 7.5 for 20 minutes, and its crosslinking is cleavable e.g., in hydroxylamine-HCl at pH 8.5 for 3 to 6 hours at 37 degrees Celsius.

In one or more embodiments, crosslinking of a cell, e.g., a non-fragile cell, with EGS may be performed at about 0.25 mM to about 10 mM, about 0.5 mM to about 7 mM, about 1 mM to about 5 mM, or about 2.5 mM; for about 10 minutes to about 24 hours, about 15 minutes to about 4 hours, about 20 minutes to about 2 hours, or about 45 minutes; at about 2 degrees Celsius to about 25 degrees Celsius; about 4 degrees Celsius to about 23 degrees Celsius, about 4 degrees Celsius to about 20 degrees Celsius or about 20 degrees Celsius, inclusive of any endpoints, values or ranges therebetween.

In one or more embodiments, crosslinking of a fragile cell with EGS may be performed at about 0.25 mM to about 10 mM, about 0.5 mM to about 5 mM, about 0.5 mM to about 4 mM, or about 1.25 mM; for about 10 minutes to about 24 hours, about 15 minutes to about 4 hours, about 15 minutes to about 1 hour, or about 30 minutes; at about 2 degrees Celsius to about 25 degrees Celsius; about 4 degrees Celsius to about 23 degrees Celsius, about 4 degrees Celsius to about 15 degrees Celsius or about 4 degrees Celsius, inclusive of any endpoints, values or ranges therebetween.

DSS has a molecular weight of 368.35, NHS ester reactive groups, an arm space length of 11.4 A, a solubility in DMSO of >9.2 mg/ml or 25 mM, can be stored at 4 degrees Celsius, can be quenched with, e.g., 20 mM Tris pH 7.5 for 20 minutes, and its crosslinking is non-cleavable.

In one or more embodiments, crosslinking of a cell, e.g., a non-fragile cell with DSS may be performed at about 0.05 mM to about 10 mM, about 0.25 mM to about 7.5 mM, about 0.5 mM to about 5 mM, or about 2.5 mM; for about 10 minutes to about 24 hours, about 15 minutes to about 4 hours, about 20 minutes to about 1 hour, or about 30 minutes; at about 2 degrees Celsius to about 25 degrees Celsius; about 4 degrees Celsius to about 23 degrees Celsius, about 4 degrees Celsius to about 20 degrees Celsius or about 20 degrees Celsius, inclusive of any endpoints, values or ranges therebetween.

In one or more embodiments, crosslinking of a fragile cell with DSS may be performed at about 0.05 mM to about 10 mM, about 0.25 mM to about 5 mM, about 0.5 mM to about 3.5 mM, or about 1.25 mM; for about 10 minutes to about 24 hours, about 15 minutes to about 4 hours, about 15 minutes to about 45 minutes, or about 20 minutes; at about 2 degrees Celsius to about 25 degrees Celsius; about 4 degrees Celsius to about 23 degrees Celsius, about 4 degrees Celsius to about 20 degrees Celsius or about 20 degrees Celsius, inclusive of any endpoints, values or ranges therebetween.

DSP has a molecular weight of 404.42, NHS ester reactive groups, an arm space length of 12A, a solubility in DMSO of 81 mg/ml or 200 mM, can be stored at 4 degrees Celsius, can be quenched with, e.g., 20 to 50 mM Tris pH 7.5 for 15 minutes, and its crosslinking is cleavable e.g., in 20 to 50 mM DTT for 30 minutes at 37 degrees Celsius.

In one or more embodiments, crosslinking of a cell, e.g., a non-fragile cell with DSP may be performed at about 0.05 mM to about 10 mM, about 0.25 mM to about 7.5 mM, about 0.5 mM to about 5 mM, or about 2.5 mM; for about 10 minutes to about 24 hours, about 15 minutes to about 4 hours, about 20 minutes to about 1 hour, or about 30 minutes; at about 2 degrees Celsius to about 25 degrees Celsius; about 4 degrees Celsius to about 23 degrees Celsius, about 4 degrees Celsius to about 20 degrees Celsius or about 20 degrees Celsius, inclusive of any endpoints, values or ranges therebetween.

In one or more embodiments, crosslinking of a fragile cell with DSP may be performed at about 0.05 mM to about 10 mM, about 0.25 mM to about 5 mM, about 0.5 mM to about 3.5 mM, or about 1.25 mM; for about 10 minutes to about 24 hours, about 15 minutes to about 4 hours, about 15 minutes to about 45 minutes, or about 20 minutes; at about 2 degrees Celsius to about 25 degrees Celsius; about 4 degrees Celsius to about 23 degrees Celsius, about 4 degrees Celsius to about 20 degrees Celsius or about 20 degrees Celsius, inclusive of any endpoints, values or ranges therebetween.

DSEB has a molecular weight of 342.39, imidazole reactive groups, solubility in DMSO, can be stored at −20 degrees Celsius in powder aliquots, and its crosslinking is cleavable e.g., in 20 to 50 mM DTT for 30 minutes at 37 degrees Celsius.

In one or more embodiments, crosslinking of a cell, e.g., a non-fragile cell with DSEB may be performed at about 0.05 mM to about 10 mM, about 0.25 mM to about 7.5 mM, about 1 mM to about 5 mM, or about 4 mM; for about 10 minutes to about 24 hours, about 15 minutes to about 4 hours, about 20 minutes to about 1 hour, or about 30 minutes; at about 2 degrees Celsius to about 25 degrees Celsius; about 4 degrees Celsius to about 23 degrees Celsius, about 4 degrees Celsius to about 20 degrees Celsius or about 20 degrees Celsius, inclusive of any endpoints, values or ranges therebetween.

In one or more embodiments, crosslinking of fragile cell with DSEB may be performed at about 0.05 mM to about 10 mM, about 0.25 mM to about 5 mM, about 0.5 mM to about 3.5 mM, or about 2.5 mM; for about 10 minutes to about 24 hours, about 15 minutes to about 4 hours, about 15 minutes to about 45 minutes, or about 30 minutes; at about 2 degrees Celsius to about 25 degrees Celsius; about 4 degrees Celsius to about 23 degrees Celsius, about 4 degrees Celsius to about 20 degrees Celsius or about 20 degrees Celsius, inclusive of any endpoints, values or ranges therebetween.

BS3 has a molecular weight of 572.43, sulfo-NHS ester reactive groups, an arm space length of 11.4 A, a solubility in water of 20 mM, can be quenched with, e.g., 10 to 20 mM Tris pH 7.5 for 15 minutes, and its crosslinking is non-cleavable.

In one or more embodiments, crosslinking of a cell, e.g., a non-fragile cell with BS3 may be performed at about 0.05 mM to about 10 mM, about 0.25 mM to about 7.5 mM, about 0.5 mM to about 3.5 mM, or about 2.5 mM; for about 10 minutes to about 24 hours, about 15 minutes to about 4 hours, about 15 minutes to about 2 hours, or about 30 minutes; at about 2 degrees Celsius to about 25 degrees Celsius; about 4 degrees Celsius to about 23 degrees Celsius, about 4 degrees Celsius to about 20 degrees Celsius or about 20 degrees Celsius, inclusive of any endpoints, values or ranges therebetween.

In one or more embodiments, crosslinking of a fragile cell with BS3 may be performed at about 0.05 mM to about 10 mM, about 0.25 mM to about 5 mM, about 0.5 mM to about 5 mM, or about 5 mM; for about 10 minutes to about 24 hours, about 15 minutes to about 4 hours, about 20 minutes to about 2 hours, or about 1 hour; at about 2 degrees Celsius to about 25 degrees Celsius; about 4 degrees Celsius to about 23 degrees Celsius, about 4 degrees Celsius to about 20 degrees Celsius or about 20 degrees Celsius, inclusive of any endpoints, values or ranges therebetween.

Sulfo-EGS has a molecular weight of 660.45, sulfo-NHS ester reactive groups, an arm space length of 16.1 A, a solubility in water of 10 mM, can be stored at −20 degrees Celsius in powder aliquots, can be quenched with, e.g., 20 mM Tris pH 7.5 for 20 minutes, and its crosslinking is cleavable e.g., in hydroxylamine-HCl pH 8.5 for 3 to 6 hours at 37 degrees Celsius.

In one or more embodiments, crosslinking of a cell, e.g., a non-fragile cell with sulfo-EGS may be performed at about 0.25 mM to about 10 mM, about 0.5 mM to about 5 mM, about 0.5 mM to about 4 mM, or about 2.5 mM; for about 10 minutes to about 24 hours, about 15 minutes to about 4 hours, about 15 minutes to about 1 hour, or about 30 minutes; at about 2 degrees Celsius to about 25 degrees Celsius; about 4 degrees Celsius to about 23 degrees Celsius, about 4 degrees Celsius to about 20 degrees Celsius or about 20 degrees Celsius, inclusive of any endpoints, values or ranges therebetween.

In one or more embodiments, crosslinking of a fragile cell with sulfo-EGS may be performed at about 0.25 mM to about 10 mM, about 0.5 mM to about 7 mM, about 1 mM to about 5 mM, or about 5 mM; for about 10 minutes to about 24 hours, about 15 minutes to about 4 hours, about 20 minutes to about 2 hours, or about 1 hour; at about 2 degrees Celsius to about 25 degrees Celsius; about 4 degrees Celsius to about 23 degrees Celsius, about 4 degrees Celsius to about 20 degrees Celsius or about 20 degrees Celsius, inclusive of any endpoints, values or ranges therebetween.

BS(PEG)9 has a molecular weight of 708.71, sulfo-NHS ester reactive groups, an arm space length of 35.7 A, a solubility in water of 250 mM, can be stored at −20 degrees Celsius in powder aliquots, can be quenched with, e.g., 20 to 50 mM Tris pH 7.5 for 15 minutes, and its crosslinking is non-cleavable.

In one or more embodiments, crosslinking of a cell, e.g., a non-fragile cell with BS(PEG)9 may be performed at about 0.25 mM to about 10 mM, about 0.5 mM to about 5 mM, about 1 mM to about 2.5 mM, or about 2.5 mM; for about 10 minutes to about 24 hours, about 15 minutes to about 4 hours, about 15 minutes to about 1 hour, or about 30 minutes; at about 2 degrees Celsius to about 25 degrees Celsius; about 4 degrees Celsius to about 23 degrees Celsius, about 4 degrees Celsius to about 20 degrees Celsius or about 20 degrees Celsius, inclusive of any endpoints, values or ranges therebetween.

In one or more embodiments, crosslinking of a fragile cell with BS(PEG)9 may be performed at about 0.25 mM to about 10 mM, about 0.5 mM to about 5 mM, about 1 mM to about 3.5 mM, or about 5 mM; for about 10 minutes to about 24 hours, about 15 minutes to about 4 hours, about 20 minutes to about 2 hours, or about 1 hour; at about 2 degrees Celsius to about 25 degrees Celsius; about 4 degrees Celsius to about 23 degrees Celsius, about 4 degrees Celsius to about 20 degrees Celsius or about 20 degrees Celsius, inclusive of any endpoints, values or ranges therebetween.

DTSSP has a molecular weight of 608.51, sulfo-NHS ester reactive groups, an arm space length of 12 A, a solubility in water of >6 mg/ml or 9.86 mM, can be stored at 4 degrees Celsius, can be quenched with e.g., 20 to 50 mM Tris pH 7.5 for 15 minutes, and its crosslinking can be reversed, e.g., with 20 to 50 mM DTT at 37 degrees Celsius for 30 minutes.

In one or more embodiments, crosslinking of a cell, e.g., a non-fragile cell with DTSSP may be performed at about 0.25 mM to about 10 mM, about 0.5 mM to about 7 mM, about 1 to about 7 mM, or about 2.5 mM; for about 10 minutes to about 24 hours, about 15 minutes to about 4 hours, about 15 minutes to about 1 hour, or about 30 minutes; at about 2 degrees Celsius to about 25 degrees Celsius; about 4 degrees Celsius to about 23 degrees Celsius, about 4 degrees Celsius to about 20 degrees Celsius or about 20 degrees Celsius, inclusive of any endpoints, values or ranges therebetween.

In one or more embodiments, crosslinking of a fragile cell with DTSSP may be performed at about 0.25 mM to about 20 mM, about 1 mM to about 10 mM, about 2 mM to about 10 mM, or about 5 mM; for about 10 minutes to about 24 hours, about 15 minutes to about 4 hours, about 20 minutes to about 2 hours, or about 1 hour; at about 2 degrees Celsius to about 25 degrees Celsius; about 4 degrees Celsius to about 23 degrees Celsius, about 4 degrees Celsius to about 20 degrees Celsius or about 20 degrees Celsius, inclusive of any endpoints, values or ranges therebetween.

DTBP has a molecular weight of 309.28, imidoester reactive groups, an arm space length of 11.9 A, is soluble in water, can be stored at 4 degrees Celsius, can be quenched with e.g., 1:4 glacial acetic acid and its crosslinking can be reversed, e.g., with 100 to 150 mM DTT at 37 degrees Celsius for 30 minutes.

In one or more embodiments, crosslinking of a cell, e.g., a non-fragile cell with DTBP may be performed at about 0.25 mM to about 10 mM, about 1 mM to about 5 mM, about 0.5 mM to about 3.5 mM, or about 2.5 mM; for about 10 minutes to about 24 hours, about 15 minutes to about 4 hours, about 15 minutes to about 1 hour, or about 30 minutes; at about 2 degrees Celsius to about 25 degrees Celsius; about 4 degrees Celsius to about 23 degrees Celsius, about 4 degrees Celsius to about 20 degrees Celsius or about 20 degrees Celsius, inclusive of any endpoints, values or ranges therebetween.

In one or more embodiments, crosslinking of a fragile cell with DTBP may be performed at about 0.25 mM to about 10 mM, about 1 mM to about 5 mM, about 0.5 mM to about 3.5 mM, or about 5 mM; for about 10 minutes to about 24 hours, about 15 minutes to about 4 hours, about 20 minutes to about 2 hours, or about 20 minutes; at about 2 degrees Celsius to about 25 degrees Celsius; about 4 degrees Celsius to about 23 degrees Celsius, about 4 degrees Celsius to about 20 degrees Celsius or about 20 degrees Celsius, inclusive of any endpoints, values or ranges therebetween.

PFA has a molecular weight of 30.026, has a solubility in water of 400 g/L, can be stored at room temperature, can be quenched with e.g., 30 to 125 mM glycine for 5 to 20 minutes and its crosslinking can be reversed e.g., by heating to about 70 to 95 degrees Celsius.

In one or more embodiments, crosslinking of a cell, e.g., a non-fragile cell with PFA may be performed at about 0.25% to about 10%, about 0.5% to about 7%, about 1% to about 5%, or about 4%; for about 5 minutes to about 24 hours, about 10 minutes to about 2 hours, about 30 minutes to about 1 hour, or about 30 minutes; at about 2 degrees Celsius to about 25 degrees Celsius; about 4 degrees Celsius to about 23 degrees Celsius, about 4 degrees Celsius to about 20 degrees Celsius or about 20 degrees Celsius, inclusive of any endpoints, values or ranges therebetween.

In one or more embodiments, crosslinking of a fragile cell with PFA may be performed at about 0.25% to about 10%, about 0.5% to about 5%, about 1% to about 4%, or about 2%; for about 5 minutes to about 24 hours, about 10 minutes to about 2 hours, about 15 minutes to about 45 minutes, or about 20 minutes; at about 2 degrees Celsius to about 25 degrees Celsius; about 4 degrees Celsius to about 23 degrees Celsius, about 4 degrees Celsius to about 15 degrees Celsius or about 4 degrees Celsius, inclusive of any endpoints, values or ranges therebetween.

Glyoxal has a molecular weight of 58.04, is water soluble at 40% w/v, can be stored at room temperature, can be quenched with a buffer exchange to neutral pH.

In one or more embodiments, crosslinking of a cell, e.g., a non-fragile cell with glyoxal may be performed at about 0.25% to about 10%, about 0.5% to about 7%, about 1% to about 5%, or about 2%; for about 10 minutes to about 24 hours, about 15 minutes to about 2 hours, about 20 minutes to about 1 hour, or about 30 minutes; at about 2 degrees Celsius to about 25 degrees Celsius; about 4 degrees Celsius to about 23 degrees Celsius, about 4 degrees Celsius to about 20 degrees Celsius or about 20 degrees Celsius, inclusive of any endpoints, values or ranges therebetween.

In one or more embodiments, crosslinking of a fragile cell with glyoxal may be performed at about 0.25% to about 10%, about 0.5% to about 5%, about 1% to about 4%, or about 1%; for about 10 minutes to about 24 hours, about 15 minutes to about 2 hours, about 15 minutes to about 45 minutes, or about 15 minutes; at about 2 degrees Celsius to about 25 degrees Celsius; about 4 degrees Celsius to about 23 degrees Celsius, about 4 degrees Celsius to about 20 degrees Celsius or about 20 degrees Celsius, inclusive of any endpoints, values or ranges therebetween.

The abovementioned conditions may be applied when using one crosslinker in combination with another, e.g., in sequential crosslinking, e.g., in using a hydrophobic crosslinker in combination with a hydrophilic crosslinker.

### Additional Embodiments

In one or more embodiments, the hydrophobic crosslinker may be ethylene glycol bis(succinimidyl succinate) (EGS), disuccinimidyl suberate (DSS), or dithiobis(succinimidyl propionate) (DSP). In one or more embodiments, the hydrophobic crosslinker may be dithiobis(succinimidyl propionate) (DSP). In one or more embodiments, the hydrophilic crosslinker may be bis(sulfosuccinimidyl)suberate (BS3), ethylene glycol bis(sulfosuccinimidyl succinate) (sulfo-EGS), 3,3′-dithiobis(sulfosuccinimidyl propionate) (DTSSP), bis-succinimide ester-activated PEG (BS(PEG)9) or dimethyl 3,3′-dithiobispropionimidate dihydrochloride (DTBP). In one or more embodiments, the hydrophilic crosslinker may be 3,3′-dithiobis(sulfosuccinimidyl propionate) (DTSSP). In one or more embodiments, the sample preparation may reduce leakage of biological components from cells. In one or more embodiments, the leakage may be reduced about 2-10-fold relative to a control cell or tissue that has not been contacted with the hydrophobic and hydrophilic crosslinkers. In one or more embodiments, the hydrophobic crosslinker may be in a first buffer and the hydrophilic crosslinker is in a second buffer. In one or more embodiments, the first and second buffers may be the same. In one or more embodiments, the first and second buffers may not be the same. In one or more embodiments, the method may further comprise a step v.) suspending the cells from the sample in a third buffer. In one or more embodiments, the third buffer may be a storage buffer.

In one or more embodiments, the hydrophobic crosslinker may comprise about 0.05 mM to 10 mM DSP. In one or more embodiments, the hydrophobic crosslinker may comprise about 0.25 mM to about 7.5 mM DSP. In one or more embodiments, the hydrophobic crosslinker may comprise about 0.5 mM to about 5 mM DSP. In one or more embodiments, the hydrophobic crosslinker may comprise about 2.5 mM DSP.

In one or more embodiments, the hydrophilic crosslinker may comprise about 0.25 mM to about 10 mM DTSSP. In one or more embodiments, the hydrophilic crosslinker may comprise about 0.5 mM to about 7 mM DTSSP. In one or more embodiments, the hydrophilic crosslinker may comprise about 1.0 mM to about 7.0 mM DTSSP. In one or more embodiments, the hydrophilic crosslinker may comprise about 2.5 mM DTSSP.

In one or more embodiments, the method may further comprise storing the sample at room temperature. In one or more embodiments, the method may further comprise storing the sample at about 2 to about 8 degrees Celsius. In one or more embodiments, the method may further comprise cryopreserving the sample.

In one or more embodiments, the first and/or the second buffer may a phosphate buffered saline (PBS)-based buffer. In one or more embodiments, the first and/or the second buffer may be a saline sodium citrate (SSC) based buffer.

In one or more embodiments, contacting the sample with the hydrophobic crosslinker may comprise incubating at about 2 to about 25 degrees Celsius for a time period ranging from about 10 minutes to about 24 hours. In one or more embodiments, contacting the sample with the hydrophobic crosslinker may comprise incubating at about 4 to about 23 degrees Celsius for a time period ranging from about 15 minutes to about 4 hours. In one or more embodiments, contacting the sample with the hydrophobic crosslinker may comprise incubating at about 4 to about 20 degrees Celsius for a time period ranging from about 20 to about 60 minutes. In one or more embodiments, contacting the sample with the hydrophobic crosslinker may comprise incubating at about 20 degrees Celsius for a time period of about 30 minutes. In one or more embodiments, contacting the sample with the hydrophilic crosslinker may comprise incubating at about 2 to about 25 degrees Celsius for a time period ranging from about 10 minutes to about 24 hours.

In one or more embodiments, contacting the sample with the hydrophilic crosslinker may comprise incubating at about 4 to about 23 degrees Celsius for a time period ranging from about 15 minutes to about 4 hours. In one or more embodiments, contacting the sample with the hydrophilic crosslinker may comprise incubating at about 4 to about 20 degrees Celsius for a time period ranging from about 15 to about 60 minutes. In one or more embodiments, contacting the sample with the hydrophilic crosslinker may comprise incubating at about 20 degrees Celsius for a time period of about 30 minutes.

In one or more embodiments, the sample is maintained in the storage buffer for a time period of about 1 hour to about 5 days.

In one or more embodiments, the method may further comprise determining the sequence of one or more nucleic acid sequences from one or more cells from the sample. In one or more embodiments, the method may further comprise partitioning the cells.

In one or more embodiments, the first buffer, the second buffer, and/or the storage buffer further may comprise a cryoprotectant reagent. In one or more embodiments, the first buffer, the second buffer, and/or the storage buffer further may comprise a stabilizing reagent. In one or more embodiments, the first buffer, the second buffer, and/or the storage buffer further may comprise an RNA stabilizer. In one or more embodiments, the first buffer, the second buffer, and/or the storage buffer may further comprise a ribonucleoside vanadyl complex (RVC). In one or more embodiments, the first buffer, the second buffer, and/or the storage buffer may further comprise an RNase inhibitor.

In one or more embodiments, the first buffer, the second buffer, and/or the storage buffer further may comprise a Tetronic® polymer/poloxamine. Tetronic® polymers or poloxamines are X-shaped amphiphilic block copolymers formed by four arms of poly(ethylene oxide)-poly(propylene oxide) (PEO-PPO) blocks bonded to a central ethylenediamine moiety (for a review see e.g., in Alvarez-Lorenzo et al., Front. Biosci. (Elite Ed) 2010, 2(2), 424-440 and Sandez-Macho et al., Biointerfaces 133 (2015) 270-277). Such a structure confers multi-responsive behavior, namely temperature and pH-sensitiveness. At relatively low concentrations but above the critical micellar concentration (CMC), poloxamines generate polymeric micelles. Due to the presence of a hydrophobic core, these nanocarriers are useful in the solubilization and stabilization of poorly water-soluble drugs. Tetronic® polymers have been shown to mitigate cyclodextrin-induced hemolysis from red blood cells (Sandez-Macho et al.). Various poloxamines/Tetronic® polymers are known in the art including, but not limited to, poloxamine 304, poloxamine 701, poloxamine 901, poloxamine 904, poloxamine 908, poloxamine 1107, poloxamine 1301, poloxamine 1304, poloxamine 1307, poloxamine 90R4, and poloxamine 150R1; and their Tetronic® varieties T304, T701, T901, T904, T908, T1107, T1301, T1304, T1307, T90R4, and T150R1, respectively. In one or more embodiments, the Tetronic® polymer may comprise a poloxamine 1107/T1107 Tetronic® polymer. In one or more embodiments, the first buffer, the second buffer, and/or the storage buffer further may comprise comprises from about 0.25 mM to about 1.25 mM of a poloxamine 1107/T1107 Tetronic® polymer.

### Implementation #2

In accordance with one or more embodiments, a method of sample preparation is described. The method of sample preparation may include the following steps in order: i) contacting a plurality of cells with a hydrophobic crosslinker comprising about 2.5 mM DSP for about 30 minutes at about 20 degrees C.; ii) removing the hydrophobic crosslinker from the cells; iii) contacting the cells with a hydrophilic crosslinker comprising about 2.5 mM DTSSP for about 30 minutes at about 20 degrees C.; iv) removing the hydrophilic crosslinker from the cells; and v) suspending the cells in a storage buffer.

In one or more embodiments, the sample preparation may reduce leakage of biological components from cells. In one or more embodiments, the leakage may be reduced about 2-10-fold relative to a control cell or tissue that has not been contacted with the hydrophobic and hydrophilic crosslinkers. In one or more embodiments, the hydrophobic crosslinker may be in a first buffer and the hydrophilic crosslinker is in a second buffer. In one or more embodiments, the first and second buffers may be the same. In one or more embodiments, the first and second buffers may not be the same. In one or more embodiments, the method may further comprise a step v.) suspending the cells from the sample in a third buffer.

In one or more embodiments, the method may further comprise cryopreserving the sample.

In one or more embodiments, the first and/or the second buffer may a phosphate buffered saline (PBS)-based buffer. In one or more embodiments, the first and/or the second buffer may be a saline sodium citrate (SSC) based buffer.

In one or more embodiments, the sample is maintained in the storage buffer for a time period of about 1 hour to about 5 days.

In one or more embodiments, the method may further comprise determining the sequence of one or more nucleic acid sequences from one or more cells from the sample. In one or more embodiments, the method may further comprise partitioning the cells.

In one or more embodiments, the first buffer, the second buffer, and/or the storage buffer further may comprise a cryoprotectant reagent. In one or more embodiments, the first buffer, the second buffer, and/or the storage buffer further may comprise a stabilizing reagent. In one or more embodiments, the first buffer, the second buffer, and/or the storage buffer further may comprise an RNA stabilizer. In one or more embodiments, the first buffer, the second buffer, and/or the storage buffer may further comprise a ribonucleoside vanadyl complex (RVC). In one or more embodiments, the first buffer, the second buffer, and/or the storage buffer may further comprise an RNase inhibitor.

In one or more embodiments, the first buffer, the second buffer, and/or the storage buffer further may comprise a poloxamine/Tetronic® polymer. In one or more embodiments, the Tetronic® polymer may comprise a T1107 Tetronic® polymer. In one or more embodiments, the first buffer, the second buffer, and/or the storage buffer further may comprise comprises from about 0.25 mM to about 1.25 mM of a T1107 Tetronic® polymer.

### Implementation #3

In accordance with one or more embodiments, a kit for sample preparation is described. The kit may include: i) a hydrophobic crosslinker; ii) a hydrophilic crosslinker; iii) a storage buffer; and iv) instructions for use thereof. In one or more embodiments, the hydrophobic crosslinker may be ethylene glycobis(succinimidylsuccinate) (EGS), disuccinimidyl suberate (DSS) or dithiobis(succinimidylpriopionate) (DSP). In one or more embodiments, the hydrophobic crosslinker may be dithiobis(succinimidylpriopionate) (DSP). In one or more embodiments, the hydrophilic crosslinker may be bis(sulfosuccinimidyl)suberate (BS3), ethylene glycobis(sulfosuccinimidylsuccinate) (sulfo-EGS), 3,3′-dithiobis(sulfosuccinimidyl priopionate) (DTSSP), bis(sulfosuccinimidyl)suberate (DSS), bis-succinimide ester-activated PEG (BS(PEG)9) or dimethyl 3,3′-dithiobispropionimidate·2HCl (DTBP). In one or more embodiments, the hydrophilic crosslinker may be 3,3′-dithiobis(sulfosuccinimidyl priopionate) (DTSSP). In one or more embodiments, the hydrophobic crosslinker may comprise about 0.05 mM to about 10 mM DSP. In one or more embodiments, the hydrophobic crosslinker may comprise about 0.25 mM to about 7.5 mM DSP. In one or more embodiments, the hydrophobic crosslinker may comprise about 0.5 mM to about 5 mM DSP. In one or more embodiments, the hydrophobic crosslinker may comprise about 1.25 to about 2.5 mM DSP.

In one or more embodiments, the hydrophilic crosslinker may comprise about 0.25 mM to about 20 mM DTSSP. In one or more embodiments, the hydrophilic crosslinker comprises about 0.5 mM to about 10 mM DTSSP. In one or more embodiments, the hydrophilic crosslinker may comprise about 1.0 mM to about 10 mM DTSSP. In one or more embodiments, the hydrophilic crosslinker may comprise about 2.5 to about 5 mM DTSSP.

In one or more embodiments, the kit may be stored at room temperature.

In one or more embodiments, the storage buffer may be a phosphate buffered saline (PBS)-based storage buffer. In one or more embodiments, the storage buffer may be a saline sodium citrate (SSC) based storage buffer. In one or more embodiments, the hydrophobic crosslinker, the hydrophilic crosslinker, and/or the storage buffer may further comprise a cryoprotectant reagent. In one or more embodiments, the hydrophobic crosslinker, the hydrophilic crosslinker, and/or the storage buffer may further comprise a stabilizing reagent. In one or more embodiments, the hydrophobic crosslinker, the hydrophilic crosslinker, and/or the storage buffer may further comprise an RNA stabilizer. In one or more embodiments, the hydrophobic crosslinker, the hydrophilic crosslinker, and/or the storage buffer may further comprise a ribonucleoside vanadyl complex (RVC). In one or more embodiments, the hydrophobic crosslinker, the hydrophilic crosslinker, and/or the storage buffer may further comprise an RNase inhibitor. In one or more embodiments, the instructions may provide for preserving and storing a plurality of cells or tissue in a manner that reduces leakage of biological components.

In one or more embodiments, the hydrophobic crosslinker, the hydrophilic crosslinker, and/or the storage buffer further may comprise a poloxamine/Tetronic® polymer. In one or more embodiments, the poloxamine/Tetronic® polymer may comprise a poloxamine 1107/T1107 Tetronic® polymer. In one or more embodiments, the hydrophobic crosslinker, the hydrophilic crosslinker, and/or the storage buffer further may comprise from about 0.25 mM to about 1.25 mM of a poloxamine 1107/T1107 Tetronic® polymer.

### Implementation #4

In accordance with one or more embodiments, a kit for sample preparation is described. The kit may include: i) a hydrophobic crosslinker comprising about 2.5 mM DSP; ii) a hydrophilic crosslinker comprising about 2.5 mM DTSSP; iii) a storage buffer; and iv) instructions for use thereof.

In one or more embodiments, the kit may be stored at room temperature.

In one or more embodiments, the storage buffer may be a phosphate buffered saline (PBS)-based storage buffer. In one or more embodiments, the storage buffer may be a saline sodium citrate (SSC) based storage buffer. In one or more embodiments, the hydrophobic crosslinker, the hydrophilic crosslinker, and/or the storage buffer may further comprise a cryoprotectant reagent. In one or more embodiments, the hydrophobic crosslinker, the hydrophilic crosslinker, and/or the storage buffer may further comprise a stabilizing reagent. In one or more embodiments, the hydrophobic crosslinker, the hydrophilic crosslinker, and/or the storage buffer may further comprise an RNA stabilizer. In one or more embodiments, the hydrophobic crosslinker, the hydrophilic crosslinker, and/or the storage buffer may further comprise a ribonucleoside vanadyl complex (RVC). In one or more embodiments, the hydrophobic crosslinker, the hydrophilic crosslinker, and/or the storage buffer may further comprise an RNase inhibitor. In one or more embodiments, the instructions may provide for preserving and storing a plurality of cells or tissue in a manner that reduces leakage of biological components.

In one or more embodiments, the hydrophobic crosslinker, the hydrophilic crosslinker, and/or the storage buffer further may comprise a poloxamine/Tetronic® polymer. In one or more embodiments, the Tetronic® polymer may comprise a poloxamine 1107/T1107 Tetronic® polymer. In one or more embodiments, the hydrophobic crosslinker, the hydrophilic crosslinker, and/or the storage buffer further may comprise from about 0.25 mM to about 1.25 mM of a poloxamine 1107/T1107 Tetronic® polymer.

### Implementation #5

In accordance with one or more embodiments, a method for reducing leakage of biological components from cells during preservation and storage is described. The method of reducing the leakage of biological components from cells may include i) contacting cells with a fixative buffer comprising a poloxamine/Tetronic® polymer; ii) removing the fixative buffer from the cells; and iii) suspending the cells in a storage buffer.

In accordance with one or more embodiments, a kit for reducing leakage of biological components from cells during preservation and storage is described. The kit may include i) a fixative buffer comprising a poloxamine/Tetronic® polymer; ii) a storage buffer; and iii) instructions for preserving and storing a plurality of cells in a manner that reduces leakage of biological components.

In one or more embodiments, the poloxamine/Tetronic® polymer may include a poloxamine 1107/T1107 Tetronic® polymer. In various embodiments, the fixative buffer may include a concentration of poloxamine 1107/T1107 Tetronic® polymer from about 0.05 mM to about 2.50 mM, from about 0.05 mM to about 2.25 mM, from about 0.05 mM to about 2.00 mM, from about 0.05 mM to about 1.75 mM, from about 0.05 mM to about 1.50 mM, from about 0.05 mM to about 1.25 mM, from about 0.05 mM to about 1.00 mM, from about 0.05 mM to about 0.75 mM, from about 0.05 mM to about 0.50 mM, from about 0.10 mM to about 2.50 mM, from about 0.10 mM to about 2.25 mM, from about 0.10 mM to about 2.00 mM, from about 0.10 mM to about 1.75 mM, from about 0.10 mM to about 1.50 mM, from about 0.10 mM to about 1.25 mM, from about 0.10 mM to about 1.00 mM, from about 0.10 mM to about 0.75 mM, from about 0.10 mM to about 0.50 mM, from about 0.15 mM to about 2.50 mM, from about 0.15 mM to about 2.25 mM, from about 0.15 mM to about 2.00 mM, from about 0.15 mM to about 1.75 mM, from about 0.15 mM to about 1.50 mM, from about 0.15 mM to about 1.25 mM, from about 0.15 mM to about 1.00 mM, from about 0.15 mM to about 0.75 mM, from about 0.15 mM to about 0.50 mM, from about 0.20 mM to about 2.50 mM, from about 0.20 mM to about 2.25 mM, from about 0.20 mM to about 2.00 mM, from about 0.20 mM to about 1.75 mM, from about 0.20 mM to about 1.50 mM, from about 0.20 mM to about 1.25 mM, from about 0.20 mM to about 1.00 mM, from about 0.20 mM to about 0.75 mM, from about 0.20 mM to about 0.50 mM, from about 0.30 mM to about 2.50 mM, from about 0.30 mM to about 2.25 mM, from about 0.30 mM to about 2.00 mM, from about 0.30 mM to about 1.75 mM, from about 0.30 mM to about 1.50 mM, from about 0.30 mM to about 1.25 mM, from about 0.30 mM to about 1.00 mM, from about 0.30 mM to about 0.75 mM, from about 0.30 mM to about 0.50 mM, from about 0.35 mM to about 2.50 mM, from about 0.35 mM to about 2.25 mM, from about 0.35 mM to about 2.00 mM, from about 0.35 mM to about 1.75 mM, from about 0.35 mM to about 1.50 mM, from about 0.35 mM to about 1.25 mM, from about 0.35 mM to about 1.00 mM, from about 0.35 mM to about 0.75 mM, or from about 0.35 mM to about 0.50 mM, inclusive of any ranges therebetween.

In one or more embodiments, the fixative buffer may include a crosslinking fixative. In one or more embodiments, the fixative buffer may include glyoxal. In one or more embodiments, the fixative buffer may include from about 0.05% glyoxal to about 1.5% glyoxal, from about 0.05% glyoxal to about 1.75% glyoxal, from about 0.05% glyoxal to about 2.00% glyoxal, from about 0.05% glyoxal to about 2.25% glyoxal, from about 0.05% glyoxal to about 2.50% glyoxal, from about 0.05% glyoxal to about 2.75% glyoxal, from about 0.05% glyoxal to about 3.00% glyoxal, from about 0.05% glyoxal to about 3.25% glyoxal, from about 0.05% glyoxal to about 3.50% glyoxal, from about 0.05% glyoxal to about 3.75% glyoxal, from about 0.05% glyoxal to about 4.00% glyoxal, from about 0.05% glyoxal to about 4.25% glyoxal, from about 0.05% glyoxal to about 4.50% glyoxal, from about 0.05% glyoxal to about 4.75% glyoxal, from about 0.05% glyoxal to about 5.00% glyoxal, from about 0.10% glyoxal to about 1.5% glyoxal, from about 0.10% glyoxal to about 1.75% glyoxal, from about 0.10% glyoxal to about 2.00% glyoxal, from about 0.10% glyoxal to about 2.25% glyoxal, from about 0.10% glyoxal to about 2.50% glyoxal, from about 0.10% glyoxal to about 2.75% glyoxal, from about 0.10% glyoxal to about 3.00% glyoxal, from about 0.10% glyoxal to about 3.25% glyoxal, from about 0.10% glyoxal to about 3.50% glyoxal, from about 0.10% glyoxal to about 3.75% glyoxal, from about 0.10% glyoxal to about 4.00% glyoxal, from about 0.10% glyoxal to about 4.25% glyoxal, from about 0.10% glyoxal to about 4.50% glyoxal, from about 0.10% glyoxal to about 4.75% glyoxal, from about 0.10% glyoxal to about 5.00% glyoxal, from about 0.15% glyoxal to about 1.5% glyoxal, from about 0.15% glyoxal to about 1.75% glyoxal, from about 0.15% glyoxal to about 2.00% glyoxal, from about 0.15% glyoxal to about 2.25% glyoxal, from about 0.15% glyoxal to about 2.50% glyoxal, from about 0.15% glyoxal to about 2.75% glyoxal, from about 0.15% glyoxal to about 3.00% glyoxal, from about 0.15% glyoxal to about 3.25% glyoxal, from about 0.15% glyoxal to about 3.50% glyoxal, from about 0.15% glyoxal to about 3.75% glyoxal, from about 0.15% glyoxal to about 4.00% glyoxal, from about 0.15% glyoxal to about 4.25% glyoxal, from about 0.15% glyoxal to about 4.50% glyoxal, from about 0.15% glyoxal to about 4.75% glyoxal, from about 0.15% glyoxal to about 5.00% glyoxal, from about 0.20% glyoxal to about 1.5% glyoxal, from about 0.20% glyoxal to about 1.75% glyoxal, from about 0.20% glyoxal to about 2.00% glyoxal, from about 0.20% glyoxal to about 2.25% glyoxal, from about 0.20% glyoxal to about 2.50% glyoxal, from about 0.20% glyoxal to about 2.75% glyoxal, from about 0.20% glyoxal to about 3.00% glyoxal, from about 0.20% glyoxal to about 3.25% glyoxal, from about 0.20% glyoxal to about 3.50% glyoxal, from about 0.20% glyoxal to about 3.75% glyoxal, from about 0.20% glyoxal to about 4.00% glyoxal, from about 0.20% glyoxal to about 4.25% glyoxal, from about 0.20% glyoxal to about 4.50% glyoxal, from about 0.20% glyoxal to about 4.75% glyoxal, from about 0.20% glyoxal to about 5.00% glyoxal, from about 0.25% glyoxal to about 1.5% glyoxal, from about 0.25% glyoxal to about 1.75% glyoxal, from about 0.25% glyoxal to about 2.00% glyoxal, from about 0.25% glyoxal to about 2.25% glyoxal, from about 0.25% glyoxal to about 2.50% glyoxal, from about 0.25% glyoxal to about 2.75% glyoxal, from about 0.25% glyoxal to about 3.00% glyoxal, from about 0.25% glyoxal to about 3.25% glyoxal, from about 0.25% glyoxal to about 3.50% glyoxal, from about 0.25% glyoxal to about 3.75% glyoxal, from about 0.25% glyoxal to about 4.00% glyoxal, from about 0.25% glyoxal to about 4.25% glyoxal, from about 0.25% glyoxal to about 4.50% glyoxal, from about 0.25% glyoxal to about 4.75% glyoxal, from about 0.25% glyoxal to about 5.00% glyoxal, from about 0.30% glyoxal to about 1.5% glyoxal, from about 0.30% glyoxal to about 1.75% glyoxal, from about 0.30% glyoxal to about 2.00% glyoxal, from about 0.30% glyoxal to about 2.25% glyoxal, from about 0.30% glyoxal to about 2.50% glyoxal, from about 0.30% glyoxal to about 2.75% glyoxal, from about 0.30% glyoxal to about 3.00% glyoxal, from about 0.30% glyoxal to about 3.25% glyoxal, from about 0.30% glyoxal to about 3.50% glyoxal, from about 0.30% glyoxal to about 3.75% glyoxal, from about 0.30% glyoxal to about 4.00% glyoxal, from about 0.30% glyoxal to about 4.25% glyoxal, from about 0.30% glyoxal to about 4.50% glyoxal, from about 0.30% glyoxal to about 4.75% glyoxal, from about 0.30% glyoxal to about 5.00% glyoxal, from about 0.35% glyoxal to about 1.5% glyoxal, from about 0.35% glyoxal to about 1.75% glyoxal, from about 0.35% glyoxal to about 2.00% glyoxal, from about 0.35% glyoxal to about 2.25% glyoxal, from about 0.35% glyoxal to about 2.50% glyoxal, from about 0.35% glyoxal to about 2.75% glyoxal, from about 0.35% glyoxal to about 3.00% glyoxal, from about 0.35% glyoxal to about 3.25% glyoxal, from about 0.35% glyoxal to about 3.50% glyoxal, from about 0.35% glyoxal to about 3.75% glyoxal, from about 0.35% glyoxal to about 4.00% glyoxal, from about 0.35% glyoxal to about 4.25% glyoxal, from about 0.35% glyoxal to about 4.50% glyoxal, from about 0.35% glyoxal to about 4.75% glyoxal, or from about 0.35% glyoxal to about 5.00% glyoxal, inclusive of any ranges therebetween.

In one or more embodiments, the fixative buffer may include DSP or DTSSP.

In various embodiments, the fixative buffer may include a concentration of DSP from about 0.05 mM to about 10 mM, from about 0.05 mM to about 7.5 mM, from about 0.05 mM to about 5.00 mM, from about 0.05 mM to about 3.5 mM, from about 0.05 mM to about 2.50 mM, from about 0.05 mM to about 1.25 mM, from about 0.05 mM to about 1.00 mM, from about 0.05 mM to about 0.75 mM, from about 0.05 mM to about 0.50 mM, 0.1 mM to about 10 mM, from about 0.1 mM to about 7.5 mM, from about 0.1 mM to about 5.00 mM, from about 0.1 mM to about 3.5 mM, from about 0.1 mM to about 2.50 mM, from about 0.1 mM to about 1.25 mM, from about 0.1 mM to about 1.00 mM, from about 0.1 mM to about 0.75 mM, from about 0.1 mM to about 0.50 mM, 0.25 mM to about 10 mM, from about 0.25 mM to about 7.5 mM, from about 0.25 mM to about 5.00 mM, from about 0.25 mM to about 3.5 mM, from about 0.25 mM to about 2.50 mM, from about 0.25 mM to about 1.25 mM, from about 0.25 mM to about 1.00 mM, from about 0.25 mM to about 0.75 mM, from about 0.25 mM to about 0.50 mM, 0.5 mM to about 10 mM, from about 0.5 mM to about 7.5 mM, from about 0.5 mM to about 5.00 mM, from about 0.5 mM to about 3.5 mM, from about 0.5 mM to about 2.50 mM, from about 0.5 mM to about 1.25 mM, from about 0.5 mM to about 1.00 mM, from about 0.5 mM to about 0.75 mM, 1.25 mM to about 10 mM, from about 1.25 mM to about 7.5 mM, from about 1.25 mM to about 5.00 mM, from about 1.25 mM to about 3.5 mM, from about 1.25 mM to about 2.50 mM, 2.5 mM to about 10 mM, from about 2.5 mM to about 7.5 mM, from about 2.5 mM to about 5.00 mM, and from about 2.5 mM to about 3.5 mM, inclusive of any ranges, values or endpoints therebetween. In various embodiments, the concentration of DSP is 2.5 mM or 1.25 mM.

In various embodiments, the fixative buffer may include a concentration of DTSSP from about 0.25 mM to about 20 mM, from about 0.25 mM to about 10 mM, from about 0.25 mM to about 7.5 mM, from about 0.25 mM to about 7.0 mM, from about 0.25 mM to about 5.00 mM, from about 0.25 mM to about 3.5 mM, from about 0.25 mM to about 2.50 mM, from about 0.25 mM to about 1.25 mM, from about 0.25 mM to about 1.00 mM, from about 0.25 mM to about 0.75 mM, from about 0.25 mM to about 0.50 mM, from about 0.5 mM to about 20 mM, from about 0.5 mM to about 10 mM, from about 0.5 mM to about 7.5 mM, from about 0.5 mM to about 7.0 mM, from about 0.5 mM to about 5.00 mM, from about 0.5 mM to about 3.5 mM, from about 0.5 mM to about 2.50 mM, from about 0.5 mM to about 1.25 mM, from about 0.5 mM to about 1.00 mM, from about 0.5 mM to about 0.75 mM, from about 1 mM to about 20 mM, from about 1 mM to about 10 mM, from about 1 mM to about 7.5 mM, from about 1 mM to about 7.0 mM, from about 1 mM to about 5.00 mM, from about 1 mM to about 3.5 mM, from about 1 mM to about 2.50 mM, from about 1 mM to about 1.25 mM, from about 2 mM to about 20 mM, from about 2 mM to about 10 mM, from about 2 mM to about 7.5 mM, from about 2 mM to about 7.0 mM, from about 2 mM to about 5.00 mM, from about 2 mM to about 3.5 mM, from about 2 mM to about 2.50 mM, from about 2.5 mM to about 20 mM, from about 2.5 mM to about 10 mM, from about 2.5 mM to about 7.5 mM, from about 2.5 mM to about 7.0 mM, from about 2.5 mM to about 5.00 mM, and from about 2.5 mM to about 3.5 mM, inclusive of any ranges, values or endpoints therebetween. In various embodiments, the concentration of DTSSP is 2.5 mM or 5 mM.

In one or more embodiments, the method of reducing the leakage of biological components from cells or instructions for preserving and storing the cells may further include storing the cells at room temperature. In one or more embodiments, the method of reducing the leakage of biological components from cells or instructions for preserving and storing the cells may further include storing the cells at about 1 degree Celsius to about 4 degrees Celsius, about 1 degree Celsius to about 5 degrees Celsius, about 1 degree Celsius to about 6 degrees Celsius, about 1 degree Celsius to about 7 degrees Celsius, about 1 degree Celsius to about 8 degrees Celsius, about 1 degree Celsius to about 9 degrees Celsius, about 1 degree Celsius to about 10 degrees Celsius, about 1 degree Celsius to about 11 degrees Celsius, about 1 degree Celsius to about 12 degrees Celsius, about 2 degrees Celsius to about 4 degrees Celsius, about 2 degrees Celsius to about 5 degrees Celsius, about 2 degrees Celsius to about 6 degrees Celsius, about 2 degrees Celsius to about 7 degrees Celsius, about 2 degrees Celsius to about 8 degrees Celsius, about 2 degrees Celsius to about 9 degrees Celsius, about 2 degrees Celsius to about 10 degrees Celsius, about 2 degrees Celsius to about 11 degrees Celsius, about 2 degrees Celsius to about 12 degrees Celsius, about 3 degrees Celsius to about 4 degrees Celsius, about 3 degrees Celsius to about 5 degrees Celsius, about 3 degrees Celsius to about 6 degrees Celsius, about 3 degrees Celsius to about 7 degrees Celsius, about 3 degrees Celsius to about 8 degrees Celsius, about 3 degrees Celsius to about 9 degrees Celsius, about 3 degrees Celsius to about 10 degrees Celsius, about 3 degrees Celsius to about 11 degrees Celsius, about 3 degrees Celsius to about 12 degrees Celsius, about 4 degrees Celsius to about 5 degrees Celsius, about 4 degrees Celsius to about 6 degrees Celsius, about 4 degrees Celsius to about 7 degrees Celsius, about 4 degrees Celsius to about 8 degrees Celsius, about 4 degrees Celsius to about 9 degrees Celsius, about 4 degrees Celsius to about 10 degrees Celsius, about 4 degrees Celsius to about 11 degrees Celsius, about 4 degrees Celsius to about 12 degrees Celsius, about 5 degrees Celsius to about 6 degrees Celsius, about 5 degrees Celsius to about 7 degrees Celsius, about 5 degrees Celsius to about 8 degrees Celsius, about 5 degrees Celsius to about 9 degrees Celsius, about 5 degrees Celsius to about 10 degrees Celsius, about 5 degrees Celsius to about 11 degrees Celsius, or about 5 degrees Celsius to about 12 degrees Celsius, inclusive of any ranges therebetween.

In one or more embodiments, the method of reducing the leakage of biological components from cells or instructions for preserving and storing the cells may further include cryopreserving the cells.

In one or more embodiments, the method of reducing the leakage of biological components from cells or instructions for preserving and storing the cells may further include cryopreserving the cells at about 0 degrees Celsius to about −196 degrees Celsius, 0 degrees Celsius to about −175 degrees Celsius, 0 degrees Celsius to about −150 degrees Celsius, 0 degrees Celsius to about −125 degrees Celsius, 0 degrees Celsius to about −100 degrees Celsius, 0 degrees Celsius to about −80 degrees Celsius, 0 degrees Celsius to about −50 degrees Celsius, 0 degrees Celsius to about −20 degrees Celsius, about −20 degrees Celsius to about −196 degrees Celsius, −20 degrees Celsius to about −175 degrees Celsius, −20 degrees Celsius to about −150 degrees Celsius, −20 degrees Celsius to about −125 degrees Celsius, −20 degrees Celsius to about −100 degrees Celsius, −20 degrees Celsius to about −80 degrees Celsius, −20 degrees Celsius to about −50 degrees Celsius, −80 degrees Celsius to about −196 degrees Celsius, −80 degrees Celsius to about −175 degrees Celsius, −80 degrees Celsius to about −150 degrees Celsius, −80 degrees Celsius to about −125 degrees Celsius, and −80 degrees Celsius to about −100 degrees Celsius, inclusive of any ranges, values or endpoints therebetween.

In one or more embodiments, the storage buffer is a phosphate buffered saline (PBS)-based storage buffer. In one or more embodiments, the storage buffer is a saline sodium citrate (SSC) based storage buffer.

In one or more embodiments, the contacting step may include incubating for 0.5 hour, 1 hour, 1.5 hours, or 2 hours at room temperature or incubating overnight at 2-8 degrees Celsius, 1-7 degrees Celsius, or 1-9 degrees Celsius.

In one or more embodiments, the cells may be maintained in the storage buffer for a time period of about 0.5 hour to about 1 day, about 0.5 hour to about 2 days, about 0.5 hour to about 3 days, about 0.5 hour to about 4 days, about 0.5 hour to about 5 days, about 0.5 hour to about 6 days, about 0.5 hour to about 7 days, about 1 hour to about 1 day, about 1 hour to about 2 days, about 1 hour to about 3 days, about 1 hour to about 4 days, about 1 hour to about 5 days, about 1 hour to about 6 days, about 1 hour to about 7 days, about 2 hours to about 1 day, about 2 hours to about 2 days, about 2 hours to about 3 days, about 2 hours to about 4 days, about 2 hours to about 5 days, about 2 hours to about 6 days, about 2 hours to about 7 days, about 3 hours to about 1 day, about 3 hours to about 2 days, about 3 hours to about 3 days, about 3 hours to about 4 days, about 3 hours to about 5 days, about 3 hours to about 6 days, or about 3 hours to about 7 days, inclusive of any ranges therebetween. In other embodiments cells may be stored for more than 1 week, such as 2 weeks, 3 weeks, 1 month or up to one year.

In one or more embodiments, the method of reducing the leakage of biological components from cells or instructions for preserving and storing the cells may further include analyzing one or more of the cells. In one or more embodiments, analyzing one or more of the cells may include conducting a molecular analysis on one or more of the cells. In one or more embodiments, analyzing one or more of the cells may include conducting a nucleic acid or protein analysis of one or more of the cells. In one or more embodiments, analyzing one or more of the cells may include conducting a ribonucleic acid (RNA) analysis of one or more of the cells. In one or more embodiments, analyzing one or more of the cells may include conducting a single cell RNA analysis of one or more of the cells. In one or more embodiments, analyzing one or more of the cells may include conducting a deoxyribonucleic acid (DNA) analysis of one or more of the cells. In one or more embodiments, analyzing one or more of the cells may include conducting a single cell deoxyribonucleic acid (DNA) analysis of one or more of the cells. In one or more embodiments, analyzing one or more of the cells may include conducting a protein analysis of one or more of the cells. In one or more embodiments, analyzing one or more of the cells may include conducting a single cell protein analysis of one or more of the cells.

In one or more embodiments, the method of reducing the leakage of biological components from cells or instructions for preserving and storing the cells may further include conducting a molecular analysis of biological components of one or more of the cells. In one or more embodiments, the molecular analysis is single cell molecular analysis.

In one or more embodiments, the method of reducing the leakage of biological components from cells or instructions for preserving and storing the cells may further include partitioning of the biological components for single cell analysis. In one or more embodiments, the partition is an aqueous droplet in oil emulsion. In one or more embodiments, the kit further includes an oil for partitioning in an aqueous droplet in oil emulsion. In one or more embodiments, the partition further includes a gel bead. In one or more embodiments, the kit further includes gel beads for the partitioning.

In one or more embodiments, the method of reducing the leakage of biological components from cells or instructions for preserving and storing the cells may further include generating single cell barcoded biological components (analytes) for single cell data processing.

In one or more embodiments, the fixative buffer, treatment buffer, or storage buffer further includes a cryoprotectant reagent. In one or more embodiments, the fixative buffer, treatment buffer, or storage buffer further includes a stabilizing reagent. In one or more embodiments, the fixative buffer, treatment buffer, or storage buffer further includes an RNA stabilizer. In one or more embodiments, the fixative buffer, treatment buffer, or storage buffer further includes a ribonucleoside vanadyl complex (RVC). In one or more embodiments, the fixative buffer, treatment buffer, or storage buffer further includes an RNase inhibitor.

In one or more embodiments, the method of reducing the leakage of biological components from cells or instructions for preserving and storing the cells may further include i) contacting the plurality of cells with a fixative buffer for 1 hour at room temperature or overnight at about 2 to about 8 degrees Celsius, wherein the fixative buffer comprises about 0.25 mM to about 1.25 mM of a poloxamine 1107/T1107 Tetronic® polymer; ii) removing the fixative buffer from the cells; and iii) suspending the cells in a storage buffer.

In one or more embodiments, the biological components comprise RNA. In one or more embodiments, the biological components comprise DNA. In one or more embodiments, the biological components comprise protein.

### Implementation #6

In accordance with one or more embodiments, a method for reducing leakage of biological components from cells during preservation and storage is described. The method of reducing the leakage of biological components from cells may include i) contacting a plurality of cells with a fixative buffer; ii) removing the fixative buffer from the cells; iii) contacting the cells with a treatment buffer comprising a poloxamine/Tetronic® polymer; iv) removing the treatment buffer from the cells; and v) suspending the cells in a storage buffer.

In accordance with one or more embodiments, a kit for reducing leakage of biological components from cells during preservation and storage is described. The kit may include i) a fixative buffer; ii) a treatment buffer comprising a poloxamine/Tetronic® polymer; iii) a storage buffer and iv) instructions for preserving and storing a plurality of cells in a manner that reduces leakage of biological components.

In one or more embodiments, the poloxamine/Tetronic® polymer may include a poloxamine 1107/T1107 Tetronic® polymer.

In one or more embodiments, the fixative buffer may include a crosslinking fixative. In one or more embodiments, the fixative buffer may include glyoxal. In one or more embodiments, the fixative buffer may include from about 0.05% glyoxal to about 1.5% glyoxal, from about 0.05% glyoxal to about 1.75% glyoxal, from about 0.05% glyoxal to about 2.00% glyoxal, from about 0.05% glyoxal to about 2.25% glyoxal, from about 0.05% glyoxal to about 2.50% glyoxal, from about 0.05% glyoxal to about 2.75% glyoxal, from about 0.05% glyoxal to about 3.00% glyoxal, from about 0.05% glyoxal to about 3.25% glyoxal, from about 0.05% glyoxal to about 3.50% glyoxal, from about 0.05% glyoxal to about 3.75% glyoxal, from about 0.05% glyoxal to about 4.00% glyoxal, from about 0.05% glyoxal to about 4.25% glyoxal, from about 0.05% glyoxal to about 4.50% glyoxal, from about 0.05% glyoxal to about 4.75% glyoxal, from about 0.05% glyoxal to about 5.00% glyoxal, from about 0.10% glyoxal to about 1.5% glyoxal, from about 0.10% glyoxal to about 1.75% glyoxal, from about 0.10% glyoxal to about 2.00% glyoxal, from about 0.10% glyoxal to about 2.25% glyoxal, from about 0.10% glyoxal to about 2.50% glyoxal, from about 0.10% glyoxal to about 2.75% glyoxal, from about 0.10% glyoxal to about 3.00% glyoxal, from about 0.10% glyoxal to about 3.25% glyoxal, from about 0.10% glyoxal to about 3.50% glyoxal, from about 0.10% glyoxal to about 3.75% glyoxal, from about 0.10% glyoxal to about 4.00% glyoxal, from about 0.10% glyoxal to about 4.25% glyoxal, from about 0.10% glyoxal to about 4.50% glyoxal, from about 0.10% glyoxal to about 4.75% glyoxal, from about 0.10% glyoxal to about 5.00% glyoxal, from about 0.15% glyoxal to about 1.5% glyoxal, from about 0.15% glyoxal to about 1.75% glyoxal, from about 0.15% glyoxal to about 2.00% glyoxal, from about 0.15% glyoxal to about 2.25% glyoxal, from about 0.15% glyoxal to about 2.50% glyoxal, from about 0.15% glyoxal to about 2.75% glyoxal, from about 0.15% glyoxal to about 3.00% glyoxal, from about 0.15% glyoxal to about 3.25% glyoxal, from about 0.15% glyoxal to about 3.50% glyoxal, from about 0.15% glyoxal to about 3.75% glyoxal, from about 0.15% glyoxal to about 4.00% glyoxal, from about 0.15% glyoxal to about 4.25% glyoxal, from about 0.15% glyoxal to about 4.50% glyoxal, from about 0.15% glyoxal to about 4.75% glyoxal, from about 0.15% glyoxal to about 5.00% glyoxal, from about 0.20% glyoxal to about 1.5% glyoxal, from about 0.20% glyoxal to about 1.75% glyoxal, from about 0.20% glyoxal to about 2.00% glyoxal, from about 0.20% glyoxal to about 2.25% glyoxal, from about 0.20% glyoxal to about 2.50% glyoxal, from about 0.20% glyoxal to about 2.75% glyoxal, from about 0.20% glyoxal to about 3.00% glyoxal, from about 0.20% glyoxal to about 3.25% glyoxal, from about 0.20% glyoxal to about 3.50% glyoxal, from about 0.20% glyoxal to about 3.75% glyoxal, from about 0.20% glyoxal to about 4.00% glyoxal, from about 0.20% glyoxal to about 4.25% glyoxal, from about 0.20% glyoxal to about 4.50% glyoxal, from about 0.20% glyoxal to about 4.75% glyoxal, from about 0.20% glyoxal to about 5.00% glyoxal, from about 0.25% glyoxal to about 1.5% glyoxal, from about 0.25% glyoxal to about 1.75% glyoxal, from about 0.25% glyoxal to about 2.00% glyoxal, from about 0.25% glyoxal to about 2.25% glyoxal, from about 0.25% glyoxal to about 2.50% glyoxal, from about 0.25% glyoxal to about 2.75% glyoxal, from about 0.25% glyoxal to about 3.00% glyoxal, from about 0.25% glyoxal to about 3.25% glyoxal, from about 0.25% glyoxal to about 3.50% glyoxal, from about 0.25% glyoxal to about 3.75% glyoxal, from about 0.25% glyoxal to about 4.00% glyoxal, from about 0.25% glyoxal to about 4.25% glyoxal, from about 0.25% glyoxal to about 4.50% glyoxal, from about 0.25% glyoxal to about 4.75% glyoxal, from about 0.25% glyoxal to about 5.00% glyoxal, from about 0.30% glyoxal to about 1.5% glyoxal, from about 0.30% glyoxal to about 1.75% glyoxal, from about 0.30% glyoxal to about 2.00% glyoxal, from about 0.30% glyoxal to about 2.25% glyoxal, from about 0.30% glyoxal to about 2.50% glyoxal, from about 0.30% glyoxal to about 2.75% glyoxal, from about 0.30% glyoxal to about 3.00% glyoxal, from about 0.30% glyoxal to about 3.25% glyoxal, from about 0.30% glyoxal to about 3.50% glyoxal, from about 0.30% glyoxal to about 3.75% glyoxal, from about 0.30% glyoxal to about 4.00% glyoxal, from about 0.30% glyoxal to about 4.25% glyoxal, from about 0.30% glyoxal to about 4.50% glyoxal, from about 0.30% glyoxal to about 4.75% glyoxal, from about 0.30% glyoxal to about 5.00% glyoxal, from about 0.35% glyoxal to about 1.5% glyoxal, from about 0.35% glyoxal to about 1.75% glyoxal, from about 0.35% glyoxal to about 2.00% glyoxal, from about 0.35% glyoxal to about 2.25% glyoxal, from about 0.35% glyoxal to about 2.50% glyoxal, from about 0.35% glyoxal to about 2.75% glyoxal, from about 0.35% glyoxal to about 3.00% glyoxal, from about 0.35% glyoxal to about 3.25% glyoxal, from about 0.35% glyoxal to about 3.50% glyoxal, from about 0.35% glyoxal to about 3.75% glyoxal, from about 0.35% glyoxal to about 4.00% glyoxal, from about 0.35% glyoxal to about 4.25% glyoxal, from about 0.35% glyoxal to about 4.50% glyoxal, from about 0.35% glyoxal to about 4.75% glyoxal, or from about 0.35% glyoxal to about 5.00% glyoxal, inclusive of any ranges therebetween.

In one or more embodiments, the fixative buffer may include DSP or DTSSP.

In various embodiments, the fixative buffer may include a concentration of DSP from about 0.05 mM to about 10 mM, from about 0.05 mM to about 7.5 mM, from about 0.05 mM to about 5.00 mM, from about 0.05 mM to about 3.5 mM, from about 0.05 mM to about 2.50 mM, from about 0.05 mM to about 1.25 mM, from about 0.05 mM to about 1.00 mM, from about 0.05 mM to about 0.75 mM, from about 0.05 mM to about 0.50 mM, 0.1 mM to about 10 mM, from about 0.1 mM to about 7.5 mM, from about 0.1 mM to about 5.00 mM, from about 0.1 mM to about 3.5 mM, from about 0.1 mM to about 2.50 mM, from about 0.1 mM to about 1.25 mM, from about 0.1 mM to about 1.00 mM, from about 0.1 mM to about 0.75 mM, from about 0.1 mM to about 0.50 mM, 0.25 mM to about 10 mM, from about 0.25 mM to about 7.5 mM, from about 0.25 mM to about 5.00 mM, from about 0.25 mM to about 3.5 mM, from about 0.25 mM to about 2.50 mM, from about 0.25 mM to about 1.25 mM, from about 0.25 mM to about 1.00 mM, from about 0.25 mM to about 0.75 mM, from about 0.25 mM to about 0.50 mM, 0.5 mM to about 10 mM, from about 0.5 mM to about 7.5 mM, from about 0.5 mM to about 5.00 mM, from about 0.5 mM to about 3.5 mM, from about 0.5 mM to about 2.50 mM, from about 0.5 mM to about 1.25 mM, from about 0.5 mM to about 1.00 mM, from about 0.5 mM to about 0.75 mM, 1.25 mM to about 10 mM, from about 1.25 mM to about 7.5 mM, from about 1.25 mM to about 5.00 mM, from about 1.25 mM to about 3.5 mM, from about 1.25 mM to about 2.50 mM, 2.5 mM to about 10 mM, from about 2.5 mM to about 7.5 mM, from about 2.5 mM to about 5.00 mM, and from about 2.5 mM to about 3.5 mM, inclusive of any ranges, values or endpoints therebetween. In various embodiments, the concentration of DSP is 2.5 mM or 1.25 mM.

In various embodiments, the fixative buffer may include a concentration of DTSSP from about 0.25 mM to about 20 mM, from about 0.25 mM to about 10 mM, from about 0.25 mM to about 7.5 mM, from about 0.25 mM to about 7.0 mM, from about 0.25 mM to about 5.00 mM, from about 0.25 mM to about 3.5 mM, from about 0.25 mM to about 2.50 mM, from about 0.25 mM to about 1.25 mM, from about 0.25 mM to about 1.00 mM, from about 0.25 mM to about 0.75 mM, from about 0.25 mM to about 0.50 mM, from about 0.5 mM to about 20 mM, from about 0.5 mM to about 10 mM, from about 0.5 mM to about 7.5 mM, from about 0.5 mM to about 7.0 mM, from about 0.5 mM to about 5.00 mM, from about 0.5 mM to about 3.5 mM, from about 0.5 mM to about 2.50 mM, from about 0.5 mM to about 1.25 mM, from about 0.5 mM to about 1.00 mM, from about 0.5 mM to about 0.75 mM, from about 1 mM to about 20 mM, from about 1 mM to about 10 mM, from about 1 mM to about 7.5 mM, from about 1 mM to about 7.0 mM, from about 1 mM to about 5.00 mM, from about 1 mM to about 3.5 mM, from about 1 mM to about 2.50 mM, from about 1 mM to about 1.25 mM, from about 2 mM to about 20 mM, from about 2 mM to about 10 mM, from about 2 mM to about 7.5 mM, from about 2 mM to about 7.0 mM, from about 2 mM to about 5.00 mM, from about 2 mM to about 3.5 mM, from about 2 mM to about 2.50 mM, from about 2.5 mM to about 20 mM, from about 2.5 mM to about 10 mM, from about 2.5 mM to about 7.5 mM, from about 2.5 mM to about 7.0 mM, from about 2.5 mM to about 5.00 mM, and from about 2.5 mM to about 3.5 mM, inclusive of any ranges, values or endpoints therebetween. In various embodiments, the concentration of DTSSP is 2.5 mM or 5 mM.

In one or more embodiments, the method of reducing the leakage of biological components from cells or instructions for preserving and storing the cells may further include storing the cells at room temperature. In one or more embodiments, the method of reducing the leakage of biological components from cells or instructions for preserving and storing the cells may further include storing the cells at about 1 degree Celsius to about 4 degrees Celsius, about 1 degree Celsius to about 5 degrees Celsius, about 1 degree Celsius to about 6 degrees Celsius, about 1 degree Celsius to about 7 degrees Celsius, about 1 degree Celsius to about 8 degrees Celsius, about 1 degree Celsius to about 9 degrees Celsius, about 1 degree Celsius to about 10 degrees Celsius, about 1 degree Celsius to about 11 degrees Celsius, about 1 degree Celsius to about 12 degrees Celsius, about 2 degrees Celsius to about 4 degrees Celsius, about 2 degrees Celsius to about 5 degrees Celsius, about 2 degrees Celsius to about 6 degrees Celsius, about 2 degrees Celsius to about 7 degrees Celsius, about 2 degrees Celsius to about 8 degrees Celsius, about 2 degrees Celsius to about 9 degrees Celsius, about 2 degrees Celsius to about 10 degrees Celsius, about 2 degrees Celsius to about 11 degrees Celsius, about 2 degrees Celsius to about 12 degrees Celsius, about 3 degrees Celsius to about 4 degrees Celsius, about 3 degrees Celsius to about 5 degrees Celsius, about 3 degrees Celsius to about 6 degrees Celsius, about 3 degrees Celsius to about 7 degrees Celsius, about 3 degrees Celsius to about 8 degrees Celsius, about 3 degrees Celsius to about 9 degrees Celsius, about 3 degrees Celsius to about 10 degrees Celsius, about 3 degrees Celsius to about 11 degrees Celsius, about 3 degrees Celsius to about 12 degrees Celsius, about 4 degrees Celsius to about 5 degrees Celsius, about 4 degrees Celsius to about 6 degrees Celsius, about 4 degrees Celsius to about 7 degrees Celsius, about 4 degrees Celsius to about 8 degrees Celsius, about 4 degrees Celsius to about 9 degrees Celsius, about 4 degrees Celsius to about 10 degrees Celsius, about 4 degrees Celsius to about 11 degrees Celsius, about 4 degrees Celsius to about 12 degrees Celsius, about 5 degrees Celsius to about 6 degrees Celsius, about 5 degrees Celsius to about 7 degrees Celsius, about 5 degrees Celsius to about 8 degrees Celsius, about 5 degrees Celsius to about 9 degrees Celsius, about 5 degrees Celsius to about 10 degrees Celsius, about 5 degrees Celsius to about 11 degrees Celsius, or about 5 degrees Celsius to about 12 degrees Celsius, inclusive of any ranges therebetween.

In one or more embodiments, the method of reducing the leakage of biological components from cells or instructions for preserving and storing the cells may further include cryopreserving the cells.

In one or more embodiments, the method of reducing the leakage of biological components from cells or instructions for preserving and storing the cells may further include cryopreserving the cells at about 0 degrees Celsius to about −196 degrees Celsius, 0 degrees Celsius to about −175 degrees Celsius, 0 degrees Celsius to about −150 degrees Celsius, 0 degrees Celsius to about −125 degrees Celsius, 0 degrees Celsius to about −100 degrees Celsius, 0 degrees Celsius to about −80 degrees Celsius, 0 degrees Celsius to about −50 degrees Celsius, 0 degrees Celsius to about −20 degrees Celsius, about −20 degrees Celsius to about −196 degrees Celsius, −20 degrees Celsius to about −175 degrees Celsius, −20 degrees Celsius to about −150 degrees Celsius, −20 degrees Celsius to about −125 degrees Celsius, −20 degrees Celsius to about −100 degrees Celsius, −20 degrees Celsius to about −80 degrees Celsius, −20 degrees Celsius to about −50 degrees Celsius, −80 degrees Celsius to about −196 degrees Celsius, −80 degrees Celsius to about −175 degrees Celsius, −80 degrees Celsius to about −150 degrees Celsius, −80 degrees Celsius to about −125 degrees Celsius, and −80 degrees Celsius to about −100 degrees Celsius, inclusive of any ranges, values or endpoints therebetween.

In one or more embodiments, the storage buffer is a phosphate buffered saline (PBS)-based storage buffer. In one or more embodiments, the storage buffer is a saline sodium citrate (SSC) based storage buffer.

In one or more embodiments, the contacting step may include incubating for 0.5 hour, 1 hour, 1.5 hours, or 2 hours at room temperature or incubating overnight at 2-8 degrees Celsius, 1-7 degrees Celsius, or 1-9 degrees Celsius.

In one or more embodiments, the cells may be maintained in the storage buffer for a time period of about 0.5 hour to about 1 day, about 0.5 hour to about 2 days, about 0.5 hour to about 3 days, about 0.5 hour to about 4 days, about 0.5 hour to about 5 days, about 0.5 hour to about 6 days, about 0.5 hour to about 7 days, about 1 hour to about 1 day, about 1 hour to about 2 days, about 1 hour to about 3 days, about 1 hour to about 4 days, about 1 hour to about 5 days, about 1 hour to about 6 days, about 1 hour to about 7 days, about 2 hours to about 1 day, about 2 hours to about 2 days, about 2 hours to about 3 days, about 2 hours to about 4 days, about 2 hours to about 5 days, about 2 hours to about 6 days, about 2 hours to about 7 days, about 3 hours to about 1 day, about 3 hours to about 2 days, about 3 hours to about 3 days, about 3 hours to about 4 days, about 3 hours to about 5 days, about 3 hours to about 6 days, or about 3 hours to about 7 days, inclusive of any ranges therebetween. In other embodiments cells may be stored for more than 1 week, such as 2 weeks, 3 weeks, 1 month or up to one year.

In one or more embodiments, the treatment buffer may include a PBS based buffer comprising a concentration of poloxamine 1107/T1107 Tetronic® polymer from about 0.05 mM to about 2.50 mM, from about 0.05 mM to about 2.25 mM, from about 0.05 mM to about 2.00 mM, from about 0.05 mM to about 1.75 mM, from about 0.05 mM to about 1.50 mM, from about 0.05 mM to about 1.25 mM, from about 0.05 mM to about 1.00 mM, from about 0.05 mM to about 0.75 mM, from about 0.05 mM to about 0.50 mM, from about 0.10 mM to about 2.50 mM, from about 0.10 mM to about 2.25 mM, from about 0.10 mM to about 2.00 mM, from about 0.10 mM to about 1.75 mM, from about 0.10 mM to about 1.50 mM, from about 0.10 mM to about 1.25 mM, from about 0.10 mM to about 1.00 mM, from about 0.10 mM to about 0.75 mM, from about 0.10 mM to about 0.50 mM, from about 0.15 mM to about 2.50 mM, from about 0.15 mM to about 2.25 mM, from about 0.15 mM to about 2.00 mM, from about 0.15 mM to about 1.75 mM, from about 0.15 mM to about 1.50 mM, from about 0.15 mM to about 1.25 mM, from about 0.15 mM to about 1.00 mM, from about 0.15 mM to about 0.75 mM, from about 0.15 mM to about 0.50 mM, from about 0.20 mM to about 2.50 mM, from about 0.20 mM to about 2.25 mM, from about 0.20 mM to about 2.00 mM, from about 0.20 mM to about 1.75 mM, from about 0.20 mM to about 1.50 mM, from about 0.20 mM to about 1.25 mM, from about 0.20 mM to about 1.00 mM, from about 0.20 mM to about 0.75 mM, from about 0.20 mM to about 0.50 mM, from about 0.30 mM to about 2.50 mM, from about 0.30 mM to about 2.25 mM, from about 0.30 mM to about 2.00 mM, from about 0.30 mM to about 1.75 mM, from about 0.30 mM to about 1.50 mM, from about 0.30 mM to about 1.25 mM, from about 0.30 mM to about 1.00 mM, from about 0.30 mM to about 0.75 mM, from about 0.30 mM to about 0.50 mM, from about 0.35 mM to about 2.50 mM, from about 0.35 mM to about 2.25 mM, from about 0.35 mM to about 2.00 mM, from about 0.35 mM to about 1.75 mM, from about 0.35 mM to about 1.50 mM, from about 0.35 mM to about 1.25 mM, from about 0.35 mM to about 1.00 mM, from about 0.35 mM to about 0.75 mM, or from about 0.35 mM to about 0.50 mM, inclusive of any ranges therebetween.

In one or more embodiments, the method of reducing the leakage of biological components from cells or instructions for preserving and storing the cells may further include analyzing one or more of the cells. In one or more embodiments, analyzing one or more of the cells may include conducting a molecular analysis on one or more of the cells. In one or more embodiments, analyzing one or more of the cells may include conducting a nucleic acid or protein analysis of one or more of the cells. In one or more embodiments, analyzing one or more of the cells may include conducting a ribonucleic acid (RNA) analysis of one or more of the cells. In one or more embodiments, analyzing one or more of the cells may include conducting a single cell RNA analysis of one or more of the cells. In one or more embodiments, analyzing one or more of the cells may include conducting a deoxyribonucleic acid (DNA) analysis of one or more of the cells. In one or more embodiments, analyzing one or more of the cells may include conducting a single cell deoxyribonucleic acid (DNA) analysis of one or more of the cells. In one or more embodiments, analyzing one or more of the cells may include conducting a protein analysis of one or more of the cells. In one or more embodiments, analyzing one or more of the cells may include conducting a single cell protein analysis of one or more of the cells.

In one or more embodiments, the method of reducing the leakage of biological components from cells or instructions for preserving and storing the cells may further include conducting a molecular analysis of biological components of one or more of the cells. In one or more embodiments, the molecular analysis is single cell molecular analysis.

In one or more embodiments, the method of reducing the leakage of biological components from cells or instructions for preserving and storing the cells may further include partitioning of the biological components for single cell analysis. In one or more embodiments, the partition is an aqueous droplet in oil emulsion. In one or more embodiments, the kit further includes an oil for partitioning in an aqueous droplet in oil emulsion. In one or more embodiments, the partition further includes a gel bead. In one or more embodiments, the kit further includes gel beads for the partitioning.

In one or more embodiments, the method of reducing the leakage of biological components from cells or instructions for preserving and storing the cells may further include generating single cell barcoded biological components (analytes) for single cell data processing.

In one or more embodiments, the fixative buffer, treatment buffer, or storage buffer further includes a cryoprotectant reagent. In one or more embodiments, the fixative buffer, treatment buffer, or storage buffer further includes a stabilizing reagent. In one or more embodiments, the fixative buffer, treatment buffer, or storage buffer further includes an RNA stabilizer. In one or more embodiments, the fixative buffer, treatment buffer, or storage buffer further includes a ribonucleoside vanadyl complex (RVC). In one or more embodiments, the fixative buffer, treatment buffer, or storage buffer further includes an RNase inhibitor.

In one or more embodiments, the biological components comprise RNA. In one or more embodiments, the biological components comprise DNA. In one or more embodiments, the biological components comprise protein.

### Implementation #7

In accordance with one or more embodiments, a method for reducing leakage of biological components from cells during preservation and storage is described. The method of reducing the leakage of biological components from cells may include i) contacting a plurality of cells with a fixative buffer; ii) removing the fixative buffer from the cells; and iii) suspending the cells in a storage buffer comprising a poloxamine/Tetronic® polymer.

In accordance with one or more embodiments, a kit for reducing leakage of biological components from cells during preservation and storage is described. The kit may include i) a fixative buffer; ii) a storage buffer comprising a poloxamine/Tetronic® polymer; and iii) instructions for preserving and storing a plurality of cells in a manner that reduces leakage of biological components.

In one or more embodiments, the poloxamine/Tetronic® polymer may include a poloxamine 1107/T1107 Tetronic® polymer.

In one or more embodiments, the fixative buffer may include a crosslinking fixative. In one or more embodiments, the fixative buffer may include glyoxal. In one or more embodiments, the fixative buffer may include from about 0.05% glyoxal to about 1.5% glyoxal, from about 0.05% glyoxal to about 1.75% glyoxal, from about 0.05% glyoxal to about 2.00% glyoxal, from about 0.05% glyoxal to about 2.25% glyoxal, from about 0.05% glyoxal to about 2.50% glyoxal, from about 0.05% glyoxal to about 2.75% glyoxal, from about 0.05% glyoxal to about 3.00% glyoxal, from about 0.05% glyoxal to about 3.25% glyoxal, from about 0.05% glyoxal to about 3.50% glyoxal, from about 0.05% glyoxal to about 3.75% glyoxal, from about 0.05% glyoxal to about 4.00% glyoxal, from about 0.05% glyoxal to about 4.25% glyoxal, from about 0.05% glyoxal to about 4.50% glyoxal, from about 0.05% glyoxal to about 4.75% glyoxal, from about 0.05% glyoxal to about 5.00% glyoxal, from about 0.10% glyoxal to about 1.5% glyoxal, from about 0.10% glyoxal to about 1.75% glyoxal, from about 0.10% glyoxal to about 2.00% glyoxal, from about 0.10% glyoxal to about 2.25% glyoxal, from about 0.10% glyoxal to about 2.50% glyoxal, from about 0.10% glyoxal to about 2.75% glyoxal, from about 0.10% glyoxal to about 3.00% glyoxal, from about 0.10% glyoxal to about 3.25% glyoxal, from about 0.10% glyoxal to about 3.50% glyoxal, from about 0.10% glyoxal to about 3.75% glyoxal, from about 0.10% glyoxal to about 4.00% glyoxal, from about 0.10% glyoxal to about 4.25% glyoxal, from about 0.10% glyoxal to about 4.50% glyoxal, from about 0.10% glyoxal to about 4.75% glyoxal, from about 0.10% glyoxal to about 5.00% glyoxal, from about 0.15% glyoxal to about 1.5% glyoxal, from about 0.15% glyoxal to about 1.75% glyoxal, from about 0.15% glyoxal to about 2.00% glyoxal, from about 0.15% glyoxal to about 2.25% glyoxal, from about 0.15% glyoxal to about 2.50% glyoxal, from about 0.15% glyoxal to about 2.75% glyoxal, from about 0.15% glyoxal to about 3.00% glyoxal, from about 0.15% glyoxal to about 3.25% glyoxal, from about 0.15% glyoxal to about 3.50% glyoxal, from about 0.15% glyoxal to about 3.75% glyoxal, from about 0.15% glyoxal to about 4.00% glyoxal, from about 0.15% glyoxal to about 4.25% glyoxal, from about 0.15% glyoxal to about 4.50% glyoxal, from about 0.15% glyoxal to about 4.75% glyoxal, from about 0.15% glyoxal to about 5.00% glyoxal, from about 0.20% glyoxal to about 1.5% glyoxal, from about 0.20% glyoxal to about 1.75% glyoxal, from about 0.20% glyoxal to about 2.00% glyoxal, from about 0.20% glyoxal to about 2.25% glyoxal, from about 0.20% glyoxal to about 2.50% glyoxal, from about 0.20% glyoxal to about 2.75% glyoxal, from about 0.20% glyoxal to about 3.00% glyoxal, from about 0.20% glyoxal to about 3.25% glyoxal, from about 0.20% glyoxal to about 3.50% glyoxal, from about 0.20% glyoxal to about 3.75% glyoxal, from about 0.20% glyoxal to about 4.00% glyoxal, from about 0.20% glyoxal to about 4.25% glyoxal, from about 0.20% glyoxal to about 4.50% glyoxal, from about 0.20% glyoxal to about 4.75% glyoxal, from about 0.20% glyoxal to about 5.00% glyoxal, from about 0.25% glyoxal to about 1.5% glyoxal, from about 0.25% glyoxal to about 1.75% glyoxal, from about 0.25% glyoxal to about 2.00% glyoxal, from about 0.25% glyoxal to about 2.25% glyoxal, from about 0.25% glyoxal to about 2.50% glyoxal, from about 0.25% glyoxal to about 2.75% glyoxal, from about 0.25% glyoxal to about 3.00% glyoxal, from about 0.25% glyoxal to about 3.25% glyoxal, from about 0.25% glyoxal to about 3.50% glyoxal, from about 0.25% glyoxal to about 3.75% glyoxal, from about 0.25% glyoxal to about 4.00% glyoxal, from about 0.25% glyoxal to about 4.25% glyoxal, from about 0.25% glyoxal to about 4.50% glyoxal, from about 0.25% glyoxal to about 4.75% glyoxal, from about 0.25% glyoxal to about 5.00% glyoxal, from about 0.30% glyoxal to about 1.5% glyoxal, from about 0.30% glyoxal to about 1.75% glyoxal, from about 0.30% glyoxal to about 2.00% glyoxal, from about 0.30% glyoxal to about 2.25% glyoxal, from about 0.30% glyoxal to about 2.50% glyoxal, from about 0.30% glyoxal to about 2.75% glyoxal, from about 0.30% glyoxal to about 3.00% glyoxal, from about 0.30% glyoxal to about 3.25% glyoxal, from about 0.30% glyoxal to about 3.50% glyoxal, from about 0.30% glyoxal to about 3.75% glyoxal, from about 0.30% glyoxal to about 4.00% glyoxal, from about 0.30% glyoxal to about 4.25% glyoxal, from about 0.30% glyoxal to about 4.50% glyoxal, from about 0.30% glyoxal to about 4.75% glyoxal, from about 0.30% glyoxal to about 5.00% glyoxal, from about 0.35% glyoxal to about 1.5% glyoxal, from about 0.35% glyoxal to about 1.75% glyoxal, from about 0.35% glyoxal to about 2.00% glyoxal, from about 0.35% glyoxal to about 2.25% glyoxal, from about 0.35% glyoxal to about 2.50% glyoxal, from about 0.35% glyoxal to about 2.75% glyoxal, from about 0.35% glyoxal to about 3.00% glyoxal, from about 0.35% glyoxal to about 3.25% glyoxal, from about 0.35% glyoxal to about 3.50% glyoxal, from about 0.35% glyoxal to about 3.75% glyoxal, from about 0.35% glyoxal to about 4.00% glyoxal, from about 0.35% glyoxal to about 4.25% glyoxal, from about 0.35% glyoxal to about 4.50% glyoxal, from about 0.35% glyoxal to about 4.75% glyoxal, or from about 0.35% glyoxal to about 5.00% glyoxal, inclusive of any ranges therebetween.

In one or more embodiments, the fixative buffer may include DSP or DTSSP.

In various embodiments, the fixative buffer may include a concentration of DSP from about 0.05 mM to about 10 mM, from about 0.05 mM to about 7.5 mM, from about 0.05 mM to about 5.00 mM, from about 0.05 mM to about 3.5 mM, from about 0.05 mM to about 2.50 mM, from about 0.05 mM to about 1.25 mM, from about 0.05 mM to about 1.00 mM, from about 0.05 mM to about 0.75 mM, from about 0.05 mM to about 0.50 mM, 0.1 mM to about 10 mM, from about 0.1 mM to about 7.5 mM, from about 0.1 mM to about 5.00 mM, from about 0.1 mM to about 3.5 mM, from about 0.1 mM to about 2.50 mM, from about 0.1 mM to about 1.25 mM, from about 0.1 mM to about 1.00 mM, from about 0.1 mM to about 0.75 mM, from about 0.1 mM to about 0.50 mM, 0.25 mM to about 10 mM, from about 0.25 mM to about 7.5 mM, from about 0.25 mM to about 5.00 mM, from about 0.25 mM to about 3.5 mM, from about 0.25 mM to about 2.50 mM, from about 0.25 mM to about 1.25 mM, from about 0.25 mM to about 1.00 mM, from about 0.25 mM to about 0.75 mM, from about 0.25 mM to about 0.50 mM, 0.5 mM to about 10 mM, from about 0.5 mM to about 7.5 mM, from about 0.5 mM to about 5.00 mM, from about 0.5 mM to about 3.5 mM, from about 0.5 mM to about 2.50 mM, from about 0.5 mM to about 1.25 mM, from about 0.5 mM to about 1.00 mM, from about 0.5 mM to about 0.75 mM, 1.25 mM to about 10 mM, from about 1.25 mM to about 7.5 mM, from about 1.25 mM to about 5.00 mM, from about 1.25 mM to about 3.5 mM, from about 1.25 mM to about 2.50 mM, 2.5 mM to about 10 mM, from about 2.5 mM to about 7.5 mM, from about 2.5 mM to about 5.00 mM, and from about 2.5 mM to about 3.5 mM, inclusive of any ranges, values or endpoints therebetween. In various embodiments, the concentration of DSP is 2.5 mM or 1.25 mM.

In various embodiments, the fixative buffer may include a concentration of DTSSP from about 0.25 mM to about 20 mM, from about 0.25 mM to about 10 mM, from about 0.25 mM to about 7.5 mM, from about 0.25 mM to about 7.0 mM, from about 0.25 mM to about 5.00 mM, from about 0.25 mM to about 3.5 mM, from about 0.25 mM to about 2.50 mM, from about 0.25 mM to about 1.25 mM, from about 0.25 mM to about 1.00 mM, from about 0.25 mM to about 0.75 mM, from about 0.25 mM to about 0.50 mM, from about 0.5 mM to about 20 mM, from about 0.5 mM to about 10 mM, from about 0.5 mM to about 7.5 mM, from about 0.5 mM to about 7.0 mM, from about 0.5 mM to about 5.00 mM, from about 0.5 mM to about 3.5 mM, from about 0.5 mM to about 2.50 mM, from about 0.5 mM to about 1.25 mM, from about 0.5 mM to about 1.00 mM, from about 0.5 mM to about 0.75 mM, from about 1 mM to about 20 mM, from about 1 mM to about 10 mM, from about 1 mM to about 7.5 mM, from about 1 mM to about 7.0 mM, from about 1 mM to about 5.00 mM, from about 1 mM to about 3.5 mM, from about 1 mM to about 2.50 mM, from about 1 mM to about 1.25 mM, from about 2 mM to about 20 mM, from about 2 mM to about 10 mM, from about 2 mM to about 7.5 mM, from about 2 mM to about 7.0 mM, from about 2 mM to about 5.00 mM, from about 2 mM to about 3.5 mM, from about 2 mM to about 2.50 mM, from about 2.5 mM to about 20 mM, from about 2.5 mM to about 10 mM, from about 2.5 mM to about 7.5 mM, from about 2.5 mM to about 7.0 mM, from about 2.5 mM to about 5.00 mM, and from about 2.5 mM to about 3.5 mM, inclusive of any ranges, values or endpoints therebetween. In various embodiments, the concentration of DTSSP is 2.5 mM or 5 mM.

In various embodiments, the storage buffer may include a concentration of poloxamine 1107/T1107 Tetronic® polymer from about 0.05 mM to about 2.50 mM, from about 0.05 mM to about 2.25 mM, from about 0.05 mM to about 2.00 mM, from about 0.05 mM to about 1.75 mM, from about 0.05 mM to about 1.50 mM, from about 0.05 mM to about 1.25 mM, from about 0.05 mM to about 1.00 mM, from about 0.05 mM to about 0.75 mM, from about 0.05 mM to about 0.50 mM, from about 0.10 mM to about 2.50 mM, from about 0.10 mM to about 2.25 mM, from about 0.10 mM to about 2.00 mM, from about 0.10 mM to about 1.75 mM, from about 0.10 mM to about 1.50 mM, from about 0.10 mM to about 1.25 mM, from about 0.10 mM to about 1.00 mM, from about 0.10 mM to about 0.75 mM, from about 0.10 mM to about 0.50 mM, from about 0.15 mM to about 2.50 mM, from about 0.15 mM to about 2.25 mM, from about 0.15 mM to about 2.00 mM, from about 0.15 mM to about 1.75 mM, from about 0.15 mM to about 1.50 mM, from about 0.15 mM to about 1.25 mM, from about 0.15 mM to about 1.00 mM, from about 0.15 mM to about 0.75 mM, from about 0.15 mM to about 0.50 mM, from about 0.20 mM to about 2.50 mM, from about 0.20 mM to about 2.25 mM, from about 0.20 mM to about 2.00 mM, from about 0.20 mM to about 1.75 mM, from about 0.20 mM to about 1.50 mM, from about 0.20 mM to about 1.25 mM, from about 0.20 mM to about 1.00 mM, from about 0.20 mM to about 0.75 mM, from about 0.20 mM to about 0.50 mM, from about 0.30 mM to about 2.50 mM, from about 0.30 mM to about 2.25 mM, from about 0.30 mM to about 2.00 mM, from about 0.30 mM to about 1.75 mM, from about 0.30 mM to about 1.50 mM, from about 0.30 mM to about 1.25 mM, from about 0.30 mM to about 1.00 mM, from about 0.30 mM to about 0.75 mM, from about 0.30 mM to about 0.50 mM, from about 0.35 mM to about 2.50 mM, from about 0.35 mM to about 2.25 mM, from about 0.35 mM to about 2.00 mM, from about 0.35 mM to about 1.75 mM, from about 0.35 mM to about 1.50 mM, from about 0.35 mM to about 1.25 mM, from about 0.35 mM to about 1.00 mM, from about 0.35 mM to about 0.75 mM, or from about 0.35 mM to about 0.50 mM, inclusive of any ranges therebetween.

In one or more embodiments, the method of reducing the leakage of biological components from cells or instructions for preserving and storing the cells may further include storing the cells at room temperature. In one or more embodiments, the method of reducing the leakage of biological components from cells or instructions for preserving and storing the cells may further include storing the cells at about 1 degree Celsius to about 4 degrees Celsius, about 1 degree Celsius to about 5 degrees Celsius, about 1 degree Celsius to about 6 degrees Celsius, about 1 degree Celsius to about 7 degrees Celsius, about 1 degree Celsius to about 8 degrees Celsius, about 1 degree Celsius to about 9 degrees Celsius, about 1 degree Celsius to about 10 degrees Celsius, about 1 degree Celsius to about 11 degrees Celsius, about 1 degree Celsius to about 12 degrees Celsius, about 2 degrees Celsius to about 4 degrees Celsius, about 2 degrees Celsius to about 5 degrees Celsius, about 2 degrees Celsius to about 6 degrees Celsius, about 2 degrees Celsius to about 7 degrees Celsius, about 2 degrees Celsius to about 8 degrees Celsius, about 2 degrees Celsius to about 9 degrees Celsius, about 2 degrees Celsius to about 10 degrees Celsius, about 2 degrees Celsius to about 11 degrees Celsius, about 2 degrees Celsius to about 12 degrees Celsius, about 3 degrees Celsius to about 4 degrees Celsius, about 3 degrees Celsius to about 5 degrees Celsius, about 3 degrees Celsius to about 6 degrees Celsius, about 3 degrees Celsius to about 7 degrees Celsius, about 3 degrees Celsius to about 8 degrees Celsius, about 3 degrees Celsius to about 9 degrees Celsius, about 3 degrees Celsius to about 10 degrees Celsius, about 3 degrees Celsius to about 11 degrees Celsius, about 3 degrees Celsius to about 12 degrees Celsius, about 4 degrees Celsius to about 5 degrees Celsius, about 4 degrees Celsius to about 6 degrees Celsius, about 4 degrees Celsius to about 7 degrees Celsius, about 4 degrees Celsius to about 8 degrees Celsius, about 4 degrees Celsius to about 9 degrees Celsius, about 4 degrees Celsius to about 10 degrees Celsius, about 4 degrees Celsius to about 11 degrees Celsius, about 4 degrees Celsius to about 12 degrees Celsius, about 5 degrees Celsius to about 6 degrees Celsius, about 5 degrees Celsius to about 7 degrees Celsius, about 5 degrees Celsius to about 8 degrees Celsius, about 5 degrees Celsius to about 9 degrees Celsius, about 5 degrees Celsius to about 10 degrees Celsius, about 5 degrees Celsius to about 11 degrees Celsius, or about 5 degrees Celsius to about 12 degrees Celsius, inclusive of any ranges therebetween.

In one or more embodiments, the method of reducing the leakage of biological components from cells or instructions for preserving and storing the cells may further include cryopreserving the cells.

In one or more embodiments, the method of reducing the leakage of biological components from cells or instructions for preserving and storing the cells may further include cryopreserving the cells at about 0 degrees Celsius to about −196 degrees Celsius, 0 degrees Celsius to about −175 degrees Celsius, 0 degrees Celsius to about −150 degrees Celsius, 0 degrees Celsius to about −125 degrees Celsius, 0 degrees Celsius to about −100 degrees Celsius, 0 degrees Celsius to about −80 degrees Celsius, 0 degrees Celsius to about −50 degrees Celsius, 0 degrees Celsius to about −20 degrees Celsius, about −20 degrees Celsius to about −196 degrees Celsius, −20 degrees Celsius to about −175 degrees Celsius, −20 degrees Celsius to about −150 degrees Celsius, −20 degrees Celsius to about −125 degrees Celsius, −20 degrees Celsius to about −100 degrees Celsius, −20 degrees Celsius to about −80 degrees Celsius, −20 degrees Celsius to about −50 degrees Celsius, −80 degrees Celsius to about −196 degrees Celsius, −80 degrees Celsius to about −175 degrees Celsius, −80 degrees Celsius to about −150 degrees Celsius, −80 degrees Celsius to about −125 degrees Celsius, and −80 degrees Celsius to about −100 degrees Celsius, inclusive of any ranges, values or endpoints therebetween.

In one or more embodiments, the storage buffer is a phosphate buffered saline (PBS)-based storage buffer. In one or more embodiments, the storage buffer is a saline sodium citrate (SSC) based storage buffer.

In one or more embodiments, the contacting step may include incubating for 0.5 hour, 1 hour, 1.5 hours, or 2 hours at room temperature or incubating overnight at 2-8 degrees Celsius, 1-7 degrees Celsius, or 1-9 degrees Celsius.

In one or more embodiments, the cells may be maintained in the storage buffer for a time period of about 0.5 hour to about 1 day, about 0.5 hour to about 2 days, about 0.5 hour to about 3 days, about 0.5 hour to about 4 days, about 0.5 hour to about 5 days, about 0.5 hour to about 6 days, about 0.5 hour to about 7 days, about 1 hour to about 1 day, about 1 hour to about 2 days, about 1 hour to about 3 days, about 1 hour to about 4 days, about 1 hour to about 5 days, about 1 hour to about 6 days, about 1 hour to about 7 days, about 2 hours to about 1 day, about 2 hours to about 2 days, about 2 hours to about 3 days, about 2 hours to about 4 days, about 2 hours to about 5 days, about 2 hours to about 6 days, about 2 hours to about 7 days, about 3 hours to about 1 day, about 3 hours to about 2 days, about 3 hours to about 3 days, about 3 hours to about 4 days, about 3 hours to about 5 days, about 3 hours to about 6 days, or about 3 hours to about 7 days, inclusive of any ranges therebetween. In other embodiments cells may be stored for more than 1 week, such as 2 weeks, 3 weeks, 1 month or up to one year.

In one or more embodiments, the method of reducing the leakage of biological components from cells or instructions for preserving and storing the cells may further include analyzing one or more of the cells. In one or more embodiments, analyzing one or more of the cells may include conducting a molecular analysis on one or more of the cells. In one or more embodiments, analyzing one or more of the cells may include conducting a nucleic acid or protein analysis of one or more of the cells. In one or more embodiments, analyzing one or more of the cells may include conducting a ribonucleic acid (RNA) analysis of one or more of the cells. In one or more embodiments, analyzing one or more of the cells may include conducting a single cell RNA analysis of one or more of the cells. In one or more embodiments, analyzing one or more of the cells may include conducting a deoxyribonucleic acid (DNA) analysis of one or more of the cells. In one or more embodiments, analyzing one or more of the cells may include conducting a single cell deoxyribonucleic acid (DNA) analysis of one or more of the cells. In one or more embodiments, analyzing one or more of the cells may include conducting a protein analysis of one or more of the cells. In one or more embodiments, analyzing one or more of the cells may include conducting a single cell protein analysis of one or more of the cells.

In one or more embodiments, the method of reducing the leakage of biological components from cells or instructions for preserving and storing the cells may further include conducting a molecular analysis of biological components of one or more of the cells. In one or more embodiments, the molecular analysis is single cell molecular analysis.

In one or more embodiments, the method of reducing the leakage of biological components from cells or instructions for preserving and storing the cells may further include partitioning of the biological components for single cell analysis. In one or more embodiments, the partition is an aqueous droplet in oil emulsion. In one or more embodiments, the kit further includes an oil for partitioning in an aqueous droplet in oil emulsion. In one or more embodiments, the partition further includes a gel bead. In one or more embodiments, the kit further includes gel beads for the partitioning.

In one or more embodiments, the method of reducing the leakage of biological components from cells or instructions for preserving and storing the cells may further include generating single cell barcoded biological components (analytes) for single cell data processing.

In one or more embodiments, the fixative buffer, treatment buffer, or storage buffer further includes a cryoprotectant reagent. In one or more embodiments, the fixative buffer, treatment buffer, or storage buffer further includes a stabilizing reagent. In one or more embodiments, the fixative buffer, treatment buffer, or storage buffer further includes an RNA stabilizer. In one or more embodiments, the fixative buffer, treatment buffer, or storage buffer further includes a ribonucleoside vanadyl complex (RVC). In one or more embodiments, the fixative buffer, treatment buffer, or storage buffer further includes an RNase inhibitor.

In one or more embodiments, the method of reducing the leakage of biological components from cells or instructions for preserving and storing the cells may further include i) contacting the plurality of cells with a fixative buffer for 1 hour at room temperature or overnight at about 2 to about 8 degrees Celsius, ii) removing the fixative buffer from the cells; and iii) suspending the cells in a storage buffer comprising about 0.25 mM to about 1.25 mM of a poloxamine 1107/T1107 Tetronic® polymer.

In one or more embodiments, the biological components comprise RNA. In one or more embodiments, the biological components comprise DNA. In one or more embodiments, the biological components comprise protein.

### Implementation #8

In accordance with one or more embodiments, a method for reducing leakage of biological components from cells during preservation and storage is described. The method of reducing the leakage of biological components from cells may include i) contacting a plurality of cells with a fixative buffer comprising a poloxamine/Tetronic® polymer; ii) removing the fixative buffer from the cells; and iii) suspending the cells in a storage buffer comprising a poloxamine/Tetronic® polymer.

In accordance with one or more embodiments, a kit for reducing leakage of biological components from cells during preservation and storage is described. The kit may include i) a fixative buffer comprising a poloxamine/Tetronic® polymer; ii) a storage buffer comprising a poloxamine/Tetronic® polymer; and iii) instructions for preserving and storing a plurality of cells in a manner that reduces leakage of biological components.

In one or more embodiments, the poloxamine/Tetronic® polymer may include a poloxamine 1107/T1107 Tetronic® polymer. In various embodiments, the fixative buffer may include a concentration of poloxamine 1107/T1107 Tetronic® polymer from about 0.05 mM to about 2.50 mM, from about 0.05 mM to about 2.25 mM, from about 0.05 mM to about 2.00 mM, from about 0.05 mM to about 1.75 mM, from about 0.05 mM to about 1.50 mM, from about 0.05 mM to about 1.25 mM, from about 0.05 mM to about 1.00 mM, from about 0.05 mM to about 0.75 mM, from about 0.05 mM to about 0.50 mM, from about 0.10 mM to about 2.50 mM, from about 0.10 mM to about 2.25 mM, from about 0.10 mM to about 2.00 mM, from about 0.10 mM to about 1.75 mM, from about 0.10 mM to about 1.50 mM, from about 0.10 mM to about 1.25 mM, from about 0.10 mM to about 1.00 mM, from about 0.10 mM to about 0.75 mM, from about 0.10 mM to about 0.50 mM, from about 0.15 mM to about 2.50 mM, from about 0.15 mM to about 2.25 mM, from about 0.15 mM to about 2.00 mM, from about 0.15 mM to about 1.75 mM, from about 0.15 mM to about 1.50 mM, from about 0.15 mM to about 1.25 mM, from about 0.15 mM to about 1.00 mM, from about 0.15 mM to about 0.75 mM, from about 0.15 mM to about 0.50 mM, from about 0.20 mM to about 2.50 mM, from about 0.20 mM to about 2.25 mM, from about 0.20 mM to about 2.00 mM, from about 0.20 mM to about 1.75 mM, from about 0.20 mM to about 1.50 mM, from about 0.20 mM to about 1.25 mM, from about 0.20 mM to about 1.00 mM, from about 0.20 mM to about 0.75 mM, from about 0.20 mM to about 0.50 mM, from about 0.30 mM to about 2.50 mM, from about 0.30 mM to about 2.25 mM, from about 0.30 mM to about 2.00 mM, from about 0.30 mM to about 1.75 mM, from about 0.30 mM to about 1.50 mM, from about 0.30 mM to about 1.25 mM, from about 0.30 mM to about 1.00 mM, from about 0.30 mM to about 0.75 mM, from about 0.30 mM to about 0.50 mM, from about 0.35 mM to about 2.50 mM, from about 0.35 mM to about 2.25 mM, from about 0.35 mM to about 2.00 mM, from about 0.35 mM to about 1.75 mM, from about 0.35 mM to about 1.50 mM, from about 0.35 mM to about 1.25 mM, from about 0.35 mM to about 1.00 mM, from about 0.35 mM to about 0.75 mM, or from about 0.35 mM to about 0.50 mM, inclusive of any ranges therebetween.

In one or more embodiments, the fixative buffer may include a crosslinking fixative. In one or more embodiments, the fixative buffer may include glyoxal. In one or more embodiments, the fixative buffer may include from about 0.05% glyoxal to about 1.5% glyoxal, from about 0.05% glyoxal to about 1.75% glyoxal, from about 0.05% glyoxal to about 2.00% glyoxal, from about 0.05% glyoxal to about 2.25% glyoxal, from about 0.05% glyoxal to about 2.50% glyoxal, from about 0.05% glyoxal to about 2.75% glyoxal, from about 0.05% glyoxal to about 3.00% glyoxal, from about 0.05% glyoxal to about 3.25% glyoxal, from about 0.05% glyoxal to about 3.50% glyoxal, from about 0.05% glyoxal to about 3.75% glyoxal, from about 0.05% glyoxal to about 4.00% glyoxal, from about 0.05% glyoxal to about 4.25% glyoxal, from about 0.05% glyoxal to about 4.50% glyoxal, from about 0.05% glyoxal to about 4.75% glyoxal, from about 0.05% glyoxal to about 5.00% glyoxal, from about 0.10% glyoxal to about 1.5% glyoxal, from about 0.10% glyoxal to about 1.75% glyoxal, from about 0.10% glyoxal to about 2.00% glyoxal, from about 0.10% glyoxal to about 2.25% glyoxal, from about 0.10% glyoxal to about 2.50% glyoxal, from about 0.10% glyoxal to about 2.75% glyoxal, from about 0.10% glyoxal to about 3.00% glyoxal, from about 0.10% glyoxal to about 3.25% glyoxal, from about 0.10% glyoxal to about 3.50% glyoxal, from about 0.10% glyoxal to about 3.75% glyoxal, from about 0.10% glyoxal to about 4.00% glyoxal, from about 0.10% glyoxal to about 4.25% glyoxal, from about 0.10% glyoxal to about 4.50% glyoxal, from about 0.10% glyoxal to about 4.75% glyoxal, from about 0.10% glyoxal to about 5.00% glyoxal, from about 0.15% glyoxal to about 1.5% glyoxal, from about 0.15% glyoxal to about 1.75% glyoxal, from about 0.15% glyoxal to about 2.00% glyoxal, from about 0.15% glyoxal to about 2.25% glyoxal, from about 0.15% glyoxal to about 2.50% glyoxal, from about 0.15% glyoxal to about 2.75% glyoxal, from about 0.15% glyoxal to about 3.00% glyoxal, from about 0.15% glyoxal to about 3.25% glyoxal, from about 0.15% glyoxal to about 3.50% glyoxal, from about 0.15% glyoxal to about 3.75% glyoxal, from about 0.15% glyoxal to about 4.00% glyoxal, from about 0.15% glyoxal to about 4.25% glyoxal, from about 0.15% glyoxal to about 4.50% glyoxal, from about 0.15% glyoxal to about 4.75% glyoxal, from about 0.15% glyoxal to about 5.00% glyoxal, from about 0.20% glyoxal to about 1.5% glyoxal, from about 0.20% glyoxal to about 1.75% glyoxal, from about 0.20% glyoxal to about 2.00% glyoxal, from about 0.20% glyoxal to about 2.25% glyoxal, from about 0.20% glyoxal to about 2.50% glyoxal, from about 0.20% glyoxal to about 2.75% glyoxal, from about 0.20% glyoxal to about 3.00% glyoxal, from about 0.20% glyoxal to about 3.25% glyoxal, from about 0.20% glyoxal to about 3.50% glyoxal, from about 0.20% glyoxal to about 3.75% glyoxal, from about 0.20% glyoxal to about 4.00% glyoxal, from about 0.20% glyoxal to about 4.25% glyoxal, from about 0.20% glyoxal to about 4.50% glyoxal, from about 0.20% glyoxal to about 4.75% glyoxal, from about 0.20% glyoxal to about 5.00% glyoxal, from about 0.25% glyoxal to about 1.5% glyoxal, from about 0.25% glyoxal to about 1.75% glyoxal, from about 0.25% glyoxal to about 2.00% glyoxal, from about 0.25% glyoxal to about 2.25% glyoxal, from about 0.25% glyoxal to about 2.50% glyoxal, from about 0.25% glyoxal to about 2.75% glyoxal, from about 0.25% glyoxal to about 3.00% glyoxal, from about 0.25% glyoxal to about 3.25% glyoxal, from about 0.25% glyoxal to about 3.50% glyoxal, from about 0.25% glyoxal to about 3.75% glyoxal, from about 0.25% glyoxal to about 4.00% glyoxal, from about 0.25% glyoxal to about 4.25% glyoxal, from about 0.25% glyoxal to about 4.50% glyoxal, from about 0.25% glyoxal to about 4.75% glyoxal, from about 0.25% glyoxal to about 5.00% glyoxal, from about 0.30% glyoxal to about 1.5% glyoxal, from about 0.30% glyoxal to about 1.75% glyoxal, from about 0.30% glyoxal to about 2.00% glyoxal, from about 0.30% glyoxal to about 2.25% glyoxal, from about 0.30% glyoxal to about 2.50% glyoxal, from about 0.30% glyoxal to about 2.75% glyoxal, from about 0.30% glyoxal to about 3.00% glyoxal, from about 0.30% glyoxal to about 3.25% glyoxal, from about 0.30% glyoxal to about 3.50% glyoxal, from about 0.30% glyoxal to about 3.75% glyoxal, from about 0.30% glyoxal to about 4.00% glyoxal, from about 0.30% glyoxal to about 4.25% glyoxal, from about 0.30% glyoxal to about 4.50% glyoxal, from about 0.30% glyoxal to about 4.75% glyoxal, from about 0.30% glyoxal to about 5.00% glyoxal, from about 0.35% glyoxal to about 1.5% glyoxal, from about 0.35% glyoxal to about 1.75% glyoxal, from about 0.35% glyoxal to about 2.00% glyoxal, from about 0.35% glyoxal to about 2.25% glyoxal, from about 0.35% glyoxal to about 2.50% glyoxal, from about 0.35% glyoxal to about 2.75% glyoxal, from about 0.35% glyoxal to about 3.00% glyoxal, from about 0.35% glyoxal to about 3.25% glyoxal, from about 0.35% glyoxal to about 3.50% glyoxal, from about 0.35% glyoxal to about 3.75% glyoxal, from about 0.35% glyoxal to about 4.00% glyoxal, from about 0.35% glyoxal to about 4.25% glyoxal, from about 0.35% glyoxal to about 4.50% glyoxal, from about 0.35% glyoxal to about 4.75% glyoxal, or from about 0.35% glyoxal to about 5.00% glyoxal, inclusive of any ranges therebetween.

In one or more embodiments, the fixative buffer may include DSP or DTSSP.

In various embodiments, the fixative buffer may include a concentration of DSP from about 0.05 mM to about 10 mM, from about 0.05 mM to about 7.5 mM, from about 0.05 mM to about 5.00 mM, from about 0.05 mM to about 3.5 mM, from about 0.05 mM to about 2.50 mM, from about 0.05 mM to about 1.25 mM, from about 0.05 mM to about 1.00 mM, from about 0.05 mM to about 0.75 mM, from about 0.05 mM to about 0.50 mM, 0.1 mM to about 10 mM, from about 0.1 mM to about 7.5 mM, from about 0.1 mM to about 5.00 mM, from about 0.1 mM to about 3.5 mM, from about 0.1 mM to about 2.50 mM, from about 0.1 mM to about 1.25 mM, from about 0.1 mM to about 1.00 mM, from about 0.1 mM to about 0.75 mM, from about 0.1 mM to about 0.50 mM, 0.25 mM to about 10 mM, from about 0.25 mM to about 7.5 mM, from about 0.25 mM to about 5.00 mM, from about 0.25 mM to about 3.5 mM, from about 0.25 mM to about 2.50 mM, from about 0.25 mM to about 1.25 mM, from about 0.25 mM to about 1.00 mM, from about 0.25 mM to about 0.75 mM, from about 0.25 mM to about 0.50 mM, 0.5 mM to about 10 mM, from about 0.5 mM to about 7.5 mM, from about 0.5 mM to about 5.00 mM, from about 0.5 mM to about 3.5 mM, from about 0.5 mM to about 2.50 mM, from about 0.5 mM to about 1.25 mM, from about 0.5 mM to about 1.00 mM, from about 0.5 mM to about 0.75 mM, 1.25 mM to about 10 mM, from about 1.25 mM to about 7.5 mM, from about 1.25 mM to about 5.00 mM, from about 1.25 mM to about 3.5 mM, from about 1.25 mM to about 2.50 mM, 2.5 mM to about 10 mM, from about 2.5 mM to about 7.5 mM, from about 2.5 mM to about 5.00 mM, and from about 2.5 mM to about 3.5 mM, inclusive of any ranges, values or endpoints therebetween. In various embodiments, the concentration of DSP is 2.5 mM or 1.25 mM.

In various embodiments, the fixative buffer may include a concentration of DTSSP from about 0.25 mM to about 20 mM, from about 0.25 mM to about 10 mM, from about 0.25 mM to about 7.5 mM, from about 0.25 mM to about 7.0 mM, from about 0.25 mM to about 5.00 mM, from about 0.25 mM to about 3.5 mM, from about 0.25 mM to about 2.50 mM, from about 0.25 mM to about 1.25 mM, from about 0.25 mM to about 1.00 mM, from about 0.25 mM to about 0.75 mM, from about 0.25 mM to about 0.50 mM, from about 0.5 mM to about 20 mM, from about 0.5 mM to about 10 mM, from about 0.5 mM to about 7.5 mM, from about 0.5 mM to about 7.0 mM, from about 0.5 mM to about 5.00 mM, from about 0.5 mM to about 3.5 mM, from about 0.5 mM to about 2.50 mM, from about 0.5 mM to about 1.25 mM, from about 0.5 mM to about 1.00 mM, from about 0.5 mM to about 0.75 mM, from about 1 mM to about 20 mM, from about 1 mM to about 10 mM, from about 1 mM to about 7.5 mM, from about 1 mM to about 7.0 mM, from about 1 mM to about 5.00 mM, from about 1 mM to about 3.5 mM, from about 1 mM to about 2.50 mM, from about 1 mM to about 1.25 mM, from about 2 mM to about 20 mM, from about 2 mM to about 10 mM, from about 2 mM to about 7.5 mM, from about 2 mM to about 7.0 mM, from about 2 mM to about 5.00 mM, from about 2 mM to about 3.5 mM, from about 2 mM to about 2.50 mM, from about 2.5 mM to about 20 mM, from about 2.5 mM to about 10 mM, from about 2.5 mM to about 7.5 mM, from about 2.5 mM to about 7.0 mM, from about 2.5 mM to about 5.00 mM, and from about 2.5 mM to about 3.5 mM, inclusive of any ranges, values or endpoints therebetween. In various embodiments, the concentration of DTSSP is 2.5 mM or 5 mM.

In various embodiments, the storage buffer may include a concentration of poloxamine 1107/T1107 Tetronic® polymer from about 0.05 mM to about 2.50 mM, from about 0.05 mM to about 2.25 mM, from about 0.05 mM to about 2.00 mM, from about 0.05 mM to about 1.75 mM, from about 0.05 mM to about 1.50 mM, from about 0.05 mM to about 1.25 mM, from about 0.05 mM to about 1.00 mM, from about 0.05 mM to about 0.75 mM, from about 0.05 mM to about 0.50 mM, from about 0.10 mM to about 2.50 mM, from about 0.10 mM to about 2.25 mM, from about 0.10 mM to about 2.00 mM, from about 0.10 mM to about 1.75 mM, from about 0.10 mM to about 1.50 mM, from about 0.10 mM to about 1.25 mM, from about 0.10 mM to about 1.00 mM, from about 0.10 mM to about 0.75 mM, from about 0.10 mM to about 0.50 mM, from about 0.15 mM to about 2.50 mM, from about 0.15 mM to about 2.25 mM, from about 0.15 mM to about 2.00 mM, from about 0.15 mM to about 1.75 mM, from about 0.15 mM to about 1.50 mM, from about 0.15 mM to about 1.25 mM, from about 0.15 mM to about 1.00 mM, from about 0.15 mM to about 0.75 mM, from about 0.15 mM to about 0.50 mM, from about 0.20 mM to about 2.50 mM, from about 0.20 mM to about 2.25 mM, from about 0.20 mM to about 2.00 mM, from about 0.20 mM to about 1.75 mM, from about 0.20 mM to about 1.50 mM, from about 0.20 mM to about 1.25 mM, from about 0.20 mM to about 1.00 mM, from about 0.20 mM to about 0.75 mM, from about 0.20 mM to about 0.50 mM, from about 0.30 mM to about 2.50 mM, from about 0.30 mM to about 2.25 mM, from about 0.30 mM to about 2.00 mM, from about 0.30 mM to about 1.75 mM, from about 0.30 mM to about 1.50 mM, from about 0.30 mM to about 1.25 mM, from about 0.30 mM to about 1.00 mM, from about 0.30 mM to about 0.75 mM, from about 0.30 mM to about 0.50 mM, from about 0.35 mM to about 2.50 mM, from about 0.35 mM to about 2.25 mM, from about 0.35 mM to about 2.00 mM, from about 0.35 mM to about 1.75 mM, from about 0.35 mM to about 1.50 mM, from about 0.35 mM to about 1.25 mM, from about 0.35 mM to about 1.00 mM, from about 0.35 mM to about 0.75 mM, or from about 0.35 mM to about 0.50 mM, inclusive of any ranges therebetween.

In one or more embodiments, the method of reducing the leakage of biological components from cells or instructions for preserving and storing the cells may further include storing the cells at room temperature. In one or more embodiments, the method of reducing the leakage of biological components from cells or instructions for preserving and storing the cells may further include storing the cells at about 1 degree Celsius to about 4 degrees Celsius, about 1 degree Celsius to about 5 degrees Celsius, about 1 degree Celsius to about 6 degrees Celsius, about 1 degree Celsius to about 7 degrees Celsius, about 1 degree Celsius to about 8 degrees Celsius, about 1 degree Celsius to about 9 degrees Celsius, about 1 degree Celsius to about 10 degrees Celsius, about 1 degree Celsius to about 11 degrees Celsius, about 1 degree Celsius to about 12 degrees Celsius, about 2 degrees Celsius to about 4 degrees Celsius, about 2 degrees Celsius to about 5 degrees Celsius, about 2 degrees Celsius to about 6 degrees Celsius, about 2 degrees Celsius to about 7 degrees Celsius, about 2 degrees Celsius to about 8 degrees Celsius, about 2 degrees Celsius to about 9 degrees Celsius, about 2 degrees Celsius to about 10 degrees Celsius, about 2 degrees Celsius to about 11 degrees Celsius, about 2 degrees Celsius to about 12 degrees Celsius, about 3 degrees Celsius to about 4 degrees Celsius, about 3 degrees Celsius to about 5 degrees Celsius, about 3 degrees Celsius to about 6 degrees Celsius, about 3 degrees Celsius to about 7 degrees Celsius, about 3 degrees Celsius to about 8 degrees Celsius, about 3 degrees Celsius to about 9 degrees Celsius, about 3 degrees Celsius to about 10 degrees Celsius, about 3 degrees Celsius to about 11 degrees Celsius, about 3 degrees Celsius to about 12 degrees Celsius, about 4 degrees Celsius to about 5 degrees Celsius, about 4 degrees Celsius to about 6 degrees Celsius, about 4 degrees Celsius to about 7 degrees Celsius, about 4 degrees Celsius to about 8 degrees Celsius, about 4 degrees Celsius to about 9 degrees Celsius, about 4 degrees Celsius to about 10 degrees Celsius, about 4 degrees Celsius to about 11 degrees Celsius, about 4 degrees Celsius to about 12 degrees Celsius, about 5 degrees Celsius to about 6 degrees Celsius, about 5 degrees Celsius to about 7 degrees Celsius, about 5 degrees Celsius to about 8 degrees Celsius, about 5 degrees Celsius to about 9 degrees Celsius, about 5 degrees Celsius to about 10 degrees Celsius, about 5 degrees Celsius to about 11 degrees Celsius, or about 5 degrees Celsius to about 12 degrees Celsius, inclusive of any ranges therebetween.

In one or more embodiments, the method of reducing the leakage of biological components from cells or instructions for preserving and storing the cells may further include cryopreserving the cells.

In one or more embodiments, the method of reducing the leakage of biological components from cells or instructions for preserving and storing the cells may further include cryopreserving the cells at about 0 degrees Celsius to about −196 degrees Celsius, 0 degrees Celsius to about −175 degrees Celsius, 0 degrees Celsius to about −150 degrees Celsius, 0 degrees Celsius to about −125 degrees Celsius, 0 degrees Celsius to about −100 degrees Celsius, 0 degrees Celsius to about −80 degrees Celsius, 0 degrees Celsius to about −50 degrees Celsius, 0 degrees Celsius to about −20 degrees Celsius, about −20 degrees Celsius to about −196 degrees Celsius, −20 degrees Celsius to about −175 degrees Celsius, −20 degrees Celsius to about −150 degrees Celsius, −20 degrees Celsius to about −125 degrees Celsius, −20 degrees Celsius to about −100 degrees Celsius, −20 degrees Celsius to about −80 degrees Celsius, −20 degrees Celsius to about −50 degrees Celsius, −80 degrees Celsius to about −196 degrees Celsius, −80 degrees Celsius to about −175 degrees Celsius, −80 degrees Celsius to about −150 degrees Celsius, −80 degrees Celsius to about −125 degrees Celsius, and −80 degrees Celsius to about −100 degrees Celsius, inclusive of any ranges, values or endpoints therebetween.

In one or more embodiments, the storage buffer is a phosphate buffered saline (PBS)-based storage buffer. In one or more embodiments, the storage buffer is a saline sodium citrate (SSC) based storage buffer.

In one or more embodiments, the contacting step may include incubating for 0.5 hour, 1 hour, 1.5 hours, or 2 hours at room temperature or incubating overnight at 2-8 degrees Celsius, 1-7 degrees Celsius, or 1-9 degrees Celsius.

In one or more embodiments, the cells may be maintained in the storage buffer for a time period of about 0.5 hour to about 1 day, about 0.5 hour to about 2 days, about 0.5 hour to about 3 days, about 0.5 hour to about 4 days, about 0.5 hour to about 5 days, about 0.5 hour to about 6 days, about 0.5 hour to about 7 days, about 1 hour to about 1 day, about 1 hour to about 2 days, about 1 hour to about 3 days, about 1 hour to about 4 days, about 1 hour to about 5 days, about 1 hour to about 6 days, about 1 hour to about 7 days, about 2 hours to about 1 day, about 2 hours to about 2 days, about 2 hours to about 3 days, about 2 hours to about 4 days, about 2 hours to about 5 days, about 2 hours to about 6 days, about 2 hours to about 7 days, about 3 hours to about 1 day, about 3 hours to about 2 days, about 3 hours to about 3 days, about 3 hours to about 4 days, about 3 hours to about 5 days, about 3 hours to about 6 days, or about 3 hours to about 7 days, inclusive of any ranges therebetween. In other embodiments cells may be stored for more than 1 week, such as 2 weeks, 3 weeks, 1 month or up to one year.

In one or more embodiments, the method of reducing the leakage of biological components from cells or instructions for preserving and storing the cells may further include analyzing one or more of the cells. In one or more embodiments, analyzing one or more of the cells may include conducting a molecular analysis on one or more of the cells. In one or more embodiments, analyzing one or more of the cells may include conducting a nucleic acid or protein analysis of one or more of the cells. In one or more embodiments, analyzing one or more of the cells may include conducting a ribonucleic acid (RNA) analysis of one or more of the cells. In one or more embodiments, analyzing one or more of the cells may include conducting a single cell RNA analysis of one or more of the cells. In one or more embodiments, analyzing one or more of the cells may include conducting a deoxyribonucleic acid (DNA) analysis of one or more of the cells. In one or more embodiments, analyzing one or more of the cells may include conducting a single cell deoxyribonucleic acid (DNA) analysis of one or more of the cells. In one or more embodiments, analyzing one or more of the cells may include conducting a protein analysis of one or more of the cells. In one or more embodiments, analyzing one or more of the cells may include conducting a single cell protein analysis of one or more of the cells.

In one or more embodiments, the method of reducing the leakage of biological components from cells or instructions for preserving and storing the cells may further include conducting a molecular analysis of biological components of one or more of the cells. In one or more embodiments, the molecular analysis is single cell molecular analysis.

In one or more embodiments, the method of reducing the leakage of biological components from cells or instructions for preserving and storing the cells may further include partitioning of the biological components for single cell analysis. In one or more embodiments, the partition is an aqueous droplet in oil emulsion. In one or more embodiments, the kit further includes an oil for partitioning in an aqueous droplet in oil emulsion. In one or more embodiments, the partition further includes a gel bead. In one or more embodiments, the kit further includes gel beads for the partitioning.

In one or more embodiments, the method of reducing the leakage of biological components from cells or instructions for preserving and storing the cells may further include generating single cell barcoded biological components (analytes) for single cell data processing.

In one or more embodiments, the fixative buffer, treatment buffer, or storage buffer further includes a cryoprotectant reagent. In one or more embodiments, the fixative buffer, treatment buffer, or storage buffer further includes a stabilizing reagent. In one or more embodiments, the fixative buffer, treatment buffer, or storage buffer further includes an RNA stabilizer. In one or more embodiments, the fixative buffer, treatment buffer, or storage buffer further includes a ribonucleoside vanadyl complex (RVC). In one or more embodiments, the fixative buffer, treatment buffer, or storage buffer further includes an RNase inhibitor.

In one or more embodiments, the method of reducing the leakage of biological components from cells or instructions for preserving and storing the cells may further include i) contacting the plurality of cells with a fixative buffer for 1 hour at room temperature or overnight at about 2 to about 8 degrees Celsius, wherein the fixative buffer comprises about 0.25 mM to about 1.25 mM of a poloxamine 1107/T1107 Tetronic® polymer, ii) removing the fixative buffer from the cells; and iii) suspending the cells in a storage buffer comprising about 0.25 mM to about 1.25 mM of a poloxamine 1107/T1107 Tetronic® polymer.

In one or more embodiments, the biological components comprise RNA. In one or more embodiments, the biological components comprise DNA. In one or more embodiments, the biological components comprise protein.

### Implementation #9

In accordance with one or more embodiments, a method for reducing leakage of biological components from cells during preservation and storage is described. The method of reducing the leakage of biological components from cells may include i) contacting a plurality of cells with a fixative buffer comprising a poloxamine/Tetronic® polymer; ii) removing the fixative buffer from the cells; iii) contacting the cells with a treatment buffer comprising a poloxamine/Tetronic® polymer; iv) removing the treatment buffer from the cells; and v) suspending the cells in a storage buffer.

In accordance with one or more embodiments, a kit for reducing leakage of biological components from cells during preservation and storage is described. The kit may include i) a fixative buffer comprising a poloxamine/Tetronic® polymer; ii) a treatment buffer comprising a poloxamine/Tetronic® polymer; iii) a storage buffer; and iv) instructions for preserving and storing a plurality of cells in a manner that reduces leakage of biological components.

In one or more embodiments, the poloxamine/Tetronic® polymer may include a poloxamine 1107/T1107 Tetronic® polymer. In various embodiments, the fixative buffer may include a concentration of poloxamine 1107/T1107 Tetronic® polymer from about 0.05 mM to about 2.50 mM, from about 0.05 mM to about 2.25 mM, from about 0.05 mM to about 2.00 mM, from about 0.05 mM to about 1.75 mM, from about 0.05 mM to about 1.50 mM, from about 0.05 mM to about 1.25 mM, from about 0.05 mM to about 1.00 mM, from about 0.05 mM to about 0.75 mM, from about 0.05 mM to about 0.50 mM, from about 0.10 mM to about 2.50 mM, from about 0.10 mM to about 2.25 mM, from about 0.10 mM to about 2.00 mM, from about 0.10 mM to about 1.75 mM, from about 0.10 mM to about 1.50 mM, from about 0.10 mM to about 1.25 mM, from about 0.10 mM to about 1.00 mM, from about 0.10 mM to about 0.75 mM, from about 0.10 mM to about 0.50 mM, from about 0.15 mM to about 2.50 mM, from about 0.15 mM to about 2.25 mM, from about 0.15 mM to about 2.00 mM, from about 0.15 mM to about 1.75 mM, from about 0.15 mM to about 1.50 mM, from about 0.15 mM to about 1.25 mM, from about 0.15 mM to about 1.00 mM, from about 0.15 mM to about 0.75 mM, from about 0.15 mM to about 0.50 mM, from about 0.20 mM to about 2.50 mM, from about 0.20 mM to about 2.25 mM, from about 0.20 mM to about 2.00 mM, from about 0.20 mM to about 1.75 mM, from about 0.20 mM to about 1.50 mM, from about 0.20 mM to about 1.25 mM, from about 0.20 mM to about 1.00 mM, from about 0.20 mM to about 0.75 mM, from about 0.20 mM to about 0.50 mM, from about 0.30 mM to about 2.50 mM, from about 0.30 mM to about 2.25 mM, from about 0.30 mM to about 2.00 mM, from about 0.30 mM to about 1.75 mM, from about 0.30 mM to about 1.50 mM, from about 0.30 mM to about 1.25 mM, from about 0.30 mM to about 1.00 mM, from about 0.30 mM to about 0.75 mM, from about 0.30 mM to about 0.50 mM, from about 0.35 mM to about 2.50 mM, from about 0.35 mM to about 2.25 mM, from about 0.35 mM to about 2.00 mM, from about 0.35 mM to about 1.75 mM, from about 0.35 mM to about 1.50 mM, from about 0.35 mM to about 1.25 mM, from about 0.35 mM to about 1.00 mM, from about 0.35 mM to about 0.75 mM, or from about 0.35 mM to about 0.50 mM, inclusive of any ranges therebetween.

In one or more embodiments, the fixative buffer may include a crosslinking fixative. In one or more embodiments, the fixative buffer may include glyoxal. In one or more embodiments, the fixative buffer may include from about 0.05% glyoxal to about 1.5% glyoxal, from about 0.05% glyoxal to about 1.75% glyoxal, from about 0.05% glyoxal to about 2.00% glyoxal, from about 0.05% glyoxal to about 2.25% glyoxal, from about 0.05% glyoxal to about 2.50% glyoxal, from about 0.05% glyoxal to about 2.75% glyoxal, from about 0.05% glyoxal to about 3.00% glyoxal, from about 0.05% glyoxal to about 3.25% glyoxal, from about 0.05% glyoxal to about 3.50% glyoxal, from about 0.05% glyoxal to about 3.75% glyoxal, from about 0.05% glyoxal to about 4.00% glyoxal, from about 0.05% glyoxal to about 4.25% glyoxal, from about 0.05% glyoxal to about 4.50% glyoxal, from about 0.05% glyoxal to about 4.75% glyoxal, from about 0.05% glyoxal to about 5.00% glyoxal, from about 0.10% glyoxal to about 1.5% glyoxal, from about 0.10% glyoxal to about 1.75% glyoxal, from about 0.10% glyoxal to about 2.00% glyoxal, from about 0.10% glyoxal to about 2.25% glyoxal, from about 0.10% glyoxal to about 2.50% glyoxal, from about 0.10% glyoxal to about 2.75% glyoxal, from about 0.10% glyoxal to about 3.00% glyoxal, from about 0.10% glyoxal to about 3.25% glyoxal, from about 0.10% glyoxal to about 3.50% glyoxal, from about 0.10% glyoxal to about 3.75% glyoxal, from about 0.10% glyoxal to about 4.00% glyoxal, from about 0.10% glyoxal to about 4.25% glyoxal, from about 0.10% glyoxal to about 4.50% glyoxal, from about 0.10% glyoxal to about 4.75% glyoxal, from about 0.10% glyoxal to about 5.00% glyoxal, from about 0.15% glyoxal to about 1.5% glyoxal, from about 0.15% glyoxal to about 1.75% glyoxal, from about 0.15% glyoxal to about 2.00% glyoxal, from about 0.15% glyoxal to about 2.25% glyoxal, from about 0.15% glyoxal to about 2.50% glyoxal, from about 0.15% glyoxal to about 2.75% glyoxal, from about 0.15% glyoxal to about 3.00% glyoxal, from about 0.15% glyoxal to about 3.25% glyoxal, from about 0.15% glyoxal to about 3.50% glyoxal, from about 0.15% glyoxal to about 3.75% glyoxal, from about 0.15% glyoxal to about 4.00% glyoxal, from about 0.15% glyoxal to about 4.25% glyoxal, from about 0.15% glyoxal to about 4.50% glyoxal, from about 0.15% glyoxal to about 4.75% glyoxal, from about 0.15% glyoxal to about 5.00% glyoxal, from about 0.20% glyoxal to about 1.5% glyoxal, from about 0.20% glyoxal to about 1.75% glyoxal, from about 0.20% glyoxal to about 2.00% glyoxal, from about 0.20% glyoxal to about 2.25% glyoxal, from about 0.20% glyoxal to about 2.50% glyoxal, from about 0.20% glyoxal to about 2.75% glyoxal, from about 0.20% glyoxal to about 3.00% glyoxal, from about 0.20% glyoxal to about 3.25% glyoxal, from about 0.20% glyoxal to about 3.50% glyoxal, from about 0.20% glyoxal to about 3.75% glyoxal, from about 0.20% glyoxal to about 4.00% glyoxal, from about 0.20% glyoxal to about 4.25% glyoxal, from about 0.20% glyoxal to about 4.50% glyoxal, from about 0.20% glyoxal to about 4.75% glyoxal, from about 0.20% glyoxal to about 5.00% glyoxal, from about 0.25% glyoxal to about 1.5% glyoxal, from about 0.25% glyoxal to about 1.75% glyoxal, from about 0.25% glyoxal to about 2.00% glyoxal, from about 0.25% glyoxal to about 2.25% glyoxal, from about 0.25% glyoxal to about 2.50% glyoxal, from about 0.25% glyoxal to about 2.75% glyoxal, from about 0.25% glyoxal to about 3.00% glyoxal, from about 0.25% glyoxal to about 3.25% glyoxal, from about 0.25% glyoxal to about 3.50% glyoxal, from about 0.25% glyoxal to about 3.75% glyoxal, from about 0.25% glyoxal to about 4.00% glyoxal, from about 0.25% glyoxal to about 4.25% glyoxal, from about 0.25% glyoxal to about 4.50% glyoxal, from about 0.25% glyoxal to about 4.75% glyoxal, from about 0.25% glyoxal to about 5.00% glyoxal, from about 0.30% glyoxal to about 1.5% glyoxal, from about 0.30% glyoxal to about 1.75% glyoxal, from about 0.30% glyoxal to about 2.00% glyoxal, from about 0.30% glyoxal to about 2.25% glyoxal, from about 0.30% glyoxal to about 2.50% glyoxal, from about 0.30% glyoxal to about 2.75% glyoxal, from about 0.30% glyoxal to about 3.00% glyoxal, from about 0.30% glyoxal to about 3.25% glyoxal, from about 0.30% glyoxal to about 3.50% glyoxal, from about 0.30% glyoxal to about 3.75% glyoxal, from about 0.30% glyoxal to about 4.00% glyoxal, from about 0.30% glyoxal to about 4.25% glyoxal, from about 0.30% glyoxal to about 4.50% glyoxal, from about 0.30% glyoxal to about 4.75% glyoxal, from about 0.30% glyoxal to about 5.00% glyoxal, from about 0.35% glyoxal to about 1.5% glyoxal, from about 0.35% glyoxal to about 1.75% glyoxal, from about 0.35% glyoxal to about 2.00% glyoxal, from about 0.35% glyoxal to about 2.25% glyoxal, from about 0.35% glyoxal to about 2.50% glyoxal, from about 0.35% glyoxal to about 2.75% glyoxal, from about 0.35% glyoxal to about 3.00% glyoxal, from about 0.35% glyoxal to about 3.25% glyoxal, from about 0.35% glyoxal to about 3.50% glyoxal, from about 0.35% glyoxal to about 3.75% glyoxal, from about 0.35% glyoxal to about 4.00% glyoxal, from about 0.35% glyoxal to about 4.25% glyoxal, from about 0.35% glyoxal to about 4.50% glyoxal, from about 0.35% glyoxal to about 4.75% glyoxal, or from about 0.35% glyoxal to about 5.00% glyoxal, inclusive of any ranges therebetween.

In one or more embodiments, the fixative buffer may include DSP or DTSSP.

In various embodiments, the fixative buffer may include a concentration of DSP from about 0.05 mM to about 10 mM, from about 0.05 mM to about 7.5 mM, from about 0.05 mM to about 5.00 mM, from about 0.05 mM to about 3.5 mM, from about 0.05 mM to about 2.50 mM, from about 0.05 mM to about 1.25 mM, from about 0.05 mM to about 1.00 mM, from about 0.05 mM to about 0.75 mM, from about 0.05 mM to about 0.50 mM, 0.1 mM to about 10 mM, from about 0.1 mM to about 7.5 mM, from about 0.1 mM to about 5.00 mM, from about 0.1 mM to about 3.5 mM, from about 0.1 mM to about 2.50 mM, from about 0.1 mM to about 1.25 mM, from about 0.1 mM to about 1.00 mM, from about 0.1 mM to about 0.75 mM, from about 0.1 mM to about 0.50 mM, 0.25 mM to about 10 mM, from about 0.25 mM to about 7.5 mM, from about 0.25 mM to about 5.00 mM, from about 0.25 mM to about 3.5 mM, from about 0.25 mM to about 2.50 mM, from about 0.25 mM to about 1.25 mM, from about 0.25 mM to about 1.00 mM, from about 0.25 mM to about 0.75 mM, from about 0.25 mM to about 0.50 mM, 0.5 mM to about 10 mM, from about 0.5 mM to about 7.5 mM, from about 0.5 mM to about 5.00 mM, from about 0.5 mM to about 3.5 mM, from about 0.5 mM to about 2.50 mM, from about 0.5 mM to about 1.25 mM, from about 0.5 mM to about 1.00 mM, from about 0.5 mM to about 0.75 mM, 1.25 mM to about 10 mM, from about 1.25 mM to about 7.5 mM, from about 1.25 mM to about 5.00 mM, from about 1.25 mM to about 3.5 mM, from about 1.25 mM to about 2.50 mM, 2.5 mM to about 10 mM, from about 2.5 mM to about 7.5 mM, from about 2.5 mM to about 5.00 mM, and from about 2.5 mM to about 3.5 mM, inclusive of any ranges, values or endpoints therebetween. In various embodiments, the concentration of DSP is 2.5 mM or 1.25 mM.

In various embodiments, the fixative buffer may include a concentration of DTSSP from about 0.25 mM to about 20 mM, from about 0.25 mM to about 10 mM, from about 0.25 mM to about 7.5 mM, from about 0.25 mM to about 7.0 mM, from about 0.25 mM to about 5.00 mM, from about 0.25 mM to about 3.5 mM, from about 0.25 mM to about 2.50 mM, from about 0.25 mM to about 1.25 mM, from about 0.25 mM to about 1.00 mM, from about 0.25 mM to about 0.75 mM, from about 0.25 mM to about 0.50 mM, from about 0.5 mM to about 20 mM, from about 0.5 mM to about 10 mM, from about 0.5 mM to about 7.5 mM, from about 0.5 mM to about 7.0 mM, from about 0.5 mM to about 5.00 mM, from about 0.5 mM to about 3.5 mM, from about 0.5 mM to about 2.50 mM, from about 0.5 mM to about 1.25 mM, from about 0.5 mM to about 1.00 mM, from about 0.5 mM to about 0.75 mM, from about 1 mM to about 20 mM, from about 1 mM to about 10 mM, from about 1 mM to about 7.5 mM, from about 1 mM to about 7.0 mM, from about 1 mM to about 5.00 mM, from about 1 mM to about 3.5 mM, from about 1 mM to about 2.50 mM, from about 1 mM to about 1.25 mM, from about 2 mM to about 20 mM, from about 2 mM to about 10 mM, from about 2 mM to about 7.5 mM, from about 2 mM to about 7.0 mM, from about 2 mM to about 5.00 mM, from about 2 mM to about 3.5 mM, from about 2 mM to about 2.50 mM, from about 2.5 mM to about 20 mM, from about 2.5 mM to about 10 mM, from about 2.5 mM to about 7.5 mM, from about 2.5 mM to about 7.0 mM, from about 2.5 mM to about 5.00 mM, and from about 2.5 mM to about 3.5 mM, inclusive of any ranges, values or endpoints therebetween. In various embodiments, the concentration of DTSSP is 2.5 mM or 5 mM.

In one or more embodiments, the method of reducing the leakage of biological components from cells or instructions for preserving and storing the cells may further include storing the cells at room temperature. In one or more embodiments, the method of reducing the leakage of biological components from cells or instructions for preserving and storing the cells may further include storing the cells at about 1 degree Celsius to about 4 degrees Celsius, about 1 degree Celsius to about 5 degrees Celsius, about 1 degree Celsius to about 6 degrees Celsius, about 1 degree Celsius to about 7 degrees Celsius, about 1 degree Celsius to about 8 degrees Celsius, about 1 degree Celsius to about 9 degrees Celsius, about 1 degree Celsius to about 10 degrees Celsius, about 1 degree Celsius to about 11 degrees Celsius, about 1 degree Celsius to about 12 degrees Celsius, about 2 degrees Celsius to about 4 degrees Celsius, about 2 degrees Celsius to about 5 degrees Celsius, about 2 degrees Celsius to about 6 degrees Celsius, about 2 degrees Celsius to about 7 degrees Celsius, about 2 degrees Celsius to about 8 degrees Celsius, about 2 degrees Celsius to about 9 degrees Celsius, about 2 degrees Celsius to about 10 degrees Celsius, about 2 degrees Celsius to about 11 degrees Celsius, about 2 degrees Celsius to about 12 degrees Celsius, about 3 degrees Celsius to about 4 degrees Celsius, about 3 degrees Celsius to about 5 degrees Celsius, about 3 degrees Celsius to about 6 degrees Celsius, about 3 degrees Celsius to about 7 degrees Celsius, about 3 degrees Celsius to about 8 degrees Celsius, about 3 degrees Celsius to about 9 degrees Celsius, about 3 degrees Celsius to about 10 degrees Celsius, about 3 degrees Celsius to about 11 degrees Celsius, about 3 degrees Celsius to about 12 degrees Celsius, about 4 degrees Celsius to about 5 degrees Celsius, about 4 degrees Celsius to about 6 degrees Celsius, about 4 degrees Celsius to about 7 degrees Celsius, about 4 degrees Celsius to about 8 degrees Celsius, about 4 degrees Celsius to about 9 degrees Celsius, about 4 degrees Celsius to about 10 degrees Celsius, about 4 degrees Celsius to about 11 degrees Celsius, about 4 degrees Celsius to about 12 degrees Celsius, about 5 degrees Celsius to about 6 degrees Celsius, about 5 degrees Celsius to about 7 degrees Celsius, about 5 degrees Celsius to about 8 degrees Celsius, about 5 degrees Celsius to about 9 degrees Celsius, about 5 degrees Celsius to about 10 degrees Celsius, about 5 degrees Celsius to about 11 degrees Celsius, or about 5 degrees Celsius to about 12 degrees Celsius, inclusive of any ranges therebetween.

In one or more embodiments, the method of reducing the leakage of biological components from cells or instructions for preserving and storing the cells may further include cryopreserving the cells.

In one or more embodiments, the method of reducing the leakage of biological components from cells or instructions for preserving and storing the cells may further include cryopreserving the cells at about 0 degrees Celsius to about −196 degrees Celsius, 0 degrees Celsius to about −175 degrees Celsius, 0 degrees Celsius to about −150 degrees Celsius, 0 degrees Celsius to about −125 degrees Celsius, 0 degrees Celsius to about −100 degrees Celsius, 0 degrees Celsius to about −80 degrees Celsius, 0 degrees Celsius to about −50 degrees Celsius, 0 degrees Celsius to about −20 degrees Celsius, about −20 degrees Celsius to about −196 degrees Celsius, −20 degrees Celsius to about −175 degrees Celsius, −20 degrees Celsius to about −150 degrees Celsius, −20 degrees Celsius to about −125 degrees Celsius, −20 degrees Celsius to about −100 degrees Celsius, −20 degrees Celsius to about −80 degrees Celsius, −20 degrees Celsius to about −50 degrees Celsius, −80 degrees Celsius to about −196 degrees Celsius, −80 degrees Celsius to about −175 degrees Celsius, −80 degrees Celsius to about −150 degrees Celsius, −80 degrees Celsius to about −125 degrees Celsius, and −80 degrees Celsius to about −100 degrees Celsius, inclusive of any ranges, values or endpoints therebetween.

In one or more embodiments, the storage buffer is a phosphate buffered saline (PBS)-based storage buffer. In one or more embodiments, the storage buffer is a saline sodium citrate (SSC) based storage buffer.

In one or more embodiments, the contacting step may include incubating for 0.5 hour, 1 hour, 1.5 hours, or 2 hours at room temperature or incubating overnight at 2-8 degrees Celsius, 1-7 degrees Celsius, or 1-9 degrees Celsius.

In one or more embodiments, the cells may be maintained in the storage buffer for a time period of about 0.5 hour to about 1 day, about 0.5 hour to about 2 days, about 0.5 hour to about 3 days, about 0.5 hour to about 4 days, about 0.5 hour to about 5 days, about 0.5 hour to about 6 days, about 0.5 hour to about 7 days, about 1 hour to about 1 day, about 1 hour to about 2 days, about 1 hour to about 3 days, about 1 hour to about 4 days, about 1 hour to about 5 days, about 1 hour to about 6 days, about 1 hour to about 7 days, about 2 hours to about 1 day, about 2 hours to about 2 days, about 2 hours to about 3 days, about 2 hours to about 4 days, about 2 hours to about 5 days, about 2 hours to about 6 days, about 2 hours to about 7 days, about 3 hours to about 1 day, about 3 hours to about 2 days, about 3 hours to about 3 days, about 3 hours to about 4 days, about 3 hours to about 5 days, about 3 hours to about 6 days, or about 3 hours to about 7 days, inclusive of any ranges therebetween. In other embodiments cells may be stored for more than 1 week, such as 2 weeks, 3 weeks, 1 month or up to one year.

In one or more embodiments, the treatment buffer may include a PBS based buffer comprising a concentration of poloxamine 1107/T1107 Tetronic® polymer from about 0.05 mM to about 2.50 mM, from about 0.05 mM to about 2.25 mM, from about 0.05 mM to about 2.00 mM, from about 0.05 mM to about 1.75 mM, from about 0.05 mM to about 1.50 mM, from about 0.05 mM to about 1.25 mM, from about 0.05 mM to about 1.00 mM, from about 0.05 mM to about 0.75 mM, from about 0.05 mM to about 0.50 mM, from about 0.10 mM to about 2.50 mM, from about 0.10 mM to about 2.25 mM, from about 0.10 mM to about 2.00 mM, from about 0.10 mM to about 1.75 mM, from about 0.10 mM to about 1.50 mM, from about 0.10 mM to about 1.25 mM, from about 0.10 mM to about 1.00 mM, from about 0.10 mM to about 0.75 mM, from about 0.10 mM to about 0.50 mM, from about 0.15 mM to about 2.50 mM, from about 0.15 mM to about 2.25 mM, from about 0.15 mM to about 2.00 mM, from about 0.15 mM to about 1.75 mM, from about 0.15 mM to about 1.50 mM, from about 0.15 mM to about 1.25 mM, from about 0.15 mM to about 1.00 mM, from about 0.15 mM to about 0.75 mM, from about 0.15 mM to about 0.50 mM, from about 0.20 mM to about 2.50 mM, from about 0.20 mM to about 2.25 mM, from about 0.20 mM to about 2.00 mM, from about 0.20 mM to about 1.75 mM, from about 0.20 mM to about 1.50 mM, from about 0.20 mM to about 1.25 mM, from about 0.20 mM to about 1.00 mM, from about 0.20 mM to about 0.75 mM, from about 0.20 mM to about 0.50 mM, from about 0.30 mM to about 2.50 mM, from about 0.30 mM to about 2.25 mM, from about 0.30 mM to about 2.00 mM, from about 0.30 mM to about 1.75 mM, from about 0.30 mM to about 1.50 mM, from about 0.30 mM to about 1.25 mM, from about 0.30 mM to about 1.00 mM, from about 0.30 mM to about 0.75 mM, from about 0.30 mM to about 0.50 mM, from about 0.35 mM to about 2.50 mM, from about 0.35 mM to about 2.25 mM, from about 0.35 mM to about 2.00 mM, from about 0.35 mM to about 1.75 mM, from about 0.35 mM to about 1.50 mM, from about 0.35 mM to about 1.25 mM, from about 0.35 mM to about 1.00 mM, from about 0.35 mM to about 0.75 mM, or from about 0.35 mM to about 0.50 mM, inclusive of any ranges therebetween.

In one or more embodiments, the method of reducing the leakage of biological components from cells or instructions for preserving and storing the cells may further include analyzing one or more of the cells. In one or more embodiments, analyzing one or more of the cells may include conducting a molecular analysis on one or more of the cells. In one or more embodiments, analyzing one or more of the cells may include conducting a nucleic acid or protein analysis of one or more of the cells. In one or more embodiments, analyzing one or more of the cells may include conducting a ribonucleic acid (RNA) analysis of one or more of the cells. In one or more embodiments, analyzing one or more of the cells may include conducting a single cell RNA analysis of one or more of the cells. In one or more embodiments, analyzing one or more of the cells may include conducting a deoxyribonucleic acid (DNA) analysis of one or more of the cells. In one or more embodiments, analyzing one or more of the cells may include conducting a single cell deoxyribonucleic acid (DNA) analysis of one or more of the cells. In one or more embodiments, analyzing one or more of the cells may include conducting a protein analysis of one or more of the cells. In one or more embodiments, analyzing one or more of the cells may include conducting a single cell protein analysis of one or more of the cells.

In one or more embodiments, the method of reducing the leakage of biological components from cells or instructions for preserving and storing the cells may further include conducting a molecular analysis of biological components of one or more of the cells. In one or more embodiments, the molecular analysis is single cell molecular analysis.

In one or more embodiments, the method of reducing the leakage of biological components from cells or instructions for preserving and storing the cells may further include partitioning of the biological components for single cell analysis. In one or more embodiments, the partition is an aqueous droplet in oil emulsion. In one or more embodiments, the kit further includes an oil for partitioning in an aqueous droplet in oil emulsion. In one or more embodiments, the partition further includes a gel bead. In one or more embodiments, the kit further includes gel beads for the partitioning.

In one or more embodiments, the method of reducing the leakage of biological components from cells or instructions for preserving and storing the cells may further include generating single cell barcoded biological components (analytes) for single cell data processing.

In one or more embodiments, the fixative buffer, treatment buffer, or storage buffer further includes a cryoprotectant reagent. In one or more embodiments, the fixative buffer, treatment buffer, or storage buffer further includes a stabilizing reagent. In one or more embodiments, the fixative buffer, treatment buffer, or storage buffer further includes an RNA stabilizer. In one or more embodiments, the fixative buffer, treatment buffer, or storage buffer further includes a ribonucleoside vanadyl complex (RVC). In one or more embodiments, the fixative buffer, treatment buffer, or storage buffer further includes an RNase inhibitor.

In one or more embodiments, the biological components comprise RNA. In one or more embodiments, the biological components comprise DNA. In one or more embodiments, the biological components comprise protein.

### Implementation #10

In accordance with one or more embodiments, a method for reducing leakage of biological components from cells during preservation and storage is described. The method of reducing the leakage of biological components from cells may include i) contacting a plurality of cells with a fixative buffer; ii) removing the fixative buffer from the cells; iii) contacting the cells with a treatment buffer comprising a poloxamine/Tetronic® polymer; iv) removing the treatment buffer from the cells; and v) suspending the cells in a storage buffer comprising a poloxamine/Tetronic® polymer.

In accordance with one or more embodiments, a kit for reducing leakage of biological components from cells during preservation and storage is described. The kit may include i) a fixative buffer; ii) a treatment buffer comprising a poloxamine/Tetronic® polymer; iii) a storage buffer comprising a poloxamine/Tetronic® polymer; and iv) instructions for preserving and storing a plurality of cells in a manner that reduces leakage of biological components.

In one or more embodiments, the poloxamine/Tetronic® polymer may include a poloxamine 1107/T1107 Tetronic® polymer.

In one or more embodiments, the fixative buffer may include a crosslinking fixative. In one or more embodiments, the fixative buffer may include glyoxal. In one or more embodiments, the fixative buffer may include from about 0.05% glyoxal to about 1.5% glyoxal, from about 0.05% glyoxal to about 1.75% glyoxal, from about 0.05% glyoxal to about 2.00% glyoxal, from about 0.05% glyoxal to about 2.25% glyoxal, from about 0.05% glyoxal to about 2.50% glyoxal, from about 0.05% glyoxal to about 2.75% glyoxal, from about 0.05% glyoxal to about 3.00% glyoxal, from about 0.05% glyoxal to about 3.25% glyoxal, from about 0.05% glyoxal to about 3.50% glyoxal, from about 0.05% glyoxal to about 3.75% glyoxal, from about 0.05% glyoxal to about 4.00% glyoxal, from about 0.05% glyoxal to about 4.25% glyoxal, from about 0.05% glyoxal to about 4.50% glyoxal, from about 0.05% glyoxal to about 4.75% glyoxal, from about 0.05% glyoxal to about 5.00% glyoxal, from about 0.10% glyoxal to about 1.5% glyoxal, from about 0.10% glyoxal to about 1.75% glyoxal, from about 0.10% glyoxal to about 2.00% glyoxal, from about 0.10% glyoxal to about 2.25% glyoxal, from about 0.10% glyoxal to about 2.50% glyoxal, from about 0.10% glyoxal to about 2.75% glyoxal, from about 0.10% glyoxal to about 3.00% glyoxal, from about 0.10% glyoxal to about 3.25% glyoxal, from about 0.10% glyoxal to about 3.50% glyoxal, from about 0.10% glyoxal to about 3.75% glyoxal, from about 0.10% glyoxal to about 4.00% glyoxal, from about 0.10% glyoxal to about 4.25% glyoxal, from about 0.10% glyoxal to about 4.50% glyoxal, from about 0.10% glyoxal to about 4.75% glyoxal, from about 0.10% glyoxal to about 5.00% glyoxal, from about 0.15% glyoxal to about 1.5% glyoxal, from about 0.15% glyoxal to about 1.75% glyoxal, from about 0.15% glyoxal to about 2.00% glyoxal, from about 0.15% glyoxal to about 2.25% glyoxal, from about 0.15% glyoxal to about 2.50% glyoxal, from about 0.15% glyoxal to about 2.75% glyoxal, from about 0.15% glyoxal to about 3.00% glyoxal, from about 0.15% glyoxal to about 3.25% glyoxal, from about 0.15% glyoxal to about 3.50% glyoxal, from about 0.15% glyoxal to about 3.75% glyoxal, from about 0.15% glyoxal to about 4.00% glyoxal, from about 0.15% glyoxal to about 4.25% glyoxal, from about 0.15% glyoxal to about 4.50% glyoxal, from about 0.15% glyoxal to about 4.75% glyoxal, from about 0.15% glyoxal to about 5.00% glyoxal, from about 0.20% glyoxal to about 1.5% glyoxal, from about 0.20% glyoxal to about 1.75% glyoxal, from about 0.20% glyoxal to about 2.00% glyoxal, from about 0.20% glyoxal to about 2.25% glyoxal, from about 0.20% glyoxal to about 2.50% glyoxal, from about 0.20% glyoxal to about 2.75% glyoxal, from about 0.20% glyoxal to about 3.00% glyoxal, from about 0.20% glyoxal to about 3.25% glyoxal, from about 0.20% glyoxal to about 3.50% glyoxal, from about 0.20% glyoxal to about 3.75% glyoxal, from about 0.20% glyoxal to about 4.00% glyoxal, from about 0.20% glyoxal to about 4.25% glyoxal, from about 0.20% glyoxal to about 4.50% glyoxal, from about 0.20% glyoxal to about 4.75% glyoxal, from about 0.20% glyoxal to about 5.00% glyoxal, from about 0.25% glyoxal to about 1.5% glyoxal, from about 0.25% glyoxal to about 1.75% glyoxal, from about 0.25% glyoxal to about 2.00% glyoxal, from about 0.25% glyoxal to about 2.25% glyoxal, from about 0.25% glyoxal to about 2.50% glyoxal, from about 0.25% glyoxal to about 2.75% glyoxal, from about 0.25% glyoxal to about 3.00% glyoxal, from about 0.25% glyoxal to about 3.25% glyoxal, from about 0.25% glyoxal to about 3.50% glyoxal, from about 0.25% glyoxal to about 3.75% glyoxal, from about 0.25% glyoxal to about 4.00% glyoxal, from about 0.25% glyoxal to about 4.25% glyoxal, from about 0.25% glyoxal to about 4.50% glyoxal, from about 0.25% glyoxal to about 4.75% glyoxal, from about 0.25% glyoxal to about 5.00% glyoxal, from about 0.30% glyoxal to about 1.5% glyoxal, from about 0.30% glyoxal to about 1.75% glyoxal, from about 0.30% glyoxal to about 2.00% glyoxal, from about 0.30% glyoxal to about 2.25% glyoxal, from about 0.30% glyoxal to about 2.50% glyoxal, from about 0.30% glyoxal to about 2.75% glyoxal, from about 0.30% glyoxal to about 3.00% glyoxal, from about 0.30% glyoxal to about 3.25% glyoxal, from about 0.30% glyoxal to about 3.50% glyoxal, from about 0.30% glyoxal to about 3.75% glyoxal, from about 0.30% glyoxal to about 4.00% glyoxal, from about 0.30% glyoxal to about 4.25% glyoxal, from about 0.30% glyoxal to about 4.50% glyoxal, from about 0.30% glyoxal to about 4.75% glyoxal, from about 0.30% glyoxal to about 5.00% glyoxal, from about 0.35% glyoxal to about 1.5% glyoxal, from about 0.35% glyoxal to about 1.75% glyoxal, from about 0.35% glyoxal to about 2.00% glyoxal, from about 0.35% glyoxal to about 2.25% glyoxal, from about 0.35% glyoxal to about 2.50% glyoxal, from about 0.35% glyoxal to about 2.75% glyoxal, from about 0.35% glyoxal to about 3.00% glyoxal, from about 0.35% glyoxal to about 3.25% glyoxal, from about 0.35% glyoxal to about 3.50% glyoxal, from about 0.35% glyoxal to about 3.75% glyoxal, from about 0.35% glyoxal to about 4.00% glyoxal, from about 0.35% glyoxal to about 4.25% glyoxal, from about 0.35% glyoxal to about 4.50% glyoxal, from about 0.35% glyoxal to about 4.75% glyoxal, or from about 0.35% glyoxal to about 5.00% glyoxal, inclusive of any ranges therebetween.

In one or more embodiments, the fixative buffer may include DSP or DTSSP.

In various embodiments, the fixative buffer may include a concentration of DSP from about 0.05 mM to about 10 mM, from about 0.05 mM to about 7.5 mM, from about 0.05 mM to about 5.00 mM, from about 0.05 mM to about 3.5 mM, from about 0.05 mM to about 2.50 mM, from about 0.05 mM to about 1.25 mM, from about 0.05 mM to about 1.00 mM, from about 0.05 mM to about 0.75 mM, from about 0.05 mM to about 0.50 mM, 0.1 mM to about 10 mM, from about 0.1 mM to about 7.5 mM, from about 0.1 mM to about 5.00 mM, from about 0.1 mM to about 3.5 mM, from about 0.1 mM to about 2.50 mM, from about 0.1 mM to about 1.25 mM, from about 0.1 mM to about 1.00 mM, from about 0.1 mM to about 0.75 mM, from about 0.1 mM to about 0.50 mM, 0.25 mM to about 10 mM, from about 0.25 mM to about 7.5 mM, from about 0.25 mM to about 5.00 mM, from about 0.25 mM to about 3.5 mM, from about 0.25 mM to about 2.50 mM, from about 0.25 mM to about 1.25 mM, from about 0.25 mM to about 1.00 mM, from about 0.25 mM to about 0.75 mM, from about 0.25 mM to about 0.50 mM, 0.5 mM to about 10 mM, from about 0.5 mM to about 7.5 mM, from about 0.5 mM to about 5.00 mM, from about 0.5 mM to about 3.5 mM, from about 0.5 mM to about 2.50 mM, from about 0.5 mM to about 1.25 mM, from about 0.5 mM to about 1.00 mM, from about 0.5 mM to about 0.75 mM, 1.25 mM to about 10 mM, from about 1.25 mM to about 7.5 mM, from about 1.25 mM to about 5.00 mM, from about 1.25 mM to about 3.5 mM, from about 1.25 mM to about 2.50 mM, 2.5 mM to about 10 mM, from about 2.5 mM to about 7.5 mM, from about 2.5 mM to about 5.00 mM, and from about 2.5 mM to about 3.5 mM, inclusive of any ranges, values or endpoints therebetween. In various embodiments, the concentration of DSP is 2.5 mM or 1.25 mM.

In various embodiments, the fixative buffer may include a concentration of DTSSP from about 0.25 mM to about 20 mM, from about 0.25 mM to about 10 mM, from about 0.25 mM to about 7.5 mM, from about 0.25 mM to about 7.0 mM, from about 0.25 mM to about 5.00 mM, from about 0.25 mM to about 3.5 mM, from about 0.25 mM to about 2.50 mM, from about 0.25 mM to about 1.25 mM, from about 0.25 mM to about 1.00 mM, from about 0.25 mM to about 0.75 mM, from about 0.25 mM to about 0.50 mM, from about 0.5 mM to about 20 mM, from about 0.5 mM to about 10 mM, from about 0.5 mM to about 7.5 mM, from about 0.5 mM to about 7.0 mM, from about 0.5 mM to about 5.00 mM, from about 0.5 mM to about 3.5 mM, from about 0.5 mM to about 2.50 mM, from about 0.5 mM to about 1.25 mM, from about 0.5 mM to about 1.00 mM, from about 0.5 mM to about 0.75 mM, from about 1 mM to about 20 mM, from about 1 mM to about 10 mM, from about 1 mM to about 7.5 mM, from about 1 mM to about 7.0 mM, from about 1 mM to about 5.00 mM, from about 1 mM to about 3.5 mM, from about 1 mM to about 2.50 mM, from about 1 mM to about 1.25 mM, from about 2 mM to about 20 mM, from about 2 mM to about 10 mM, from about 2 mM to about 7.5 mM, from about 2 mM to about 7.0 mM, from about 2 mM to about 5.00 mM, from about 2 mM to about 3.5 mM, from about 2 mM to about 2.50 mM, from about 2.5 mM to about 20 mM, from about 2.5 mM to about 10 mM, from about 2.5 mM to about 7.5 mM, from about 2.5 mM to about 7.0 mM, from about 2.5 mM to about 5.00 mM, and from about 2.5 mM to about 3.5 mM, inclusive of any ranges, values or endpoints therebetween. In various embodiments, the concentration of DTSSP is 2.5 mM or 5 mM.

In various embodiments, the storage buffer may include a concentration of poloxamine 1107/T1107 Tetronic® polymer from about 0.05 mM to about 2.50 mM, from about 0.05 mM to about 2.25 mM, from about 0.05 mM to about 2.00 mM, from about 0.05 mM to about 1.75 mM, from about 0.05 mM to about 1.50 mM, from about 0.05 mM to about 1.25 mM, from about 0.05 mM to about 1.00 mM, from about 0.05 mM to about 0.75 mM, from about 0.05 mM to about 0.50 mM, from about 0.10 mM to about 2.50 mM, from about 0.10 mM to about 2.25 mM, from about 0.10 mM to about 2.00 mM, from about 0.10 mM to about 1.75 mM, from about 0.10 mM to about 1.50 mM, from about 0.10 mM to about 1.25 mM, from about 0.10 mM to about 1.00 mM, from about 0.10 mM to about 0.75 mM, from about 0.10 mM to about 0.50 mM, from about 0.15 mM to about 2.50 mM, from about 0.15 mM to about 2.25 mM, from about 0.15 mM to about 2.00 mM, from about 0.15 mM to about 1.75 mM, from about 0.15 mM to about 1.50 mM, from about 0.15 mM to about 1.25 mM, from about 0.15 mM to about 1.00 mM, from about 0.15 mM to about 0.75 mM, from about 0.15 mM to about 0.50 mM, from about 0.20 mM to about 2.50 mM, from about 0.20 mM to about 2.25 mM, from about 0.20 mM to about 2.00 mM, from about 0.20 mM to about 1.75 mM, from about 0.20 mM to about 1.50 mM, from about 0.20 mM to about 1.25 mM, from about 0.20 mM to about 1.00 mM, from about 0.20 mM to about 0.75 mM, from about 0.20 mM to about 0.50 mM, from about 0.30 mM to about 2.50 mM, from about 0.30 mM to about 2.25 mM, from about 0.30 mM to about 2.00 mM, from about 0.30 mM to about 1.75 mM, from about 0.30 mM to about 1.50 mM, from about 0.30 mM to about 1.25 mM, from about 0.30 mM to about 1.00 mM, from about 0.30 mM to about 0.75 mM, from about 0.30 mM to about 0.50 mM, from about 0.35 mM to about 2.50 mM, from about 0.35 mM to about 2.25 mM, from about 0.35 mM to about 2.00 mM, from about 0.35 mM to about 1.75 mM, from about 0.35 mM to about 1.50 mM, from about 0.35 mM to about 1.25 mM, from about 0.35 mM to about 1.00 mM, from about 0.35 mM to about 0.75 mM, or from about 0.35 mM to about 0.50 mM, inclusive of any ranges therebetween.

In one or more embodiments, the method of reducing the leakage of biological components from cells or instructions for preserving and storing the cells may further include storing the cells at room temperature. In one or more embodiments, the method of reducing the leakage of biological components from cells or instructions for preserving and storing the cells may further include storing the cells at about 1 degree Celsius to about 4 degrees Celsius, about 1 degree Celsius to about 5 degrees Celsius, about 1 degree Celsius to about 6 degrees Celsius, about 1 degree Celsius to about 7 degrees Celsius, about 1 degree Celsius to about 8 degrees Celsius, about 1 degree Celsius to about 9 degrees Celsius, about 1 degree Celsius to about 10 degrees Celsius, about 1 degree Celsius to about 11 degrees Celsius, about 1 degree Celsius to about 12 degrees Celsius, about 2 degrees Celsius to about 4 degrees Celsius, about 2 degrees Celsius to about 5 degrees Celsius, about 2 degrees Celsius to about 6 degrees Celsius, about 2 degrees Celsius to about 7 degrees Celsius, about 2 degrees Celsius to about 8 degrees Celsius, about 2 degrees Celsius to about 9 degrees Celsius, about 2 degrees Celsius to about 10 degrees Celsius, about 2 degrees Celsius to about 11 degrees Celsius, about 2 degrees Celsius to about 12 degrees Celsius, about 3 degrees Celsius to about 4 degrees Celsius, about 3 degrees Celsius to about 5 degrees Celsius, about 3 degrees Celsius to about 6 degrees Celsius, about 3 degrees Celsius to about 7 degrees Celsius, about 3 degrees Celsius to about 8 degrees Celsius, about 3 degrees Celsius to about 9 degrees Celsius, about 3 degrees Celsius to about 10 degrees Celsius, about 3 degrees Celsius to about 11 degrees Celsius, about 3 degrees Celsius to about 12 degrees Celsius, about 4 degrees Celsius to about 5 degrees Celsius, about 4 degrees Celsius to about 6 degrees Celsius, about 4 degrees Celsius to about 7 degrees Celsius, about 4 degrees Celsius to about 8 degrees Celsius, about 4 degrees Celsius to about 9 degrees Celsius, about 4 degrees Celsius to about 10 degrees Celsius, about 4 degrees Celsius to about 11 degrees Celsius, about 4 degrees Celsius to about 12 degrees Celsius, about 5 degrees Celsius to about 6 degrees Celsius, about 5 degrees Celsius to about 7 degrees Celsius, about 5 degrees Celsius to about 8 degrees Celsius, about 5 degrees Celsius to about 9 degrees Celsius, about 5 degrees Celsius to about 10 degrees Celsius, about 5 degrees Celsius to about 11 degrees Celsius, or about 5 degrees Celsius to about 12 degrees Celsius, inclusive of any ranges therebetween.

In one or more embodiments, the method of reducing the leakage of biological components from cells or instructions for preserving and storing the cells may further include cryopreserving the cells.

In one or more embodiments, the method of reducing the leakage of biological components from cells or instructions for preserving and storing the cells may further include cryopreserving the cells at about 0 degrees Celsius to about −196 degrees Celsius, 0 degrees Celsius to about −175 degrees Celsius, 0 degrees Celsius to about −150 degrees Celsius, 0 degrees Celsius to about −125 degrees Celsius, 0 degrees Celsius to about −100 degrees Celsius, 0 degrees Celsius to about −80 degrees Celsius, 0 degrees Celsius to about −50 degrees Celsius, 0 degrees Celsius to about −20 degrees Celsius, about −20 degrees Celsius to about −196 degrees Celsius, −20 degrees Celsius to about −175 degrees Celsius, −20 degrees Celsius to about −150 degrees Celsius, −20 degrees Celsius to about −125 degrees Celsius, −20 degrees Celsius to about −100 degrees Celsius, −20 degrees Celsius to about −80 degrees Celsius, −20 degrees Celsius to about −50 degrees Celsius, −80 degrees Celsius to about −196 degrees Celsius, −80 degrees Celsius to about −175 degrees Celsius, −80 degrees Celsius to about −150 degrees Celsius, −80 degrees Celsius to about −125 degrees Celsius, and −80 degrees Celsius to about −100 degrees Celsius, inclusive of any ranges, values or endpoints therebetween.

In one or more embodiments, the storage buffer is a phosphate buffered saline (PBS)-based storage buffer. In one or more embodiments, the storage buffer is a saline sodium citrate (SSC) based storage buffer.

In one or more embodiments, the contacting step may include incubating for 0.5 hour, 1 hour, 1.5 hours, or 2 hours at room temperature or incubating overnight at 2-8 degrees Celsius, 1-7 degrees Celsius, or 1-9 degrees Celsius.

In one or more embodiments, the cells may be maintained in the storage buffer for a time period of about 0.5 hour to about 1 day, about 0.5 hour to about 2 days, about 0.5 hour to about 3 days, about 0.5 hour to about 4 days, about 0.5 hour to about 5 days, about 0.5 hour to about 6 days, about 0.5 hour to about 7 days, about 1 hour to about 1 day, about 1 hour to about 2 days, about 1 hour to about 3 days, about 1 hour to about 4 days, about 1 hour to about 5 days, about 1 hour to about 6 days, about 1 hour to about 7 days, about 2 hours to about 1 day, about 2 hours to about 2 days, about 2 hours to about 3 days, about 2 hours to about 4 days, about 2 hours to about 5 days, about 2 hours to about 6 days, about 2 hours to about 7 days, about 3 hours to about 1 day, about 3 hours to about 2 days, about 3 hours to about 3 days, about 3 hours to about 4 days, about 3 hours to about 5 days, about 3 hours to about 6 days, or about 3 hours to about 7 days, inclusive of any ranges therebetween. In other embodiments cells may be stored for more than 1 week, such as 2 weeks, 3 weeks, 1 month or up to one year.

In one or more embodiments, the treatment buffer may include a PBS based buffer comprising a concentration of T1107 Tetronic® polymer from about 0.05 mM to about 2.50 mM, from about 0.05 mM to about 2.25 mM, from about 0.05 mM to about 2.00 mM, from about 0.05 mM to about 1.75 mM, from about 0.05 mM to about 1.50 mM, from about 0.05 mM to about 1.25 mM, from about 0.05 mM to about 1.00 mM, from about 0.05 mM to about 0.75 mM, from about 0.05 mM to about 0.50 mM, from about 0.10 mM to about 2.50 mM, from about 0.10 mM to about 2.25 mM, from about 0.10 mM to about 2.00 mM, from about 0.10 mM to about 1.75 mM, from about 0.10 mM to about 1.50 mM, from about 0.10 mM to about 1.25 mM, from about 0.10 mM to about 1.00 mM, from about 0.10 mM to about 0.75 mM, from about 0.10 mM to about 0.50 mM, from about 0.15 mM to about 2.50 mM, from about 0.15 mM to about 2.25 mM, from about 0.15 mM to about 2.00 mM, from about 0.15 mM to about 1.75 mM, from about 0.15 mM to about 1.50 mM, from about 0.15 mM to about 1.25 mM, from about 0.15 mM to about 1.00 mM, from about 0.15 mM to about 0.75 mM, from about 0.15 mM to about 0.50 mM, from about 0.20 mM to about 2.50 mM, from about 0.20 mM to about 2.25 mM, from about 0.20 mM to about 2.00 mM, from about 0.20 mM to about 1.75 mM, from about 0.20 mM to about 1.50 mM, from about 0.20 mM to about 1.25 mM, from about 0.20 mM to about 1.00 mM, from about 0.20 mM to about 0.75 mM, from about 0.20 mM to about 0.50 mM, from about 0.30 mM to about 2.50 mM, from about 0.30 mM to about 2.25 mM, from about 0.30 mM to about 2.00 mM, from about 0.30 mM to about 1.75 mM, from about 0.30 mM to about 1.50 mM, from about 0.30 mM to about 1.25 mM, from about 0.30 mM to about 1.00 mM, from about 0.30 mM to about 0.75 mM, from about 0.30 mM to about 0.50 mM, from about 0.35 mM to about 2.50 mM, from about 0.35 mM to about 2.25 mM, from about 0.35 mM to about 2.00 mM, from about 0.35 mM to about 1.75 mM, from about 0.35 mM to about 1.50 mM, from about 0.35 mM to about 1.25 mM, from about 0.35 mM to about 1.00 mM, from about 0.35 mM to about 0.75 mM, or from about 0.35 mM to about 0.50 mM, inclusive of any ranges therebetween.

In one or more embodiments, the method of reducing the leakage of biological components from cells or instructions for preserving and storing the cells may further include analyzing one or more of the cells. In one or more embodiments, analyzing one or more of the cells may include conducting a molecular analysis on one or more of the cells. In one or more embodiments, analyzing one or more of the cells may include conducting a nucleic acid or protein analysis of one or more of the cells. In one or more embodiments, analyzing one or more of the cells may include conducting a ribonucleic acid (RNA) analysis of one or more of the cells. In one or more embodiments, analyzing one or more of the cells may include conducting a single cell RNA analysis of one or more of the cells. In one or more embodiments, analyzing one or more of the cells may include conducting a deoxyribonucleic acid (DNA) analysis of one or more of the cells. In one or more embodiments, analyzing one or more of the cells may include conducting a single cell deoxyribonucleic acid (DNA) analysis of one or more of the cells. In one or more embodiments, analyzing one or more of the cells may include conducting a protein analysis of one or more of the cells. In one or more embodiments, analyzing one or more of the cells may include conducting a single cell protein analysis of one or more of the cells.

In one or more embodiments, the method of reducing the leakage of biological components from cells or instructions for preserving and storing the cells may further include conducting a molecular analysis of biological components of one or more of the cells. In one or more embodiments, the molecular analysis is single cell molecular analysis.

In one or more embodiments, the method of reducing the leakage of biological components from cells or instructions for preserving and storing the cells may further include partitioning of the biological components for single cell analysis. In one or more embodiments, the partition is an aqueous droplet in oil emulsion. In one or more embodiments, the kit further includes an oil for partitioning in an aqueous droplet in oil emulsion. In one or more embodiments, the partition further includes a gel bead. In one or more embodiments, the kit further includes gel beads for the partitioning.

In one or more embodiments, the method of reducing the leakage of biological components from cells or instructions for preserving and storing the cells may further include generating single cell barcoded biological components (analytes) for single cell data processing.

In one or more embodiments, the fixative buffer, treatment buffer, or storage buffer further includes a cryoprotectant reagent. In one or more embodiments, the fixative buffer, treatment buffer, or storage buffer further includes a stabilizing reagent. In one or more embodiments, the fixative buffer, treatment buffer, or storage buffer further includes an RNA stabilizer. In one or more embodiments, the fixative buffer, treatment buffer, or storage buffer further includes a ribonucleoside vanadyl complex (RVC). In one or more embodiments, the fixative buffer, treatment buffer, or storage buffer further includes an RNase inhibitor.

In one or more embodiments, the biological components comprise RNA. In one or more embodiments, the biological components comprise DNA. In one or more embodiments, the biological components comprise protein.

### Implementation #11

In accordance with one or more embodiments, a method for reducing leakage of biological components from cells during preservation and storage is described. The method of reducing the leakage of biological components from cells may include i) contacting a plurality of cells with a fixative buffer comprising a poloxamine/Tetronic® polymer; ii) removing the fixative buffer from the cells; iii) contacting the cells with a treatment buffer comprising a poloxamine/Tetronic® polymer; iv) removing the treatment buffer from the cells; and v) suspending the cells in a storage buffer comprising a poloxamine/Tetronic® polymer.

In accordance with one or more embodiments, a kit for reducing leakage of biological components from cells during preservation and storage is described. The kit may include i) a fixative buffer comprising a poloxamine/Tetronic® polymer; ii) a treatment buffer comprising a poloxamine/Tetronic® polymer; iii) a storage buffer comprising a poloxamine/Tetronic® polymer; and iv) instructions for preserving and storing a plurality of cells in a manner that reduces leakage of biological components.

In one or more embodiments, the poloxamine/Tetronic® polymer may include a poloxamine 1107/T1107 Tetronic® polymer. In various embodiments, the fixative buffer may include a concentration of poloxamine 1107/T1107 Tetronic® polymer from about 0.05 mM to about 2.50 mM, from about 0.05 mM to about 2.25 mM, from about 0.05 mM to about 2.00 mM, from about 0.05 mM to about 1.75 mM, from about 0.05 mM to about 1.50 mM, from about 0.05 mM to about 1.25 mM, from about 0.05 mM to about 1.00 mM, from about 0.05 mM to about 0.75 mM, from about 0.05 mM to about 0.50 mM, from about 0.10 mM to about 2.50 mM, from about 0.10 mM to about 2.25 mM, from about 0.10 mM to about 2.00 mM, from about 0.10 mM to about 1.75 mM, from about 0.10 mM to about 1.50 mM, from about 0.10 mM to about 1.25 mM, from about 0.10 mM to about 1.00 mM, from about 0.10 mM to about 0.75 mM, from about 0.10 mM to about 0.50 mM, from about 0.15 mM to about 2.50 mM, from about 0.15 mM to about 2.25 mM, from about 0.15 mM to about 2.00 mM, from about 0.15 mM to about 1.75 mM, from about 0.15 mM to about 1.50 mM, from about 0.15 mM to about 1.25 mM, from about 0.15 mM to about 1.00 mM, from about 0.15 mM to about 0.75 mM, from about 0.15 mM to about 0.50 mM, from about 0.20 mM to about 2.50 mM, from about 0.20 mM to about 2.25 mM, from about 0.20 mM to about 2.00 mM, from about 0.20 mM to about 1.75 mM, from about 0.20 mM to about 1.50 mM, from about 0.20 mM to about 1.25 mM, from about 0.20 mM to about 1.00 mM, from about 0.20 mM to about 0.75 mM, from about 0.20 mM to about 0.50 mM, from about 0.30 mM to about 2.50 mM, from about 0.30 mM to about 2.25 mM, from about 0.30 mM to about 2.00 mM, from about 0.30 mM to about 1.75 mM, from about 0.30 mM to about 1.50 mM, from about 0.30 mM to about 1.25 mM, from about 0.30 mM to about 1.00 mM, from about 0.30 mM to about 0.75 mM, from about 0.30 mM to about 0.50 mM, from about 0.35 mM to about 2.50 mM, from about 0.35 mM to about 2.25 mM, from about 0.35 mM to about 2.00 mM, from about 0.35 mM to about 1.75 mM, from about 0.35 mM to about 1.50 mM, from about 0.35 mM to about 1.25 mM, from about 0.35 mM to about 1.00 mM, from about 0.35 mM to about 0.75 mM, or from about 0.35 mM to about 0.50 mM, inclusive of any ranges therebetween.

In one or more embodiments, the fixative buffer may include a crosslinking fixative. In one or more embodiments, the fixative buffer may include glyoxal. In one or more embodiments, the fixative buffer may include from about 0.05% glyoxal to about 1.5% glyoxal, from about 0.05% glyoxal to about 1.75% glyoxal, from about 0.05% glyoxal to about 2.00% glyoxal, from about 0.05% glyoxal to about 2.25% glyoxal, from about 0.05% glyoxal to about 2.50% glyoxal, from about 0.05% glyoxal to about 2.75% glyoxal, from about 0.05% glyoxal to about 3.00% glyoxal, from about 0.05% glyoxal to about 3.25% glyoxal, from about 0.05% glyoxal to about 3.50% glyoxal, from about 0.05% glyoxal to about 3.75% glyoxal, from about 0.05% glyoxal to about 4.00% glyoxal, from about 0.05% glyoxal to about 4.25% glyoxal, from about 0.05% glyoxal to about 4.50% glyoxal, from about 0.05% glyoxal to about 4.75% glyoxal, from about 0.05% glyoxal to about 5.00% glyoxal, from about 0.10% glyoxal to about 1.5% glyoxal, from about 0.10% glyoxal to about 1.75% glyoxal, from about 0.10% glyoxal to about 2.00% glyoxal, from about 0.10% glyoxal to about 2.25% glyoxal, from about 0.10% glyoxal to about 2.50% glyoxal, from about 0.10% glyoxal to about 2.75% glyoxal, from about 0.10% glyoxal to about 3.00% glyoxal, from about 0.10% glyoxal to about 3.25% glyoxal, from about 0.10% glyoxal to about 3.50% glyoxal, from about 0.10% glyoxal to about 3.75% glyoxal, from about 0.10% glyoxal to about 4.00% glyoxal, from about 0.10% glyoxal to about 4.25% glyoxal, from about 0.10% glyoxal to about 4.50% glyoxal, from about 0.10% glyoxal to about 4.75% glyoxal, from about 0.10% glyoxal to about 5.00% glyoxal, from about 0.15% glyoxal to about 1.5% glyoxal, from about 0.15% glyoxal to about 1.75% glyoxal, from about 0.15% glyoxal to about 2.00% glyoxal, from about 0.15% glyoxal to about 2.25% glyoxal, from about 0.15% glyoxal to about 2.50% glyoxal, from about 0.15% glyoxal to about 2.75% glyoxal, from about 0.15% glyoxal to about 3.00% glyoxal, from about 0.15% glyoxal to about 3.25% glyoxal, from about 0.15% glyoxal to about 3.50% glyoxal, from about 0.15% glyoxal to about 3.75% glyoxal, from about 0.15% glyoxal to about 4.00% glyoxal, from about 0.15% glyoxal to about 4.25% glyoxal, from about 0.15% glyoxal to about 4.50% glyoxal, from about 0.15% glyoxal to about 4.75% glyoxal, from about 0.15% glyoxal to about 5.00% glyoxal, from about 0.20% glyoxal to about 1.5% glyoxal, from about 0.20% glyoxal to about 1.75% glyoxal, from about 0.20% glyoxal to about 2.00% glyoxal, from about 0.20% glyoxal to about 2.25% glyoxal, from about 0.20% glyoxal to about 2.50% glyoxal, from about 0.20% glyoxal to about 2.75% glyoxal, from about 0.20% glyoxal to about 3.00% glyoxal, from about 0.20% glyoxal to about 3.25% glyoxal, from about 0.20% glyoxal to about 3.50% glyoxal, from about 0.20% glyoxal to about 3.75% glyoxal, from about 0.20% glyoxal to about 4.00% glyoxal, from about 0.20% glyoxal to about 4.25% glyoxal, from about 0.20% glyoxal to about 4.50% glyoxal, from about 0.20% glyoxal to about 4.75% glyoxal, from about 0.20% glyoxal to about 5.00% glyoxal, from about 0.25% glyoxal to about 1.5% glyoxal, from about 0.25% glyoxal to about 1.75% glyoxal, from about 0.25% glyoxal to about 2.00% glyoxal, from about 0.25% glyoxal to about 2.25% glyoxal, from about 0.25% glyoxal to about 2.50% glyoxal, from about 0.25% glyoxal to about 2.75% glyoxal, from about 0.25% glyoxal to about 3.00% glyoxal, from about 0.25% glyoxal to about 3.25% glyoxal, from about 0.25% glyoxal to about 3.50% glyoxal, from about 0.25% glyoxal to about 3.75% glyoxal, from about 0.25% glyoxal to about 4.00% glyoxal, from about 0.25% glyoxal to about 4.25% glyoxal, from about 0.25% glyoxal to about 4.50% glyoxal, from about 0.25% glyoxal to about 4.75% glyoxal, from about 0.25% glyoxal to about 5.00% glyoxal, from about 0.30% glyoxal to about 1.5% glyoxal, from about 0.30% glyoxal to about 1.75% glyoxal, from about 0.30% glyoxal to about 2.00% glyoxal, from about 0.30% glyoxal to about 2.25% glyoxal, from about 0.30% glyoxal to about 2.50% glyoxal, from about 0.30% glyoxal to about 2.75% glyoxal, from about 0.30% glyoxal to about 3.00% glyoxal, from about 0.30% glyoxal to about 3.25% glyoxal, from about 0.30% glyoxal to about 3.50% glyoxal, from about 0.30% glyoxal to about 3.75% glyoxal, from about 0.30% glyoxal to about 4.00% glyoxal, from about 0.30% glyoxal to about 4.25% glyoxal, from about 0.30% glyoxal to about 4.50% glyoxal, from about 0.30% glyoxal to about 4.75% glyoxal, from about 0.30% glyoxal to about 5.00% glyoxal, from about 0.35% glyoxal to about 1.5% glyoxal, from about 0.35% glyoxal to about 1.75% glyoxal, from about 0.35% glyoxal to about 2.00% glyoxal, from about 0.35% glyoxal to about 2.25% glyoxal, from about 0.35% glyoxal to about 2.50% glyoxal, from about 0.35% glyoxal to about 2.75% glyoxal, from about 0.35% glyoxal to about 3.00% glyoxal, from about 0.35% glyoxal to about 3.25% glyoxal, from about 0.35% glyoxal to about 3.50% glyoxal, from about 0.35% glyoxal to about 3.75% glyoxal, from about 0.35% glyoxal to about 4.00% glyoxal, from about 0.35% glyoxal to about 4.25% glyoxal, from about 0.35% glyoxal to about 4.50% glyoxal, from about 0.35% glyoxal to about 4.75% glyoxal, or from about 0.35% glyoxal to about 5.00% glyoxal, inclusive of any ranges therebetween.

In one or more embodiments, the fixative buffer may include DSP or DTSSP.

In various embodiments, the fixative buffer may include a concentration of DSP from about 0.05 mM to about 10 mM, from about 0.05 mM to about 7.5 mM, from about 0.05 mM to about 5.00 mM, from about 0.05 mM to about 3.5 mM, from about 0.05 mM to about 2.50 mM, from about 0.05 mM to about 1.25 mM, from about 0.05 mM to about 1.00 mM, from about 0.05 mM to about 0.75 mM, from about 0.05 mM to about 0.50 mM, 0.1 mM to about 10 mM, from about 0.1 mM to about 7.5 mM, from about 0.1 mM to about 5.00 mM, from about 0.1 mM to about 3.5 mM, from about 0.1 mM to about 2.50 mM, from about 0.1 mM to about 1.25 mM, from about 0.1 mM to about 1.00 mM, from about 0.1 mM to about 0.75 mM, from about 0.1 mM to about 0.50 mM, 0.25 mM to about 10 mM, from about 0.25 mM to about 7.5 mM, from about 0.25 mM to about 5.00 mM, from about 0.25 mM to about 3.5 mM, from about 0.25 mM to about 2.50 mM, from about 0.25 mM to about 1.25 mM, from about 0.25 mM to about 1.00 mM, from about 0.25 mM to about 0.75 mM, from about 0.25 mM to about 0.50 mM, 0.5 mM to about 10 mM, from about 0.5 mM to about 7.5 mM, from about 0.5 mM to about 5.00 mM, from about 0.5 mM to about 3.5 mM, from about 0.5 mM to about 2.50 mM, from about 0.5 mM to about 1.25 mM, from about 0.5 mM to about 1.00 mM, from about 0.5 mM to about 0.75 mM, 1.25 mM to about 10 mM, from about 1.25 mM to about 7.5 mM, from about 1.25 mM to about 5.00 mM, from about 1.25 mM to about 3.5 mM, from about 1.25 mM to about 2.50 mM, 2.5 mM to about 10 mM, from about 2.5 mM to about 7.5 mM, from about 2.5 mM to about 5.00 mM, and from about 2.5 mM to about 3.5 mM, inclusive of any ranges, values or endpoints therebetween. In various embodiments, the concentration of DSP is 2.5 mM or 1.25 mM.

In various embodiments, the fixative buffer may include a concentration of DTSSP from about 0.25 mM to about 20 mM, from about 0.25 mM to about 10 mM, from about 0.25 mM to about 7.5 mM, from about 0.25 mM to about 7.0 mM, from about 0.25 mM to about 5.00 mM, from about 0.25 mM to about 3.5 mM, from about 0.25 mM to about 2.50 mM, from about 0.25 mM to about 1.25 mM, from about 0.25 mM to about 1.00 mM, from about 0.25 mM to about 0.75 mM, from about 0.25 mM to about 0.50 mM, from about 0.5 mM to about 20 mM, from about 0.5 mM to about 10 mM, from about 0.5 mM to about 7.5 mM, from about 0.5 mM to about 7.0 mM, from about 0.5 mM to about 5.00 mM, from about 0.5 mM to about 3.5 mM, from about 0.5 mM to about 2.50 mM, from about 0.5 mM to about 1.25 mM, from about 0.5 mM to about 1.00 mM, from about 0.5 mM to about 0.75 mM, from about 1 mM to about 20 mM, from about 1 mM to about 10 mM, from about 1 mM to about 7.5 mM, from about 1 mM to about 7.0 mM, from about 1 mM to about 5.00 mM, from about 1 mM to about 3.5 mM, from about 1 mM to about 2.50 mM, from about 1 mM to about 1.25 mM, from about 2 mM to about 20 mM, from about 2 mM to about 10 mM, from about 2 mM to about 7.5 mM, from about 2 mM to about 7.0 mM, from about 2 mM to about 5.00 mM, from about 2 mM to about 3.5 mM, from about 2 mM to about 2.50 mM, from about 2.5 mM to about 20 mM, from about 2.5 mM to about 10 mM, from about 2.5 mM to about 7.5 mM, from about 2.5 mM to about 7.0 mM, from about 2.5 mM to about 5.00 mM, and from about 2.5 mM to about 3.5 mM, inclusive of any ranges, values or endpoints therebetween. In various embodiments, the concentration of DTSSP is 2.5 mM or 5 mM.

In various embodiments, the storage buffer may include a concentration of poloxamine 1107/T1107 Tetronic® polymer from about 0.05 mM to about 2.50 mM, from about 0.05 mM to about 2.25 mM, from about 0.05 mM to about 2.00 mM, from about 0.05 mM to about 1.75 mM, from about 0.05 mM to about 1.50 mM, from about 0.05 mM to about 1.25 mM, from about 0.05 mM to about 1.00 mM, from about 0.05 mM to about 0.75 mM, from about 0.05 mM to about 0.50 mM, from about 0.10 mM to about 2.50 mM, from about 0.10 mM to about 2.25 mM, from about 0.10 mM to about 2.00 mM, from about 0.10 mM to about 1.75 mM, from about 0.10 mM to about 1.50 mM, from about 0.10 mM to about 1.25 mM, from about 0.10 mM to about 1.00 mM, from about 0.10 mM to about 0.75 mM, from about 0.10 mM to about 0.50 mM, from about 0.15 mM to about 2.50 mM, from about 0.15 mM to about 2.25 mM, from about 0.15 mM to about 2.00 mM, from about 0.15 mM to about 1.75 mM, from about 0.15 mM to about 1.50 mM, from about 0.15 mM to about 1.25 mM, from about 0.15 mM to about 1.00 mM, from about 0.15 mM to about 0.75 mM, from about 0.15 mM to about 0.50 mM, from about 0.20 mM to about 2.50 mM, from about 0.20 mM to about 2.25 mM, from about 0.20 mM to about 2.00 mM, from about 0.20 mM to about 1.75 mM, from about 0.20 mM to about 1.50 mM, from about 0.20 mM to about 1.25 mM, from about 0.20 mM to about 1.00 mM, from about 0.20 mM to about 0.75 mM, from about 0.20 mM to about 0.50 mM, from about 0.30 mM to about 2.50 mM, from about 0.30 mM to about 2.25 mM, from about 0.30 mM to about 2.00 mM, from about 0.30 mM to about 1.75 mM, from about 0.30 mM to about 1.50 mM, from about 0.30 mM to about 1.25 mM, from about 0.30 mM to about 1.00 mM, from about 0.30 mM to about 0.75 mM, from about 0.30 mM to about 0.50 mM, from about 0.35 mM to about 2.50 mM, from about 0.35 mM to about 2.25 mM, from about 0.35 mM to about 2.00 mM, from about 0.35 mM to about 1.75 mM, from about 0.35 mM to about 1.50 mM, from about 0.35 mM to about 1.25 mM, from about 0.35 mM to about 1.00 mM, from about 0.35 mM to about 0.75 mM, or from about 0.35 mM to about 0.50 mM, inclusive of any ranges therebetween.

In one or more embodiments, the method of reducing the leakage of biological components from cells or instructions for preserving and storing the cells may further include storing the cells at room temperature. In one or more embodiments, the method of reducing the leakage of biological components from cells or instructions for preserving and storing the cells may further include storing the cells at about 1 degree Celsius to about 4 degrees Celsius, about 1 degree Celsius to about 5 degrees Celsius, about 1 degree Celsius to about 6 degrees Celsius, about 1 degree Celsius to about 7 degrees Celsius, about 1 degree Celsius to about 8 degrees Celsius, about 1 degree Celsius to about 9 degrees Celsius, about 1 degree Celsius to about 10 degrees Celsius, about 1 degree Celsius to about 11 degrees Celsius, about 1 degree Celsius to about 12 degrees Celsius, about 2 degrees Celsius to about 4 degrees Celsius, about 2 degrees Celsius to about 5 degrees Celsius, about 2 degrees Celsius to about 6 degrees Celsius, about 2 degrees Celsius to about 7 degrees Celsius, about 2 degrees Celsius to about 8 degrees Celsius, about 2 degrees Celsius to about 9 degrees Celsius, about 2 degrees Celsius to about 10 degrees Celsius, about 2 degrees Celsius to about 11 degrees Celsius, about 2 degrees Celsius to about 12 degrees Celsius, about 3 degrees Celsius to about 4 degrees Celsius, about 3 degrees Celsius to about 5 degrees Celsius, about 3 degrees Celsius to about 6 degrees Celsius, about 3 degrees Celsius to about 7 degrees Celsius, about 3 degrees Celsius to about 8 degrees Celsius, about 3 degrees Celsius to about 9 degrees Celsius, about 3 degrees Celsius to about 10 degrees Celsius, about 3 degrees Celsius to about 11 degrees Celsius, about 3 degrees Celsius to about 12 degrees Celsius, about 4 degrees Celsius to about 5 degrees Celsius, about 4 degrees Celsius to about 6 degrees Celsius, about 4 degrees Celsius to about 7 degrees Celsius, about 4 degrees Celsius to about 8 degrees Celsius, about 4 degrees Celsius to about 9 degrees Celsius, about 4 degrees Celsius to about 10 degrees Celsius, about 4 degrees Celsius to about 11 degrees Celsius, about 4 degrees Celsius to about 12 degrees Celsius, about 5 degrees Celsius to about 6 degrees Celsius, about 5 degrees Celsius to about 7 degrees Celsius, about 5 degrees Celsius to about 8 degrees Celsius, about 5 degrees Celsius to about 9 degrees Celsius, about 5 degrees Celsius to about 10 degrees Celsius, about 5 degrees Celsius to about 11 degrees Celsius, or about 5 degrees Celsius to about 12 degrees Celsius, inclusive of any ranges therebetween.

In one or more embodiments, the method of reducing the leakage of biological components from cells or instructions for preserving and storing the cells may further include cryopreserving the cells.

In one or more embodiments, the method of reducing the leakage of biological components from cells or instructions for preserving and storing the cells may further include cryopreserving the cells at about 0 degrees Celsius to about −196 degrees Celsius, 0 degrees Celsius to about −175 degrees Celsius, 0 degrees Celsius to about −150 degrees Celsius, 0 degrees Celsius to about −125 degrees Celsius, 0 degrees Celsius to about −100 degrees Celsius, 0 degrees Celsius to about −80 degrees Celsius, 0 degrees Celsius to about −50 degrees Celsius, 0 degrees Celsius to about −20 degrees Celsius, about −20 degrees Celsius to about −196 degrees Celsius, −20 degrees Celsius to about −175 degrees Celsius, −20 degrees Celsius to about −150 degrees Celsius, −20 degrees Celsius to about −125 degrees Celsius, −20 degrees Celsius to about −100 degrees Celsius, −20 degrees Celsius to about −80 degrees Celsius, −20 degrees Celsius to about −50 degrees Celsius, −80 degrees Celsius to about −196 degrees Celsius, −80 degrees Celsius to about −175 degrees Celsius, −80 degrees Celsius to about −150 degrees Celsius, −80 degrees Celsius to about −125 degrees Celsius, and −80 degrees Celsius to about −100 degrees Celsius, inclusive of any ranges, values or endpoints therebetween.

In one or more embodiments, the storage buffer is a phosphate buffered saline (PBS)-based storage buffer. In one or more embodiments, the storage buffer is a saline sodium citrate (SSC) based storage buffer.

In one or more embodiments, the contacting step may include incubating for 0.5 hour, 1 hour, 1.5 hours, or 2 hours at room temperature or incubating overnight at 2-8 degrees Celsius, 1-7 degrees Celsius, or 1-9 degrees Celsius.

In one or more embodiments, the cells may be maintained in the storage buffer for a time period of about 0.5 hour to about 1 day, about 0.5 hour to about 2 days, about 0.5 hour to about 3 days, about 0.5 hour to about 4 days, about 0.5 hour to about 5 days, about 0.5 hour to about 6 days, about 0.5 hour to about 7 days, about 1 hour to about 1 day, about 1 hour to about 2 days, about 1 hour to about 3 days, about 1 hour to about 4 days, about 1 hour to about 5 days, about 1 hour to about 6 days, about 1 hour to about 7 days, about 2 hours to about 1 day, about 2 hours to about 2 days, about 2 hours to about 3 days, about 2 hours to about 4 days, about 2 hours to about 5 days, about 2 hours to about 6 days, about 2 hours to about 7 days, about 3 hours to about 1 day, about 3 hours to about 2 days, about 3 hours to about 3 days, about 3 hours to about 4 days, about 3 hours to about 5 days, about 3 hours to about 6 days, or about 3 hours to about 7 days, inclusive of any ranges therebetween. In other embodiments cells may be stored for more than 1 week, such as 2 weeks, 3 weeks, 1 month or up to one year.

In one or more embodiments, the treatment buffer may include a PBS based buffer comprising a concentration of poloxamine 1107/T1107 Tetronic® polymer from about 0.05 mM to about 2.50 mM, from about 0.05 mM to about 2.25 mM, from about 0.05 mM to about 2.00 mM, from about 0.05 mM to about 1.75 mM, from about 0.05 mM to about 1.50 mM, from about 0.05 mM to about 1.25 mM, from about 0.05 mM to about 1.00 mM, from about 0.05 mM to about 0.75 mM, from about 0.05 mM to about 0.50 mM, from about 0.10 mM to about 2.50 mM, from about 0.10 mM to about 2.25 mM, from about 0.10 mM to about 2.00 mM, from about 0.10 mM to about 1.75 mM, from about 0.10 mM to about 1.50 mM, from about 0.10 mM to about 1.25 mM, from about 0.10 mM to about 1.00 mM, from about 0.10 mM to about 0.75 mM, from about 0.10 mM to about 0.50 mM, from about 0.15 mM to about 2.50 mM, from about 0.15 mM to about 2.25 mM, from about 0.15 mM to about 2.00 mM, from about 0.15 mM to about 1.75 mM, from about 0.15 mM to about 1.50 mM, from about 0.15 mM to about 1.25 mM, from about 0.15 mM to about 1.00 mM, from about 0.15 mM to about 0.75 mM, from about 0.15 mM to about 0.50 mM, from about 0.20 mM to about 2.50 mM, from about 0.20 mM to about 2.25 mM, from about 0.20 mM to about 2.00 mM, from about 0.20 mM to about 1.75 mM, from about 0.20 mM to about 1.50 mM, from about 0.20 mM to about 1.25 mM, from about 0.20 mM to about 1.00 mM, from about 0.20 mM to about 0.75 mM, from about 0.20 mM to about 0.50 mM, from about 0.30 mM to about 2.50 mM, from about 0.30 mM to about 2.25 mM, from about 0.30 mM to about 2.00 mM, from about 0.30 mM to about 1.75 mM, from about 0.30 mM to about 1.50 mM, from about 0.30 mM to about 1.25 mM, from about 0.30 mM to about 1.00 mM, from about 0.30 mM to about 0.75 mM, from about 0.30 mM to about 0.50 mM, from about 0.35 mM to about 2.50 mM, from about 0.35 mM to about 2.25 mM, from about 0.35 mM to about 2.00 mM, from about 0.35 mM to about 1.75 mM, from about 0.35 mM to about 1.50 mM, from about 0.35 mM to about 1.25 mM, from about 0.35 mM to about 1.00 mM, from about 0.35 mM to about 0.75 mM, or from about 0.35 mM to about 0.50 mM, inclusive of any ranges therebetween.

In one or more embodiments, the method of reducing the leakage of biological components from cells or instructions for preserving and storing the cells may further include analyzing one or more of the cells. In one or more embodiments, analyzing one or more of the cells may include conducting a molecular analysis on one or more of the cells. In one or more embodiments, analyzing one or more of the cells may include conducting a nucleic acid or protein analysis of one or more of the cells. In one or more embodiments, analyzing one or more of the cells may include conducting a ribonucleic acid (RNA) analysis of one or more of the cells. In one or more embodiments, analyzing one or more of the cells may include conducting a single cell RNA analysis of one or more of the cells. In one or more embodiments, analyzing one or more of the cells may include conducting a deoxyribonucleic acid (DNA) analysis of one or more of the cells. In one or more embodiments, analyzing one or more of the cells may include conducting a single cell deoxyribonucleic acid (DNA) analysis of one or more of the cells. In one or more embodiments, analyzing one or more of the cells may include conducting a protein analysis of one or more of the cells. In one or more embodiments, analyzing one or more of the cells may include conducting a single cell protein analysis of one or more of the cells.

In one or more embodiments, the method of reducing the leakage of biological components from cells or instructions for preserving and storing the cells may further include conducting a molecular analysis of biological components of one or more of the cells. In one or more embodiments, the molecular analysis is single cell molecular analysis.

In one or more embodiments, the method of reducing the leakage of biological components from cells or instructions for preserving and storing the cells may further include partitioning of the biological components for single cell analysis. In one or more embodiments, the partition is an aqueous droplet in oil emulsion. In one or more embodiments, the kit further includes an oil for partitioning in an aqueous droplet in oil emulsion. In one or more embodiments, the partition further includes a gel bead. In one or more embodiments, the kit further includes gel beads for the partitioning.

In one or more embodiments, the method of reducing the leakage of biological components from cells or instructions for preserving and storing the cells may further include generating single cell barcoded biological components (analytes) for single cell data processing.

In one or more embodiments, the fixative buffer, treatment buffer, or storage buffer further includes a cryoprotectant reagent. In one or more embodiments, the fixative buffer, treatment buffer, or storage buffer further includes a stabilizing reagent. In one or more embodiments, the fixative buffer, treatment buffer, or storage buffer further includes an RNA stabilizer. In one or more embodiments, the fixative buffer, treatment buffer, or storage buffer further includes a ribonucleoside vanadyl complex (RVC). In one or more embodiments, the fixative buffer, treatment buffer, or storage buffer further includes an RNase inhibitor.

In one or more embodiments, the biological components comprise RNA. In one or more embodiments, the biological components comprise DNA. In one or more embodiments, the biological components comprise protein.

The compositions and processes of the present invention will be better understood in connection with the following examples, which are intended as an illustration only and not limiting of the scope of the invention. Various changes and modifications to the disclosed embodiments will be apparent to those skilled in the art and such changes and modifications including, without limitation, those relating to the processes, formulations and/or methods of the invention may be made without departing from the spirit of the invention and the scope of the appended claims.

### EXAMPLES

### Example 1

Fixation is necessary to freeze biological processes and preserve biological samples (e.g., cultured cells and tissues) to be processed later. Chemical crosslinking is one of the most common methods of fixation through the use of chemical compounds that react and form stable bonds between biomolecules. The most common crosslinker used in biology is formaldehyde due to its extremely strong crosslinking efficiency and reliability. However, formaldehyde is oftentimes too strong a crosslinker, such that many biomolecules become inaccessible or overly altered after crosslinking. Formaldehyde also leads to efficient nucleic acid-protein and protein-protein crosslinking and thus can be considered a pan-crosslinker (crosslinks almost everything). While formaldehyde crosslinking can be reversed with heating, this reversal step can be detrimental to certain biomolecules, especially RNAs. Therefore, other types of chemical crosslinkers that favor protein-protein crosslinking may be used to reduce nucleic acid-protein crosslinking interactions.

Dithiobis(succinimidyl propionate) (DSP or Lomant's Reagent) is a crosslinker whose action is reversible using reducing agents (rather than heating), and as a result, is milder on RNA. DSP is not particularly water-soluble, and therefore more water-soluble analogs of DSP have been made, one of which is the 3,3′-dithiobis(sulfosuccinidmidyl proprionate) (DTSSP). These crosslinkers are alternatives to formaldehyde, which is currently still the most widely used crosslinker in the field. Unlike formaldehyde, which has robust crosslinking efficiency, DSP alone, however, may not be sufficient to give adequate crosslinking stability. Therefore, combinations of these crosslinkers may be necessary. The manner in which these crosslinkers are combined is important due to their different water solubility profiles.

Chemical crosslinkers can be envisioned as drugs that cross the cellular membrane using the principles of cellular pharmacology. Cells are often less permeable to water-soluble (hydrophilic) compounds as opposed to non-water soluble (hydrophobic) compounds. In order to get water-soluble compounds into cells, detergents (e.g., tween, saponin) are often used. However, these detergents can also compromise the integrity of the cell membrane, resulting in leakage of intracellular biomolecules. As such, there exists a need in the art for methods of fixing cells for later analysis, wherein molecules to be detected at a later time are efficiently preserved.

The present disclosure provides a novel and advantageous strategy for fixing cells through a sequential two-step crosslinking approach (FIG. 11). Advantages of sequential crosslinking include: (1) obtaining high-quality RNA by preventing over-crosslinking of RNA within cells; (2) getting better recovery of cells by saturating extracellular crosslinking to form a shell and keep the membrane intact; and (3) more stable and consistent crosslinking in a shorter time.

Various methods can be used to test the effectiveness of crosslinking. Flow cytometry can be performed on cells stained with RNA dye (e.g., SYTO-RNA select) and cell viability dye (e.g., DNA dye 7aad). Flow cytometry is useful for looking at cell population parameters, gives a very nice overview, and shows the distribution of cell morphology. However, the permeability of the dye can potentially be impacted by crosslinking conditions, there can be autofluorescence from fixation conditions, and results may be difficult to interpret. Analysis can alternatively be done ungated rather than gated for 7aad+ cells since different fixation conditions can impact permeability of 7aad.

Poly-dT beads can be used for mRNA pulldown. This method allows for a quick look at the distribution of mRNA size, and one can indirectly infer some RNA crosslinking activity; however, interpretation can be confounded by false positives caused by non-specific crosslinking via protein to rRNA and false negatives caused by incompatibility with extraction (i.e., with formaldehyde), resulting in loss of RNA during extraction.

The following experiments demonstrate that DSP/DTSSP is a solid and consistent crosslinking combination. DSP/DTSSP sequential crosslinking demonstrates: (1) minimal cell loss during storage (˜0% and 15% for fixed vs 50% for unfixed in one week); (2) retention of high crosslinking levels for higher dose treatment (strong mRNA band even on Day 7); (3) minimal RNA loss in higher dose treatment conditions; and (4) that sufficient crosslinking is achievable without overnight crosslinking. Another strategy for decrosslinking includes DTT, which is already integrated in the single cell workflow.

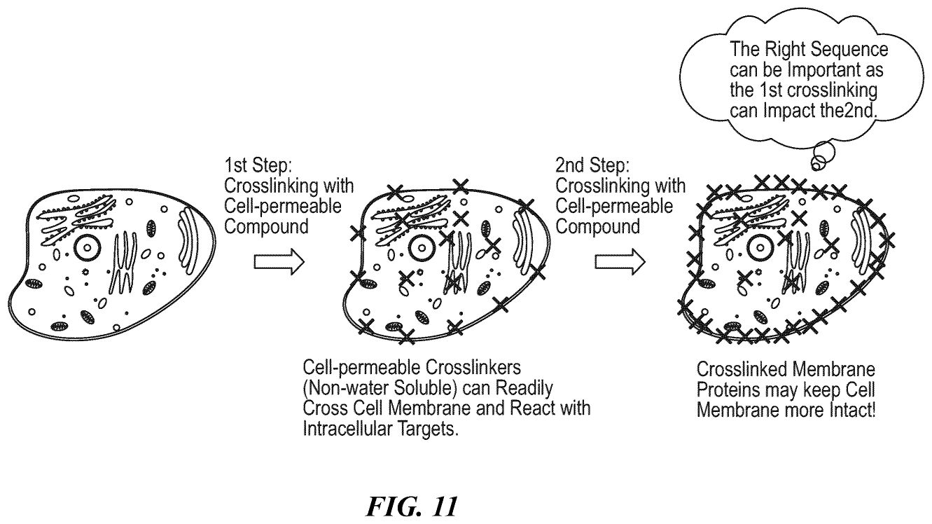

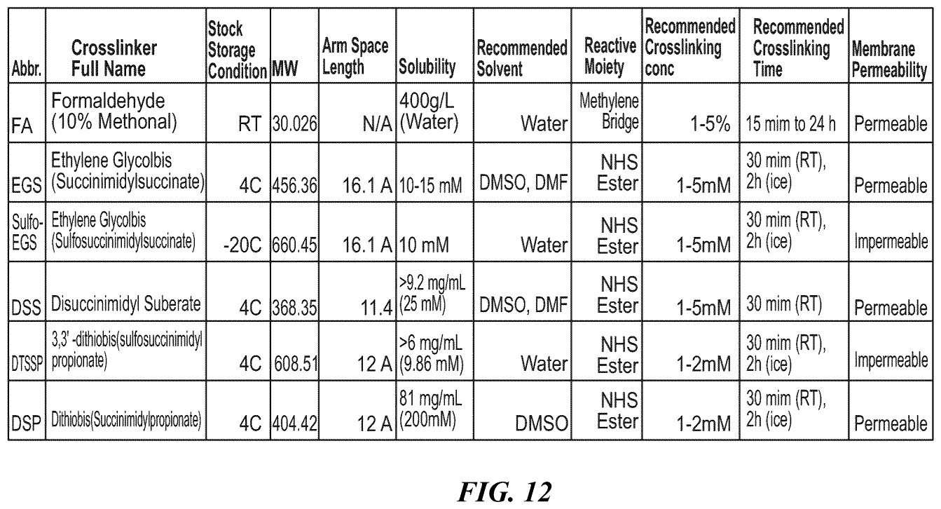

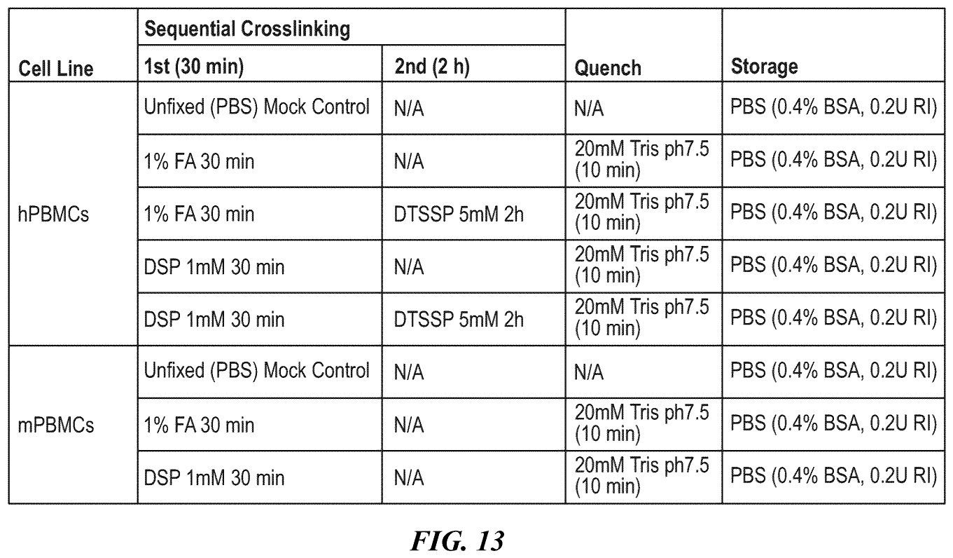

FIG. 12 shows properties of various cross-linkers used in a sequential crosslinking test and FIG. 13 is an experimental set-up for that test. The rationale for the test was that crosslinkers have different cell-permeability properties based on lipophilicity (i.e., hydrophobicity). Water-soluble compounds often do not enter the cell efficiently or are nearly cell-impermeable while water-insoluble compounds will usually enter the cell and pass through the cell membrane.

In a first step, human and mouse peripheral blood mononuclear cells (PBMCs) (250,000-300,000 cells) were either unfixed, treated with formaldehyde, or treated with 1 mM DSP for 30 minutes. In a second step, hPBMCs treated with formaldehyde or DSP were either untreated, or treated with 5 mM DTSSP for 2 hours. Quenching was performed in 20 mM Tris pH 7.5 for 10 minutes, and cells were stored at room temperature or 4° C. in PBS containing 0.4% BSA, 0.2 U RNase inhibitor.

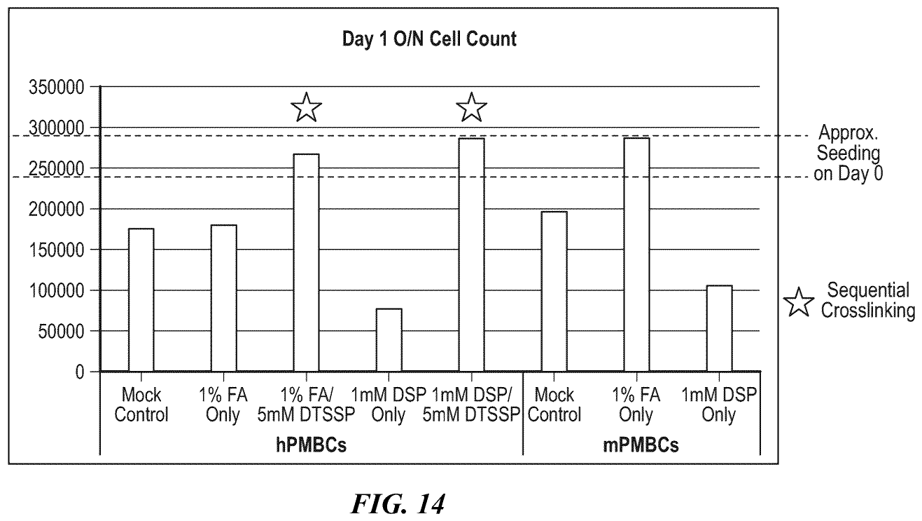

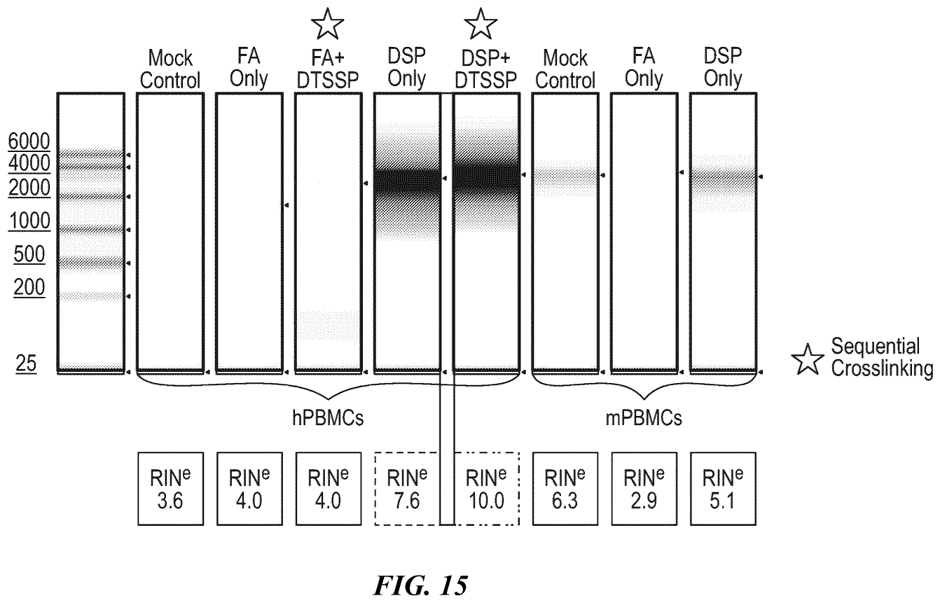

FIG. 14 shows results from the sequential crosslinking test. Cell counts of hPBMCs and mPBMCs were performed after overnight storage for the different combinations of cross-linkers. Similar cell counts were seen in samples treated with formaldehyde and those treated with the sequential combination of DSP and DTSSP. FIG. 15 shows RNA pulldown in the sample treated with DSP alone, and greater RNA pulldown in the sample treated with the sequential combination of DSP and DTSSP.

### Example 2

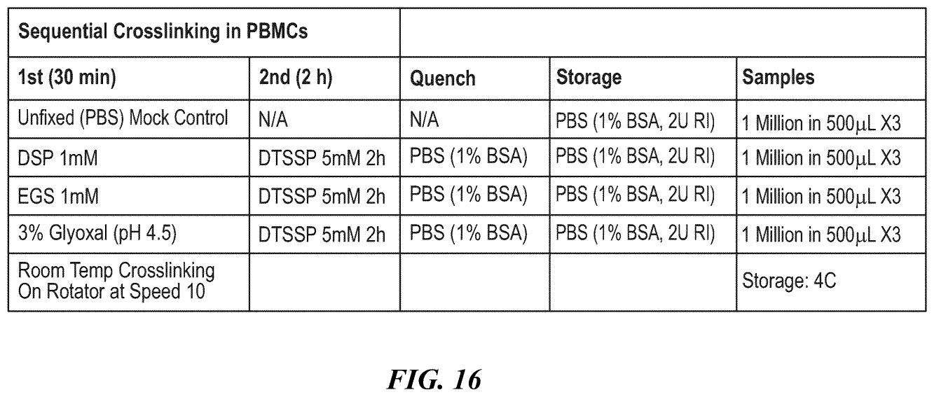

FIG. 16 depicts an experimental set-up for a sequential crosslinking experiment in PBMCs. In a first step, PBMCs (1,000,000 cells in 500 μL X3 at room temperature) were either unfixed, treated with 1 mM DSP, 1 mM EGS, or 3% glyoxal pH 4.5 for 30 minutes at room temperature with a rotator at speed 10. In a second step, PBMCs were either untreated, or treated with 5 mM DTSSP for 2 hours. Quenching was performed in PBS containing 1% BSA, and cells were stored at 4° C. in PBS containing 0.4% BSA, 0.2 U RNase inhibitor.

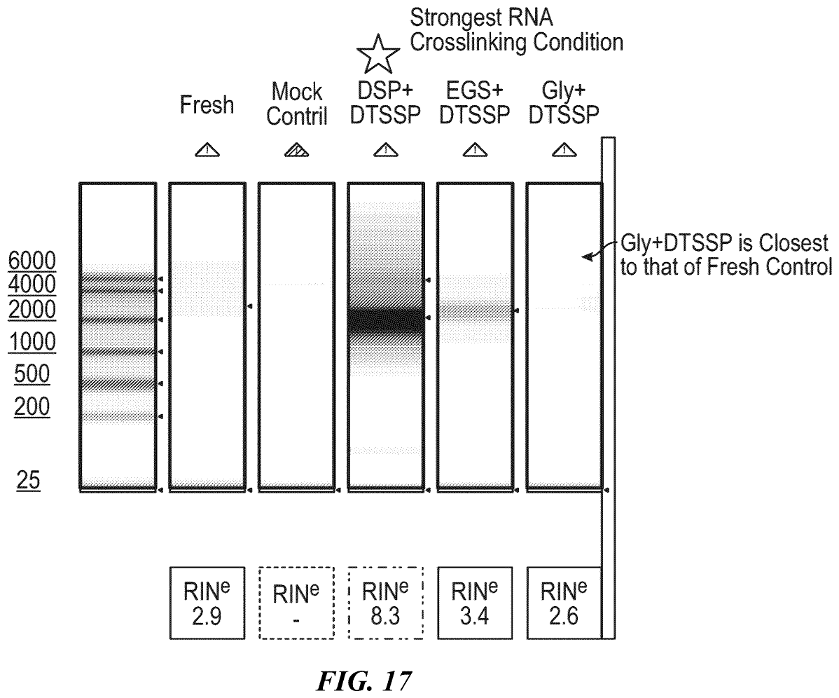

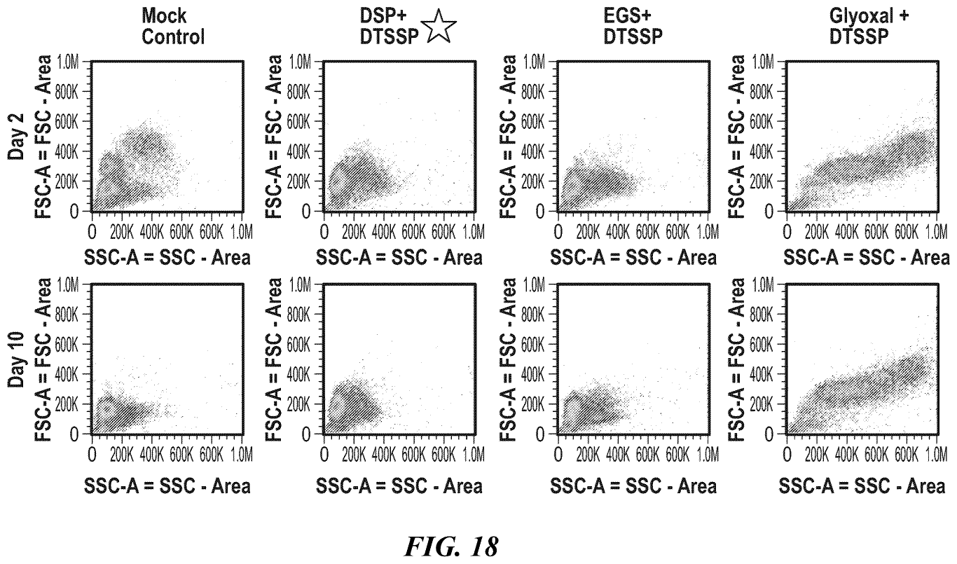

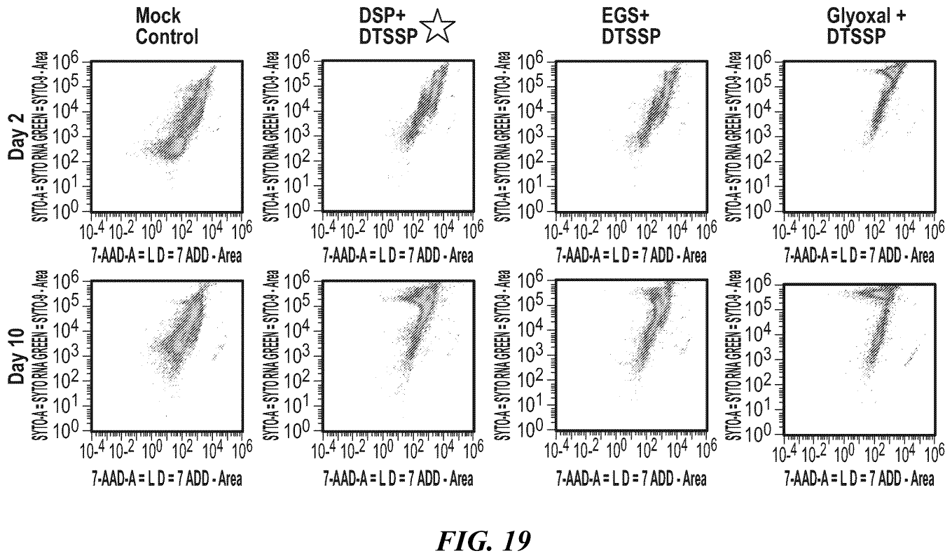

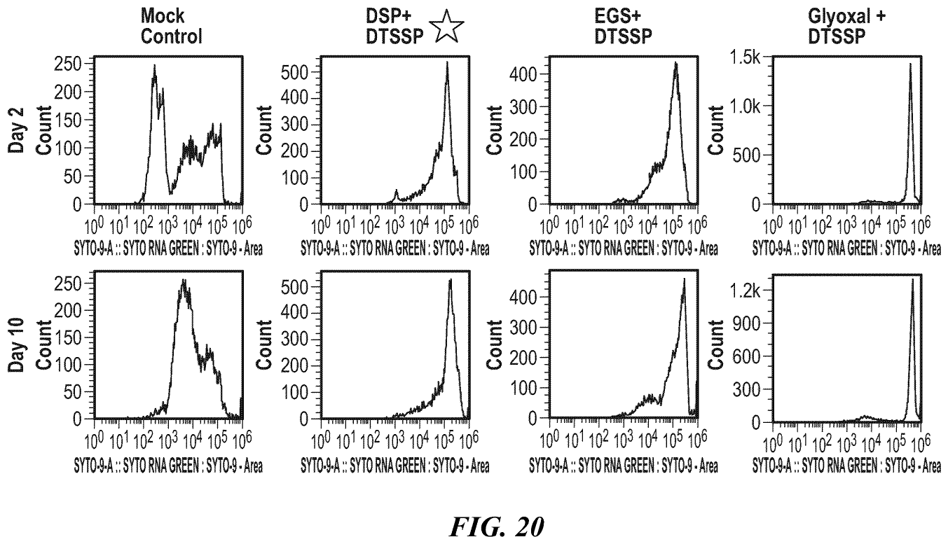

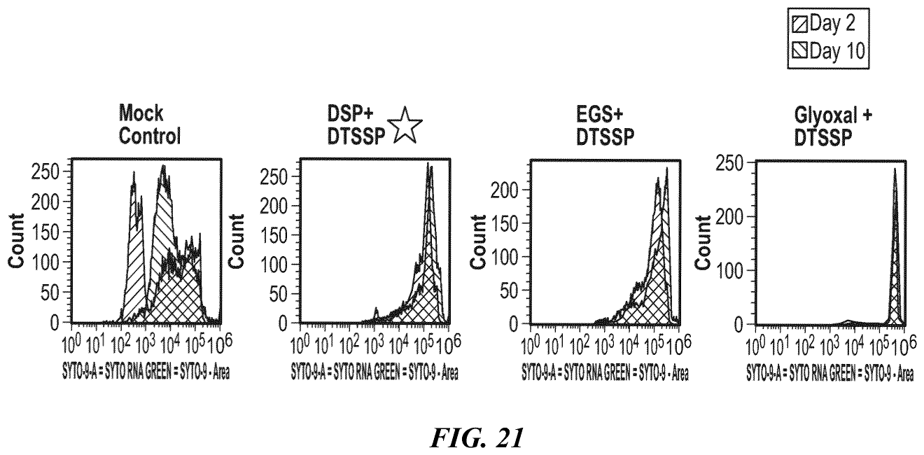

FIG. 17 shows that the most effective RNA pulldown was in the sample treated sequentially with DSP and DTSSP, although some RNA was pulled down from the EGS/DTSSP sample. The sample treated with glyoxal and DTSSP was the closest to the fresh control. FIG. 18 shows flow cytometry data (side scatter—SSC and forward scatter—FSC) at days 2 and 10 from the treated cells, showing the best results with the DSP/DTSSP combination. FIG. 19 shows flow cytometry data (staining with SYTO RNA vs. 7aad) at days 2 and 10 from the treated cells, again showing the best results with the sequential DSP/DTSSP combination. Likewise, FIGS. 20 and 21, which show flow cytometry flow data histograms and histogram overlaps, respectively (staining with SYTO RNA) at days 2 and 10, again show the best results with the sequential DSP/DTSSP combination.

### Example 3

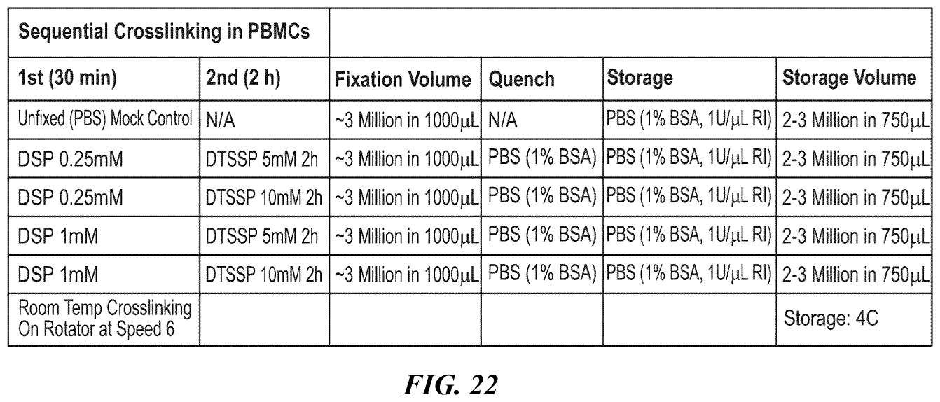

FIG. 22 depicts an experimental set-up for a sequential crosslinking experiment in PBMCs with DSP/DTSSP high and low factorial testing. In a first step, PBMCs (2-3,000,000 cells in 1000 μL at room temperature with a rotator at speed 6) were either unfixed, treated with 0.25 mM or 1 mM DSP. In a second step, PBMCs were either untreated, or treated with 5 mM or 10 mM DTSSP for 2 hours. Quenching was performed in PBS containing 1% BSA, and cells were stored at 4° C. in PBS containing 0.4% BSA, 0.2 U RNase inhibitor.

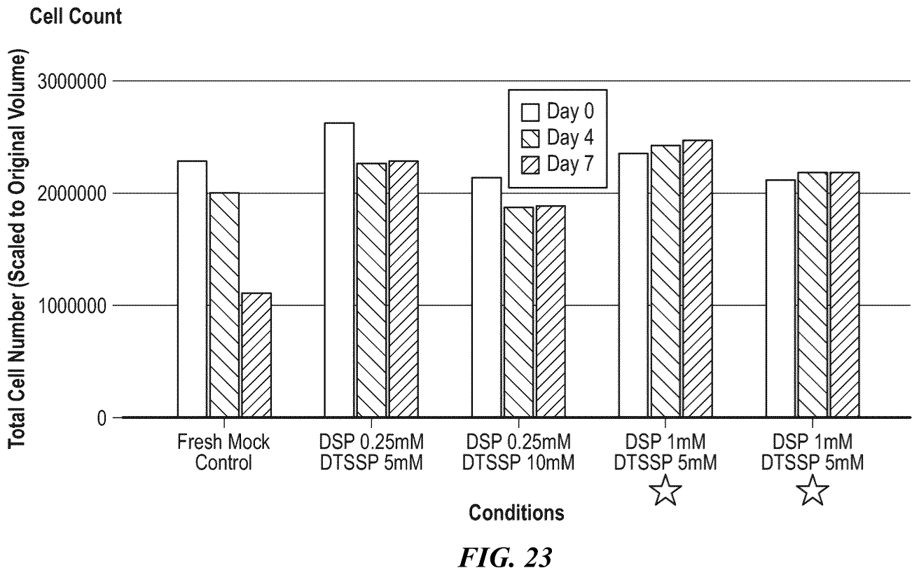

FIG. 23 shows that the best cell counts were obtained at days 4 and 7 with the sequential combination of 1 mM DSP and 5 mM DTSSP. There was negligible cell loss in storage for fixed samples, whereas fresh mock control cells showed substantial loss after a week. There was a slight cell loss with low dose DSP and nearly no cell loss in higher dose DSP conditions.

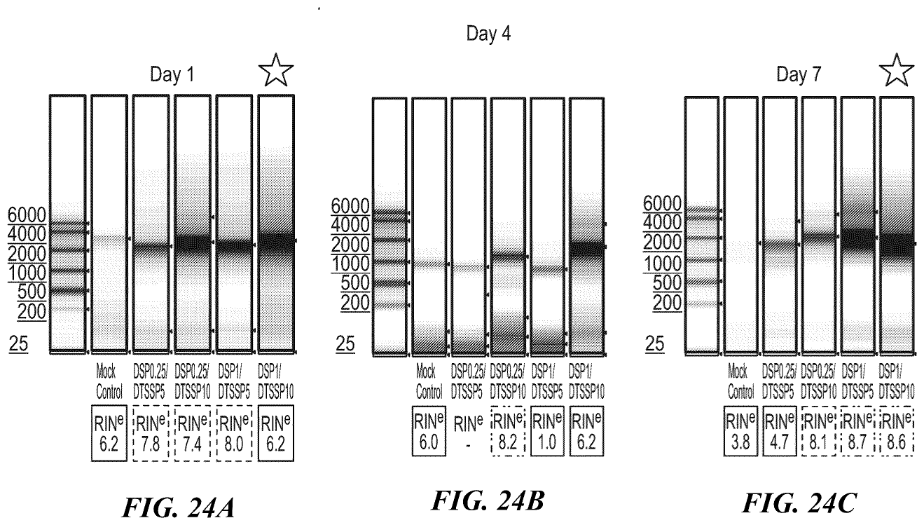

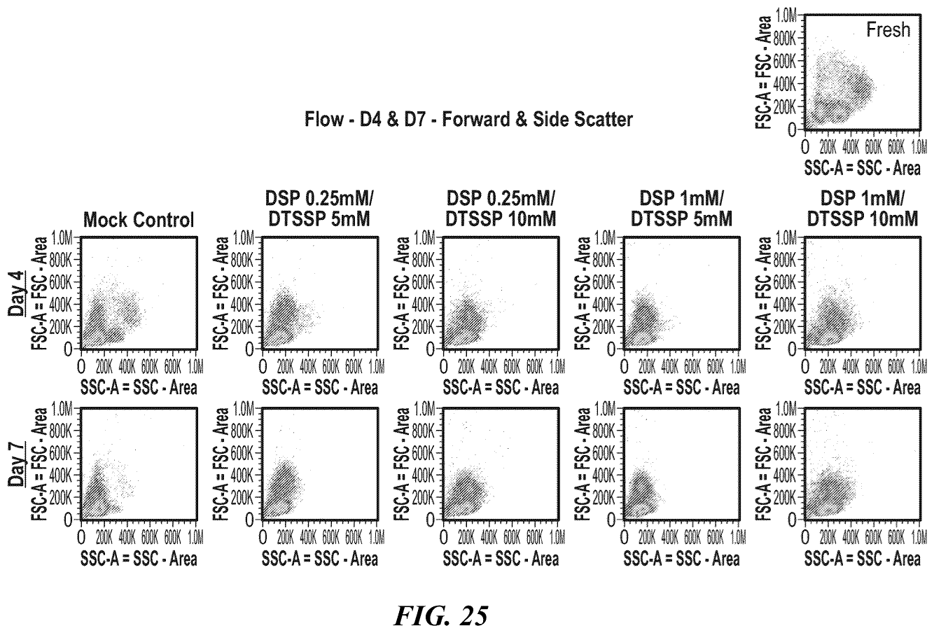

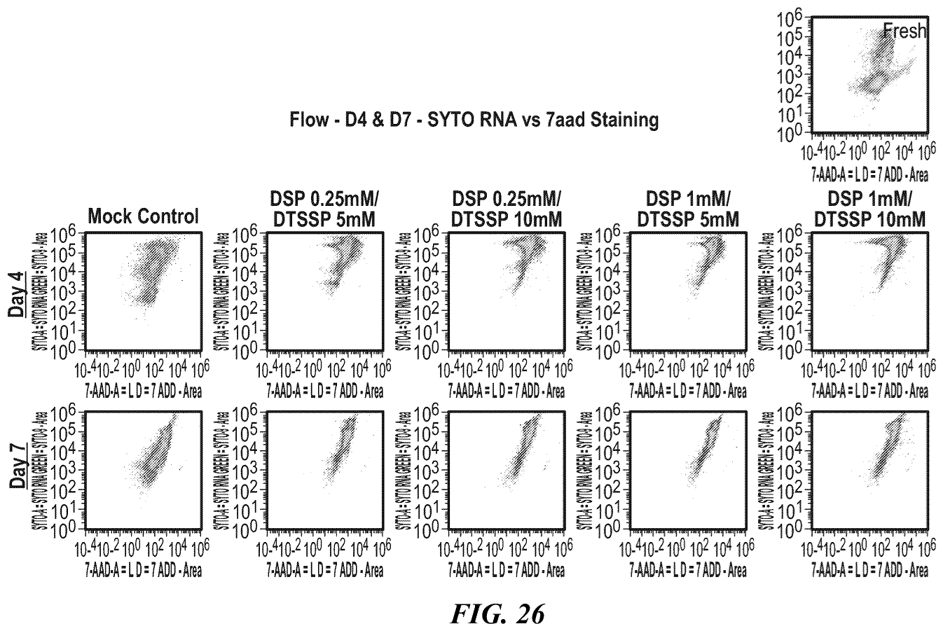

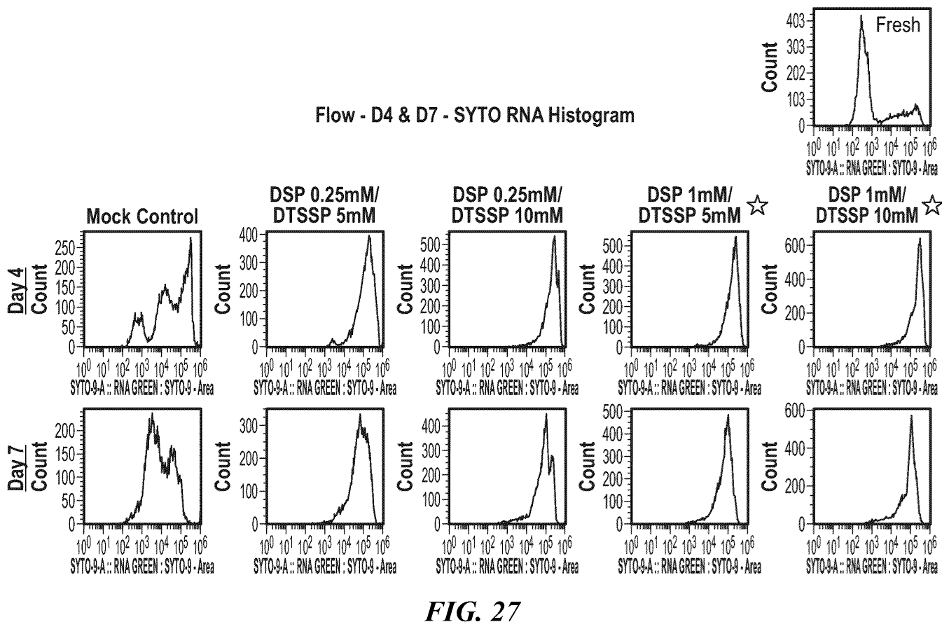

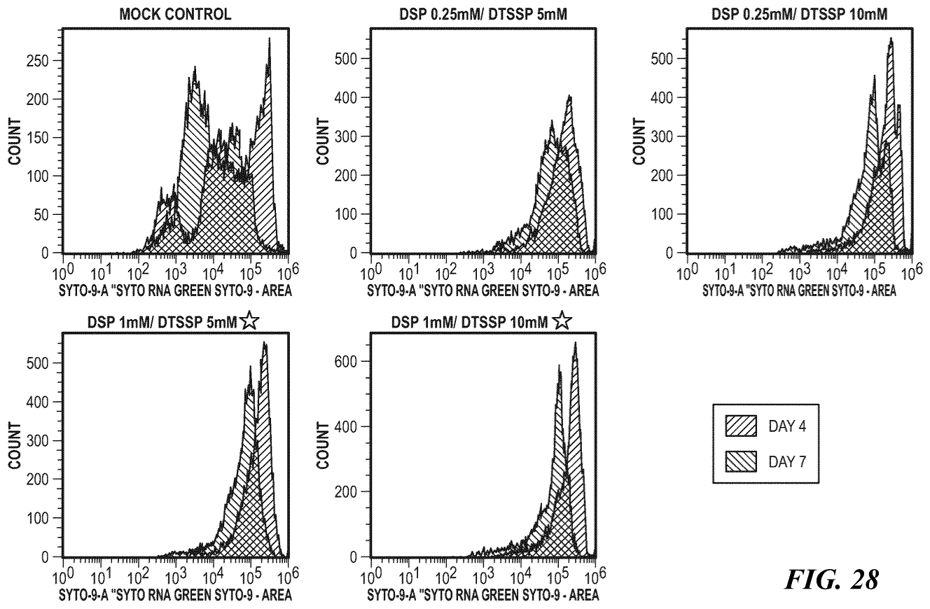

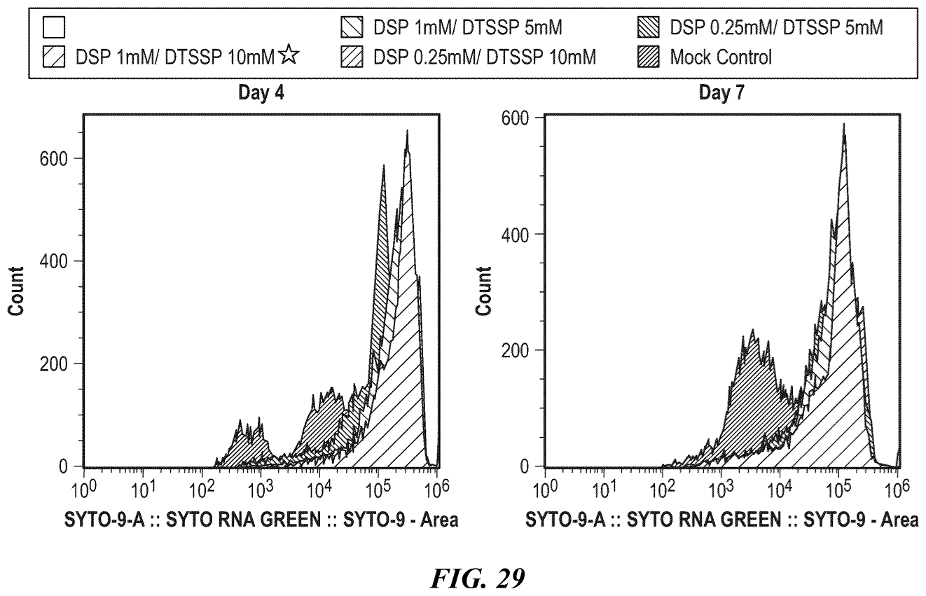

FIGS. 24A-24C show that the most effective RNA pulldown was in the sample treated with the sequential combination of 1 mM DSP and 10 mM DTSSP. FIG. 25 shows flow cytometry data (side scatter—SSC and forward scatter—FSC) at days 4 and 7 from the treated cells, showing the best results with the sequential combination of 1 mM DSP and 5 mM DTSSP. FIG. 26 shows flow cytometry data (staining with SYTO RNA vs. 7aad) at days 4 and 7 from the treated cells, showing optimal results with the sequential combinations of 1 mM DSP and 5 mM DTSSP, and 1 mM DSP and 10 mM DTSSP. Likewise, FIGS. 27-29 (staining with SYTO RNA) at days 4 and 7, show the best results with the sequential combinations of 1 mM DSP and 5 mM DTSSP, and 1 mM DSP and 10 mM DTSSP.

### Example 4

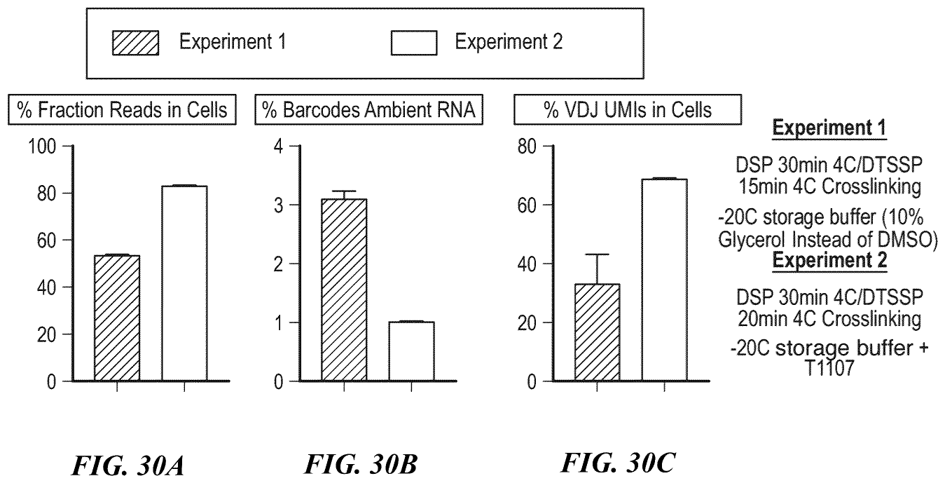

To investigate whether sequential crosslinking combined with storage with a Tetronic® polymer would be advantageous, splenocytes (approximately 250,000-3,000,000 cells) were crosslinked with 2.5 mM DSP for 30 minutes at 4° C., followed by 2.5 mM DTSSP for 15 minutes at 4° C. Cells were stored at −20° C. either in storage buffer with 10% glycerol or storage buffer with the T1107 Tetronic® polymer. Percent fraction reads in cells, percent barcodes ambient RNA, and percent variable/diversity/joining (VDJ) unique molecular identifiers (UMIs) post storage after sequential crosslinking with DSP/DTSSP in combination with Tetronic® polymers are shown in FIGS. 30A-30C. Results show that inclusion of T1107 Tetronic® polymer increases single cell sequencing reads, decreases ambient RNA leakage, and increases VDJ UMI sequencing sensitivity.

### Example 5

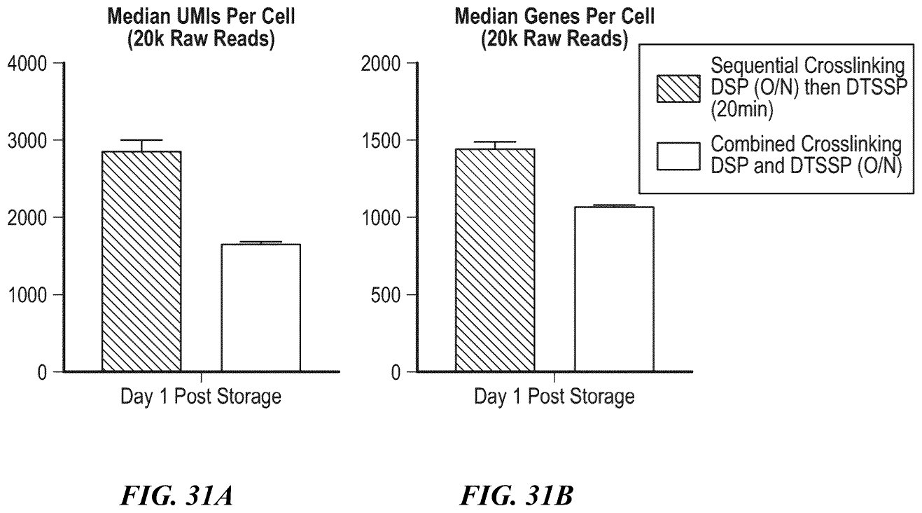

To determine whether sequential or combined DSP/DTSSP provided better results, cells were treated either with 2.5 mM DSP overnight and then with 2.5 mM DTSSP for 20 minutes or with a combination of the two overnight. Median UMIs per cell and median genes per cell are shown in FIGS. 31A-31B. Results confirm that sequential crosslinking provides better results than combination cross-linking.

### Example 6

Cells crosslinked as in any of Examples 1-5 are individually attached to a plurality of barcoded spots on a surface and immobilized by applying a permeable polymer coating over the fixed single biological cells. A reverse crosslinking agent is added to the polymer coating, which permeates the polymer coating to de-crosslink the fixed single biological cells. mRNA originating from the single biological cells is hybridized with a reverse transcription primer associated with the barcoded spots, and the hybridized mRNA from the single biological cells is imaged. Further description of such methods is discussed above, and can also be found in U.S. Pat. No. 11,926,863, which is incorporated herein by reference in its entirety.

### Example 7

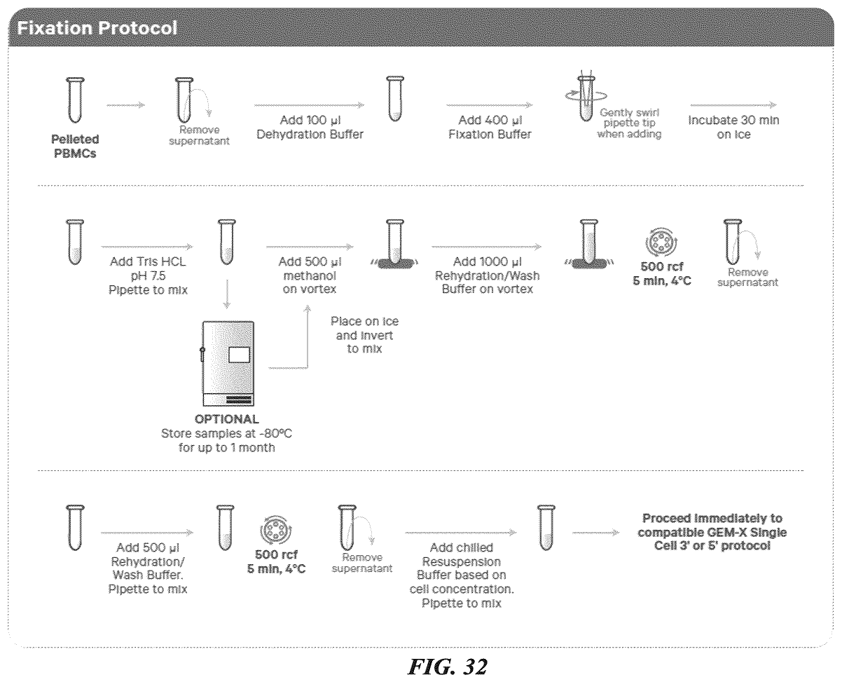

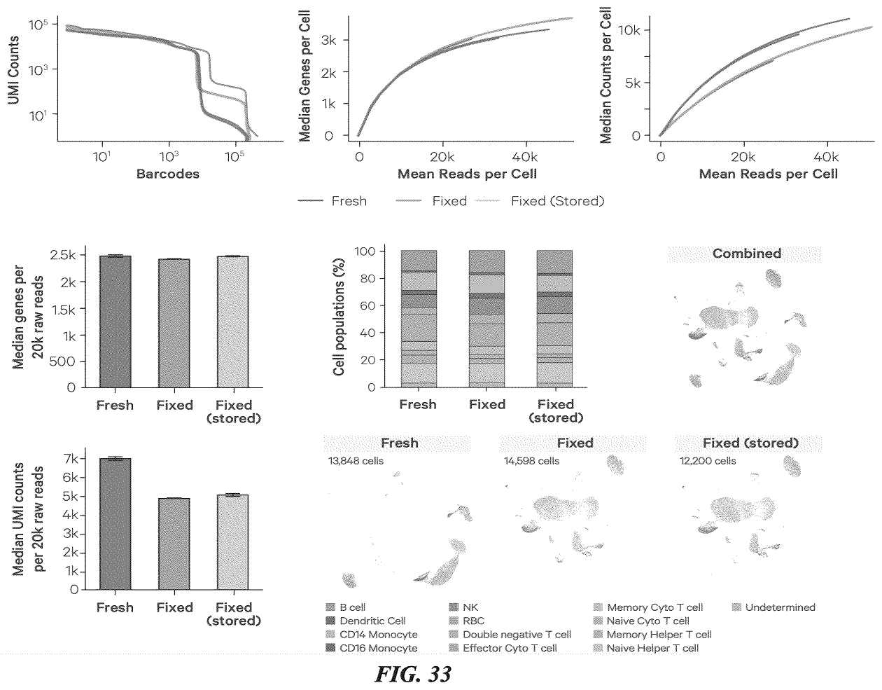

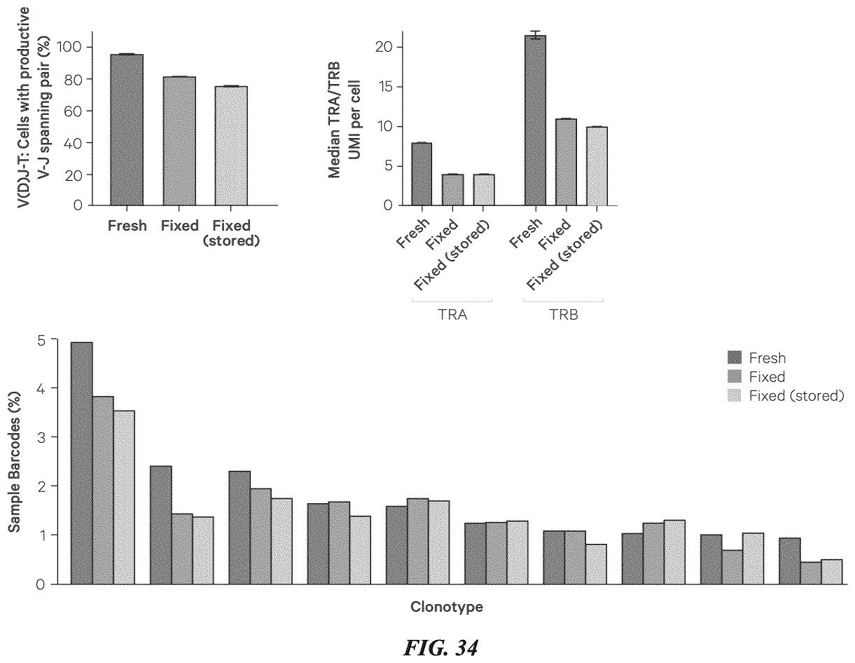

A streamlined fixation protocol was developed for freshly isolated or thawed cells (e.g., PBMCs), which is compatible with single cell analyses (e.g., single cell RNA sequencing, e.g., the reverse transcription-based Chromium™ GEM-X Single Cell 3′ v4 and Chromium™ GEM-X Single Cell 5′ v3 assays) (FIG. 32). Cells (e.g., PBMCs) are fixed in a solution containing methanol and the reversible protein crosslinker DSP. Concurrent dehydration by methanol and crosslinking by DSP enables storage of the samples for at least one month at −80° C. in the quenched fixation buffer without reducing performance metrics. The high alcohol content inhibits nucleases and ice crystal formation, minimizing both enzymatic and physical degradation of RNA and cell morphology. DSP crosslinks are reversed by reducing agents in the GEM-X emulsion droplets, ensuring stability until individual cells have been partitioned. scRNA-seq shows that this method offers sample stability and compatibility with reverse transcription-based assays and V(D)J sequencing (FIG. 34). Fixation yields comparable gene detection, cell type proportions, and clonotype discovery and frequency to unfixed PBMCs (FIG. 33). Averaged across multiple donors, at least 87% of V(D)J cells in fixed samples yield full length V-J spanning contigs compared to 95% of unfixed samples.

A cell fixation protocol developed herein for single cell analysis comprises the following steps:
a. Prepare a single cell suspension (e.g., PBMC single cell suspension) and determine cell concentration. Transfer 100,000-1×10⁶ cells to a 2-ml Eppendorf tube.
b. Centrifuge cell suspension at 300-400 rcf for 5 min at 4° C.
c. Using a pipette tip (or disposable transfer pipette), remove the supernatant without disturbing the pellet. Maintain the cell pellet on ice.
d. Prepare Fixation Buffer (dissolve 104.25 mg DSP in 2.5 ml DMSO, then add 12.5 μl DSP to 387.5 μl pre-chilled methanol (for a final concentration of 3.125 mM DSP)) and maintain on ice.
e. Resuspend the cell pellet in 100 μl Dehydration Buffer (300 mM sucrose, 1×PBS, 3 mM MgCl2, RNase-free water) (50,000-100,000 cells/100 μl) and immediately proceed to Fixation.
f. Using a pipette, slowly dispense 400 μl Fixation Buffer (over 10-15 sec) by placing the pipette tip in the cell suspension and dispensing directly into the cell suspension while gently swirling the pipette tip in the tube to mix.
g. Using a wide-bore pipette tip (pipette set at 400 μl) gently pipette mix until the suspension looks uniform. Once the buffer is fully mixed, the solution will appear clear.
h. Incubate for 30 min. on ice.
i. Add 10 μl 1M Tris-HCl (pH 7.5) per 500 μl reaction volume. Pipette mix.
j. Proceed either directly to the Rehydration (step k) or store cells at −80° C.
k. Set a vortex mixer to an optimal low speed setting: Add 500 μl Methanol (80%) to a 2-ml Eppendorf tube. This will only be used for optimizing vortex speed. Turn the vortex mixer “ON” and set the speed to 1 (lowest setting). Firmly grasp the 2-ml tube (cap open) with Methanol and set on the vortex mixer. Slowly increase the speed of the vortex until the liquid reaches the 0.75-1.0 ml line of the tube (this is typically speed level 2-3 setting or −700 rpm for most vortex mixers). Use this vortex setting for the next step.
l. With one hand, firmly hold the tube with the fixed cells from step j (cap open) on top of the vortex mixer that has been turned on at the setting described above. With the other hand, using a pipette, slowly (over ˜10-15 sec) add dropwise 1 ml Rehydration/Wash Buffer (45 mM sodium citrate pH 6, 450 mM sodium chloride, 1% BSA, 0.2 U/μl, water) directly to the fixed cells while vortexing the tube.
m. Centrifuge at 500 rcf for 5 min. at 4° C.
n. Using a pipette (or disposable transfer pipette), remove the supernatant without disturbing the pellet.
o. Add 500 μl Rehydration/Wash buffer and gently pipette mix to resuspend the pellet.
p. Centrifuge at 500 rcf for 5 min. at 4° C.
q. Using a pipette (or disposable transfer pipette), remove the supernatant without disturbing the pellet.
r. Based on the starting cell concentration and assuming ˜40-50% cell loss, add an appropriate volume (˜100-500 μl) of chilled Resuspension Buffer (1×PBS, 1% BSA, 1 U/μl RNase inhibitor, water). Pipette mix to resuspend the pellet, aiming for a final cell concentration of 700-1,200 cells/μl.
s. Proceed immediately to compatible single cell analysis (i.e., GEM-X Single Cell 3/′ or 5′ protocol).

### Example 8

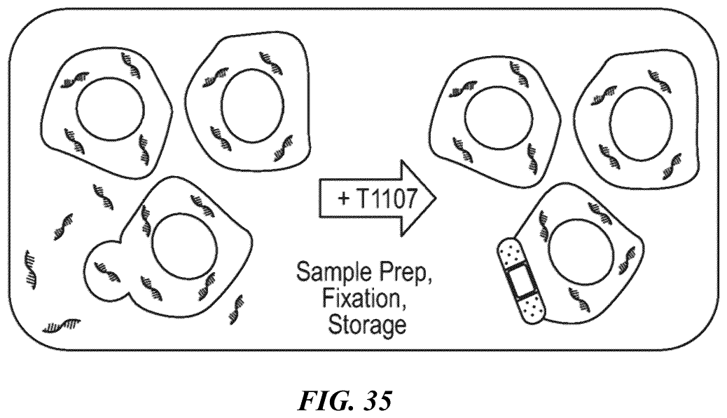

Leakage of RNA is a problem during both crosslinking incubation and storage and undermines single cell (SC) data quality/metrics of preserved samples and contributes to loss of sequencing detail and complexity. Tetronic® polymers can be used as an additive in crosslinking reagents, treatment compositions and storage buffers to mitigate leakage from crosslinked cells. Tetronic® polymers can be used to mitigate leakage by sealing damaged cell membranes in a way that is orthogonal to covalent crosslinking of RNA/protein (PFA, glyoxal, DSP, DSEB, etc.) (FIG. 35).

Inclusion of Tetronic® polymers into single cell workflows improves sequencing performance metrics.

T1107 polymer can significantly mitigate leakage of cellular components from glyoxal or DSP/DTSSP crosslinked Jurkat cells, Raji cells, HuPBMC, and mouse splenocytes. For example, including 0.5 mM T1107 polymer in crosslinking solution significantly improves cell recovery after crosslinking, and inclusion of 0.5 mM T1107 in sample storage buffer has no negative impact on the downstream assay/RT enzymatic activity.

Crosslinking/fixation of cells with T1107 polymer significantly improves single cell metrics, for all cells tested, including, for example, fragile cells such as mouse splenocytes (a fragile and RNAse-rich cell type). T1107 in has been tested in various components of cell fixation/crosslinking and storage work flows, including, for example in: (1) −20 C storage buffer for PBMCs, Jurkat cells, Raji cells, and splenocytes; and (2) in crosslinking buffers for PBMCs; and (3) in crosslinking buffers for splenocytes. Tetronic® polymer T1107 decreases leakage of cellular components as demonstrated, for example, by measuring molecular materials, such as nucleic acids including RNA, present in supernatant (leaked) vs. pelleted cells, and improves single cell sequencing results including improved metrics for sequencing sensitivity.

### Example 9

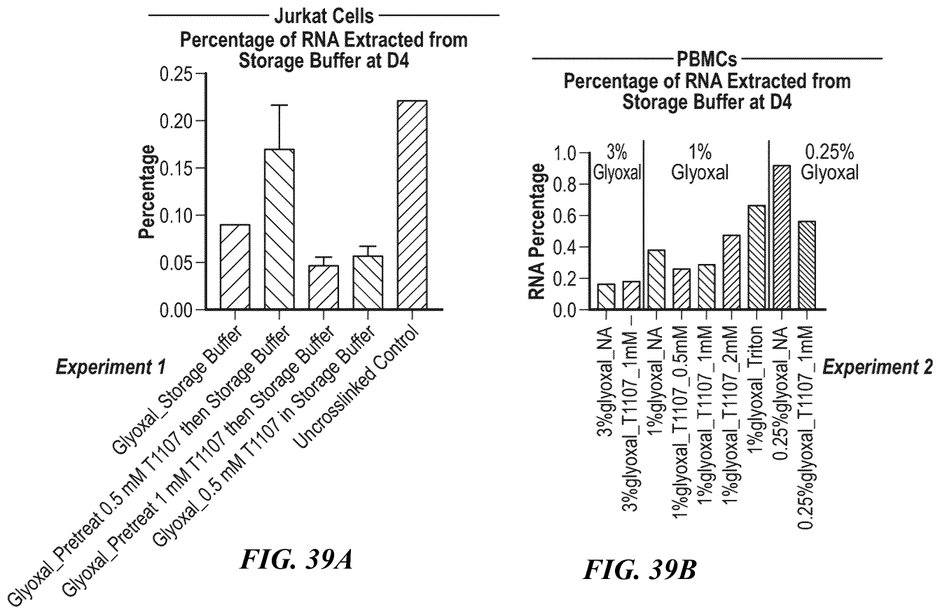

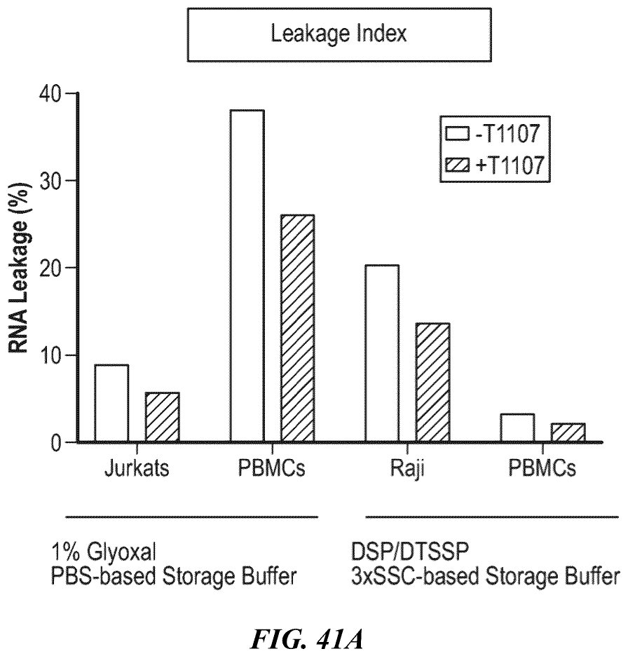

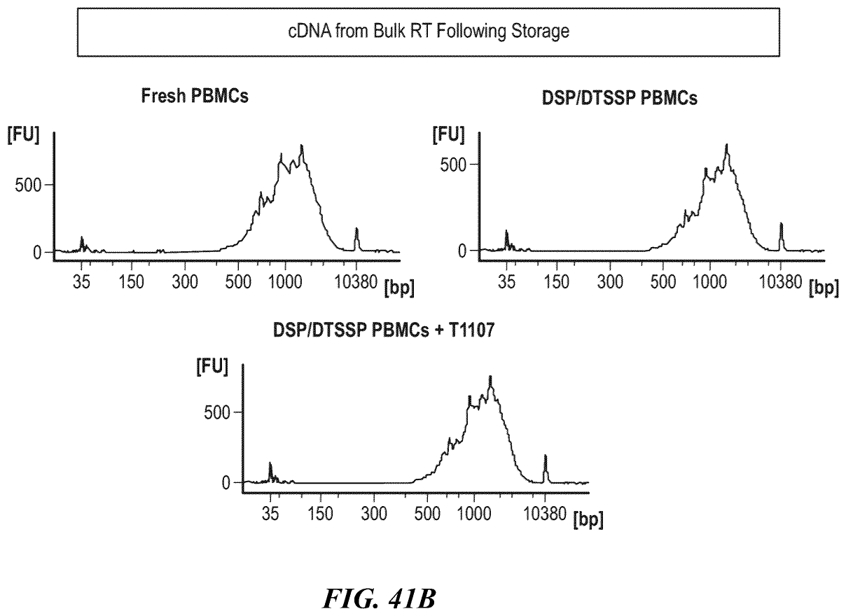

PBMCs (approximately 250,000-3,000,000 cells) were crosslinked with 0.25%, 1% or 3% glyoxal buffer. Cells were stored at −20 C in storage buffer that contained 0.5 mM, 1 mM or 2 mM T1107 in 3×SSC, 3% BSA, 10% DMSO, 300 mM Trehalose (stabilizer), 5 mM ribonucleoside vanadyl complex (RVC) at pH: 6. Percentage of RNA extracted from storage buffer at day 4 is shown in FIG. 39B and RNA leakage data are provided in FIG. 41A, which shows that T1107 polymer in storage buffers mitigates RNA leakage, and in FIG. 41B, which shows that T1107 polymer in storage buffer does not impact reverse transcriptase (RT) activity.

### Example 10

Jurkat cells (approximately 250,000-3,000,000 cells) were crosslinked with 0.25%, 1% or 3% glyoxal buffer. Cells were stored at −20 C in storage buffer that contained 0.5 mM, 1 mM or 2 mM T1107 in 3×SSC, 3% BSA, 10% DMSO, 300 mM Trehalose (stabilizer), 5 mM ribonucleoside vanadyl complex (RVC) at pH: 6. Percentage of RNA extracted from storage buffer at day 4 is shown in FIG. 39A and percent RNA leakage data are provided in FIG. 41A.

### Example 11

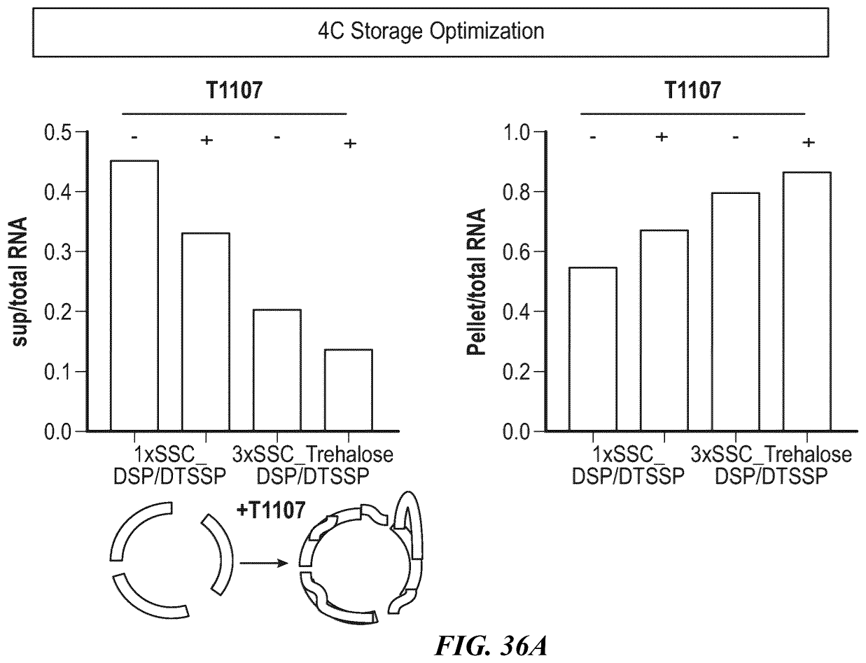

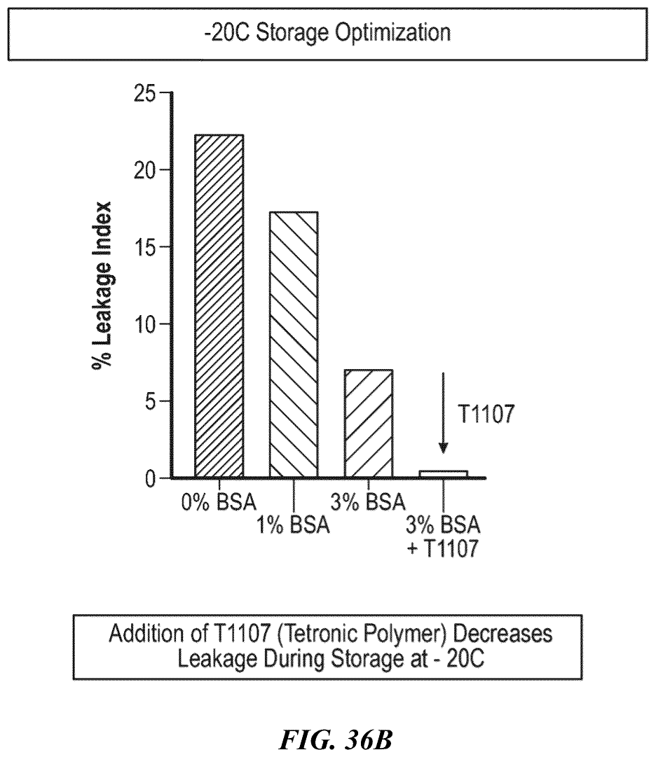

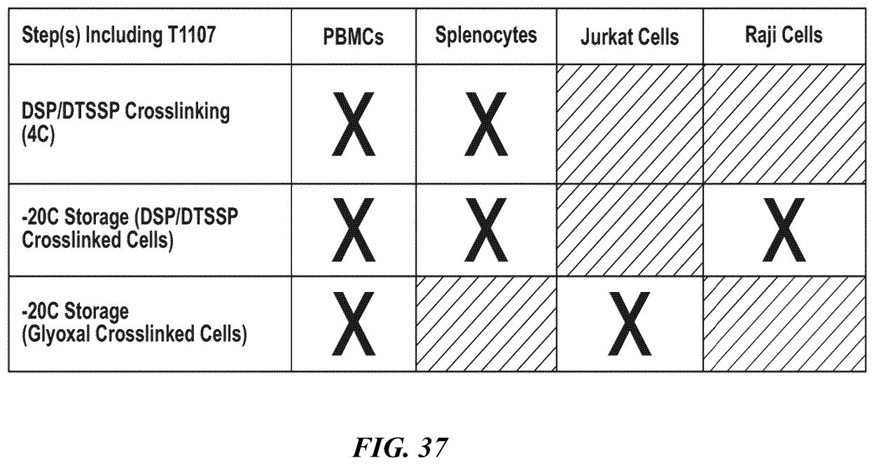

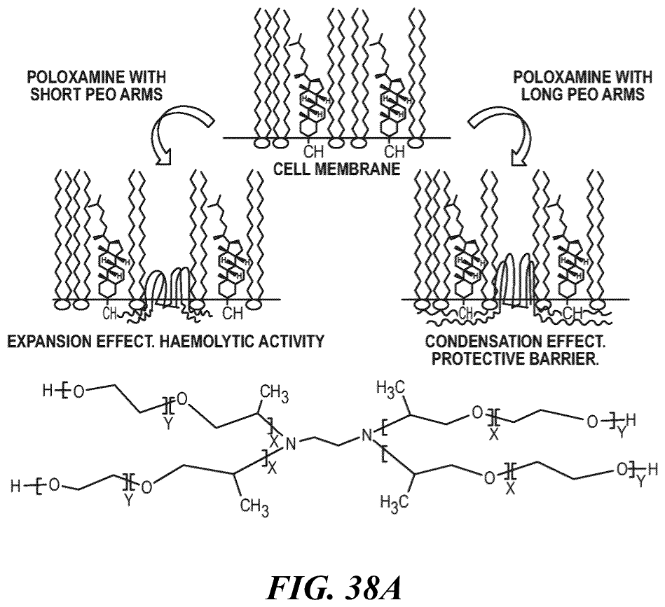

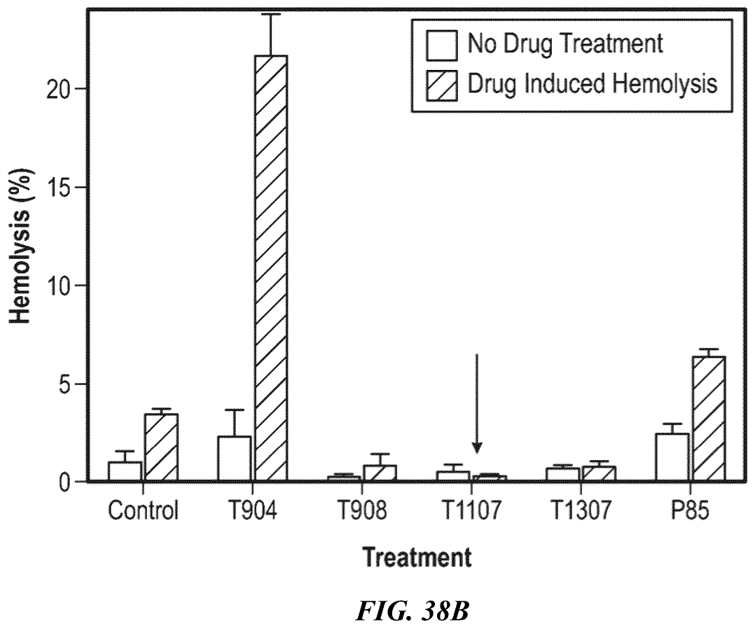

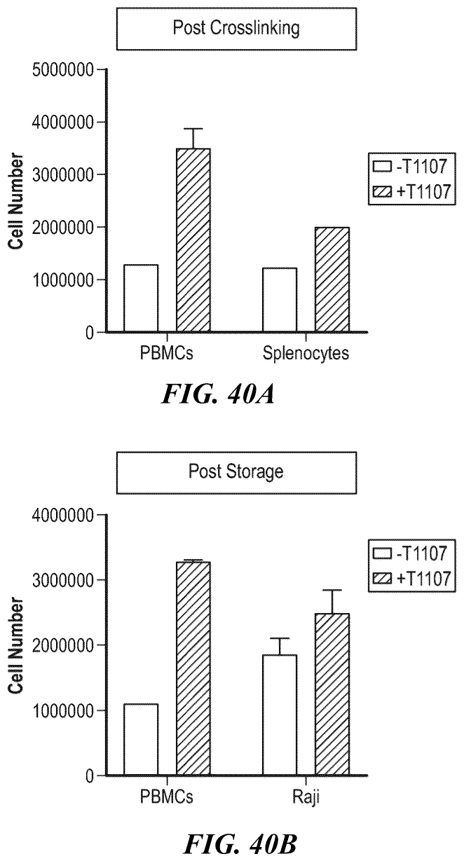

Raji cells (approximately 250,000-3,000,000 cells) were crosslinked with 2.5 mM DSP in 1×SSC or 3×SSC buffer including Trehalose for 30 minutes at 4 C, followed by 2.5 mM DTSSP in 1×SSC or 3×SSC buffer including Trehalose for 15 minutes at 4 C. Cells were stored at 4 C in storage buffer which contained 3% BSA, 10% DMSO, +/−300 mM Trehalose (stabilizer), 5 mM ribonucleoside vanadyl complex (RVC), pH: 6 and +/−0.5 mM T1107. Results for total RNA in the supernatant or pellet are show in FIG. 36A. Results for cell number post-storage are shown in FIG. 40B. Percent RNA leakage is shown in FIG. 41A. Inclusion of T1107 increased the number of cells present after storage and decreased RNA leakage.

### Example 12

PBMCs (approximately 250,000-3,000,000 cells) were crosslinked at 4 C with 2.5 mM DSP in PBS buffer containing 1.0 mM T1107 for 30 minutes at 4 C, followed by 2.5 mM DTSSP in PBS buffer containing 1.0 mM T1107 for 15 minutes at 4 C. Results showing the cell number post crosslinking and post storage with and without T1107 are shown in FIG. 40A-40B.

### Example 13

PBMCs (approximately 250,000-3,000,000 cells) were crosslinked at 4 C with 2.5 mM DSP in PBS buffer for 30 minutes at 4 C, followed by 2.5 mM DTSSP in PBS buffer for 15 minutes at 4 C. Cells were stored at −20 C in storage buffer, which was composed of 3×SSC, either 1% BSA, 3% BSA, or 3% BSA with 0.5 mM T1107, and also including 10% DMSO, 300 mM Trehalose (stabilizer), 5 mM ribonucleoside vanadyl complex (RVC), pH: 6. Results showing percent leakage are shown in FIG. 36B.

### Example 14

PBMCs (approximately 250,000-3,000,000 cells) were crosslinked at 4 C with 2.5 mM DSP in PBS buffer +T1107 for 30 minutes at 4 C, followed by 2.5 mM DTSSP +T1107 in PBS buffer for 15 minutes at 4 C. Cells were stored at −20 C in storage buffer, which was composed of 3×SSC, either 1% BSA, 3% BSA, or 3% BSA with 0.5 mM T1107, and also including 10% DMSO, 300 mM Trehalose (stabilizer), 5 mM ribonucleoside vanadyl complex (RVC), pH: 6.

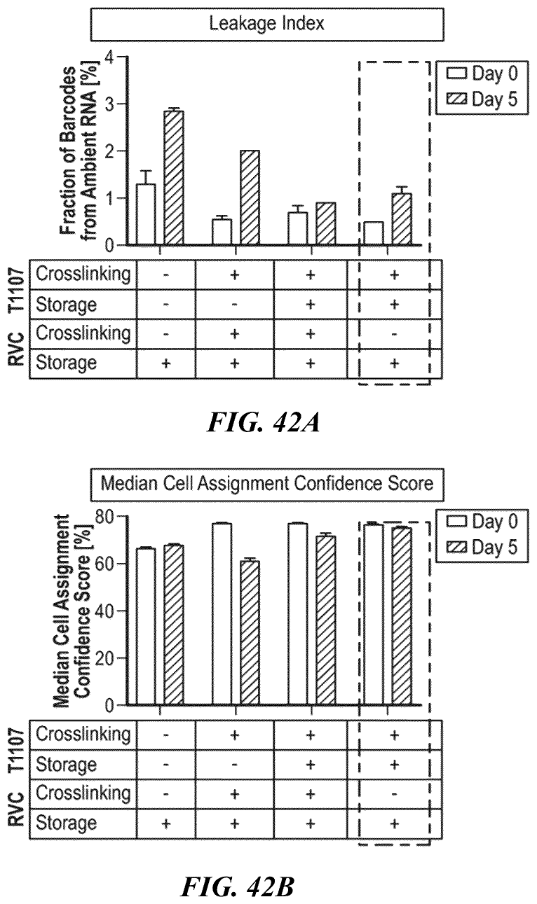

Results are shown in FIG. 42A-42B.

### Example 15

Splenocytes (approximately 250,000-3,000,000 cells) were crosslinked at 4 C with 2.5 mM DSP in PBS buffer +T1107 for 30 minutes at 4 C, followed by 2.5 mM DTSSP +T1107 in PBS buffer for 15 minutes at 4 C.

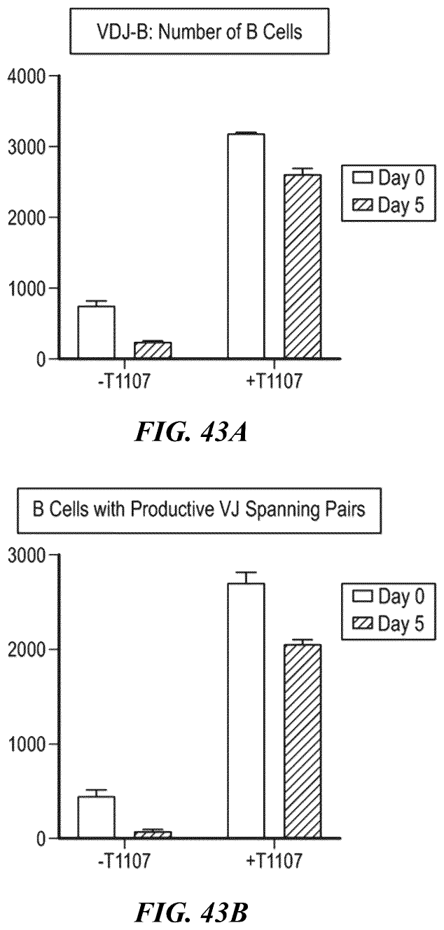

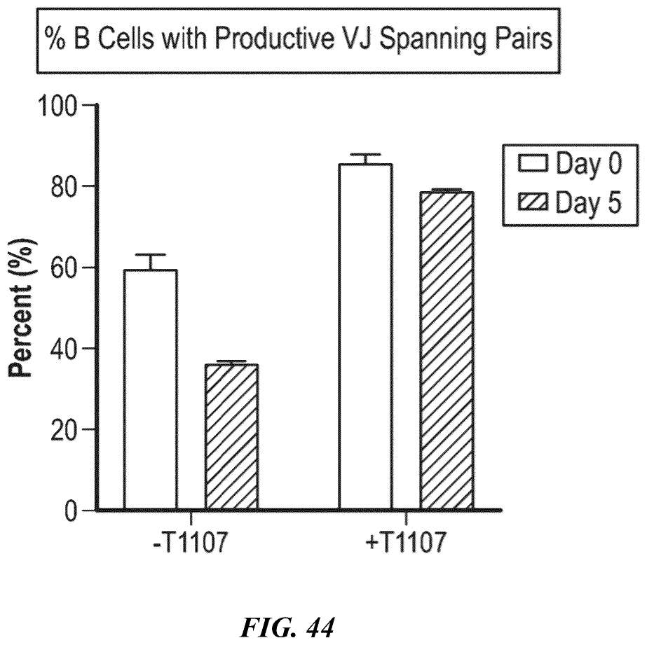

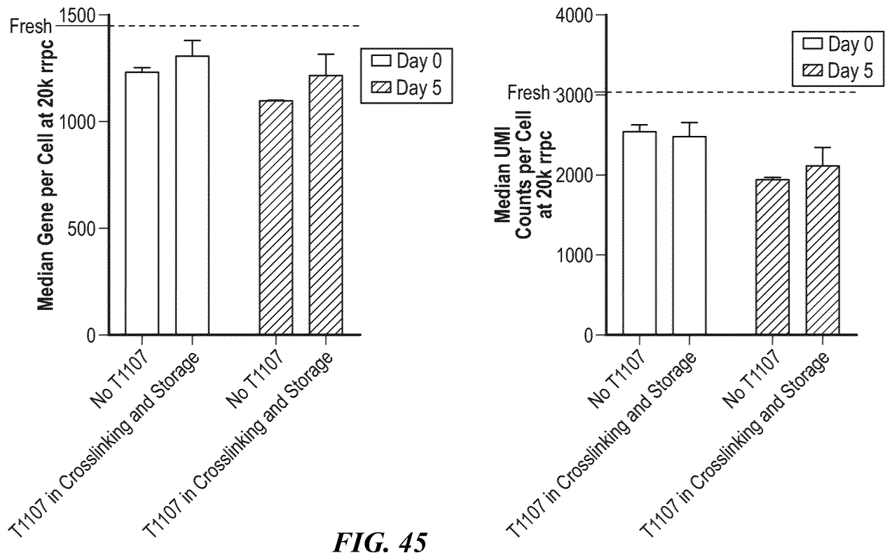

Cells were stored at −20 C in storage buffer which was composed of 3×SSC, either 1% BSA, 3% BSA, or 3% BSA with 0.5 mM T1107, and also including 10% DMSO, 300 mM Trehalose (stabilizer), 5 mM ribonucleoside vanadyl complex (RVC), pH: 6. Results for cell numbers are shown in FIG. 40A, for leakage index in FIG. 42A, for median cell assignment confidence score in FIG. 42B, for number of B cells VDJ-B in FIGS. 43A and 43B cells with productive VJ spanning pairs in FIG. 43B, for percent B cells with productive VJ spanning pairs in FIG. 44, and for median gene and UMI per cell FIG. 45.

While preferred aspects of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

Embodiments of the invention, which are not meant to be limiting, are described in the numbered paragraphs below. The embodiments of the invention described herein are exemplary, and various modifications and improvements can be made without departing from the spirit and scope of the invention. The scope of the invention is defined by the appended claims, and all changes that fall within the meaning and range of equivalents are intended to be embraced therein.

1. A method for sample preparation, the method comprising the following steps in order:
i) contacting a sample comprising at least one cell or tissue with a hydrophobic crosslinker;
ii) removing the hydrophobic crosslinker from the sample;
iii) contacting the sample with a hydrophilic crosslinker; and
iv) removing the hydrophilic crosslinker from the sample.

2. The method of claim 1, wherein the hydrophobic crosslinker is ethylene glycol bis(succinimidyl succinate) (EGS), disuccinimidyl suberate (DSS), or dithiobis(succinimidyl propionate) (DSP).

3. The method of claim 2, wherein the hydrophobic crosslinker is dithiobis(succinimidyl propionate) (DSP).

4. The method of claim 1, wherein the hydrophilic crosslinker is bis(sulfosuccinimidyl)suberate (BS3), ethylene glycol bis(sulfosuccinimidyl succinate) (sulfo-EGS), 3,3′-dithiobis(sulfosuccinimidyl propionate) (DTSSP), bis-succinimide ester-activated PEG (BS(PEG)9) or dimethyl 3,3′-dithiobispropionimidate dihydrochloride (DTBP).

5. The method of claim 4, wherein the hydrophilic crosslinker is 3,3′-dithiobis(sulfosuccinimidyl propionate) (DTSSP).

6. The method of claim 1, wherein the sample preparation reduces leakage of biological components from cells, wherein the leakage is reduced about 2-10-fold relative to a control cell or tissue that has not been contacted with the hydrophobic and hydrophilic crosslinkers.

7. The method of claim 1, wherein the hydrophobic crosslinker is in a first buffer and the hydrophilic crosslinker is in a second buffer.

8. The method of claim 1, wherein the hydrophobic crosslinker comprises about 0.05 mM to 10 mM DSP.

9. The method of claim 1, wherein the hydrophilic crosslinker comprises about 0.25 mM to about 10 mM DTSSP.

10. The method of claim 1, wherein contacting the sample with the hydrophobic crosslinker comprises incubating at about 2 to about 25 degrees Celsius for a time period ranging from about 10 minutes to about 24 hours.

11. The method of claim 1, wherein contacting the sample with the hydrophilic crosslinker comprises incubating at about 2 to about 25 degrees Celsius for a time period ranging from about 10 minutes to about 24 hours.

12. The method of claim 1, further comprising determining the sequence of one or more nucleic acid sequences from one or more cells from the sample.

13. The method of claim 1, further comprising contacting the sample with a poloxamine.

14. A method for reducing leakage of biological components from cells during preservation and storage, the method comprising:
i) contacting a plurality of cells with a fixative buffer comprising a poloxamine;
ii) removing the fixative buffer from the cells; and
iii) suspending the cells in a storage buffer.

15. A method for reducing leakage of biological components from cells during preservation and storage, the method comprising:
i) contacting a plurality of cells with a fixative buffer;
ii) removing the fixative buffer from the cells;
iii) contacting the cells with a treatment buffer comprising a poloxamine;
iv) removing the treatment buffer from the cells; and
v) suspending the cells in a storage buffer.

16. A method for reducing leakage of biological components from cells during preservation and storage, the method comprising:
i) contacting a plurality of cells with a fixative buffer;
ii) removing the fixative buffer from the cells; and
iii) suspending the cells in a storage buffer comprising a poloxamine.

17. A method for reducing leakage of biological components from cells during preservation and storage, the method comprising:
i) contacting a plurality of cells with a fixative buffer comprising a poloxamine;
ii) removing the fixative buffer from the cells; and
iii) suspending the cells in a storage buffer comprising a poloxamine.

18. A method for reducing leakage of biological components from cells during preservation and storage, the method comprising:
i) contacting a plurality of cells with a fixative buffer comprising a poloxamine;
ii) removing the fixative buffer from the cells;
iii) contacting the cells with a treatment buffer comprising a poloxamine;
iv) removing the treatment buffer from the cells; and
v) suspending the cells in a storage buffer.

19. The method of claim 13, wherein the poloxamine comprises a poloxamine 1107.

20. The method of claim 14, wherein the fixative buffer comprises from about 0.25 mM to about 1.25 mM of a poloxamine 1107.

21. The method of claim 14, wherein the fixative buffer comprises DSP or DTSSP.
